(19) United States
(12) Patent Application Publication
(10) Pub. No.: US 2026/0109777 A1
(43) Pub. Date: Apr. 23, 2026

(54) METHODS OF TREATING CANCER USING SUBCUTANEOUS DOSING OF MOSUNETUZUMAB AS A MONOTHERAPY OR IN COMBINATION WITH LENALIDOMIDE

(71) Applicants: Genentech, Inc., South San Francisco, CA (US); Hoffmann-La Roche Inc., Little Falls, NJ (US)

(72) Inventors: Samuel Dean TRACY, San Francisco, CA (US); Hong WANG, San Carlos, CA (US); Jue WANG, San Jose, CA (US); Brendan Christian BENDER, New York, NY (US); Yi Meng CHANG, Oakville (CA); Iraj HOSSEINI, San Carlos, CA (US); Chi-Chung LI, Burlingame, CA (US); Carol Elaine O'HEAR, Burlingame, CA (US); Juliana Marques Lagrasta BIONDO, South San Francisco, CA (US); Nai Shun YAO, South San Francisco, CA (US); Ian W. FLINN, Brentwood, TN (US); John M. BURKE, Aurora, CO (US); Michael Ching-sun WEI, South San Francisco, CA (US); Mei Shan WU, South San Francisco, CA (US); Antonia Pui-Yue KWAN, South San Francisco, CA (US); Yong Jun MUN, South San Francisco, CA (US)

(21) Appl. No.: 19/249,597

(22) Filed: Jun. 25, 2025

Related U.S. Application Data
(60) Provisional application No. 63/663,941, filed on Jun. 25, 2024.
(60) Provisional application No. 63/792,634, filed on Apr. 22, 2025.
(60) Provisional application No. 63/813,743, filed on May 29, 2025.
(60) Provisional application No. 63/822,412, filed on Jun. 12, 2025.

(51) Int. Cl. C07K 16/28; A61K 9/00; A61K 31/138; A61K 31/167; A61K 31/454; A61K 31/573; A61K 39/00; A61P 35/00

(52) U.S. Cl. CPC C07K 16/2887; A61K 9/0019; A61K 9/0053; A61K 31/138; A61K 31/167; A61K 31/454; A61K 31/573; A61P 35/00; C07K 16/2809; C07K 16/2866; A61K 2039/505; A61K 2039/54; A61K 2039/545; C07K 2317/31

(57) ABSTRACT

The present invention relates to the treatment of subjects having CD20-positive cell proliferative disorders (e.g., B cell proliferative disorders, such as non-Hodgkin's lymphomas or chronic lymphocytic leukemia). More specifically, the invention pertains to the treatment of subjects having a B cell proliferative disorder by subcutaneous administration of mosunetuzumab as a monotherapy or in combination with lenalidomide.

### SEQUENCE LISTING

The instant application contains a Sequence Listing which has been submitted electronically in XML format and is hereby incorporated by reference in its entirety. Said XML copy, created on Jun. 20, 2025, is named 50474-355005_Sequence_Listing_6_20_25.xml and is 34,026 bytes in size.

### FIELD OF THE INVENTION

The present invention relates to the treatment of CD20-positive cell proliferative disorders. More specifically, the invention pertains to treatment of subjects having a CD20-positive cell proliferative disorders by subcutaneous administration of mosunetuzumab, either alone or in combination with lenalidomide.

### BACKGROUND

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Cancer care thus represents a significant and ever-increasing societal burden.

CD20-positive cell proliferative disorders, such as B cell proliferative disorders, are a leading cause of cancer-related deaths. For example, non-Hodgkin's lymphoma (NHL) advances quickly and is fatal if untreated. In the United States, B-cell lymphomas constitute approximately 80%-85% of all cases of NHL. Diffuse large B-cell lymphoma (DLBCL) is the most common type of NHL accounting for approximately 30%-40% of all NHL diagnosis, followed by follicular lymphoma (FL; 20%-25% of all NHL diagnosis) and mantle cell lymphoma (MCL; 6%-10% of all NHL diagnosis). B-cell chronic lymphocytic leukemia (CLL) is the most common leukemia in adults, with approximately 15,000 new cases per year in the United States (American Cancer Society 2015).

Bispecific antibodies are capable of simultaneously binding cell surface antigens on cytotoxic cells (e.g., T cells, via binding to cluster of differentiation 3 (CD3)) and cancer cells (e.g., B cells, via binding to CD20), with the intent that the bound cytotoxic cell will destroy the bound cancer cell. However, such antibody-based immunotherapies may be limited by unwanted effects, including cytokine-driven toxicities (e.g., cytokine release syndrome (CRS)), infusion-related reactions (IRRs), severe tumor lysis syndrome (TLS), and central nervous system (CNS) toxicities.

Thus, there is an unmet need in the field for the development of efficacious methods of dosing therapeutic bispecific antibodies (e.g., mosunetuzumab) for the treatment of a CD20-positive cell proliferative disorder, e.g., a B cell proliferative disorder (e.g., non-Hodgkin's lymphoma (NHL) (e.g., a previously untreated (1 L) NHL, a diffuse-large B cell lymphoma (DLBCL) (e.g., 1 L DLBCL, a relapsed and/or refractory DLBCL, or a Richter's transformation), a follicular lymphoma (FL) (e.g., a 1 L FL, a relapsed and/or refractory FL, or a transformed FL), a mantle cell lymphoma (MCL), a high-grade B cell lymphoma, or a primary mediastinal (thymic) large B cell lymphoma (PMLBCL)) or a chronic lymphoid leukemia (CLL)) that achieve a more favorable benefit-risk profile.

### SUMMARY OF THE INVENTION

The present invention relates to methods of treating a subject having a CD20-positive cell proliferative disorder (e.g., a B cell proliferative disorder) by subcutaneous administration of mosunetuzumab as a monotherapy or in combination with lenalidomide, wherein the treatment comprising subcutaneous administration of mosunetuzumab as a monotherapy or in combination with lenalidomide exhibits non-inferiority or an improved outcome as measured using pharmacokinetics (PK), efficacy, safety, or a combination thereof, as compared to intravenous administration of mosunetuzumab as a monotherapy or in combination with lenalidomide, respectively.

In one aspect, the invention features a method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In some embodiments, the efficacy response is a CRR, and wherein the CRR or reference CRR is the proportion of the subjects receiving the corresponding treatment whose best overall response is a complete response (CR).

In some embodiments, the improved response in CRR is an increase in CRR compared to the reference CRR of between 1% and 25% (e.g., between 5% and 25%, between 10% and 25%, between 15% and 25%, between 20% and 25%, between 5% and 10%, between 5% and 15%, between 5% and 20%, between 10% and 20%, between 10% and 15%, or between 15% and 20%; e.g., about 1%, 5%, 10%, 15%, 20%, or 25%). In some embodiments, the efficacy response in CRR is non-inferior compared to the reference CRR.

In some embodiments, the efficacy response is an ORR, and wherein the ORR or reference ORR is the proportion of the subjects receiving the corresponding treatment whose best overall response is a CR or a partial response (PR). In some embodiments, the improved response in ORR is an increase in ORR compared to the reference ORR of between 1% and 24% (e.g., between 5% and 24%, between 10% and 24%, between 15% and 24%, between 20% and 24%, between 5% and 10%, between 5% and 15%, between 5% and 20%, between 10% and 20%, between 10% and 15%, or between 15% and 20%; e.g., about 1%, 5%, 10%, 15%, 20%, or 24%). In some embodiments, the efficacy response in ORR is non-inferior compared to the reference ORR.

In some embodiments, the efficacy response is a DOR, and wherein the DOR or the reference DOR is measured starting from the time from the first occurrence of a documented CR or PR to disease progression or relapse or death from any cause, whichever occurs first. In some embodiments, the DOR or the reference DOR is the median DOR of the plurality of subjects receiving the corresponding treatment. In some embodiments, the improvement of the median DOR is an increase in the DOR compared to the reference DOR of between 1 and 22 months (e.g., between 1 month and 5 months, between 5 months and 22 months, between 10 months and 22 months, between 15 months and 22 months, between 5 months and 10 months, between 5 months and 15 months, between 10 months and 22 months, between 10 months and 15 months, or between 15 months and 22 months; e.g., about 1 months, 5 months, 10 months, 15 months, 20 months, or 22 months). In some embodiments, the efficacy response in DOR is non-inferior compared to the reference DOR.

In some embodiments, the efficacy response is a DOCR, and wherein the DOCR or the reference DOCR is measured starting from the time from the first occurrence of a documented CR to disease progression or relapse or death from any cause, whichever occurs first. In some embodiments, the DOCR or the reference DOCR is the median DOCR of the plurality of subjects receiving the corresponding treatment. In some embodiments, the improvement of the median DOCR is an increase in the DOCR compared to the reference DOCR of between 1 and 22 months (e.g., between 1 month and 5 months, between 5 months and 22 months, between 10 months and 22 months, between 15 months and 22 months, between 5 months and 10 months, between 5 months and 15 months, between 10 months and 22 months, between 10 months and 15 months, or between 15 months and 22 months; e.g., about 1 months, 5 months, 10 months, 15 months, 20 months, or 22 months). In some embodiments, the efficacy response in DOCR is non-inferior compared to the reference DOCR.

In some embodiments, the CR or PR is determined by PET/computed tomography (CT). In some embodiments, the CR or PR is determined based on the Lugano Response Criteria for Malignant Lymphoma (Cheson et al., 2014).

In some embodiments, the efficacy response is an OS, wherein the OS or the reference OS is measured starting from the time from first treatment (e.g., first dose of mosunetuzumab or first dose of lenalidomide; e.g., Day 1 of dosing cycle 1) to death from any cause. In some embodiments, the OS or the reference OS is the median OS of the plurality of subjects receiving the corresponding treatment. In some embodiments, the improvement of the median OS is an increase in the OS compared to the reference OS of between 1 and 28 months (e.g., between 1 month and 5 months, between 5 months and 28 months, between 10 months and 28 months, between 15 months and 28 months, between 20 and 28 months, between 25 and 28 months, between 5 months and 10 months, between 5 months and 15 months, between 5 months and 20 months, between 5 months and 25 months, between 10 months and 20 months, between 10 months and 25 months, between 10 months and 15 months, between 15 months and 25 months, or between 20 months and 28 months; e.g., about 1 months, 5 months, 10 months, 15 months, 20 months, 25 months, or 28 months). In some embodiments, the efficacy response in OS is non-inferior compared to the reference OS.

In some embodiments, the efficacy response is a PFS, and wherein the PFS or the reference PFS is measured starting from the time from first treatment (e.g., first dose of mosunetuzumab or first dose of lenalidomide; e.g., Day 1 of dosing cycle 1) to the time of a first occurrence of disease progression or death from any cause. In some embodiments, the PFS or the reference PFS is the median PFS of the plurality of subjects receiving the corresponding treatment. In some embodiments, the improvement of the median PFS is an increase in the PFS compared to the reference PFS of between 1 and 26 months (e.g., between 1 month and 5 months, between 5 months and 26 months, between 10 months and 26 months, between 15 months and 26 months, between 5 months and 10 months, between 5 months and 15 months, between 10 months and 26 months, between 10 months and 15 months, between 15 months and 20 months, or between 20 months and 26 months; e.g., about 1 months, 5 months, 10 months, 15 months, 20 months, or 26 months). In some embodiments, the efficacy response in PFS is non-inferior compared to the reference PFS.

In another aspect, the invention features a method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In some embodiments, the safety response is the rate of AE, and the rate of AE or the reference rate of AE is the rate of AE in the plurality of subjects receiving the corresponding treatment. In some embodiments, the safety response in rate of AE is non-inferior compared to the reference rate of AE.

In some embodiments, the safety response is the rate of CRS, and wherein the rate of CRS or the reference rate of CRS is the rate of CRS in the plurality of subjects receiving the corresponding treatment. In some embodiments, the improved rate of CRS is a decrease in the rate of CRS compared to the reference rate of CRS of between 7% and 17% (e.g., between 7% and 12%, between 12% and 17%, or between 10% and 14%; e.g., about 8%, 9%, 10%, 11%, 12%, 13%, 14%, 15%, 16%, or 17%). In some embodiments, the improved rate of CRS is a decrease in the rate of CRS compared to the reference rate of CRS of about 12%. In some embodiments, the rate of CRS is the rate of Grade 1 or 2 CRS in the plurality of patients receiving the corresponding treatment. In some embodiments, the rate of Grade 3+ CRS is non-inferior compared to the reference rate of Grade 3+ CRS. In some embodiments, the Grade of CRS is determined based on the American Society of Transplantation and Cellular Therapy (ASTCT) Consensus Grading for Cytokine-Release Syndrome (Lee et al., 2019).

In some embodiments, the safety response is the rate of SAE, and wherein the rate of SAE or the reference rate of SAE is the rate of SAE in the plurality of subjects receiving the corresponding treatment. In some embodiments, the safety response in rate of SAE is non-inferior compared to the reference rate of SAE.

In another aspect, the invention features a method for treating follicular lymphoma (FL) in a subject in need thereof, comprising administering to the subject a combination treatment comprising subcutaneously administered mosunetuzumab and orally administered lenalidomide according to a dosing regimen comprising a first 21-day dosing cycle and three 28-day dosing cycles, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In some embodiments, one or both of the scAUC₀₋₇₇ and the ivAUC₀₋₇₇ is determined using population PK (popPK) model-predicted individual Empirical Bayesian Estimates (EBEs).

In some embodiments, the combination treatment comprises subcutaneously administering mosunetuzumab and orally administering lenalidomide according to a dosing regimen comprising at least a first dosing cycle and a second dosing cycle, wherein the first dosing cycle is a 21-day dosing cycle and the second dosing cycle is a 28-day dosing cycle, and wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg±0.5 mg), the scC1D2 is about 45 mg (e.g., 45 mg±10%; 45 mg±4.5 mg), and the scC1D3 is about 45 mg (e.g., 45 mg±10%; 45 mg±4.5 mg); and (b) the second dosing cycle comprises: (i) a first subcutaneous dose (scC2D1) of mosunetuzumab administered on Day 1 of the second dosing cycle, wherein the scC2D1 is about 45 mg (e.g., 45 mg±10%; 45 mg±4.5 mg); and (ii) twenty-one (21) oral doses of lenalidomide administered on Days 1-21 of the second dosing cycle, wherein each oral dose of lenalidomide is about 20 mg (e.g., 20 mg±10%; 20 mg±2 mg). In particular embodiments, the combination treatment comprises subcutaneously administering mosunetuzumab and orally administering lenalidomide according to a dosing regimen comprising at least a first dosing cycle and a second dosing cycle, wherein the first dosing cycle is a 21-day dosing cycle and the second dosing cycle is a 28-day dosing cycle, and wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is 5 mg, the scC1D2 is 45 mg, and the scC1D3 is 45 mg; and (b) the second dosing cycle comprises: (i) a first subcutaneous dose (scC2D1) of mosunetuzumab administered on Day 1 of the second dosing cycle, wherein the scC2D1 is 45 mg; and (ii) twenty-one (21) oral doses of lenalidomide administered on Days 1-21 of the second dosing cycle, wherein each oral dose of lenalidomide is 20 mg.

In some embodiments, the dosing regimen of the combination treatment further comprises one or more additional 28-day dosing cycles. In some embodiments, the dosing regimen of the combination treatment comprises ten additional 28-day dosing cycles.

In some embodiments, each additional dosing cycle comprises: (a) a subcutaneous dose of mosunetuzumab administered on Day 1 of the additional dosing cycle, wherein the subcutaneous dose of mosunetuzumab is about 45 mg (e.g., 45 mg±10%; 45 mg±4.5 mg); and (b) twenty-one (21) oral doses of lenalidomide administered on Days 1-21 of the additional dosing cycle, wherein each oral dose of lenalidomide is about 20 mg (e.g., 20 mg±10%; 20 mg±2 mg). In particular embodiments, each additional dosing cycle comprises: (a) a subcutaneous dose of mosunetuzumab administered on Day 1 of the additional dosing cycle, wherein the subcutaneous dose of mosunetuzumab is 45 mg; and (b) twenty-one (21) oral doses of lenalidomide administered on Days 1-21 of the additional dosing cycle, wherein each oral dose of lenalidomide is 20 mg.

In some embodiments, the dosing regimen of the combination treatment further comprises one to nine (e.g., one, two, three, four, five, six, seven, eight, or nine) subcutaneous maintenance doses of mosunetuzumab.

In another aspect, the invention features a method for treating follicular lymphoma (FL) in a subject in need thereof, comprising administering to the subject a combination treatment comprising subcutaneously administered mosunetuzumab and orally administered lenalidomide, wherein the combination treatment comprises subcutaneously administering mosunetuzumab and orally administering lenalidomide according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine (e.g., one, two, three, four, five, six, seven, eight, or nine) subcutaneous maintenance doses of mosunetuzumab, wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg±0.5 mg), the scC1D2 is about 45 mg (e.g., 45 mg±10%; 45 mg±4.5 mg), and the scC1D3 is about 45 mg (e.g., 45 mg±10%; 45 mg±4.5 mg); (b) the second dosing cycle comprises: (i) a first subcutaneous dose (scC2D1) of mosunetuzumab administered on Day 1 of the second dosing cycle, wherein the scC2D1 is about 45 mg (e.g., 45 mg±10%; 45 mg±4.5 mg); and (ii) twenty-one (21) oral doses of lenalidomide administered on Days 1-21 of the second dosing cycle, wherein each oral dose of lenalidomide is about 20 mg (e.g., 20 mg±10%; 20 mg±2 mg); and (c) the ten additional dosing cycles each comprises: (i) a first subcutaneous dose (scC3D1-scC12D1) of mosunetuzumab administered on Day 1 of each additional dosing cycle, wherein the scC3D1-scC12D1 are each about 45 mg (e.g., 45 mg±10%; 45 mg±4.5 mg); and (ii) twenty-one (21) oral doses of lenalidomide administered on Days 1-21 of each additional dosing cycle, wherein each oral dose of lenalidomide is about 20 mg (e.g., 20 mg±10%; 20 mg±2 mg).

In some embodiments, the dosing regimen of the combination treatment further comprises nine subcutaneous maintenance doses of mosunetuzumab.

In some embodiments, the one to nine (e.g., one, two, three, four, five, six, seven, eight, or nine) subcutaneous maintenance doses of mosunetuzumab are administered every eight weeks starting eight weeks after Day 1 of the tenth additional 28-day dosing cycle. In some embodiments, each maintenance dose is about 45 mg (e.g., 45 mg±10%; 45 mg±4.5 mg). In particular embodiments, each maintenance dose is 45 mg.

In some embodiments, the control treatment comprises intravenously administering mosunetuzumab and orally administering lenalidomide according to a dosing regimen comprising at least a first 21-day dosing cycle and a second 28-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg (e.g., 1 mg±10%), the ivC1D2 is about 2 mg (e.g., 2 mg±10%), and the ivC1D3 is about 30 mg (e.g., 30 mg±10%); and (b) the second dosing cycle comprises: (i) a first intravenous dose (ivC2D1) of mosunetuzumab administered on Day 1 of the second dosing cycle, wherein the ivC2D1 is about 30 mg (e.g., 30 mg±10%); and (ii) 21 oral doses of lenalidomide administered on Days 1-21 of the second dosing cycle, wherein each oral dose of lenalidomide is about 20 mg (e.g., 20 mg±10%; 20 mg±2 mg). In particular embodiments, the control treatment comprises intravenously administering mosunetuzumab and orally administering lenalidomide according to a dosing regimen comprising at least a first 21-day dosing cycle and a second 28-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is 1 mg, the ivC1D2 is 2 mg, and the ivC1D3 is 30 mg; and (b) the second dosing cycle comprises: (i) a first intravenous dose (ivC2D1) of mosunetuzumab administered on Day 1 of the second dosing cycle, wherein the ivC2D1 is 30 mg; and (ii) 21 oral doses of lenalidomide administered on Days 1-21 of the second dosing cycle, wherein each oral dose of lenalidomide is 20 mg. In some embodiments, the dosing regimen of the control treatment further comprises one or more additional 28-day dosing cycles. In some embodiments, the dosing regimen of the control treatment comprises ten additional 28-day dosing cycles. In some embodiments, each additional dosing cycle comprises: (a) an intravenous dose of mosunetuzumab administered on Day 1 of the additional dosing cycle, wherein the intravenous dose of mosunetuzumab is about 30 mg (e.g., 30 mg±10%); and (b) twenty-one (21) oral doses of lenalidomide administered on Days 1-21 of the additional dosing cycle, wherein each oral dose of lenalidomide is about 20 mg (e.g., 20 mg±10%; 20 mg±2 mg). In particular embodiments, each additional dosing cycle comprises: (a) an intravenous dose of mosunetuzumab administered on Day 1 of the additional dosing cycle, wherein the intravenous dose of mosunetuzumab is 30 mg; and (b) twenty-one (21) oral doses of lenalidomide administered on Days 1-21 of the additional dosing cycle, wherein each oral dose of lenalidomide is 20 mg.

In some embodiments, the subject has an Eastern Cooperative Oncology Group (ECOG) Performance Status of 0, 1, or 2. In some embodiments, the subject has not been previously treated for FL. In some embodiments, the subject has relapsed after or is refractory to one or more prior lines of systemic therapy. In some embodiments, the one or more prior lines of systemic therapy comprises an immunotherapy or a chemoimmunotherapy. In some embodiments, the one or more prior lines of systemic therapy comprises an anti-CD20 antibody. In some embodiments, the anti-CD20 antibody is rituximab.

In some embodiments, the FL is Grade 1, 2, or 3a.

In another aspect, the invention features a method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).

In some embodiments, the efficacy response is a CRR, and wherein the CRR or reference CRR is the proportion of the subjects receiving the corresponding treatment whose best overall response is a complete response (CR). In some embodiments, the improved response in CRR is an increase in CRR compared to the reference CRR of between 1% and 20% (e.g., between 5% and 20%, between 10% and 20%, between 15% and 20%, between 1% and 15%, between 1% and 10%, between 1% and 5%, between 5% and 15%, or between 10% and 20%; e.g., about 1%, about 5%, about 10%, about 15%, or about 20%). In some embodiments, the efficacy response in CRR is non-inferior compared to the reference rate CRR.

In some embodiments, the efficacy response is an ORR, and wherein the ORR or reference ORR is the proportion of the subjects receiving the corresponding treatment whose best overall response is a CR or a partial response (PR). In some embodiments, the improved response in ORR is an increase in ORR compared to the reference ORR of between 1% and 13% (e.g., between 1% and 5%, between 5% and 13%, or between 3% and 10%; e.g., about 1%, about 5%, about 10%, or about 13%). In some embodiments, the efficacy response in ORR is non-inferior compared to the reference ORR.

In some embodiments, the efficacy response is a DOR, and wherein the DOR or the reference DOR is measured starting from the time from the first occurrence of a documented CR or PR to disease progression or relapse or death from any cause, whichever occurs first. In some embodiments, the DOR or the reference DOR is the median DOR of the plurality of subjects receiving the corresponding treatment. In some embodiments, the improvement of the median DOR is an increase in the DOR compared to the reference DOR of between 1 and 13 months (e.g., between 1 and 5 months, between 5 and 13 months, or between 3 and 10 months; e.g., about 1 month, 5 months, 10 months, or 13 months). In some embodiments, the efficacy response in DOR is non-inferior compared to reference DOR. In some embodiments, the improvement of the DOR is an increase in the rate of DOR at 12 months compared to the rate of reference DOR at 12 months of between 1% and 31% (e.g., between 1% and 25%, between 1% and 20%, between 1% and 15%, between 1% and 10%, between 1% and 5%, between 5% and 10%, between 5% and 15%, between 5% and 20%, between 5% and 25%, between 5% and 31%, between 1% and 10%, between 10% and 20%, between 20% and 31%, between 15% and 31%, between 1% and 20%, or between 10% and 31%; e.g., about 1%, 5%, 10%, 15%, 20%, 25%, or 31%). In some embodiments, the improvement of the DOR is an increase in the rate of DOR at 12 months compared to the rate of reference DOR at 12 months of about 8%.

In some embodiments, the efficacy response is a DOCR, and wherein the DOCR or the reference DOCR is measured starting from the time from the first occurrence of a documented CR to disease progression or relapse or death from any cause, whichever occurs first. In some embodiments, the DOCR or the reference DOCR is the median DOCR of the plurality of subjects receiving the corresponding treatment. In some embodiments, the improvement of the DOCR is an increase in the rate of DOCR at 12 months compared to the rate of reference DOCR at 12 months of between 1% and 27% (e.g., between 1% and 25%, between 1% and 20%, between 1% and 15%, between 1% and 10%, between 1% and 5%, between 5% and 10%, between 5% and 15%, between 5% and 20%, between 5% and 25%, between 5% and 27%, between 1% and 10%, between 10% and 20%, between 20% and 27%, between 15% and 27%, between 1% and 20%, or between 10% and 27%; e.g., about 1%, 5%, 10%, 15%, 20%, 25%, or 27%). In some embodiments, the efficacy response in DOCR is non-inferior compared to the reference DOCR.

In some embodiments, the CR or PR is determined by PET/computed tomography (CT). In some embodiments, the CR or PR is determined based on the Revised Response Criteria for Malignant Lymphoma (Cheson et al., 2007).

In some embodiments, the efficacy response is a PFS, and wherein the PFS or the reference PFS is measured starting from the time from first treatment (e.g., first dose of mosunetuzumab or first dose of lenalidomide; e.g., Day 1 of dosing cycle 1) to the time of a first occurrence of disease progression or death from any cause. In some embodiments, the PFS or the reference PFS is the median PFS of the plurality of subjects receiving the corresponding treatment. In some embodiments, the improvement of the median PFS is an increase in the PFS compared to the reference PFS of between 1 and 14 months (e.g., between 1 and 5 months, between 5 and 14 months, or between 3 and 10 months; e.g., about 1 month, 5 months, 10 months, or 14 months). In some embodiments, the efficacy response in PFS is non-inferior compared to the reference PFS. In some embodiments, the improvement of the PFS is in an increase in the rate of PFS compared to the rate of reference PFS at 12 months of between 1% and 26% (e.g., between 1% and 20%, between 1% and 15%, between 1% and 10%, between 1% and 5%, between 5% and 10%, between 5% and 15%, between 5% and 20%, between 5% and 26%, between 1% and 10%, between 10% and 20%, between 20% and 26%, between 15% and 26%, between 1% and 20%, or between 10% and 26%; e.g., about 1%, 5%, 10%, 15%, 20%, or 26%). In some embodiments, the improvement of the PFS is in an increase in the rate of PFS compared to the rate of reference PFS at 12 months of about 5%.

In some embodiments, the efficacy response is an OS, wherein the OS or the reference OS is measured starting from the time from first treatment (e.g., first dose of mosunetuzumab or first dose of lenalidomide; e.g., Day 1 of dosing cycle 1) to death from any cause. In some embodiments, the improvement of the OS is an increase in the rate of OS compared to the rate of reference OS at 12 months of between 1% and 9% (e.g., about 1%, 2%, 3%, 4%, 5%, 6%, 7%, 8%, or 9%). In some embodiments, the efficacy response in OS is non-inferior compared to the reference OS.

In another aspect, the invention features a method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In some embodiments, the safety response is the rate of AE, and wherein the rate of AE or the reference rate of AE is the rate of AE in the plurality of subjects receiving the corresponding treatment. In some embodiments, the safety response in rate of AE is non-inferior compared to the reference rate of AE. In some embodiments, the improved rate of AE is a decrease in the rate of Grade 3 or 4 AE compared to the reference rate of Grade 3 or 4 AE of between 11% and 21% (e.g., between 11% and 16%, between 16% and 21%, or between 13% to 19%; e.g., about 11%, about 16%, or about 21%). In some embodiments, the improved rate of AE is a decrease in the rate of Grade 3 or 4 AE compared to the reference rate of Grade 3 or 4 AE of about 16%. In some embodiments, the rate of AE is the rate of Grade 5 AE in the plurality of subjects receiving the corresponding treatment, and wherein the rate of AE is non-inferior compared to the reference rate of AE. In some embodiments, the Grade of AE is determined based on the National Cancer Institute Common Terminology Criteria for Adverse Events (NCI CTCAE), v4.0.

In some embodiments, the safety response is the rate of SAE, and wherein the rate of SAE or the reference rate of SAE is the rate of SAE in the plurality of subjects receiving the corresponding treatment. In some embodiments, the improved safety response in rate of SAE is a decrease in the rate of SAE compared to the reference rate of SAE of between 9% and 19% (e.g., between 9% and 14%, between 14% and 19%, or between 11% and 17%; e.g., about 9%, about 14%, or about 19%). In some embodiments, the improved safety response in rate of SAE is a decrease in the rate of SAE compared to the reference rate of SAE of about 14%.

In some embodiments, the safety response is the rate of CRS, and wherein the rate of CRS or the reference rate of CRS is the rate of CRS in the plurality of subjects receiving the corresponding treatment. In some embodiments, the improved rate of CRS is a decrease in the rate of CRS compared to the reference rate of CRS of between 9% and 19% (e.g., between 9% and 14%, between 14% and 19%, or between 11% and 17%; e.g., about 9%, about 14%, or about 19%). In some embodiments, the improved rate of CRS is a decrease in the rate of CRS compared to the reference rate of CRS of about 14%. In some embodiments, the improved rate of CRS is a decrease in the rate of Grade 2+ CRS compared to the reference rate of Grade 2+ CRS of between 3% and 13% (e.g., between 3% and 8%, between 8% and 13%, or between 5% and 11%; e.g., about 3%, about 8%, or about 13%). In some embodiments, the improved rate of CRS is a decrease in the rate of Grade 2+ CRS compared to the reference rate of Grade 2+ CRS of about 8%. In some embodiments, the Grade of CRS is determined based on the Modified Cytokine Release Syndrome Grading System (Lee et al., 2014).

In another aspect, the invention features a method for treating Non-Hodgkin Lymphoma (NHL) or chronic lymphocytic leukemia (CLL) in a subject in need thereof, comprising administering to the subject a treatment comprising subcutaneously administered mosunetuzumab according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In some embodiments, one or more of the scAUC₀₋₈₄ or the ivAUC₀₋₈₄ is determined using population PK (popPK) model-predicted individual Empirical Bayesian Estimates (EBEs).

In some embodiments, the treatment comprises subcutaneously administering mosunetuzumab according to a dosing regimen comprising at least a first 21-day dosing cycle and a second 21-day dosing cycle, wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg+10%; 5 mg±0.5 mg), the scC1D2 is about 15 mg (e.g., 15 mg±10%; 15 mg±1.5 mg) or about 45 mg (e.g., 45 mg±10%; 45 mg±4.5 mg), and the scC1D3 is about 45 mg (e.g., 45 mg±10%; 45 mg±4.5 mg); and (b) the second dosing cycle comprises a first subcutaneous dose (scC2D1) of mosunetuzumab administered on Day 1 of the second dosing cycle, wherein the scC2D1 is about 45 mg (e.g., 45 mg±10%; 45 mg±4.5 mg). In particular embodiments, the scC1D2 is about 45 mg (e.g., 45 mg±10%; 45 mg+4.5 mg). In particular embodiments, the treatment comprises subcutaneously administering mosunetuzumab according to a dosing regimen comprising at least a first 21-day dosing cycle and a second 21-day dosing cycle, wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is 5 mg, the scC1D2 is 15 mg or 45 mg, and the scC1D3 is 45 mg; and (b) the second dosing cycle comprises a first subcutaneous dose (scC2D1) of mosunetuzumab administered on Day 1 of the second dosing cycle, wherein the scC2D1 is 45 mg. In particular embodiments, the scC1D2 is 45 mg.

In some embodiments, the dosing regimen of the treatment further comprises one or more additional 21-day dosing cycles. In some embodiments, the dosing regimen of the treatment comprises six, fifteen, or twenty-three additional 21-day dosing cycles. In some embodiments, each additional dosing cycle comprises a subcutaneous dose of mosunetuzumab administered on Day 1 of the additional dosing cycle, wherein the subcutaneous dose of mosunetuzumab is about 45 mg (e.g., 45 mg±10%; 45 mg±4.5 mg). In particular embodiments, each additional dosing cycle comprises a subcutaneous dose of mosunetuzumab administered on Day 1 of the additional dosing cycle, wherein the subcutaneous dose of mosunetuzumab is 45 mg.

In some embodiments, the control treatment comprises intravenously administering mosunetuzumab according to a dosing regimen comprising at least a first 21-day dosing cycle and a second 21-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg (e.g., 1 mg±10%), the ivC1D2 is about 2 mg (e.g., 2 mg±10%), and the ivC1D3 is about 60 mg (e.g., 60 mg±10%); and (b) the second dosing cycle comprises a first intravenous dose (ivC2D1) of mosunetuzumab administered on Day 1 of the second dosing cycle, wherein the ivC2D1 is about 60 mg (e.g., 60 mg±10%). In particular embodiments, the control treatment comprises intravenously administering mosunetuzumab according to a dosing regimen comprising at least a first 21-day dosing cycle and a second 21-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is 1 mg, the ivC1D2 is 2 mg, and the ivC1D3 is 60 mg; and (b) the second dosing cycle comprises a first intravenous dose (ivC2D1) of mosunetuzumab administered on Day 1 of the second dosing cycle, wherein the ivC2D1 is 60 mg. In some embodiments, the dosing regimen of the control treatment further comprises one or more additional 21-day dosing cycles. In some embodiments, the dosing regimen of the control treatment comprises six, fifteen, or twenty-three additional 21-day dosing cycles. In some embodiments, each additional dosing cycle comprises an intravenous dose of mosunetuzumab administered on Day 1 of the additional dosing cycle, wherein the intravenous dose of mosunetuzumab is about 30 mg (e.g., 30 mg±10%). In particular embodiments, each additional dosing cycle comprises an intravenous dose of mosunetuzumab administered on Day 1 of the additional dosing cycle, wherein the intravenous dose of mosunetuzumab is about 30 mg.

In some embodiments, the subject has an Eastern Cooperative Oncology Group (ECOG) Performance Status of 0 or 1. In some embodiments, the subject has relapsed after or is refractory to one or more prior lines of systemic therapy. In some embodiments, the subject has relapsed after or is refractory to two or more prior lines of systemic therapy. In some embodiments, the one or more prior lines of systemic therapy comprises an anti-CD20 antibody, an alkylating agent, a Bruton's tyrosine kinase (BTK) inhibitor, an anthracycline, or a combination thereof.

In some embodiments, the NHL is a follicular lymphoma (FL), a marginal zone lymphoma (MZL), a diffuse large B cell lymphoma (DLBCL), a transformed FL (trFL), a high grade B cell lymphoma (HGBL), a primary mediastinal B cell lymphoma (PMLBCL), a transformed indolent NHL, a mantle cell lymphoma (MCL), a Richter's transformation, or a small lymphocytic lymphoma (SLL). In some embodiments, the NHL is an FL, and the FL is Grade 1-3b FL or a transformed FL. In some embodiments, the FL is Grade 1-3a FL, and the subject has relapsed after or is refractory to two or more prior lines of systemic therapy comprising an anti-CD20 antibody and an alkylating agent. In some embodiments, the NHL is a DLBCL or a trFL. In some embodiments, the NHL is a DLBCL or a trFL, and the subject has relapsed after or is refractory to two or more prior lines of systemic therapy comprising an anthracycline and an anti-CD20 antibody. In some embodiments, the NHL is a Richter's transformation. In some embodiments, the NHL is a Richter's transformation, and the subject has relapsed after or is refractory to one or more prior lines of therapy comprising an anthracycline and an anti-CD20 antibody. In some embodiments, the anti-CD20 antibody is rituximab. In some embodiments, the NHL is an MCL. In some embodiments, the NHL is an MCL, and the subject has relapsed after or is refractory to one or more prior lines of systemic therapy comprising a BTK inhibitor.

In some embodiments, the method comprises administering an additional therapeutic agent. In some embodiments, the additional therapeutic agent comprises a corticosteroid, an antihistamine, an antipyretic, or an IL-6R antagonist. In some embodiments, the additional therapeutic agent is a corticosteroid, and wherein the corticosteroid comprises prednisone, methylprednisolone, or dexamethasone. In some embodiments, the additional therapeutic agent is an antihistamine, and wherein the antihistamine comprises diphenhydramine hydrochloride or equivalent. In some embodiments, the additional therapeutic agent is an antipyretic, and wherein the antipyretic is acetaminophen. In some embodiments, the additional therapeutic agent is an IL-6R antagonist, and wherein the IL-6R antagonist is tocilizumab.

In one aspect, the invention features use of mosunetuzumab in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In one aspect, the invention features use of lenalidomide in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In one aspect, the invention features use of mosunetuzumab and lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In one aspect, the invention features use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In one aspect, the invention features use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In one aspect, the invention features use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In one aspect, the invention features mosunetuzumab for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In one aspect, the invention features lenalidomide for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In one aspect, the invention features mosunetuzumab and lenalidomide for use for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In one aspect, the invention features use of mosunetuzumab in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In one aspect, the invention features use of lenalidomide in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In one aspect, the invention features use of mosunetuzumab and lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In one aspect, the invention features use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In one aspect, the invention features use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In one aspect, the invention features use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In one aspect, the invention features mosunetuzumab for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In one aspect, the invention features lenalidomide for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In one aspect, the invention features mosunetuzumab and lenalidomide for use for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In one aspect, the invention features use of mosunetuzumab in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In one aspect, the invention features use of lenalidomide in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In one aspect, the invention features use of mosunetuzumab and lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In one aspect, the invention features use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In one aspect, the invention features use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In one aspect, the invention features use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In one aspect, the invention features mosunetuzumab for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In one aspect, the invention features lenalidomide for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In one aspect, the invention features mosunetuzumab and lenalidomide for use for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In one aspect, the invention features use of mosunetuzumab in combination with lenalidomide for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
   (i) a first subcutaneous dose (scC2D1) of mosunetuzumab administered on Day 1 of the second dosing cycle, wherein the scC2D1 is about 45 mg; and
   (ii) twenty-one (21) oral doses of lenalidomide administered on Days 1-21 of the second dosing cycle, wherein each oral dose of lenalidomide is about 20 mg; and
(c) the ten additional dosing cycles each comprises:
   (i) a first subcutaneous dose (scC3D1-scC12D1) of mosunetuzumab administered on Day 1 of each additional dosing cycle, wherein the scC3D1-scC12D1 are each about 45 mg; and
   (ii) twenty-one (21) oral doses of lenalidomide administered on Days 1-21 of each additional dosing cycle, wherein each oral dose of lenalidomide is about 20 mg.

In one aspect, the invention features use of lenalidomide in combination with mosunetuzumab for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
   (i) a first subcutaneous dose (scC2D1) of mosunetuzumab administered on Day 1 of the second dosing cycle, wherein the scC2D1 is about 45 mg; and
   (ii) twenty-one (21) oral doses of lenalidomide administered on Days 1-21 of the second dosing cycle, wherein each oral dose of lenalidomide is about 20 mg; and
(c) the ten additional dosing cycles each comprises:
   (i) a first subcutaneous dose (scC3D1-scC12D1) of mosunetuzumab administered on Day 1 of each additional dosing cycle, wherein the scC3D1-scC12D1 are each about 45 mg; and
   (ii) twenty-one (21) oral doses of lenalidomide administered on Days 1-21 of each additional dosing cycle, wherein each oral dose of lenalidomide is about 20 mg.

In one aspect, the invention features use of mosunetuzumab and lenalidomide for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
   (i) a first subcutaneous dose (scC2D1) of mosunetuzumab administered on Day 1 of the second dosing cycle, wherein the scC2D1 is about 45 mg; and
   (ii) twenty-one (21) oral doses of lenalidomide administered on Days 1-21 of the second dosing cycle, wherein each oral dose of lenalidomide is about 20 mg; and
(c) the ten additional dosing cycles each comprises:
   (i) a first subcutaneous dose (scC3D1-scC12D1) of mosunetuzumab administered on Day 1 of each additional dosing cycle, wherein the scC3D1-scC12D1 are each about 45 mg; and
   (ii) twenty-one (21) oral doses of lenalidomide administered on Days 1-21 of each additional dosing cycle, wherein each oral dose of lenalidomide is about 20 mg.

In one aspect, the invention features use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
(b) the second dosing cycle comprises:
   (i) a first subcutaneous dose (scC2D1) of mosunetuzumab administered on Day 1 of the second dosing cycle, wherein the scC2D1 is about 45 mg; and
   (ii) twenty-one (21) oral doses of lenalidomide administered on Days 1-21 of the second dosing cycle, wherein each oral dose of lenalidomide is about 20 mg; and
(c) the ten additional dosing cycles each comprises:
   (i) a first subcutaneous dose (scC3D1-scC12D1) of mosunetuzumab administered on Day 1 of each additional dosing cycle, wherein the scC3D1-scC12D1 are each about 45 mg; and
   (ii) twenty-one (21) oral doses of lenalidomide administered on Days 1-21 of each additional dosing cycle, wherein each oral dose of lenalidomide is about 20 mg.

In one aspect, the invention features use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
   (i) a first subcutaneous dose (scC2D1) of mosunetuzumab administered on Day 1 of the second dosing cycle, wherein the scC2D1 is about 45 mg; and
   (ii) twenty-one (21) oral doses of lenalidomide administered on Days 1-21 of the second dosing cycle, wherein each oral dose of lenalidomide is about 20 mg; and
(c) the ten additional dosing cycles each comprises:
   (i) a first subcutaneous dose (scC3D1-scC12D1) of mosunetuzumab administered on Day 1 of each additional dosing cycle, wherein the scC3D1-scC12D1 are each about 45 mg; and
   (ii) twenty-one (21) oral doses of lenalidomide administered on Days 1-21 of each additional dosing cycle, wherein each oral dose of lenalidomide is about 20 mg.

In one aspect, the invention features use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
   (i) a first subcutaneous dose (scC2D1) of mosunetuzumab administered on Day 1 of the second dosing cycle, wherein the scC2D1 is about 45 mg; and
   (ii) twenty-one (21) oral doses of lenalidomide administered on Days 1-21 of the second dosing cycle, wherein each oral dose of lenalidomide is about 20 mg; and
(c) the ten additional dosing cycles each comprises:
   (i) a first subcutaneous dose (scC3D1-scC12D1) of mosunetuzumab administered on Day 1 of each additional dosing cycle, wherein the scC3D1-scC12D1 are each about 45 mg; and
   (ii) twenty-one (21) oral doses of lenalidomide administered on Days 1-21 of each additional dosing cycle, wherein each oral dose of lenalidomide is about 20 mg.

In one aspect, the invention features mosunetuzumab for use in combination with lenalidomide for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
   (i) a first subcutaneous dose (scC2D1) of mosunetuzumab administered on Day 1 of the second dosing cycle, wherein the scC2D1 is about 45 mg; and
   (ii) twenty-one (21) oral doses of lenalidomide administered on Days 1-21 of the second dosing cycle, wherein each oral dose of lenalidomide is about 20 mg; and
(c) the ten additional dosing cycles each comprises:
   (i) a first subcutaneous dose (scC3D1-scC12D1) of mosunetuzumab administered on Day 1 of each additional dosing cycle, wherein the scC3D1-scC12D1 are each about 45 mg; and
   (ii) twenty-one (21) oral doses of lenalidomide administered on Days 1-21 of each additional dosing cycle, wherein each oral dose of lenalidomide is about 20 mg.

In one aspect, the invention features lenalidomide for use in combination with mosunetuzumab for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
   (i) a first subcutaneous dose (scC2D1) of mosunetuzumab administered on Day 1 of the second dosing cycle, wherein the scC2D1 is about 45 mg; and
   (ii) twenty-one (21) oral doses of lenalidomide administered on Days 1-21 of the second dosing cycle, wherein each oral dose of lenalidomide is about 20 mg; and
(c) the ten additional dosing cycles each comprises:
   (i) a first subcutaneous dose (scC3D1-scC12D1) of mosunetuzumab administered on Day 1 of each additional dosing cycle, wherein the scC3D1-scC12D1 are each about 45 mg; and
   (ii) twenty-one (21) oral doses of lenalidomide administered on Days 1-21 of each additional dosing cycle, wherein each oral dose of lenalidomide is about 20 mg.

In one aspect, the invention features mosunetuzumab and lenalidomide for use for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
(b) the second dosing cycle comprises:
   (i) a first subcutaneous dose (scC2D1) of mosunetuzumab administered on Day 1 of the second dosing cycle, wherein the scC2D1 is about 45 mg; and
   (ii) twenty-one (21) oral doses of lenalidomide administered on Days 1-21 of the second dosing cycle, wherein each oral dose of lenalidomide is about 20 mg; and
(c) the ten additional dosing cycles each comprises:
   (i) a first subcutaneous dose (scC3D1-scC12D1) of mosunetuzumab administered on Day 1 of each additional dosing cycle, wherein the scC3D1-scC12D1 are each about 45 mg; and
   (ii) twenty-one (21) oral doses of lenalidomide administered on Days 1-21 of each additional dosing cycle, wherein each oral dose of lenalidomide is about 20 mg.

In one aspect, the invention features use of mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).

In one aspect, the invention features use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).

In one aspect, the invention features mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered response rate (ORR), a duration of response (DOR), a duration of complete response (DOCR), progression free survival (PFS), or overall survival (OS).

In one aspect, the invention features use of mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In one aspect, the invention features use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In one aspect, the invention features mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects r results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In one aspect, the invention features use of mosunetuzumab for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In one aspect, the invention features use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In one aspect, the invention features mosunetuzumab for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In some embodiments, administering the combination treatment comprising subcutaneously administered mosunetuzumab and orally administered lenalidomide to a plurality of subjects having FL results in an ORR in the plurality of subjects of between about 81% to about 99% (e.g., 81%, 82%, 83%, 84%, 85%, 86%, 87%, 88%, 89%, 90%, 91%, 92%, 93%, 94%, 95%, 96%, 97%, 98%, or 99%). In some embodiments, the ORR is about 90%. In some embodiments, administering the combination treatment to a plurality of subjects having FL results in a CRR in the plurality of subjects of between about 79% to about 97% (e.g., 79%, 80%, 81%, 82%, 83%, 84%, 85%, 86%, 87%, 88%, 89%, 90%, 91%, 92%, 93%, 94%, 95%, 96%, or 97%). In some embodiments, the CRR is about 88%. In some embodiments, administering the combination treatment to a plurality of subjects having FL results in a 12-month event-free rate for DOR in the plurality of subjects of between about 86% to about 100% (e.g., 86%, 87%, 88%, 89%, 90%, 91%, 92%, 93%, 94%, 95%, 96%, 97%, 98%, 99%, or 100%). In some embodiments, the 12-month event-free rate for DOR is about 94%. In some embodiments, administering the combination treatment to a plurality of subjects having FL results in a 12-month event-free rate for DOCR in the plurality of subjects of between about 86% to about 100% (e.g., 86%, 87%, 88%, 89%, 90%, 91%, 92%, 93%, 94%, 95%, 96%, 97%, 98%, 99%, or 100%). In some embodiments, the 12-month event-free rate for DOCR is about 94%. In some embodiments, administering the combination treatment to a plurality of subjects having FL results in a 12-month event-free rate for PFS in the plurality of subjects of between about 76% to about 98% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98%). In some embodiments, the 12-month event-free rate for PFS is about 87%. In some embodiments, administering the combination treatment to a plurality of subjects having FL results in a 12-month event-free rate for OS in the plurality of subjects of between about 92% to about 100% (e.g., 92%, 93%, 94%, 95%, 96%, 97%, 98%, 99%, or 100%). In some embodiments, the 12-month event-free rate for OS is about 97%. In some embodiments, the FL is previously untreated (1 L) FL.

In some embodiments, administering the treatment comprising subcutaneous mosunetuzumab to a plurality of subjects having R/R FL results in an ORR of between about 66% to about 85% (e.g., 66%, 67%, 68%, 69%, 70%, 71%, 72%, 73%, 74%, 75%, 76%, 77%, 78%, 79%, 80%, 81%, 82%, 83%, 84%, or 85%). In some embodiments, the ORR is about 77%. In some embodiments, administering the treatment to a plurality of subjects having R/R FL results in a CRR in the plurality of subjects of between about 51% to about 72% (e.g., 51%, 52%, 53%, 54%, 55%, 56%, 57%, 58%, 59%, 60%, 61%, 62%, 63%, 64%, 65%, 66%, 67%, 68%, 69%, 70%, 71%, or 72%). In some embodiments, the CRR is about 62%. In some embodiments, administering the treatment to a plurality of subjects having R/R FL results in an 18-month event-free rate for DOCR in the plurality of subjects of between about 57% to about 83% (e.g., 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, or 83%). In some embodiments, the 18-month event-free rate for DOCR is about 70%. In some embodiments, administering the treatment to a plurality of subjects having R/R FL results in a median DOCR in the plurality of subjects of at least about 20 months (e.g., 20, 24, 28, 32, 36, 40, 44, 48, 60, 72 months, or more). In some embodiments, the median DOCR is about 35 months. In some embodiments, administering the treatment to a plurality of subjects having R/R FL results in an 18-month event-free rate for PFS in the plurality of subjects of between about 46% to about 68% (e.g., 46%, 47%, 48%, 49%, 50%, 51%, 52%, 53%, 54%, 55%, 56%, 57%, 58%, 59%, 60%, 61%, 62%, 63%, 64%, 65%, 66%, 67%, or 68%). In some embodiments, the 18-month event-free rate for PFS is about 57%. In some embodiments, administering the treatment to a plurality of subjects having R/R FL results in a median PFS in the plurality of subjects of at least 14 months (e.g., 14, 18, 22, 24, 28, 30, 34, 38, 42 months, or more). In some embodiments, the median PFS is about 24 months.

In some embodiments, administering the treatment comprising subcutaneous mosunetuzumab to a plurality of subjects having 1 L FL results in an ORR of between about 76% to about 96% (e.g., 76%, 77%, 78%, 79%, 80%, 81%, 82%, 83%, 84%, 85%, 86%, 87%, 88%, 89%, 90%, 91%, 92%, 93%, 94%, 95%, or 96%). In some embodiments, the ORR is about 87%. In some embodiments, administering the treatment to a plurality of subjects having 1 L FL results in a CRR in the plurality of subjects of between about 55% to about 67% (e.g., 55%, 56%, 57%, 58%, 59%, 60%, 61%, 62%, 63%, 64%, 65%, 66%, or 67%). In some embodiments, the CRR is about 61%. In some embodiments, administering the treatment to a plurality of subjects having 1 L FL results in a 12-month event-free rate for PFS in the plurality of subjects of between about 73% to about 90% (e.g., 73%, 74%, 75%, 76%, 77%, 78%, 79%, 80%, 81%, 82%, 83%, 84%, 85%, 86%, 87%, 88%, 89%, or 90%). In some embodiments, the 12-month event-free rate for PFS is about 83%. In some embodiments, administering the treatment to a plurality of subjects having 1 L FL results in a median time to response (TTR) in the plurality of subjects exhibiting a response of between about 1 month to about 6 months (e.g., about 1, 2, 3, 4, 5, or 6 months). In some embodiments, the median TTR is about 3 months. In some embodiments, the subjects of the plurality have a high tumor burden, per the Groupe d'Etude des Lymphomes Folliculaires (GELF) criteria.

In some embodiments, administering the treatment comprising subcutaneous mosunetuzumab to a plurality of subjects having 1 L MZL results in an ORR of between about 58% to about 88% (e.g., 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, or 88%). In some embodiments, the ORR is about 75%. In some embodiments, administering the treatment to a plurality of subjects having 1 L MZL results in a CRR in the plurality of subjects of between about 44% to about 77% (e.g., 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, or 77%). In some embodiments, the CRR is about 61%. In some embodiments, administering the treatment to a plurality of subjects having 1 L MZL results in a median TTR in the plurality of subjects exhibiting a response of between about 2 months to about 6 months (e.g., about 2, 3, 4, 5, or 6 months). In some embodiments, the median TTR is about 3 months.

### BRIEF DESCRIPTION OF THE DRAWINGS

FIG. 1 is a schematic diagram showing the design of the dose escalation portion of the GO29781 study in Example 1. Initially, mosunetuzumab is given as a single non-fractionated intravenous (IV) dose on Day 1 of each cycle (Group A). Cycle 1 dosing is subsequently modified such that Group A dose escalation stops and mosunetuzumab dose escalation is conducted as follows: Group B: mosunetuzumab dose escalation utilizing a Cycle 1 step-up IV dosing scheme; Group D: mosunetuzumab dose escalation utilizing a Cycle 1 non-fractionated subcutaneous (SC) dosing scheme; Group F: mosunetuzumab dose escalation utilizing a Cycle 1 step-up SC dosing scheme. C=Cycle; D=Day; DL=dose level; MAD=maximum assessed dose.

FIG. 2 is a schematic diagram showing the design of the non-Hodgkin's lymphoma (NHL) expansion cohorts and the chronic lymphocytic leukemia (CLL) dose escalation/expansion cohorts of the GO29781 study. DLBCL=diffuse large B-cell lymphoma; FL=follicular lymphoma; MCL=mantle cell lymphoma; NHL=Non-Hodgkin's Lymphoma; RP2D=recommended Phase II dose; R/R=relapsed/refractory; trFL=transformed follicular lymphoma. ^{a}Multiple expansion cohorts based on Groups A, B, D, and F dose escalations may be tested. ^{b}Expansion cohorts in R/R DLBCL/trFL enroll up to about 20 patients except for expansion cohort based on Group B RP2D, which enrolls up to about 80 patients. ^{c}Expansion cohorts in R/R FL enroll up to about 20 patients except for expansion cohort based on Group B RP2D, which enrolls up to about 80 patients. ^{d}Expansion cohort based on Group B dose escalation only is tested. ^{e}Dose escalation conducted similarly to that for NHL (see FIG. 1). ^{f}Multiple expansion cohorts based on Groups B, D, and F dose escalations may be tested.

FIG. 3 is a schematic diagram showing assessment windows in Group B of the GO29781 study.

FIG. 4 is a schematic diagram showing the duration of initial study treatment in the GO29781 study and options for re-treatment or continued study treatment. CR=complete response; PD=progressive disease; PR=partial response; SD=stable disease. ^{a}Additional rounds of re-treatment permitted, follow treatment flow for initial treatment. ^{b}Scan should be scheduled to avoid/minimize any dose delay between Cycles 8 and 9 as much as possible.

FIG. 5 is a schematic of the dosing regimen described in Example 2 for the dosing regimen of the combination therapy of IV mosunetuzumab and lenalidomide. Mosunetuzumab is administered intravenously via infusion and lenalidomide is administered orally. Len=lenalidomide; Cycle=dosing cycle.

FIG. 6 is a schematic of the dosing regimen described in Example 2 for the dosing regimen of the combination therapy of SC mosunetuzumab and lenalidomide. Mosunetuzumab is administered subcutaneously via injection and lenalidomide is administered orally. Len=lenalidomide; Cycle=dosing cycle.

FIG. 7 is a graph and accompanying statistical analysis of duration of response results comparing B11 (IV mosunetuzumab) and F2 (SC mosunetuzumab) cohort patients.

FIG. 8 is a graph and accompanying statistical analysis of duration of complete response results comparing B11 (IV mosunetuzumab) and F2 (SC mosunetuzumab) cohort patients.

FIG. 9 is a graph and accompanying statistical analysis of progression free survival results comparing B11 (IV mosunetuzumab) and F2 (SC mosunetuzumab) cohort patients.

FIG. 10 is a graph and accompanying statistical analysis of overall survival results comparing B11 (IV mosunetuzumab) and F2 (SC mosunetuzumab) cohort patients.

FIG. 11 summarizes the demographics and baseline characteristics of Arm A (IV mosun+len) and Arm B (SC mosun+len) patients.

FIG. 12 summarizes the previously administered therapies of Arm A (IV mosun+len) and Arm B (SC mosun+len) patients.

FIG. 13 summarizes the baseline characteristics of the diseases of the Arm A (IV mosun+len) and Arm B (SC mosun+len) patients and the FL history.

FIG. 14 summarizes the complete response analysis results of various patient subgroups of the Arm A (IV mosun+len) and Arm B (SC mosun+len) patients.

FIG. 15 summarizes the overall response analysis results of various patient subgroups of the Arm A (IV mosun+len) and Arm B (SC mosun+len) patients.

FIG. 16 is a Kaplan-Meier plot and summary table showing duration of complete response results of the Arm A (IV mosun+len) and Arm B (SC mosun+len) patients.

FIG. 17 is a Kaplan-Meier plot and summary table showing duration of complete response results of the Arm A (IV mosun+len) and Arm B (SC mosun+len) patients that have been treated with 2 or more (2 L+) prior lines of therapy.

FIG. 18 is a Kaplan-Meier plot and summary table showing duration of response results of the Arm A (IV mosun+len) and Arm B (SC mosun+len) patients.

FIG. 19 is a Kaplan-Meier plot and summary table showing duration of response results of the Arm A (IV mosun+len) and Arm B (SC mosun+len) patients that have been treated with 2 or more (2 L+) prior lines of therapy.

FIG. 20 is a Kaplan-Meier plot and summary table showing overall survival results of the Arm A (IV mosun+len) and Arm B (SC mosun+len) patients.

FIG. 21 is a Kaplan-Meier plot and summary table showing overall survival results of the Arm A (IV mosun+len) and Arm B (SC mosun+len) patients that have been treated with 2 or more (2 L+) prior lines of therapy.

FIG. 22 is a Kaplan-Meier plot and summary table showing progression free survival results of the Arm A (IV mosun+len) and Arm B (SC mosun+len) patients.

FIG. 23 is a Kaplan-Meier plot and summary table showing progression free survival results of the Arm A (IV mosun+len) and Arm B (SC mosun+len) patients that have been treated with 2 or more (2 L+) prior lines of therapy.

FIG. 24 summarizes the overall safety results for Arm A (IV mosun+len) and Arm B (SC mosun+len).

FIG. 25 summarizes the overall safety results for Arm A (IV mosun+len) and Arm B (SC mosun+len) in patients who had received two or more prior lines of therapy.

FIG. 26 shows a breakdown of Grade 5 adverse events for Arm A (IV mosun+len) and Arm B (SC mosun+len).

FIG. 27 summarizes CRS events that occurred for Arm A (IV mosun+len) and Arm B (SC mosun+len), as well as the time to onset and duration of CRS events, as well as a summary of CRS events by dose/cycle.

FIG. 28 summarizes the rate of adverse events of special interest in Arm A (IV mosun+len) and Arm B (SC mosun+len) patients.

FIG. 29 shows a comparison of the efficacy of SC or IV mosun+len to other therapies in treating patients with FL who have been treated with 2 or more prior lines of therapy.

FIG. 30 shows a comparison of the safety of SC or IV mosun+len to other therapies in treating patients with FL who have been treated with 2 or more prior lines of therapy.

FIG. 31 is a Swimlane plot of individual response to Mosun SC treatment in patients with 1 L MZL. 1 L, first-line; Mosun, mosunetuzumab; MZL, marginal zone lymphoma; PET, positron emission tomography; SC, subcutaneous.

FIG. 32 is a bar chart showing a breakdown by patient/subject subgroup of complete metabolic response rate (CMR rate or complete response (CR) rate) and partial metabolic response rate (PMR rate or partial response (PR) rate) for the patients described in Example 7. Overall response rate (ORR) is shown in the number above each bar. Patients were divided by age (>65 years or <65 years); Follicular Lymphoma International Prognostic Index (FLIPI) score (0 or 1, 2, or 3-5); elevated lactate dehydrogenase (LDH) (yes or no); B symptoms (yes or no); bulky disease (yes or no), or FL Grade (1-2 or 3a).

### DETAILED DESCRIPTION

The present invention involves methods of treating a subject (or a population of subjects) having a CD20-positive cell proliferative disorder, e.g., a B cell proliferative disorder (e.g., non-Hodgkin's lymphoma (NHL) (e.g., a previously untreated (1 L) NHL, a diffuse-large B cell lymphoma (DLBCL) (e.g., 1 L DLBCL, a relapsed and/or refractory DLBCL, or a Richter's transformation), a follicular lymphoma (FL) (e.g., a 1 L FL, a relapsed and/or refractory FL, or a transformed FL), a mantle cell lymphoma (MCL), a high-grade B cell lymphoma, or a primary mediastinal (thymic) large B cell lymphoma (PMLBCL)) or a chronic lymphoid leukemia (CLL)) by subcutaneously administering to the subject a mosunetuzumab in a dosing regimen comprising at least a first dosing cycle and a second dosing cycle, wherein the treatment comprising subcutaneous administration of mosunetuzumab as a monotherapy or in combination with lenalidomide exhibits non-inferiority or an improved outcome as measured using pharmacokinetics (PK), efficacy, safety, or a combination thereof, as compared to intravenous administration of mosunetuzumab as a monotherapy or in combination with lenalidomide, respectively.

### I. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. DEFINITIONS

It is to be understood that aspects and embodiments of the invention described herein include “comprising,” “consisting,” and “consisting essentially of” aspects and embodiments.

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. In some embodiments, the term “about” a value or parameter refers to that value or parameter±10%.

The terms “cancer” and “cancerous” refer to or describe the physiological condition in mammals that is typically characterized by unregulated cell growth. Examples of cancer include, but are not limited to, hematologic cancers, such as mature B cell cancers, excluding Hodgkin's lymphoma, but including non-Hodgkin's lymphoma (NHL), such as diffuse large B cell lymphoma (DLBCL), which may be relapsed or refractory DLBCL or a Richter's transformation. Other specific examples of cancer also include germinal-center B cell-like (GCB) diffuse large B cell lymphoma (DLBCL), activated B cell-like (ABC) DLBCL, follicular lymphoma (FL), transformed FL, mantle cell lymphoma (MCL), acute myeloid leukemia (AML), chronic lymphoid leukemia (CLL), marginal zone lymphoma (MZL), transformed MZL, high-grade B cell lymphoma, primary mediastinal (thymic) large B cell lymphoma (PMLBCL), small lymphocytic leukemia (SLL), lymphoplasmacytic lymphoma (LL), transformed LL, Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, heavy chain diseases, a heavy chain disease, y heavy chain disease, u heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle centre lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of cancer include, but are not limited to, carcinoma, lymphoma, blastoma, sarcoma, and leukemia or lymphoid malignancies, including B cell lymphomas. More particular examples of such cancers include, but are not limited to, multiple myeloma (MM); low-grade/follicular NHL; small lymphocytic (SL) NHL; intermediate-grade/follicular NHL; intermediate-grade diffuse NHL; high-grade immunoblastic NHL; high-grade lymphoblastic NHL; high-grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD).

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder,” and “tumor” are not mutually exclusive as referred to herein.

A “disorder” is any condition that would benefit from treatment including, but not limited to, chronic and acute disorders or diseases including those pathological conditions which predispose the mammal to the disorder in question.

The terms “cell proliferative disorder” and “proliferative disorder” refer to disorders that are associated with some degree of abnormal cell proliferation. In one embodiment, the cell proliferative disorder is cancer. In another embodiment, the cell proliferative disorder is a tumor.

The terms “B cell proliferative disorder” or “B cell malignancy” refer to disorders that are associated with some degree of abnormal B cell proliferation and include, for example, lymphomas, leukemias, myelomas, and myelodysplastic syndromes. In one embodiment, the B cell proliferative disorder is a lymphoma, such as non-Hodgkin's lymphoma (NHL), including, for example, diffuse large B cell lymphoma (DLBCL) (e.g., a relapsed or refractory DLBCL or a Richter's transformation), FL (e.g., a relapsed and/or refractory FL or transformed FL), MCL, high-grade B cell lymphoma, or PMLBCL). In another embodiment, the B cell proliferative disorder is a leukemia, such as chronic lymphocytic leukemia (CLL). In some instances, a subject having a relapsed or refractory disease has relapsed after or is refractory to two or more prior lines of systemic therapy. In some embodiments, the FL is a previously untreated FL. In a particular embodiment, a subject having a previously untreated FL has been determined based on the Groupe d'Etude des Lymphomes Folliculaires (GELF) criteria (Brice et al. J Clin Oncol. 15 (3): 1110-1117, 1997) to be in need of systemic therapy to treat the previously untreated FL.

The terms “Groupe d'Etude des Lymphomes Folliculaires criteria” and “GELF criteria” refer to criteria for determining whether immediate therapy for follicular lymphoma is required. In particular, a subject or individual satisfying one of more GELF criteria is determined to be in need of treatment. GELF criteria include (i) any nodal or extranodal tumor mass>7 cm in diameter; (ii) involvement of at least 3 nodal sites, each with diameter>3 cm; (iii) presence of B symptoms (e.g., fever, night sweats, and weight loss); (iv) splenomegaly (>16 cm on computer tomography (CT) scan); (v) risk of local compressive symptoms that may result in organ compromise; (vi) pleural effusion or peritoneal ascites; (vii) leukemic phase (>5×10⁹/L circulating malignant cells); and (viii) cytopenia (granulocye count<1×10⁹/L and/or platelets<100×10⁹/L). See Brice et al. J Clin Oncol. 15 (3): 1110-1117, 1997. In some embodiments, a subject satisfying one or more GELF criteria is considered to have a high tumor burden.

The term “B symptoms” refers to systemic symptoms associated with Hodgkin's lymphoma and some NHLs. B symptoms may act as a prognostic factor for certain lymphomas. B symptoms include night sweats (drenching); unexplained fever (above 38° C.); and unexplained weight loss (generally greater than 10% of body weight, e.g., in the six months before admission (for the lymphoma)). See North American Association of Central Cancer Registries (NAACCR) Data Dictionary, Data Item #3812: B symptoms (cancer.gov).

By “Follicular Lymphoma International Prognostic Index” or “FLIPI” is meant a scoring system or index for determining prognostic risk of patients (e.g., with cancer; e.g., an NHL; e.g., a follicular lymphoma (FL)). FLIPI score ranges from 0-5, depending on how many of the five conditions or risk factors a patient may have among the following: (i) age>60 years; (ii) Ann Arbor stage III-IV; hemoglobin level≤120 g/L; serum lactate dehydrogenase (LDH) level>upper limit of normal (ULN) (e.g., >280 units/L); and (v) number of nodal sites>4. FLIPI risk groups are defined as follows: (a) 0 or 1 FLIPI risk factors=low risk group; (b) 2 FLIPI risk factors=intermediate risk group; (c) 3-5 FLIPI risk factors=high risk group. See e.g., Solal-Céligny et al. Blood. 2004; 104 (5): 1258-1265. at Table 4.

As used herein, the terms “Ann Arbor staging” or “Ann Arbor stages” refers to a system for classification of stages of lymphoma (e.g., NHLs). Lymphomas (e.g., NHLs) can be classified as one of four Ann Arbor stages. Stage I refers to lymphomas exhibiting involvement of a single lymph node region or of a single extralymphatic organ or site. Stage II refers to lymphomas exhibiting involvement of 2 or more lymph node regions on the same side of the diaphragm. Stage III refers to lymphomas exhibiting involvement of lymph node regions on both sides of the diaphragm (III), which may also be accompanied by localized involvement of extralymphatic organ or site or by involvement of the spleen, or both. Stage IV refers to lymphomas exhibiting diffuse or disseminated involvement of 1 or more extralymphatic organs or tissues with or without associated lymph node enlargement. Liver involvement is always considered to be diffuse, and, thus, always considered Ann Arbor stage IV. Lymphatic structures include the lymph nodes, thymus, spleen, appendix, Waldeyer's ring, and Peyer's patches. See Carbone, P. P. et al., Cancer Res. 1971, 31 (11): 1860-1861.

As used herein, “treatment” (and grammatical variations thereof, such as “treat” or “treating”) refers to clinical intervention in an attempt to alter the natural course of the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

As used herein, “delaying progression” of a disorder or disease means to defer, hinder, slow, retard, stabilize, and/or postpone development of the disease or disorder (e.g., a CD20-positive cell proliferative disorder, e.g., a B cell proliferative disorder, e.g., NHL, e.g., DLBCL or FL). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

By “reduce” or “inhibit” is meant the ability to cause an overall decrease, for example, of 20% or greater, of 50% or greater, or of 75%, 85%, 90%, 95%, or greater. In certain embodiments, reduce or inhibit can refer to the reduction or inhibition of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with mosunetuzumab, alone or in combination with lenalidomide, using the methods described herein. In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP). In other embodiments, reduce or inhibit can refer to the symptoms of the disorder being treated (e.g., CD20-positive cell proliferative disorder (e.g., a B cell proliferative disorder (e.g., an NHL (e.g., a previously untreated (1 L) NHL, a DLBCL (e.g., a 1 L DLBCL, a relapsed and/or refractory DLBCL, or a Richter's transformation), an FL (e.g., a 1 L FL, a relapsed and/or refractory FL, or a transformed FL), an MCL, a high-grade B cell lymphoma, or a PMLBCL) or a CLL)), the presence or size of metastases, or the size of the primary tumor.

As used herein, “administering” is meant a method of giving a dosage of a compound (e.g., mosunetuzumab or lenalidomide) or a composition (e.g., a pharmaceutical composition, e.g., a pharmaceutical composition including mosunetuzumab and/or lenalidomide) to a subject. The compounds and/or compositions utilized in the methods described herein can be administered subcutaneously (e.g., by subcutaneous injection; e.g., mosunetuzumab), intravenously (e.g., by intravenous infusion; e.g., mosunetuzumab), or orally (e.g., lenalidomide).

A “fixed” or “flat” dose of a therapeutic agent (e.g., a mosunetuzumab or lenalidomide) herein refers to a dose that is administered to a patient without regard for the weight or body surface area (BSA) of the patient. The fixed or flat dose is therefore not provided as a mg/kg dose or a mg/m² dose, but rather as an absolute amount of the therapeutic agent (e.g., mg).

A “subject” or an “individual” is a mammal. Mammals include, but are not limited to, primates (e.g., humans and non-human primates such as monkeys), domesticated animals (e.g., cows, sheep, cats, dogs, and horses), rabbits, and rodents (e.g., mice and rats). In certain embodiments, the subject or individual is a human.

“Individual response” or “response” can be assessed using any endpoint indicating a benefit to the subject, including, without limitation, (1) inhibition, to some extent, of disease progression (e.g., progression of a B cell proliferative disorder; e.g., NHL or CLL), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the B cell proliferative disorder (e.g., NHL or CLL); (6) increase or extend in the length of survival, including overall survival and progression-free survival; and/or (9) decreased mortality at a given point of time following treatment. In some embodiments, response to treatment (e.g., a complete response (CR) or a partial response (PR)) of a CD20-positive cell proliferative disorder (e.g., a B cell proliferative disorder; e.g., an NHL or a CLL) is determined based on the Revised Response Criteria for Malignant Lymphoma (Cheson et al., 2007). In some embodiments, response to treatment (e.g., a CR or a PR) of a CD20-positive cell proliferative disorder (e.g., a B cell proliferative disorder; e.g., an NHL or a CLL) is evaluated using the Lugano response criteria for malignant lymphoma (Cheson B D, et al. J Clin Oncol 2014; 32:1-9).

As used herein, “complete response” or “CR” refers to disappearance of all target lesions (i.e., all evidence of disease). In some instances, target nodes/nodal masses regress to ≤1.5 cm in longest transverse diameter. In some instances, a complete response is a complete metabolic response.

As used herein, “partial response” or “PR” refers to at least a 50% decrease in the sum of the product of the diameters (SPD) of target lesions, e.g., of up to 6 target measurable nodes and extranodal sites, taking as reference the baseline SPD. In some instances, a partial response is a partial metabolic response.

As used herein, “objective response rate” (ORR) refers to the sum of complete response (CR) rate and partial response (PR) rate.

As used herein, “duration of objective response” (DOR) is defined as the time from the first occurrence of a documented objective response to disease progression, relapse, or death from any cause, whichever occurs first.

As used herein, “duration of complete response” (DOCR) is defined as the time from the first occurrence of a documented complete response to disease progression, relapse, or death from any cause, whichever occurs first.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

An “effective response” of a subject or a subject's “responsiveness” to treatment with a medicament and similar wording refers to the clinical or therapeutic benefit imparted to a subject as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A subject who “does not have an effective response” to treatment refers to a subject who does not have any one of extending survival (including overall survival and progression free survival); resulting in an objective response (including a complete response or a partial response); or improving signs or symptoms of cancer.

As used herein, “survival” refers to the patient remaining alive, and includes overall survival as well as progression-free survival.

As used herein, “overall survival” (OS) refers to the time or duration from first study treatment administered to death from any cause.

As used herein, “progression-free survival” (PFS) refers to the length of time during and after treatment during which the disease being treated (e.g., CD20-positive cell proliferative disorder (e.g., a B cell proliferative disorder (e.g., an NHL (e.g., a previously untreated (1 L) NHL, a DLBCL (e.g., a 1 L DLBCL, a relapsed and/or refractory DLBCL, or a Richter's transformation), an FL (e.g., a 1 L FL, a relapsed and/or refractory FL, or a transformed FL), an MCL, a high-grade B cell lymphoma, or a PMLBCL) or a CLL)) does not get worse. Progression-free survival may include the amount of time patients have experienced a complete response or a partial response, as well as the amount of time patients have experienced stable disease. In some instances, PFS refers to the time or duration from first study treatment administered to the first occurrence of disease progression or death from any cause, whichever occurs first.

As used herein, “stable disease” or “SD” refers to neither sufficient shrinkage of target lesions to qualify for PR, nor sufficient increase to qualify for PD, taking as reference the smallest SLD since the treatment started. In some instances, stable disease refers to <50% decrease from baseline in SPD, e.g., of up to 6 dominant, measurable nodes and extranodal sites.

As used herein, “progressive disease” or “PD” refers to at least a 50% increase in the SLD of any previously identified lesion, taking as reference the smallest SLD, or at least a 50% increase in the SPD of target legions, taking as reference the smallest SPD, recorded since the treatment started or the presence of one or more new lesions.

As used herein, “delaying progression” of a disorder or disease means to defer, hinder, slow, retard, stabilize, and/or postpone development of the disease or disorder (e.g., CD20-positive cell proliferative disorder (e.g., a B cell proliferative disorder (e.g., an NHL (e.g., a previously untreated (1 L) NHL, a DLBCL (e.g., a 1 L DLBCL, a relapsed and/or refractory DLBCL, or a Richter's transformation), an FL (e.g., a 1 L FL, a relapsed and/or refractory FL, or a transformed FL), an MCL, a high-grade B cell lymphoma, or a PMLBCL) or a CLL)). This delay can be of varying lengths of time, depending on the history of the disease and/or subject being treated. As is evident to one skilled in the art, a sufficient or significant delay can, in effect, encompass prevention, in that the subject does not develop the disease. For example, in a late-stage cancer, development of central nervous system (CNS) metastasis, may be delayed.

As used herein, the term “reducing or inhibiting cancer relapse” means to reduce or inhibit tumor or cancer relapse, or tumor or cancer progression.

By “extending survival” is meant increasing overall or progression free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including complete response (CR) or partial response (PR).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “full-length antibody,” “intact antibody,” and “whole antibody” are used herein interchangeably to refer to an antibody having a structure substantially similar to a native antibody structure or having heavy chains that contain an Fc region as defined herein.

By “binding domain” is meant a part of a compound or a molecule that specifically binds to a target epitope, antigen, ligand, or receptor. Binding domains include but are not limited to antibodies (e.g., monoclonal, polyclonal, recombinant, humanized, and chimeric antibodies), antibody fragments or portions thereof (e.g., Fab fragments, Fab′₂, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term IgG “isotype” or “subclass” as used herein is meant any of the subclasses of immunoglobulins defined by the chemical and antigenic characteristics of their constant regions.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol., 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “variable region” or “variable domain” refers to the domain of an antibody heavy or light chain that is involved in binding the antibody (i.e., mosunetuzumab) to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
   (b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD3 antibody binds to an epitope of CD3 that is conserved among CD3 from different species.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The terms “anti-CD20 antibody” and “an antibody that binds to CD20” refer to an antibody that is capable of binding CD20 with sufficient affinity such that the antibody is useful as a diagnostic and/or therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD20 antibody binds to an epitope of CD20 that is conserved among CD20 from different species.

The term “cluster of differentiation 20” or “CD20,” as used herein, refers to any native CD20 from any vertebrate source, including mammals such as primates (e.g., humans) and rodents (e.g., mice and rats), unless otherwise indicated. The term encompasses “full-length,” unprocessed CD20, as well as any form of CD20 that results from processing in the cell. The term also encompasses naturally occurring variants of CD20, including, for example, splice variants or allelic variants. CD20 includes, for example, human CD20 protein (see, e.g., NCBI RefSeq Nos. NP_068769.2 and NP_690605.1), which is 297 amino acids in length and may be generated, for example, from variant mRNA transcripts that lack a portion of the 5′ UTR (see, e.g., NCBI RefSeq No. NM_021950.3) or longer variant mRNA transcripts (see, e.g., NCBI RefSeq No. NM_152866.2).

The terms “anti-CD20/anti-CD3 bispecific antibody,” “bispecific anti-CD20/anti-CD3 antibody,” and “antibody that binds to CD20 and CD3,” or variants thereof, refer to mosunetuzumab.

As used herein, the term “mosunetuzumab” refers to an anti-CD20/anti-CD3 bispecific antibody having the International Nonproprietary Names for Pharmaceutical Substances (INN) List 117 (WHO Drug Information, Vol. 31, No. 2, 2017, p. 304-305), or the CAS Registry Number 1905409-39-3. Further it is understood that the term “mosunetuzumab” as used herein also encompasses any biosimilar version as approved by regulatory authorities, e.g., the US FDA or European EMA. In addition, mosunetuzumab may refer to a bispecific anti-CD3/anti-CD20 bispecific antibody comprising the HVR sequences, VH and VL sequences, and/or heavy and light chain sequences described in Table 1 below, as well as any variants thereof comprising post-translational modifications.

As used herein, the term “lenalidomide” refers to a compound having the CAS Registry Number 191732-72-6 and IUPAC name (3RS)-3-(4-Amino-1-oxo-1,3-dihydro-2H-isoindol-2-yl) piperidine-2,6-dione. Lenalidomide is also known by tradenames including REVLIMID®, linamid, and lenalid. Lenalidomide has the DrugBank Accession Number DB00480, PubChem CID 216326, and chemical formula C₁₃H₁₃N₃O₃.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (K_{D}) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a Ko in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “chemotherapeutic agent” refers to a compound useful in the treatment of cancer, such as a CD20-positive cell proliferative disorder (e.g., a B cell proliferative disorder (e.g., a relapsed or refractory B cell proliferative disorder), e.g., a non-Hodgkin's lymphoma (NHL; e.g., a diffuse large B cell lymphoma (DLBCL; e.g., a Richter's Transformation), a follicular lymphoma (FL; e.g., a Grade 1 FL, a Grade 2 FL, a Grade 3 FL (e.g., a Grade 3a FL, Grade 3b FL), or a transformed FL), a mantle cell lymphoma (MCL), or a marginal zone lymphoma (MZL)) or a chronic lymphoid leukemia (CLL), e.g., a relapsed or refractory NHL (e.g., a relapsed or refractory DLBCL, a relapsed or refractory FL, a relapsed or refractory MCL, or a marginal zone lymphoma (MZL)) or a relapsed or refractory CLL). Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl) propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy) quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl) methoxy]phenyl]-6 [5 [[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino) phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5a-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, dacarbazine, mechlorethamine, cisplatin, methotrexate, 5-fluorouracil, and ara-C); and (x) pharmaceutically acceptable salts, acids, prodrugs, and derivatives of any of the above.

The term “chemo-immunotherapy” refers to combination therapy that includes both chemotherapy drugs and immunotherapeutic agents. In some embodiments, chemo-immunotherapy is used to treat a cancer, e.g., a CD20-positive cancer, e.g., a NHL, e.g., a FL. In some embodiments, immunotherapeutic agents include an antibody, e.g., an anti-CD20 antibody (e.g., an anti-CD20 monoclonal antibody). In some embodiments, the anti-CD20 antibody or anti-CD20 monoclonal antibody is rituximab or obinutuzumab. In some embodiments, chemo-immunotherapy includes R-CHOP.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy) quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor. In one instance, the PI3K inhibitor inhibits delta isoform of PI3K (i.e., P110δ). In some instances, the PI3K inhibitor is 5-Fluoro-3-phenyl-2-[(1S)-1-(7H-purin-6-ylamino) propyl]-4 (3H)-quinazolinone and salts thereof. In some instances, the PI3K inhibitor is idelalisib (CAS #: 870281-82-6). In one instance, the PI3K inhibitor inhibits alpha and delta isoforms of PI3K. In some instances, the PI3K inhibitor is 2-{3-[2-(1-Isopropyl-3-methyl-1H-1,2-4-triazol-5-yl)-5,6-dihydrobenzo[f] imidazo[1,2-d][1,4]oxazepin-9-yl]-1H-pyrazol-1-yl}-2-methylpropanamide and salts thereof. In some instance, the PI3K inhibitor is taselisib (CAS #: 1282512-48-4). In some instances, the PI3K inhibitor is 2-amino-N-[2,3-dihydro-7-methoxy-8-[3-(4-morpholinyl) propoxy] imidazo[1,2-c] quinazolin-5-yl]-5-pyrimidinecarboxamide and salts thereof. In some instance, the PI3K inhibitor is copanlisib (CAS #: 1032568-63-0). In some instances, the PI3K inhibitor is 8-chloro-2-phenyl-3-[(1S)-1-(9H-purin-6-ylamino)ethyl]-1 (2H)-isoquinolinone and salts thereof. In some instance, the PI3K inhibitor is duvelisib (CAS #: 1201438-56-3). In some instances, the PI3K inhibitor is (2S)-N1-[4-methyl-5-[2-(2,2,2-trifluoro-1,1-dimethylethyl)-4-pyridinyl]-2-thiazolyl]-1,2-pyrrolidinedicarboxamide and salts thereof. In some instance, the PI3K inhibitor is alpelisib (CAS #: 1217486-61-7). In some instances, the PI3K inhibitor is 2-[(1S)-1-[4-amino-3-[3-fluoro-4-(1-methylethoxy)phenyl]-1H-pyrazolo[3,4-d]pyrimidin-1-yl] ethyl]-6-fluoro-3-(3-fluorophenyl)-4H-1-benzopyran-4-one and salts thereof. In some instance, the PI3K inhibitor is umbralisib (CAS #: 1532533-67-7).

The term “package insert” is used to refer to instructions customarily included in commercial packages of therapeutic products, that contain information about the indications, usage, dosage, administration, combination therapy, contraindications and/or warnings concerning the use of such therapeutic products.

### III. THERAPEUTIC METHODS

Provided herein are methods of treating a subject having a CD20-positive cell proliferative disorder (e.g., a B cell proliferative disorder, e.g., an NHL (e.g., a DLBCL or an FL) or a CLL) by subcutaneously administering to the subject mosunetuzumab in a dosing regimen comprising at least a first dosing cycle and a second dosing cycle, wherein the treatment comprising subcutaneous administration of mosunetuzumab as a monotherapy or in combination with lenalidomide exhibits non-inferiority or an improved outcome as measured using pharmacokinetics (PK), efficacy, safety, or a combination thereof, as compared to intravenous administration of mosunetuzumab as a monotherapy or in combination with lenalidomide, respectively.

A. Therapeutic Methods for Dosing of Mosunetuzumab in Combination with Lenalidomide

In one aspect, the invention features a method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

In some embodiments, the efficacy response is a CRR, and wherein the CRR or reference CRR is the proportion of the subjects receiving the corresponding treatment whose best overall response is a complete response (CR).

In some embodiments, the improved response in CRR is an increase in CRR compared to the reference CRR of between 1% and 25% (e.g., between 5% and 25%, between 10% and 25%, between 15% and 25%, between 20% and 25%, between 5% and 10%, between 5% and 15%, between 5% and 20%, between 10% and 20%, between 10% and 15%, or between 15% and 20%; e.g., about 1%, 5%, 10%, 15%, 20%, or 25%). In some embodiments, the efficacy response in CRR is non-inferior compared to the reference CRR.

In some embodiments, the efficacy response is an ORR, and wherein the ORR or reference ORR is the proportion of the subjects receiving the corresponding treatment whose best overall response is a CR or a partial response (PR). In some embodiments, the improved response in ORR is an increase in ORR compared to the reference ORR of between 1% and 24% (e.g., between 5% and 24%, between 10% and 24%, between 15% and 24%, between 20% and 24%, between 5% and 10%, between 5% and 15%, between 5% and 20%, between 10% and 20%, between 10% and 15%, or between 15% and 20%; e.g., about 1%, 5%, 10%, 15%, 20%, or 24%). In some embodiments, the efficacy response in ORR is non-inferior compared to the reference ORR.

In some embodiments, the efficacy response is a DOR, and wherein the DOR or the reference DOR is measured starting from the time from the first occurrence of a documented CR or PR to disease progression or relapse or death from any cause, whichever occurs first. In some embodiments, the DOR or the reference DOR is the median DOR of the plurality of subjects receiving the corresponding treatment. In some embodiments, the improvement of the median DOR is an increase in the DOR compared to the reference DOR of between 1 and 22 months (e.g., between 1 month and 5 months, between 5 months and 22 months, between 10 months and 22 months, between 15 months and 22 months, between 5 months and 10 months, between 5 months and 15 months, between 10 months and 22 months, between 10 months and 15 months, or between 15 months and 22 months; e.g., about 1 months, 5 months, 10 months, 15 months, 20 months, or 22 months). In some embodiments, the efficacy response in DOR is non-inferior compared to the reference DOR.

In some embodiments, the efficacy response is a DOCR, and wherein the DOCR or the reference DOCR is measured starting from the time from the first occurrence of a documented CR to disease progression or relapse or death from any cause, whichever occurs first. In some embodiments, the DOCR or the reference DOCR is the median DOCR of the plurality of subjects receiving the corresponding treatment. In some embodiments, the improvement of the median DOCR is an increase in the DOCR compared to the reference DOCR of between 1 and 22 months (e.g., between 1 month and 5 months, between 5 months and 22 months, between 10 months and 22 months, between 15 months and 22 months, between 5 months and 10 months, between 5 months and 15 months, between 10 months and 22 months, between 10 months and 15 months, or between 15 months and 22 months; e.g., about 1 months, 5 months, 10 months, 15 months, 20 months, or 22 months). In some embodiments, the efficacy response in DOCR is non-inferior compared to the reference DOCR.

In some embodiments, the CR or PR is determined by PET/computed tomography (CT). In some embodiments, the CR or PR is determined based on the Lugano Response Criteria for Malignant Lymphoma (Cheson et al., 2014).

In some embodiments, the efficacy response is an OS, wherein the OS or the reference OS is measured starting from the time from first treatment (e.g., first dose of mosunetuzumab or first dose of lenalidomide; e.g., Day 1 of dosing cycle 1) to death from any cause. In some embodiments, the OS or the reference OS is the median OS of the plurality of subjects receiving the corresponding treatment. In some embodiments, the improvement of the median OS is an increase in the OS compared to the reference OS of between 1 and 28 months (e.g., between 1 month and 5 months, between 5 months and 28 months, between 10 months and 28 months, between 15 months and 28 months, between 20 and 28 months, between 25 and 28 months, between 5 months and 10 months, between 5 months and 15 months, between 5 months and 20 months, between 5 months and 25 months, between 10 months and 20 months, between 10 months and 25 months, between 10 months and 15 months, between 15 months and 25 months, or between 20 months and 28 months; e.g., about 1 months, 5 months, 10 months, 15 months, 20 months, 25 months, or 28 months). In some embodiments, the efficacy response in OS is non-inferior compared to the reference OS.

In some embodiments, the efficacy response is a PFS, and wherein the PFS or the reference PFS is measured starting from the time from first treatment (e.g., first dose of mosunetuzumab or first dose of lenalidomide; e.g., Day 1 of dosing cycle 1) to the time of a first occurrence of disease progression or death from any cause. In some embodiments, the PFS or the reference PFS is the median PFS of the plurality of subjects receiving the corresponding treatment. In some embodiments, the improvement of the median PFS is an increase in the PFS compared to the reference PFS of between 1 and 26 months (e.g., between 1 month and 5 months, between 5 months and 26 months, between 10 months and 26 months, between 15 months and 26 months, between 5 months and 10 months, between 5 months and 15 months, between 10 months and 26 months, between 10 months and 15 months, between 15 months and 20 months, or between 20 months and 26 months; e.g., about 1 months, 5 months, 10 months, 15 months, 20 months, or 26 months). In some embodiments, the efficacy response in PFS is non-inferior compared to the reference PFS.

In another aspect, the invention features a method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

In some embodiments, the safety response is the rate of AE, and the rate of AE or the reference rate of AE is the rate of AE in the plurality of subjects receiving the corresponding treatment. In some embodiments, the safety response in rate of AE is non-inferior compared to the reference rate of AE. In some embodiments, the safety response is the rate of CRS, and wherein the rate of CRS or the reference rate of CRS is the rate of CRS in the plurality of subjects receiving the corresponding treatment. In some embodiments, the improved rate of CRS is a decrease in the rate of CRS compared to the reference rate of CRS of between 7% and 17% (e.g., between 7% and 12%, between 12% and 17%, or between 10% and 14%; e.g., about 8%, 9%, 10%, 11%, 12%, 13%, 14%, 15%, 16%, or 17%). In some embodiments, the improved rate of CRS is a decrease in the rate of CRS compared to the reference rate of CRS of about 12%. In some embodiments, the rate of CRS is the rate of Grade 1 or 2 CRS in the plurality of patients receiving the corresponding treatment. In some embodiments, the rate of Grade 3+

CRS is non-inferior compared to the reference rate of Grade 3+ CRS. In some embodiments, the Grade of CRS is determined based on the American Society of Transplantation and Cellular Therapy (ASTCT) Consensus Grading for Cytokine-Release Syndrome (Lee et al., 2019).

In some embodiments, the safety response is the rate of SAE, and wherein the rate of SAE or the reference rate of SAE is the rate of SAE in the plurality of subjects receiving the corresponding treatment. In some embodiments, the safety response in rate of SAE is non-inferior compared to the reference rate of SAE.

In another aspect, the invention features a method for treating follicular lymphoma (FL) in a subject in need thereof, comprising administering to the subject a combination treatment comprising subcutaneously administered mosunetuzumab and orally administered lenalidomide according to a dosing regimen comprising a first 21-day dosing cycle and three 28-day dosing cycles, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

In some embodiments, one or both of the scAUC₀₋₇₇ and the ivAUC₀₋₇₇ is determined using population PK (popPK) model-predicted individual Empirical Bayesian Estimates (EBEs).

In some embodiments, the combination treatment comprises subcutaneously administering mosunetuzumab and orally administering lenalidomide according to a dosing regimen comprising at least a first dosing cycle and a second dosing cycle, wherein the first dosing cycle is a 21-day dosing cycle and the second dosing cycle is a 28-day dosing cycle, and wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg±0.5 mg), the scC1D2 is about 45 mg (e.g., 45 mg±10%; 45 mg±4.5 mg), and the scC1D3 is about 45 mg (e.g., 45 mg±10%; 45 mg±4.5 mg); and (b) the second dosing cycle comprises: (i) a first subcutaneous dose (scC2D1) of mosunetuzumab administered on Day 1 of the second dosing cycle, wherein the scC2D1 is about 45 mg (e.g., 45 mg±10%; 45 mg±4.5 mg); and (ii) twenty-one (21) oral doses of lenalidomide administered on Days 1-21 of the second dosing cycle, wherein each oral dose of lenalidomide is about 20 mg (e.g., 20 mg±10%; 20 mg±2 mg). In particular embodiments, the combination treatment comprises subcutaneously administering mosunetuzumab and orally administering lenalidomide according to a dosing regimen comprising at least a first dosing cycle and a second dosing cycle, wherein the first dosing cycle is a 21-day dosing cycle and the second dosing cycle is a 28-day dosing cycle, and wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is 5, the scC1D2 is 45 mg, and the scC1D3 is 45 mg; and (b) the second dosing cycle comprises: (i) a first subcutaneous dose (scC2D1) of mosunetuzumab administered on Day 1 of the second dosing cycle, wherein the scC2D1 is 45 mg; and (ii) twenty-one (21) oral doses of lenalidomide administered on Days 1-21 of the second dosing cycle, wherein each oral dose of lenalidomide is 20 mg.

In some embodiments, the dosing regimen of the combination treatment further comprises one or more additional 28-day dosing cycles. In some embodiments, the dosing regimen of the combination treatment comprises ten additional 28-day dosing cycles.

In some embodiments, each additional dosing cycle comprises: (a) a subcutaneous dose of mosunetuzumab administered on Day 1 of the additional dosing cycle, wherein the subcutaneous dose of mosunetuzumab is about 45 mg (e.g., 45 mg±10%; 45 mg±4.5 mg); and (b) twenty-one (21) oral doses of lenalidomide administered on Days 1-21 of the additional dosing cycle, wherein each oral dose of lenalidomide is about 20 mg (e.g., 20 mg±10%; 20 mg±2 mg). In particular embodiments, each additional dosing cycle comprises: (a) a subcutaneous dose of mosunetuzumab administered on Day 1 of the additional dosing cycle, wherein the subcutaneous dose of mosunetuzumab is 45 mg; and (b) twenty-one (21) oral doses of lenalidomide administered on Days 1-21 of the additional dosing cycle, wherein each oral dose of lenalidomide is 20 mg.

In some embodiments, the dosing regimen of the combination treatment further comprises one to nine (e.g., one, two, three, four, five, six, seven, eight, or nine) subcutaneous maintenance doses of mosunetuzumab.

In another aspect, the invention features a method for treating follicular lymphoma (FL) in a subject in need thereof, comprising administering to the subject a combination treatment comprising subcutaneously administered mosunetuzumab and orally administered lenalidomide, wherein the combination treatment comprises subcutaneously administering mosunetuzumab and orally administering lenalidomide according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine (e.g., one, two, three, four, five, six, seven, eight, or nine) subcutaneous maintenance doses of mosunetuzumab, wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg±0.5 mg), the scC1D2 is about 45 mg (e.g., 45 mg±10%; 45 mg±4.5 mg), and the scC1D3 is about 45 mg (e.g., 45 mg±10%; 45 mg±4.5 mg); (b) the second dosing cycle comprises: (i) a first subcutaneous dose (scC2D1) of mosunetuzumab administered on Day 1 of the second dosing cycle, wherein the scC2D1 is about 45 mg (e.g., 45 mg±10%; 45 mg±4.5 mg); and (ii) twenty-one (21) oral doses of lenalidomide administered on Days 1-21 of the second dosing cycle, wherein each oral dose of lenalidomide is about 20 mg (e.g., 20 mg±10%; 20 mg±2 mg); and (c) the ten additional dosing cycles each comprises: (i) a first subcutaneous dose (scC3D1-scC12D1) of mosunetuzumab administered on Day 1 of each additional dosing cycle, wherein the scC3D1-scC12D1 are each about 45 mg (e.g., 45 mg±10%; 45 mg±4.5 mg); and (ii) twenty-one (21) oral doses of lenalidomide administered on Days 1-21 of each additional dosing cycle, wherein each oral dose of lenalidomide is about 20 mg (e.g., 20 mg±10%; 20 mg±2 mg).

In some embodiments, the dosing regimen of the combination treatment further comprises nine subcutaneous maintenance doses of mosunetuzumab.

In some embodiments, the one to nine (e.g., one, two, three, four, five, six, seven, eight, or nine) subcutaneous maintenance doses of mosunetuzumab are administered every eight weeks starting eight weeks after Day 1 of the tenth additional 28-day dosing cycle. In some embodiments, each maintenance dose is about 45 mg (e.g., 45 mg±10%; 45 mg±4.5 mg). In particular embodiments, each maintenance dose is 45 mg.

In some embodiments, the control treatment comprises intravenously administering mosunetuzumab and orally administering lenalidomide according to a dosing regimen comprising at least a first 21-day dosing cycle and a second 28-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg (e.g., 1 mg±10%), the ivC1D2 is about 2 mg (e.g., 2 mg±10%), and the ivC1D3 is about 30 mg (e.g., 30 mg±10%); and (b) the second dosing cycle comprises: (i) a first intravenous dose (ivC2D1) of mosunetuzumab administered on Day 1 of the second dosing cycle, wherein the ivC2D1 is about 30 mg (e.g., 30 mg±10%); and (ii) 21 oral doses of lenalidomide administered on Days 1-21 of the second dosing cycle, wherein each oral dose of lenalidomide is about 20 mg (e.g., 20 mg±10%; 20 mg±2 mg). In particular embodiments, the control treatment comprises intravenously administering mosunetuzumab and orally administering lenalidomide according to a dosing regimen comprising at least a first 21-day dosing cycle and a second 28-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is 1 mg, the ivC1D2 is 2 mg, and the ivC1D3 is 30 mg; and (b) the second dosing cycle comprises: (i) a first intravenous dose (ivC2D1) of mosunetuzumab administered on Day 1 of the second dosing cycle, wherein the ivC2D1 is 30 mg; and (ii) 21 oral doses of lenalidomide administered on Days 1-21 of the second dosing cycle, wherein each oral dose of lenalidomide is 20 mg. In some embodiments, the dosing regimen of the control treatment further comprises one or more additional 28-day dosing cycles. In some embodiments, the dosing regimen of the control treatment comprises ten additional 28-day dosing cycles. In some embodiments, each additional dosing cycle comprises: (a) an intravenous dose of mosunetuzumab administered on Day 1 of the additional dosing cycle, wherein the intravenous dose of mosunetuzumab is about 30 mg (e.g., 30 mg±10%); and (b) twenty-one (21) oral doses of lenalidomide administered on Days 1-21 of the additional dosing cycle, wherein each oral dose of lenalidomide is about 20 mg (e.g., 20 mg±10%; 20 mg±2 mg). In particular embodiments, each additional dosing cycle comprises: (a) an intravenous dose of mosunetuzumab administered on Day 1 of the additional dosing cycle, wherein the intravenous dose of mosunetuzumab is 30 mg; and (b) twenty-one (21) oral doses of lenalidomide administered on Days 1-21 of the additional dosing cycle, wherein each oral dose of lenalidomide is 20 mg.

In one aspect, the invention features a method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS), wherein the combination treatment comprises subcutaneously administering mosunetuzumab and orally administering lenalidomide according to a dosing regimen comprising at least a first dosing cycle and a second dosing cycle, wherein the first dosing cycle is a 21-day dosing cycle and the second dosing cycle is a 28-day dosing cycle, and wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg±0.5 mg), the scC1D2 is about 45 mg (e.g., 45 mg±10%; 45 mg±4.5 mg), and the scC1D3 is about 45 mg (e.g., 45 mg±10%; 45 mg±4.5 mg); and (b) the second dosing cycle comprises: (i) a first subcutaneous dose (scC2D1) of mosunetuzumab administered on Day 1 of the second dosing cycle, wherein the scC2D1 is about 45 mg (e.g., 45 mg±10%; 45 mg±4.5 mg); and (ii) twenty-one (21) oral doses of lenalidomide administered on Days 1-21 of the second dosing cycle, wherein each oral dose of lenalidomide is about 20 mg (e.g., 20 mg±10%; 20 mg±2 mg) and/or the control treatment comprises intravenously administering mosunetuzumab and orally administering lenalidomide according to a dosing regimen comprising at least a first 21-day dosing cycle and a second 28-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg (e.g., 1 mg±10%), the ivC1D2 is about 2 mg (e.g., 2 mg±10%), and the ivC1D3 is about 30 mg (e.g., 30 mg±10%); and (b) the second dosing cycle comprises: (i) a first intravenous dose (ivC2D1) of mosunetuzumab administered on Day 1 of the second dosing cycle, wherein the ivC2D1 is about 30 mg (e.g., 30 mg±10%); and (ii) 21 oral doses of lenalidomide administered on Days 1-21 of the second dosing cycle, wherein each oral dose of lenalidomide is about 20 mg (e.g., 20 mg±10%; 20 mg±2 mg).

In another aspect, the invention features a method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE), wherein the combination treatment comprises subcutaneously administering mosunetuzumab and orally administering lenalidomide according to a dosing regimen comprising at least a first dosing cycle and a second dosing cycle, wherein the first dosing cycle is a 21-day dosing cycle and the second dosing cycle is a 28-day dosing cycle, and wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg±0.5 mg), the scC1D2 is about 45 mg (e.g., 45 mg±10%; 45 mg±4.5 mg), and the scC1D3 is about 45 mg (e.g., 45 mg±10%; 45 mg±4.5 mg); and (b) the second dosing cycle comprises: (i) a first subcutaneous dose (scC2D1) of mosunetuzumab administered on Day 1 of the second dosing cycle, wherein the scC2D1 is about 45 mg (e.g., 45 mg±10%; 45 mg±4.5 mg); and (ii) twenty-one (21) oral doses of lenalidomide administered on Days 1-21 of the second dosing cycle, wherein each oral dose of lenalidomide is about 20 mg (e.g., 20 mg±10%; 20 mg±2 mg) and/or the control treatment comprises intravenously administering mosunetuzumab and orally administering lenalidomide according to a dosing regimen comprising at least a first 21-day dosing cycle and a second 28-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg (e.g., 1 mg±10%), the ivC1D2 is about 2 mg (e.g., 2 mg±10%), and the ivC1D3 is about 30 mg (e.g., 30 mg±10%); and (b) the second dosing cycle comprises: (i) a first intravenous dose (ivC2D1) of mosunetuzumab administered on Day 1 of the second dosing cycle, wherein the ivC2D1 is about 30 mg (e.g., 30 mg±10%); and (ii) 21 oral doses of lenalidomide administered on Days 1-21 of the second dosing cycle, wherein each oral dose of lenalidomide is about 20 mg (e.g., 20 mg±10%; 20 mg±2 mg).

In another aspect, the invention features a method for treating follicular lymphoma (FL) in a subject in need thereof, comprising administering to the subject a combination treatment comprising subcutaneously administered mosunetuzumab and orally administered lenalidomide according to a dosing regimen comprising a first 21-day dosing cycle and three 28-day dosing cycles, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇), wherein the combination treatment comprises subcutaneously administering mosunetuzumab and orally administering lenalidomide according to a dosing regimen comprising at least a first dosing cycle and a second dosing cycle, wherein the first dosing cycle is a 21-day dosing cycle and the second dosing cycle is a 28-day dosing cycle, and wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg+0.5 mg), the scC1D2 is about 45 mg (e.g., 45 mg±10%; 45 mg±4.5 mg), and the scC1D3 is about 45 mg (e.g., 45 mg±10%; 45 mg±4.5 mg); and (b) the second dosing cycle comprises: (i) a first subcutaneous dose (scC2D1) of mosunetuzumab administered on Day 1 of the second dosing cycle, wherein the scC2D1 is about 45 mg (e.g., 45 mg±10%; 45 mg±4.5 mg); and (ii) twenty-one (21) oral doses of lenalidomide administered on Days 1-21 of the second dosing cycle, wherein each oral dose of lenalidomide is about 20 mg (e.g., 20 mg±10%; 20 mg±2 mg) and/or the control treatment comprises intravenously administering mosunetuzumab and orally administering lenalidomide according to a dosing regimen comprising at least a first 21-day dosing cycle and a second 28-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg (e.g., 1 mg±10%), the ivC1D2 is about 2 mg (e.g., 2 mg±10%), and the ivC1D3 is about 30 mg (e.g., 30 mg±10%); and (b) the second dosing cycle comprises: (i) a first intravenous dose (ivC2D1) of mosunetuzumab administered on Day 1 of the second dosing cycle, wherein the ivC2D1 is about 30 mg (e.g., 30 mg±10%); and (ii) 21 oral doses of lenalidomide administered on Days 1-21 of the second dosing cycle, wherein each oral dose of lenalidomide is about 20 mg (e.g., 20 mg±10%; 20 mg±2 mg).

In some embodiments, the dosing regimen of the combination treatment further comprises one or more additional 28-day dosing cycles. In some embodiments, the dosing regimen of the combination treatment comprises ten additional 28-day dosing cycles.

In some embodiments, each additional dosing cycle comprises: (a) a subcutaneous dose of mosunetuzumab administered on Day 1 of the additional dosing cycle, wherein the subcutaneous dose of mosunetuzumab is about 45 mg (e.g., 45 mg±10%; 45 mg±4.5 mg); and (b) twenty-one (21) oral doses of lenalidomide administered on Days 1-21 of the additional dosing cycle, wherein each oral dose of lenalidomide is about 20 mg (e.g., 20 mg±10%; 20 mg±2 mg).

In some embodiments, the dosing regimen of the combination treatment further comprises one to nine (e.g., one, two, three, four, five, six, seven, eight, or nine) subcutaneous maintenance doses of mosunetuzumab. In some embodiments, the one to nine (e.g., one, two, three, four, five, six, seven, eight, or nine) subcutaneous maintenance doses of mosunetuzumab are administered every eight weeks starting eight weeks after Day 1 of the tenth additional 28-day dosing cycle. In some embodiments, each maintenance dose is about 45 mg (e.g., 45 mg±10%; 45 mg±4.5 mg).

In some embodiments, the dosing regimen of the control treatment further comprises one or more additional 28-day dosing cycles. In some embodiments, the dosing regimen of the control treatment comprises ten additional 28-day dosing cycles. In some embodiments, each additional dosing cycle comprises: (a) an intravenous dose of mosunetuzumab administered on Day 1 of the additional dosing cycle, wherein the intravenous dose of mosunetuzumab is about 30 mg (e.g., 30 mg±10%); and (b) twenty-one (21) oral doses of lenalidomide administered on Days 1-21 of the additional dosing cycle, wherein each oral dose of lenalidomide is about 20 mg (e.g., 20 mg±10%; 20 mg±2 mg). In some embodiments, the subject has an Eastern Cooperative Oncology Group (ECOG) Performance Status of 0, 1, or 2. In some embodiments, the subject has not been previously treated for FL. In some embodiments, the subject has relapsed after or is refractory to one or more prior lines of systemic therapy. In some embodiments, the one or more prior lines of systemic therapy comprises an immunotherapy or a chemoimmunotherapy. In some embodiments, the one or more prior lines of systemic therapy comprises an anti-CD20 antibody. In some embodiments, the anti-CD20 antibody is rituximab.

In some embodiments, the FL is Grade 1, 2, or 3a.

In some embodiments, the method comprises administering an additional therapeutic agent. In some embodiments, the additional therapeutic agent comprises a corticosteroid, an antihistamine, an antipyretic, or an IL-6R antagonist. In some embodiments, the additional therapeutic agent is a corticosteroid, and wherein the corticosteroid comprises prednisone, methylprednisolone, or dexamethasone. In some embodiments, the additional therapeutic agent is an antihistamine, and wherein the antihistamine comprises diphenhydramine hydrochloride or equivalent. In some embodiments, the additional therapeutic agent is an antipyretic, and wherein the antipyretic is acetaminophen. In some embodiments, the additional therapeutic agent is an IL-6R antagonist, and wherein the IL-6R antagonist is tocilizumab. In some embodiments, the additional therapeutic agent is administered in accordance with the methods described below in Part C (Dosing Strategies for Mitigation of Adverse Events (e.g., Cytokine Release Syndrome).

In one aspect, the invention features use of mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered response rate (ORR), a duration of response (DOR), a duration of complete response (DOCR), progression free survival (PFS), or overall survival (OS).

In one aspect, the invention features use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).

In one aspect, the invention features mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).

In one aspect, the invention features use of mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In one aspect, the invention features use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In one aspect, the invention features mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects r results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In one aspect, the invention features use of mosunetuzumab for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In one aspect, the invention features use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In one aspect, the invention features mosunetuzumab for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In some embodiments, administering the combination treatment comprising subcutaneously administered mosunetuzumab and orally administered lenalidomide to a plurality of subjects having FL results in an ORR in the plurality of subjects of between about 81% to about 99% (e.g., 81%, 82%, 83%, 84%, 85%, 86%, 87%, 88%, 89%, 90%, 91%, 92%, 93%, 94%, 95%, 96%, 97%, 98%, or 99%). In some embodiments, the ORR is about 90%. In some embodiments, administering the combination treatment to a plurality of subjects having FL results in a CRR in the plurality of subjects of between about 79% to about 97% (e.g., 79%, 80%, 81%, 82%, 83%, 84%, 85%, 86%, 87%, 88%, 89%, 90%, 91%, 92%, 93%, 94%, 95%, 96%, or 97%). In some embodiments, the CRR is about 88%. In some embodiments, administering the combination treatment to a plurality of subjects having FL results in a 12-month event-free rate for DOR in the plurality of subjects of between about 86% to about 100% (e.g., 86%, 87%, 88%, 89%, 90%, 91%, 92%, 93%, 94%, 95%, 96%, 97%, 98%, 99%, or 100%). In some embodiments, the 12-month event-free rate for DOR is about 94%. In some embodiments, administering the combination treatment to a plurality of subjects having FL results in a 12-month event-free rate for DOCR in the plurality of subjects of between about 86% to about 100% (e.g., 86%, 87%, 88%, 89%, 90%, 91%, 92%, 93%, 94%, 95%, 96%, 97%, 98%, 99%, or 100%). In some embodiments, the 12-month event-free rate for DOCR is about 94%. In some embodiments, administering the combination treatment to a plurality of subjects having FL results in a 12-month event-free rate for PFS in the plurality of subjects of between about 76% to about 98% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98%). In some embodiments, the 12-month event-free rate for PFS is about 87%. In some embodiments, administering the combination treatment to a plurality of subjects having FL results in a 12-month event-free rate for OS in the plurality of subjects of between about 92% to about 100% (e.g., 92%, 93%, 94%, 95%, 96%, 97%, 98%, 99%, or 100%). In some embodiments, the 12-month event-free rate for OS is about 97%. In some embodiments, the FL is previously untreated (1 L) FL.

### B. Therapeutic Methods for Dosing of Mosunetuzumab Monotherapy

In one aspect, the invention features a method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).

In some embodiments, the efficacy response is a CRR, and wherein the CRR or reference CRR is the proportion of the subjects receiving the corresponding treatment whose best overall response is a complete response (CR). In some embodiments, the improved response in CRR is an increase in CRR compared to the reference CRR of between 1% and 20% (e.g., between 5% and 20%, between 10% and 20%, between 15% and 20%, between 1% and 15%, between 1% and 10%, between 1% and 5%, between 5% and 15%, or between 10% and 20%; e.g., about 1%, about 5%, about 10%, about 15%, or about 20%). In some embodiments, the efficacy response in CRR is non-inferior compared to the reference rate CRR.

In some embodiments, the efficacy response is an ORR, and wherein the ORR or reference ORR is the proportion of the subjects receiving the corresponding treatment whose best overall response is a CR or a partial response (PR). In some embodiments, the improved response in ORR is an increase in ORR compared to the reference ORR of between 1% and 13% (e.g., between 1% and 5%, between 5% and 13%, or between 3% and 10%; e.g., about 1%, about 5%, about 10%, or about 13%). In some embodiments, the efficacy response in ORR is non-inferior compared to the reference ORR.

In some embodiments, the efficacy response is a DOR, and wherein the DOR or the reference DOR is measured starting from the time from the first occurrence of a documented CR or PR to disease progression or relapse or death from any cause, whichever occurs first. In some embodiments, the DOR or the reference DOR is the median DOR of the plurality of subjects receiving the corresponding treatment. In some embodiments, the improvement of the median DOR is an increase in the DOR compared to the reference DOR of between 1 and 13 months (e.g., between 1 and 5 months, between 5 and 13 months, or between 3 and 10 months; e.g., about 1 month, 5 months, 10 months, or 13 months). In some embodiments, the efficacy response in DOR is non-inferior compared to reference DOR. In some embodiments, the improvement of the DOR is an increase in the rate of DOR at 12 months compared to the rate of reference DOR at 12 months of between 1% and 31% (e.g., between 1% and 25%, between 1% and 20%, between 1% and 15%, between 1% and 10%, between 1% and 5%, between 5% and 10%, between 5% and 15%, between 5% and 20%, between 5% and 25%, between 5% and 31%, between 1% and 10%, between 10% and 20%, between 20% and 31%, between 15% and 31%, between 1% and 20%, or between 10% and 31%; e.g., about 1%, 5%, 10%, 15%, 20%, 25%, or 31%). In some embodiments, the improvement of the DOR is an increase in the rate of DOR at 12 months compared to the rate of reference DOR at 12 months of about 8%.

In some embodiments, the efficacy response is a DOCR, and wherein the DOCR or the reference DOCR is measured starting from the time from the first occurrence of a documented CR to disease progression or relapse or death from any cause, whichever occurs first. In some embodiments, the DOCR or the reference DOCR is the median DOCR of the plurality of subjects receiving the corresponding treatment. In some embodiments, the improvement of the DOCR is an increase in the rate of DOCR at 12 months compared to the rate of reference DOCR at 12 months of between 1% and 27% (e.g., between 1% and 25%, between 1% and 20%, between 1% and 15%, between 1% and 10%, between 1% and 5%, between 5% and 10%, between 5% and 15%, between 5% and 20%, between 5% and 25%, between 5% and 27%, between 1% and 10%, between 10% and 20%, between 20% and 27%, between 15% and 27%, between 1% and 20%, or between 10% and 27%; e.g., about 1%, 5%, 10%, 15%, 20%, 25%, or 27%). In some embodiments, the efficacy response in DOCR is non-inferior compared to the reference DOCR.

In some embodiments, the CR or PR is determined by PET/computed tomography (CT). In some embodiments, the CR or PR is determined based on the Revised Response Criteria for Malignant Lymphoma (Cheson et al., 2007).

In some embodiments, the efficacy response is a PFS, and wherein the PFS or the reference PFS is measured starting from the time from first treatment (e.g., first dose of mosunetuzumab or first dose of lenalidomide; e.g., Day 1 of dosing cycle 1) to the time of a first occurrence of disease progression or death from any cause. In some embodiments, the PFS or the reference PFS is the median PFS of the plurality of subjects receiving the corresponding treatment. In some embodiments, the improvement of the median PFS is an increase in the PFS compared to the reference PFS of between 1 and 14 months (e.g., between 1 and 5 months, between 5 and 14 months, or between 3 and 10 months; e.g., about 1 month, 5 months, 10 months, or 14 months). In some embodiments, the efficacy response in PFS is non-inferior compared to the reference PFS. In some embodiments, the improvement of the PFS is in an increase in the rate of PFS compared to the rate of reference PFS at 12 months of between 1% and 26% (e.g., between 1% and 20%, between 1% and 15%, between 1% and 10%, between 1% and 5%, between 5% and 10%, between 5% and 15%, between 5% and 20%, between 5% and 26%, between 1% and 10%, between 10% and 20%, between 20% and 26%, between 15% and 26%, between 1% and 20%, or between 10% and 26%; e.g., about 1%, 5%, 10%, 15%, 20%, or 26%). In some embodiments, the improvement of the PFS is in an increase in the rate of PFS compared to the rate of reference PFS at 12 months of about 5%.

In some embodiments, the efficacy response is an OS, wherein the OS or the reference OS is measured starting from the time from first treatment (e.g., first dose of mosunetuzumab or first dose of lenalidomide; e.g., Day 1 of dosing cycle 1) to death from any cause. In some embodiments, the improvement of the OS is an increase in the rate of OS compared to the rate of reference OS at 12 months of between 1% and 9% (e.g., about 1%, 2%, 3%, 4%, 5%, 6%, 7%, 8%, or 9%). In some embodiments, the efficacy response in OS is non-inferior compared to the reference OS.

In another aspect, the invention features a method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In some embodiments, the safety response is the rate of AE, and wherein the rate of AE or the reference rate of AE is the rate of AE in the plurality of subjects receiving the corresponding treatment. In some embodiments, the safety response in rate of AE is non-inferior compared to the reference rate of AE. In some embodiments, the improved rate of AE is a decrease in the rate of Grade 3 or 4 AE compared to the reference rate of Grade 3 or 4 AE of between 11% and 21% (e.g., between 11% and 16%, between 16% and 21%, or between 13% to 19%; e.g., about 11%, about 16%, or about 21%). In some embodiments, the improved rate of AE is a decrease in the rate of Grade 3 or 4 AE compared to the reference rate of Grade 3 or 4 AE of about 16%. In some embodiments, the rate of AE is the rate of

Grade 5 AE in the plurality of subjects receiving the corresponding treatment, and wherein the rate of AE is non-inferior compared to the reference rate of AE. In some embodiments, the Grade of AE is determined based on the National Cancer Institute Common Terminology Criteria for Adverse Events (NCI CTCAE), v4.0.

In some embodiments, the safety response is the rate of SAE, and wherein the rate of SAE or the reference rate of SAE is the rate of SAE in the plurality of subjects receiving the corresponding treatment. In some embodiments, the improved safety response in rate of SAE is a decrease in the rate of SAE compared to the reference rate of SAE of between 9% and 19% (e.g., between 9% and 14%, between 14% and 19%, or between 11% and 17%; e.g., about 9%, about 14%, or about 19%). In some embodiments, the improved safety response in rate of SAE is a decrease in the rate of SAE compared to the reference rate of SAE of about 14%.

In some embodiments, the safety response is the rate of CRS, and wherein the rate of CRS or the reference rate of CRS is the rate of CRS in the plurality of subjects receiving the corresponding treatment. In some embodiments, the improved rate of CRS is a decrease in the rate of CRS compared to the reference rate of CRS of between 9% and 19% (e.g., between 9% and 14%, between 14% and 19%, or between 11% and 17%; e.g., about 9%, about 14%, or about 19%). In some embodiments, the improved rate of CRS is a decrease in the rate of CRS compared to the reference rate of CRS of about 14%. In some embodiments, the improved rate of CRS is a decrease in the rate of Grade 2+ CRS compared to the reference rate of Grade 2+ CRS of between 3% and 13% (e.g., between 3% and 8%, between 8% and 13%, or between 5% and 11%; e.g., about 3%, about 8%, or about 13%). In some embodiments, the improved rate of CRS is a decrease in the rate of Grade 2+ CRS compared to the reference rate of Grade 2+ CRS of about 8%. In some embodiments, the Grade of CRS is determined based on the Modified Cytokine Release Syndrome Grading System (Lee et al., 2014).

In another aspect, the invention features a method for treating Non-Hodgkin Lymphoma (NHL) or chronic lymphocytic leukemia (CLL) in a subject in need thereof, comprising administering to the subject a treatment comprising subcutaneously administered mosunetuzumab according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In some embodiments, one or more of the scAUC₀₋₈₄ or the ivAUC₀₋₈₄ is determined using population PK (popPK) model-predicted individual Empirical Bayesian Estimates (EBEs).

In some embodiments, the treatment comprises subcutaneously administering mosunetuzumab according to a dosing regimen comprising at least a first 21-day dosing cycle and a second 21-day dosing cycle, wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg±0.5 mg), the scC1D2 is about 15 mg (e.g., 15 mg±10%; 15 mg±1.5 mg) or about 45 mg (e.g., 45 mg±10%; 45 mg±4.5 mg), and the scC1D3 is about 45 mg (e.g., 45 mg±10%; 45 mg±4.5 mg); and (b) the second dosing cycle comprises a first subcutaneous dose (scC2D1) of mosunetuzumab administered on Day 1 of the second dosing cycle, wherein the scC2D1 is about 45 mg (e.g., 45 mg±10%; 45 mg±4.5 mg). In particular embodiments, the scC1D2 is about 45 mg (e.g., 45 mg±10%; 45 mg±4.5 mg). In particular embodiments, the treatment comprises subcutaneously administering mosunetuzumab according to a dosing regimen comprising at least a first 21-day dosing cycle and a second 21-day dosing cycle, wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is 5 mg, the scC1D2 is 15 mg or 45 mg, and the scC1D3 is 45 mg; and (b) the second dosing cycle comprises a first subcutaneous dose (scC2D1) of mosunetuzumab administered on Day 1 of the second dosing cycle, wherein the scC2D1 is 45 mg. In particular embodiments, the scC1D2 is 45 mg.

In some embodiments, the dosing regimen of the treatment further comprises one or more additional 21-day dosing cycles. In some embodiments, the dosing regimen of the treatment comprises six, fifteen, or twenty-three additional 21-day dosing cycles. In some embodiments, each additional dosing cycle comprises a subcutaneous dose of mosunetuzumab administered on Day 1 of the additional dosing cycle, wherein the subcutaneous dose of mosunetuzumab is about 45 mg (e.g., 45 mg±10%; 45 mg±4.5 mg). In particular embodiments, each additional dosing cycle comprises a subcutaneous dose of mosunetuzumab administered on Day 1 of the additional dosing cycle, wherein the subcutaneous dose of mosunetuzumab is 45 mg.

In some embodiments, the control treatment comprises intravenously administering mosunetuzumab according to a dosing regimen comprising at least a first 21-day dosing cycle and a second 21-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg (e.g., 1 mg±10%), the ivC1D2 is about 2 mg (e.g., 2 mg±10%), and the ivC1D3 is about 60 mg (e.g., 60 mg±10%); and (b) the second dosing cycle comprises a first intravenous dose (ivC2D1) of mosunetuzumab administered on Day 1 of the second dosing cycle, wherein the ivC2D1 is about 60 mg (e.g., 60 mg±10%). In particular embodiments, the control treatment comprises intravenously administering mosunetuzumab according to a dosing regimen comprising at least a first 21-day dosing cycle and a second 21-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is 1 mg, the ivC1D2 is 2 mg, and the ivC1D3 is 60 mg; and (b) the second dosing cycle comprises a first intravenous dose (ivC2D1) of mosunetuzumab administered on Day 1 of the second dosing cycle, wherein the ivC2D1 is 60 mg. In some embodiments, the dosing regimen of the control treatment further comprises one or more additional 21-day dosing cycles. In some embodiments, the dosing regimen of the control treatment comprises six, fifteen, or twenty-three additional 21-day dosing cycles. In some embodiments, each additional dosing cycle comprises an intravenous dose of mosunetuzumab administered on Day 1 of the additional dosing cycle, wherein the intravenous dose of mosunetuzumab is about 30 mg (e.g., 30 mg±10%). In particular embodiments, each additional dosing cycle comprises an intravenous dose of mosunetuzumab administered on Day 1 of the additional dosing cycle, wherein the intravenous dose of mosunetuzumab is about 30 mg.

In one aspect, the invention features a method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS), wherein the treatment comprises subcutaneously administering mosunetuzumab according to a dosing regimen comprising at least a first 21-day dosing cycle and a second 21-day dosing cycle, wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg±0.5 mg), the scC1D2 is about 15 mg (e.g., 15 mg±10%; 15 mg±1.5 mg) or about 45 mg (e.g., 45 mg±10%; 45 mg±4.5 mg), and the scC1D3 is about 45 mg (e.g., 45 mg±10%; 45 mg±4.5 mg); and (b) the second dosing cycle comprises a first subcutaneous dose (scC2D1) of mosunetuzumab administered on Day 1 of the second dosing cycle, wherein the scC2D1 is about 45 mg (e.g., 45 mg±10%; 45 mg±4.5 mg), and/or the control treatment comprises intravenously administering mosunetuzumab according to a dosing regimen comprising at least a first 21-day dosing cycle and a second 21-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg (e.g., 1 mg±10%), the ivC1D2 is about 2 mg (e.g., 2 mg±10%), and the ivC1D3 is about 60 mg (e.g., 60 mg±10%); and (b) the second dosing cycle comprises a first intravenous dose (ivC2D1) of mosunetuzumab administered on Day 1 of the second dosing cycle, wherein the ivC2D1 is about 60 mg (e.g., 60 mg±10%). In particular embodiments, the scC1D2 is about 45 mg (e.g., 45 mg±10%; 45 mg±4.5 mg).

In another aspect, the invention features a method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE), wherein the treatment comprises subcutaneously administering mosunetuzumab according to a dosing regimen comprising at least a first 21-day dosing cycle and a second 21-day dosing cycle, wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg±0.5 mg), the scC1D2 is about 15 mg (e.g., 15 mg±10%; 15 mg±1.5 mg) or about 45 mg (e.g., 45 mg±10%; 45 mg±4.5 mg), and the scC1D3 is about 45 mg (e.g., 45 mg±10%; 45 mg±4.5 mg); and (b) the second dosing cycle comprises a first subcutaneous dose (scC2D1) of mosunetuzumab administered on Day 1 of the second dosing cycle, wherein the scC2D1 is about 45 mg (e.g., 45 mg±10%; 45 mg±4.5 mg), and/or the control treatment comprises intravenously administering mosunetuzumab according to a dosing regimen comprising at least a first 21-day dosing cycle and a second 21-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg (e.g., 1 mg±10%), the ivC1D2 is about 2 mg (e.g., 2 mg±10%), and the ivC1D3 is about 60 mg (e.g., 60 mg±10%); and (b) the second dosing cycle comprises a first intravenous dose (ivC2D1) of mosunetuzumab administered on Day 1 of the second dosing cycle, wherein the ivC2D1 is about 60 mg (e.g., 60 mg±10%). In particular embodiments, the scC1D2 is about 45 mg (e.g., 45 mg±10%; 45 mg±4.5 mg).

In another aspect, the invention features a method for treating Non-Hodgkin Lymphoma (NHL) or chronic lymphocytic leukemia (CLL) in a subject in need thereof, comprising administering to the subject a treatment comprising subcutaneously administered mosunetuzumab according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄), wherein the treatment comprises subcutaneously administering mosunetuzumab according to a dosing regimen comprising at least a first 21-day dosing cycle and a second 21-day dosing cycle, wherein: (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg (e.g., 5 mg±10%; 5 mg±0.5 mg), the scC1D2 is about 15 mg (e.g., 15 mg±10%; 15 mg±1.5 mg) or about 45 mg (e.g., 45 mg±10%; 45 mg±4.5 mg), and the scC1D3 is about 45 mg (e.g., 45 mg±10%; 45 mg±4.5 mg); and (b) the second dosing cycle comprises a first subcutaneous dose (scC2D1) of mosunetuzumab administered on Day 1 of the second dosing cycle, wherein the scC2D1 is about 45 mg (e.g., 45 mg±10%; 45 mg±4.5 mg), and/or the control treatment comprises intravenously administering mosunetuzumab according to a dosing regimen comprising at least a first 21-day dosing cycle and a second 21-day dosing cycle, wherein: (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg (e.g., 1 mg±10%), the ivC1D2 is about 2 mg (e.g., 2 mg±10%), and the ivC1D3 is about 60 mg (e.g., 60 mg±10%); and (b) the second dosing cycle comprises a first intravenous dose (ivC2D1) of mosunetuzumab administered on Day 1 of the second dosing cycle, wherein the ivC2D1 is about 60 mg (e.g., 60 mg±10%). In particular embodiments, the scC1D2 is about 45 mg (e.g., 45 mg±10%; 45 mg±4.5 mg).

In some embodiments, the dosing regimen of the treatment further comprises one or more additional 21-day dosing cycles. In some embodiments, the dosing regimen of the treatment comprises six, fifteen, or twenty-three additional 21-day dosing cycles. In some embodiments, each additional dosing cycle comprises a subcutaneous dose of mosunetuzumab administered on Day 1 of the additional dosing cycle, wherein the subcutaneous dose of mosunetuzumab is about 45 mg (e.g., 45 mg±10%; 45 mg±4.5 mg).

In some embodiments, the dosing regimen of the control treatment further comprises one or more additional 21-day dosing cycles. In some embodiments, the dosing regimen of the control treatment comprises six, fifteen, or twenty-three additional 21-day dosing cycles. In some embodiments, each additional dosing cycle comprises an intravenous dose of mosunetuzumab administered on Day 1 of the additional dosing cycle, wherein the intravenous dose of mosunetuzumab is about 30 mg (e.g., 30 mg±10%).

In some embodiments, the subject has an Eastern Cooperative Oncology Group (ECOG) Performance Status of 0 or 1. In some embodiments, the subject has relapsed after or is refractory to one or more prior lines of systemic therapy. In some embodiments, the subject has relapsed after or is refractory to two or more prior lines of systemic therapy. In some embodiments, the one or more prior lines of systemic therapy comprises an anti-CD20 antibody, an alkylating agent, a Bruton's tyrosine kinase (BTK) inhibitor, an anthracycline, or a combination thereof.

In some embodiments, the NHL is a follicular lymphoma (FL), a marginal zone lymphoma (MZL), a diffuse large B cell lymphoma (DLBCL), a transformed FL (trFL), a high-grade B cell lymphoma (HGBL), a primary mediastinal B cell lymphoma (PMLBCL), a transformed indolent NHL, a mantle cell lymphoma (MCL), a Richter's transformation, or a small lymphocytic lymphoma (SLL). In some embodiments, the NHL is an FL, and the FL is Grade 1-3b FL or a transformed FL. In some embodiments, the FL is Grade 1-3a FL, and the subject has relapsed after or is refractory to two or more prior lines of systemic therapy comprising an anti-CD20 antibody and an alkylating agent. In some embodiments, the NHL is a DLBCL or a trFL. In some embodiments, the NHL is a DLBCL or a trFL, and the subject has relapsed after or is refractory to two or more prior lines of systemic therapy comprising an anthracycline and an anti-CD20 antibody. In some embodiments, the NHL is a Richter's transformation. In some embodiments, the NHL is a Richter's transformation, and the subject has relapsed after or is refractory to one or more prior lines of therapy comprising an anthracycline and an anti-CD20 antibody. In some embodiments, the anti-CD20 antibody is rituximab. In some embodiments, the NHL is an MCL. In some embodiments, the NHL is an

MCL, and the subject has relapsed after or is refractory to one or more prior lines of systemic therapy comprising a BTK inhibitor.

In some embodiments, the alkylating agent is cyclophosphamide (CAS #: 50-18-0) or bendamustine (CAS #: 3543-75-7). In some embodiments, the anthracycline is daunomycin (CAS #: 20830-81-3) or doxorubicin (CAS #: 23214-92-8). In some embodiments, the BTK inhibitor is 1-[(3R)-3-[4-Amino-3-(4-phenoxyphenyl)-1H-pyrazolo[3,4-d]pyrimidin-1-yl]piperidin-1-yl]prop-2-en-1-one and salts thereof, ibrutinib (CAS #: 936563-96-1), acalabrutinib (CAS #: 1420477-60-6), or zanubrutinib (CAS #: 1691249-45-2).

In some embodiments, the method comprises administering an additional therapeutic agent. In some embodiments, the additional therapeutic agent comprises a corticosteroid, an antihistamine, an antipyretic, or an IL-6R antagonist. In some embodiments, the additional therapeutic agent is a corticosteroid, and wherein the corticosteroid comprises prednisone, methylprednisolone, or dexamethasone. In some embodiments, the additional therapeutic agent is an antihistamine, and wherein the antihistamine comprises diphenhydramine hydrochloride or equivalent. In some embodiments, the additional therapeutic agent is an antipyretic, and wherein the antipyretic is acetaminophen. In some embodiments, the additional therapeutic agent is an IL-6R antagonist, and wherein the IL-6R antagonist is tocilizumab. In some embodiments, the additional therapeutic agent is administered in accordance with the methods described below in Part C (Dosing Strategies for Mitigation of Adverse Events (e.g., Cytokine Release Syndrome).

In one aspect, the invention features use of mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).

In one aspect, the invention features use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).

In one aspect, the invention features mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered response rate (ORR), a duration of response (DOR), a duration of complete response (DOCR), progression free survival (PFS), or overall survival (OS).

In one aspect, the invention features use of mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In one aspect, the invention features use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In one aspect, the invention features mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects r results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

In one aspect, the invention features use of mosunetuzumab for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In one aspect, the invention features use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In one aspect, the invention features mosunetuzumab for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference

PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

In some embodiments, administering the treatment comprising subcutaneous mosunetuzumab to a plurality of subjects having R/R FL results in an ORR of between about 66% to about 85% (e.g., 66%, 67%, 68%, 69%, 70%, 71%, 72%, 73%, 74%, 75%, 76%, 77%, 78%, 79%, 80%, 81%, 82%, 83%, 84%, or 85%). In some embodiments, the ORR is about 77%. In some embodiments, administering the treatment to a plurality of subjects having R/R FL results in a CRR in the plurality of subjects of between about 51% to about 72% (e.g., 51%, 52%, 53%, 54%, 55%, 56%, 57%, 58%, 59%, 60%, 61%, 62%, 63%, 64%, 65%, 66%, 67%, 68%, 69%, 70%, 71%, or 72%). In some embodiments, the CRR is about 62%. In some embodiments, administering the treatment to a plurality of subjects having R/R FL results in an 18-month event-free rate for DOCR in the plurality of subjects of between about 57% to about 83% (e.g., 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, or 83%). In some embodiments, the 18-month event-free rate for DOCR is about 70%. In some embodiments, wherein administering the treatment to a plurality of subjects having R/R FL results in a median DOCR in the plurality of subjects of at least about 20 months (e.g., 20, 24, 28, 32, 36, 40, 44, 48, 60, 72 months, or more). In some embodiments, the median DOCR is about 35 months. In some embodiments, administering the treatment to a plurality of subjects having R/R FL results in an 18-month event-free rate for PFS in the plurality of subjects of between about 46% to about 68% (e.g., 46%, 47%, 48%, 49%, 50%, 51%, 52%, 53%, 54%, 55%, 56%, 57%, 58%, 59%, 60%, 61%, 62%, 63%, 64%, 65%, 66%, 67%, or 68%). In some embodiments, the 18-month event-free rate for PFS is about 57%. In some embodiments, administering the treatment to a plurality of subjects having R/R FL results in a median PFS in the plurality of subjects of at least 14 months (e.g., 14, 18, 22, 24, 28, 30, 34, 38, 42 months, or more). In some embodiments, the median PFS is about 24 months.

In some embodiments, administering the treatment comprising subcutaneous mosunetuzumab to a plurality of subjects having 1 L FL results in an ORR of between about 76% to about 96% (e.g., 76%, 77%, 78%, 79%, 80%, 81%, 82%, 83%, 84%, 85%, 86%, 87%, 88%, 89%, 90%, 91%, 92%, 93%, 94%, 95%, or 96%). In some embodiments, administering the treatment comprising subcutaneous mosunetuzumab to a plurality of subjects having 1 L FL results in an ORR of between about 72% to about 96% (e.g., 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, or 96%). In some embodiments, the ORR is about 87%. In some embodiments, administering the treatment to a plurality of subjects having 1 L FL results in a CRR in the plurality of subjects of between about 55% to about 67% (e.g., 55%, 56%, 57%, 58%, 59%, 60%, 61%, 62%, 63%, 64%, 65%, 66%, or 67%). In some embodiments, administering the treatment to a plurality of subjects having 1 L FL results in a CRR in the plurality of subjects of between about 44% to about 77% (e.g., 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, or 77%). In some embodiments, the CRR is about 61%. In some embodiments, administering the treatment to a plurality of subjects having 1 L FL results in a 12-month event-free rate for PFS in the plurality of subjects of between about 73% to about 90% (e.g., 73%, 74%, 75%, 76%, 77%, 78%, 79%, 80%, 81%, 82%, 83%, 84%, 85%, 86%, 87%, 88%, 89%, or 90%). In some embodiments, the 12-month event-free rate for PFS is about 83%. In some embodiments, administering the treatment to a plurality of subjects having 1 L FL results in a median time to response (TTR) in the plurality of subjects exhibiting a response of between about 1 month to about 6 months (e.g., about 1, 2, 3, 4, 5, or 6 months). In some embodiments, the median TTR is about 3 months. In some embodiments, the subjects of the plurality have a high tumor burden, per the Groupe d'Etude des Lymphomes Folliculaires (GELF) criteria. In some embodiments, the plurality of subjects having 1 L FL and exhibiting the ORR or CRR described above are aged>65 years. In some embodiments, the plurality of subjects having 1 L FL and exhibiting the ORR or CRR described above are aged<65 years. In some embodiments, the plurality of subjects having 1 L FL and exhibiting the ORR or CRR described above have a FLIPI score of 0 or 1. In some embodiments, the plurality of subjects having 1 L FL and exhibiting the ORR or CRR described above have a FLIPI score of 2. In some embodiments, the plurality of subjects having 1 L FL and exhibiting the ORR or CRR described above have a FLIPI score of 3-5. In some embodiments, the plurality of subjects having 1 L FL and exhibiting the ORR or CRR described above have elevated lactate dehydrogenase (LDH) levels (i.e., >upper limit of normal (ULN) (e.g., >280 units/L)). In some embodiments, the plurality of subjects having 1 L FL and exhibiting the ORR or CRR described above do not have elevated LDH. In some embodiments, the plurality of subjects having 1 L FL and exhibiting the ORR or CRR described above have B symptoms (e.g., fever, night sweats, and weight loss). In some embodiments, the plurality of subjects having 1 L FL and exhibiting the ORR or CRR described above do not have B symptoms. In some embodiments, the plurality of subjects having 1 L FL and exhibiting the ORR or CRR described above have bulky disease (i.e., defined in the context of TLS as any lesion≥10 cm on the screening CT scan). In some embodiments, the plurality of subjects having 1 L FL and exhibiting the ORR or CRR described above do not have bulky disease. In some embodiments, the plurality of subjects having 1 L FL and exhibiting the ORR or CRR described above have Grade 1 or 2 FL. In some embodiments, the plurality of subjects having 1 L FL and exhibiting the ORR or CRR described above have Grade 3a FL.

In some embodiments, administering the treatment comprising subcutaneous mosunetuzumab to a plurality of subjects having 1 L MZL results in an ORR of between about 58% to about 88% (e.g., 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, or 88%). In some embodiments, the ORR is about 75%. In some embodiments, administering the treatment to a plurality of subjects having 1 L MZL results in a CRR in the plurality of subjects of between about 44% to about 77% (e.g., 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, or 77%). In some embodiments, the CRR is about 61%. In some embodiments, administering the treatment to a plurality of subjects having 1 L MZL results in a median TTR in the plurality of subjects exhibiting a response of between about 2 months to about 6 months (e.g., about 2, 3, 4, 5, or 6 months). In some embodiments, the median TTR is about 3 months.

### C. Dosing Strategies for Mitigation of Adverse Events (e.g., Cytokine Release Syndrome)

In some embodiments, administration of mosunetuzumab and/or lenalidomide to a subject according to the methods herein may result in adverse events in the subject. Described herein are methods for administering one or more additional therapeutic agents for mitigating (e.g., reducing the severity or duration of) and/or preventing adverse events in the subject.

In some embodiments, the first dosing cycle further comprises administration of a corticosteroid. In some embodiments, the second dosing cycle further comprises administration of a corticosteroid. In some embodiments, any of the one or more additional dosing cycles comprises administration of a corticosteroid. In some embodiments, a single dose of the corticosteroid is administered to the subject prior to the administration of any dose of mosunetuzumab. In some embodiments, the corticosteroid comprises dexamethasone or methylprednisolone. In some embodiments, the corticosteroid is administered intravenously or orally. In some embodiments, the corticosteroid comprises dexamethasone and is administered at a dose of about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg). In some embodiments, the corticosteroid comprises methylprednisolone and is administered at a dose of about 80 mg (e.g., 80 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, ±10 mg, ±12 mg, ±14 mg, or ±16 mg; e.g., 80 mg).

In some embodiments, the first dosing cycle further comprises administration of an antihistamine. In some embodiments, the second dosing cycle further comprises administration of an antihistamine. In some embodiments, any of the one or more additional dosing cycles comprises administration of an antihistamine. In some embodiments, a single dose of the antihistamine is administered to the subject prior to (e.g., 30, 35, 40, 45, 50, 60 70, 80, 90, 120, 150, 180 minutes, or more prior to) the administration of any dose of mosunetuzumab. In some embodiments, the antihistamine is administered to the subject at least 30 minutes (e.g., 30, 35, 40, 45, 50, 60 70, 80, 90, 120, 150, 180 minutes, or more) prior to the administration of any dose of mosunetuzumab. In some embodiments, the antihistamine is administered orally or intravenously. In some embodiments, the antihistamine comprises diphenhydramine hydrochloride and is administered at a dose of about 50-100 mg (e.g., 50-90 mg, 50-80 mg, 50-70 mg, 50-60 mg, 60-100 mg, 70-100 mg, 80-100 mg, 90-100 mg, 70-80 mg, 60-90 mg, 60-80 mg, 70-90 mg, or 65-85 mg; e.g., about 50 mg, about 55 mg, about 60 mg, about 65 mg, about 70 mg, about 75 mg, about 80 mg, about 85 mg, about 90 mg, about 95 mg, or about 100 mg).

In some embodiments, the first dosing cycle further comprises administration of an anti-pyretic. In some embodiments, the second dosing cycle further comprises administration of an anti-pyretic. In some embodiments, any of the one or more additional dosing cycles comprises administration of an anti-pyretic. In some embodiments, a single dose of the anti-pyretic is administered to the subject prior to the administration of any dose of mosunetuzumab. In some embodiments, the anti-pyretic is administered to the subject at least 30 minutes (e.g., 30, 35, 40, 45, 50, 60 70, 80, 90, 120, 150, 180 minutes, or more) prior to the administration of any dose of mosunetuzumab. In some embodiments, the anti-pyretic is administered orally. In some embodiments, the anti-pyretic comprises acetaminophen and is administered at a dose of about 500-1000 mg (e.g., 500-900 mg, 500-800 mg, 500-700 mg, 500-600 mg, 600-1000 mg, 700-1000 mg, 800-1000 mg, 900-1000 mg, 700-800 mg, 600-900 mg, 600-800 mg, 700-900 mg, or 650-850 mg; e.g., about 500 mg, about 550 mg, about 600 mg, about 650 mg, about 700 mg, about 750 mg, about 800 mg, about 850 mg, about 900 mg, about 950 mg, or about 1000 mg).

In some embodiments, the first dosing cycle further comprises administration of an initial dose of a prophylactic agent against tumor lysis syndrome (TLS). In some embodiments, the second dosing cycle further comprises administration of an initial dose of a prophylactic agent against TLS. In some embodiments, any of the one or more additional dosing cycles comprises administration of an initial dose of a prophylactic agent against TLS. In some embodiments, the initial dose of the prophylactic agent against TLS is administered to the subject prior to the administration of any dose of mosunetuzumab. In some embodiments, the prophylactic agent against TLS comprises allopurinol. In some embodiments, the initial dose of allopurinol is administered about 72 hours (e.g., 72±0.5 hours, ±1 hours, ±2 hours, ±3 hours, ±4 hours, ±8 hours, ±12 hours, or ±16 hours; e.g., 72 hours) prior to the administration of any dose of mosunetuzumab. In some embodiments, additional single doses of allopurinol are administered daily for 6-10 days (±1 day) after the administration of the initial dose. In some embodiments, the initial dose of allopurinol is about 300 mg (e.g., 300 mg±5 mg, ±10 mg, ±15 mg, ±20 mg, ±25 mg, ±30 mg, ±45 mg, or ±60 mg; e.g., 300 mg). In some embodiments, each additional single dose of allopurinol is about 300 mg (e.g., 300 mg±5 mg, ±10 mg, ±15 mg, ±20 mg, ±25 mg, ±30 mg, ±45 mg, or ±60 mg; e.g., 300 mg). In some embodiments, allopurinol is administered orally. In some embodiments, the prophylactic agent against TLS comprises rasburicase. In some embodiments, the initial dose of rasburicase is administered about 30 minutes (e.g., 30±0.5 minutes, 1 minutes, ±2 minutes, ±3 minutes, ±4 minutes, ±5 minutes, or ±6 minutes; e.g., 30 minutes) prior to the administration of any dose of mosunetuzumab. In some embodiments, additional single doses of rasburicase are administered daily for 1-5 days (±1 day) after the administration of the initial dose. In some embodiments, the initial dose of rasburicase is about 0.2 mg/kg (e.g., 0.2±0.005 mg/kg, ±0.01 mg/kg, ±0.02 mg/kg, ±0.03 mg/kg, or ±0.04 mg/kg; e.g., 0.2 mg/kg). In some embodiments, each additional single dose of rasburicase is about 0.2 mg/kg (e.g., 0.2±0.005 mg/kg, ±0.01 mg/kg, ±0.02 mg/kg, ±0.03 mg/kg, or ±0.04 mg/kg; e.g., 0.2 mg/kg). In some embodiments, rasburicase is administered intravenously.

In some embodiments, an IL-6R antagonist may be administered to the subject treated with the methods of the invention, e.g., when the subject exhibits CRS, e.g., after being administered any dose of mosunetuzumab. In some embodiments, the IL-6R antagonist is tocilizumab. In some embodiments, tocilizumab is administered to the subject as a single dose of about 8 mg/kg (e.g., 8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, ±1 mg/kg, ±1.5 mg/kg, or ±2 mg/kg; e.g., 8 mg/kg), and wherein the single dose does not exceed 800 mg. In some embodiments, tocilizumab is administered to the subject as a single dose of about 12 mg/kg (e.g., 12 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, ±1 mg/kg, ±1.5 mg/kg, or ±2 mg/kg; e.g., 12 mg/kg), and wherein the single dose does not exceed 800 mg. In some embodiments, tocilizumab is administered intravenously.

The methods described herein may result in an acceptable safety profile for subjects having a CD20-positive cell proliferative disorder (e.g., a B cell proliferative disorder; e.g., an NHL or CLL) being treated with mosunetuzumab monotherapy or a combination therapy of mosunetuzumab and lenalidomide. In some instances, treatment using the methods described herein that include administration of mosunetuzumab and/or oral administration of lenalidomide results in a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater; e.g., between 20% and 100%, between 20% and 90%, between 20% and 80%, between 20% and 70%, between 20% and 60%, between 20% and 50%, between 20% and 40%, between 20% and 30%, between 40% and 100%, between 60% and 100%, between 80% and 100%, between 30% and 70%, between 40% and 60%, between 30% and 50%, between 50% and 80%, or between 90% and 100%; e.g., about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or about 100%) or complete inhibition (100% reduction) of undesirable adverse events, such as cytokine-driven toxicities (e.g., cytokine release syndrome (CRS)), infusion-related reactions (IRRs), macrophage activation syndrome (MAS), neurologic toxicities, tumor lysis syndrome (TLS), neutropenia, thrombocytopenia, elevated liver enzymes, and/or hepatotoxicities.

In some instances, subcutaneous administration of mosunetuzumab as a monotherapy or as a combination therapy with lenalidomide according to the methods described herein results in a non-inferiority or a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater; e.g., between 20% and 100%, between 20% and 90%, between 20% and 80%, between 20% and 70%, between 20% and 60%, between 20% and 50%, between 20% and 40%, between 20% and 30%, between 40% and 100%, between 60% and 100%, between 80% and 100%, between 30% and 70%, between 40% and 60%, between 30% and 50%, between 50% and 80%, or between 90% and 100%; e.g., about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or about 100%) or complete inhibition (100% reduction) of undesirable adverse events, such as cytokine-driven toxicities (e.g., cytokine release syndrome (CRS)), infusion-related reactions (IRRs), macrophage activation syndrome (MAS), neurologic toxicities, tumor lysis syndrome (TLS), neutropenia, thrombocytopenia, elevated liver enzymes, and/or hepatotoxicities as compared to intravenous administration of mosunetuzumab as a monotherapy or as a combination therapy with lenalidomide according to the methods described herein.

### IV. THERAPEUTIC AGENTS

### A. Mosunetuzumab

The invention provides mosunetuzumab, a bispecific antibody that binds to CD20 and CD3, useful for treating a CD20-positive cell proliferative disorder, e.g., a B cell proliferative disorder (e.g., non-Hodgkin's lymphoma (NHL) (e.g., a previously untreated (1 L) NHL, a diffuse-large B cell lymphoma (DLBCL) (e.g., 1 L DLBCL, a relapsed and/or refractory DLBCL, or a Richter's transformation), a follicular lymphoma (FL) (e.g., a 1 L FL, a relapsed and/or refractory FL, or a transformed FL), a mantle cell lymphoma (MCL), a high-grade B cell lymphoma, or a primary mediastinal (thymic) large B cell lymphoma (PMLBCL)) or a chronic lymphoid leukemia (CLL)).

Mosunetuzumab has an anti-CD20 arm having a first binding domain comprising the following six hypervariable regions (HVRs): (a) an HVR-H1 comprising the amino acid sequence of GYTFTSYNMH (SEQ ID NO: 1); (b) an HVR-H2 comprising the amino acid sequence of AIYPGNGDTSYNQKFKG (SEQ ID NO: 2); (c) an HVR-H3 comprising the amino acid sequence of VVYYSNSYWYFDV (SEQ ID NO: 3); (d) an HVR-L1 comprising the amino acid sequence of RASSSVSYMH (SEQ ID NO: 4); (e) an HVR-L2 comprising the amino acid sequence of APSNLAS (SEQ ID NO: 5); and (f) an HVR-L3 comprising the amino acid sequence of QQWSFNPPT (SEQ ID NO: 6). Mosunetuzumab comprises an anti-CD20 arm comprising a first binding domain comprising the heavy chain framework regions FR-H1, FR-H2, FR-H3, and FR-H4 comprising the sequences of SEQ ID NOs: 17-20, respectively, and the light chain framework regions FR-L1, FR-L2, FR-L3, and FR-L4 comprising the sequences of SEQ ID NOs: 21-24, respectively. Mosunetuzumab comprises an anti-CD20 arm comprising a first binding domain comprising (a) a heavy chain variable (VH) domain comprising the amino acid sequence of SEQ ID NO: 7; and (b) a light chain variable (VL) domain comprising the amino acid sequence of SEQ ID NO: 8.

Mosunetuzumab has an anti-CD3 arm having a second binding domain comprising the following six HVRs: (a) an HVR-H1 comprising the amino acid sequence of NYYIH (SEQ ID NO: 9); (b) an HVR-H2 comprising the amino acid sequence of WIYPGDGNTKYNEKFKG (SEQ ID NO: 10); (c) an HVR-H3 comprising the amino acid sequence of DSYSNYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WASTRES (SEQ ID NO: 13); and (f) an HVR-L3 comprising the amino acid sequence of TQSFILRT (SEQ ID NO: 14). Mosunetuzumab comprises an anti-CD3 arm comprising a second binding domain comprising the heavy chain framework regions FR-H1, FR-H2, FR-H3, and FR-H4 comprising the sequences of SEQ ID NOs: 25-28, respectively, and the light chain framework regions FR-L1, FR-L2, FR-L3, and FR-L4 comprising the sequences of SEQ ID NOs: 29-32, respectively. Mosunetuzumab comprises an anti-CD3 arm comprising a second binding domain comprising (a) a VH domain comprising an amino acid sequence having the amino acid sequence of SEQ ID NO: 15; and (b) a VL domain comprising the amino acid sequence of SEQ ID NO: 16.

Mosunetuzumab has the International Nonproprietary Names for Pharmaceutical Substances (INN) List 117 (WHO Drug Information, Vol. 31, No. 2, 2017, p. 304-305), or CAS Registry No. 1905409-39-3, and having (1) an anti-CD20 arm comprising the heavy chain and light chain sequences of SEQ ID NOs: 33 and 34, respectively; and (2) an anti-CD3 arm comprising the heavy chain and light chain sequences of SEQ ID NOs: 35 and 36, respectively. Mosunetuzumab comprises (1) an anti-CD20 arm comprising a first binding domain comprising a heavy chain comprising the amino acid sequence of SEQ ID NO: 33 and a light chain comprising the amino acid sequence of SEQ ID NO: 34 and (2) an anti-CD3 arm comprising a second binding domain comprising a heavy chain comprising the amino acid sequence of SEQ ID NO: 35 and a light chain comprising the amino acid sequence of SEQ ID NO: 36.

Amino acid sequences of mosunetuzumab are summarized in Table 1 below.

TABLE 1
Sequence IDs for Mosunetuzumab

| | |
|---|---|
| CD3 Arm | CD20 Arm |

| SEQ | | SEQ | |
|---|---|---|---|
| ID NO: | Description | ID NO: | Description |
| 9 | CD3 HVR-H1 | 1 | CD20 HVR-H1 |
| 10 | CD3 HVR-H2 | 2 | CD20 HVR-H2 |
| 11 | CD3 HVR-H3 | 3 | CD20 HVR-H3 |
| 12 | CD3 HVR-L1 | 4 | CD20 HVR-L1 |
| 13 | CD3 HVR-L2 | 5 | CD20 HVR-L2 |
| 14 | CD3 HVR-L3 | 6 | CD20 HVR-L3 |
| 15 | CD3 VH | 7 | CD20 VH |
| 16 | CD3 VL | 8 | CD20 VL |
| 35 | CD3 heavy chain | 33 | CD20 heavy chain |
| 36 | CD3 light chain | 34 | CD20 light chain |

Mosunetuzumab may be produced using recombinant methods and compositions, for example, as described in U.S. Pat. No. 4,816,567.

### B. Lenalidomide

Lenalidomide is an immunomodulatory (IMiD) imide drug that binds to cereblon, an E3 ubiquitin ligase protein (Gribben et al. J. Clin. Oncol. 33 (25): 2803-2811, 2015). The immunomodulatory activity of lenalidomide is not completely understood; however, lenalidomide has been shown to enhance CD4+ and CD8+ T-cell co-stimulation, induce T-cell proliferation, and enhance IL-2 and IFN-γ (Haslett et al. J. Exp. Med. 187 (11): 1885-1892, 1998; Davies et al. Blood 98 (1): 210-216, 2001).

Lenalidomide has the CAS Registry Number 191732-72-6 and IUPAC name (3RS)-3-(4-Amino-1-oxo-1,3-dihydro-2H-isoindol-2-yl) piperidine-2,6-dione. Lenalidomide is also known by tradenames including REVLIMIDO, linamid, and lenalid. Lenalidomide has the DrugBank Accession Number DB00480, PubChem CID 216326, and chemical formula C₁₃H₁₃N₃O₃.

### C. Additional Therapeutic Agents

In some instances, the methods described herein include administering mosunetuzumab and lenalidomide in combination with one or more additional therapeutic agents.

In some instances, the one or more additional therapeutic agents may prevent, reduce the rate of, or reduce the severity of cytokine release syndrome (CRS) and/or symptoms associated with CRS. In particular instances, the additional therapeutic agent used to reduce the rate or severity of CRS or prevent symptoms associated with CRS is a corticosteroid (e.g., dexamethasone (CAS #: 50-02-2), prednisone (CAS #: 53-03-2), prednisolone (CAS #: 50-42-8), or methylprednisolone (CAS #: 83-43-2)), antihistamine (e.g., diphenhydramine (CAS #: 58-73-1) and pharmaceutically acceptable salts thereof, e.g., diphenhydramine hydrochloride (CAS #: 147-24-0)) or an interleukin-6 receptor (IL-6R) antagonist (e.g., tocilizumab (CAS #: 375823-41-9), sarilumab (CAS #: 1189541-98-7), vobarilizumab (ALX-0061; CAS #: 1628814-88-9), satralizumab (SA-237; CAS #: 1535963-91-7), and alternatives thereof). In some instances, the additional therapeutic agent is a corticosteroid. In particular instances, the corticosteroid is dexamethasone or methylprednisolone. In a particular instance, the antihistamine is diphenhydramine hydrochloride. In a particular instance, the anti-pyretic is acetaminophen (i.e., paracetamol) or pharmaceutically acceptable salts thereof. In a particular instance, the IL-6R antagonist is tocilizumab.

In some instances, the one or more additional therapeutic agents may be used in the treatment of neutropenia. In some instances, the additional therapeutic agents may be prevent symptoms associated with neutropenia. In some instances, the additional therapeutic agents may reduce the rate or severity of neutropenia. In particular instances, the additional therapeutic agent is granulocyte colony-stimulating factor (G-CSF or GCSF) or colony-stimulating factor 3 (CSF 3). The mRNA sequence of human G-CSF/CSF 3 includes, e.g., NCBI RefSeq No. NM_000759, NM_001178147, NM_172219, and NM_172220, and the protein amino acid sequence of human G-CSF/CSF 3 includes, e.g., NCBI RefSeq No. NP_000750, NP_001171618, NP_757373, and NP_757374.

In some instances, the one or more additional therapeutic agents is a prophylactic agent against, e.g., to prevent, reduce the rate of, or reduce the severity of tumor lysis syndrome (TLS). In some instances, the prophylactic agent against TLS is allopurinol (CAS #: 315-30-0), rasburicase (Elitek™; CAS #: 134774-45-1), or suitable alternatives and/or pharmaceutically acceptable salts thereof. In some instances, the prophylactic agent against TLS reduces serum uric acid (e.g., reduces elevated uric acid levels, e.g., relative to a baseline or reference value).

In some instances, additional therapeutic agents useful in the present invention include therapeutic antibodies, such as alemtuzumab (CAMPATH®), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (BEXXAR®, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, tafasitamab, talizumab, tefibazumab, tocilizumab, toralizumab, tucotuzumab celmoleukin, tucusituzumab, umavizumab, urtoxazumab, ustekinumab, visilizumab, and briakinumab.

### V. PHARMACEUTICAL COMPOSITIONS AND FORMULATIONS

Mosunetuzumab, lenalidomide, and other therapeutic agents described herein can be used in pharmaceutical compositions and formulations. Pharmaceutical compositions and formulations of mosunetuzumab, lenalidomide, and/or other agents describe herein (e.g., corticosteroids, antihistamines, anti-pyretics, prophylactic agents against TLS, and/or IL-6R antagonists) can be prepared by mixing one, two, three, or more agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Corticosteroids, antihistamines, anti-pyretics, prophylactic agents against TLS, and/or IL-6R antagonists may also be formulated according to standard formulation and/or manufacturing practices. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

In some embodiments, mosunetuzumab is formulated for administration subcutaneously. In some embodiments, mosunetuzumab is formulated for administration intravenously. In some embodiments, lenalidomide is formulated for administration orally. In some embodiments, dexamethasone is formulated for administration orally or intravenously. In some embodiments, methylprednisolone is formulated for administration orally or intravenously. In some embodiments, allopurinol is formulated for administration orally. In some embodiments, rasburicase is formulated for administration intravenously. In some embodiments, tocilizumab is formulated for administration intravenously.

### VI. KITS AND ARTICLES OF MANUFACTURE

In another aspect of the invention, a kit or an article of manufacture containing materials useful for the treatment, prevention, and/or diagnosis of the disorders described above is provided. The kit or article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be a vial having a stopper pierceable by a hypodermic injection needle). At least one active agent in the composition is mosunetuzumab and/or lenalidomide described herein. The label or package insert indicates that the composition is used for treating the condition of choice (e.g., a CD20-positive cell proliferative disorder, e.g., a B cell proliferative disorder (e.g., non-Hodgkin's lymphoma (NHL) (e.g., a previously untreated (1 L) NHL, a diffuse-large B cell lymphoma (DLBCL) (e.g., 1 L DLBCL, a relapsed and/or refractory DLBCL, or a Richter's transformation), a follicular lymphoma (FL) (e.g., a 1 L FL, a relapsed and/or refractory FL, or a transformed FL), a mantle cell lymphoma (MCL), a high-grade B cell lymphoma, or a primary mediastinal (thymic) large B cell lymphoma (PMLBCL)) or a chronic lymphoid leukemia (CLL))) and further includes information related to at least one of the dosing regimens described herein. Moreover, the kit or article of manufacture may comprise (a) a first container with a composition contained therein, wherein the composition comprises mosunetuzumab and/or lenalidomide described herein; and (b) a second container with a composition contained therein, wherein the composition comprises a further cytotoxic or otherwise therapeutic agent. Alternatively, or additionally, the kit or article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### VII. EMBODIMENTS

Some embodiments of the technology described herein can be defined according to any of the following numbered embodiments:
1. A method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).
2. Use of mosunetuzumab in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).
3. Use of lenalidomide in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).
4. Use of mosunetuzumab and lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).
5. Use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).
6. Use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).
7. Use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).
8. Mosunetuzumab for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).
9. Lenalidomide for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).
10. Mosunetuzumab and lenalidomide for use for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).
11. The method, use, mosunetuzumab for use, lenalidomide for use, or mosunetuzumab and lenalidomide for use of any one of embodiments 1-10, wherein the efficacy response is a CRR, and wherein the CRR or reference CRR is the proportion of the subjects receiving the corresponding treatment whose best overall response is a complete response (CR).
12. The method, use, mosunetuzumab for use, lenalidomide for use, or mosunetuzumab and lenalidomide for use of embodiment 11, wherein the improved response in CRR is an increase in CRR compared to the reference CRR of between 1% and 25%.
13. The method, use, mosunetuzumab for use, lenalidomide for use, or mosunetuzumab and lenalidomide for use of embodiment 11, wherein the efficacy response in CRR is non-inferior compared to the reference CRR.
14. The method, use, mosunetuzumab for use, lenalidomide for use, or mosunetuzumab and lenalidomide for use of any one of embodiments 1-10, wherein the efficacy response is an ORR, and wherein the ORR or reference ORR is the proportion of the subjects receiving the corresponding treatment whose best overall response is a CR or a partial response (PR).
15. The method, use, mosunetuzumab for use, lenalidomide for use, or mosunetuzumab and lenalidomide for use of embodiment 14, wherein the improved response in ORR is an increase in ORR compared to the reference ORR of between 1% and 24%.
16. The method, use, mosunetuzumab for use, lenalidomide for use, or mosunetuzumab and lenalidomide for use of embodiment 14, wherein the efficacy response in ORR is non-inferior compared to the reference ORR.
17. The method, use, mosunetuzumab for use, lenalidomide for use, or mosunetuzumab and lenalidomide for use of any one of embodiments 1-10, wherein the efficacy response is a DOR, and wherein the DOR or the reference DOR is measured starting from the time from the first occurrence of a documented CR or PR to disease progression or relapse or death from any cause, whichever occurs first.
18. The method, use, mosunetuzumab for use, lenalidomide for use, or mosunetuzumab and lenalidomide for use of embodiment 17, wherein the DOR or the reference DOR is the median DOR of the plurality of subjects receiving the corresponding treatment.
19. The method, use, mosunetuzumab for use, lenalidomide for use, or mosunetuzumab and lenalidomide for use of embodiment 17 or 18, wherein the improvement of the median DOR is an increase in the DOR compared to the reference DOR of between 1 and 22 months.
20. The method, use, mosunetuzumab for use, lenalidomide for use, or mosunetuzumab and lenalidomide for use of embodiment 17, wherein the efficacy response in DOR is non-inferior compared to the reference DOR.
21. The method, use, mosunetuzumab for use, lenalidomide for use, or mosunetuzumab and lenalidomide for use of any one of embodiments 1-10, wherein the efficacy response is a DOCR, and wherein the DOCR or the reference DOCR is measured starting from the time from the first occurrence of a documented CR to disease progression or relapse or death from any cause, whichever occurs first.
22. The method, use, mosunetuzumab for use, lenalidomide for use, or mosunetuzumab and lenalidomide for use of embodiment 21, wherein the DOCR or the reference DOCR is the median DOCR of the plurality of subjects receiving the corresponding treatment.
23. The method, use, mosunetuzumab for use, lenalidomide for use, or mosunetuzumab and lenalidomide for use of embodiment 21 or 22, wherein the improvement of the median DOCR is an increase in the DOCR compared to the reference DOCR of between 1 and 22 months.
24. The method, use, mosunetuzumab for use, lenalidomide for use, or mosunetuzumab and lenalidomide for use of embodiment 21, wherein the efficacy response in DOCR is non-inferior compared to the reference DOCR.
25. The method, use, mosunetuzumab for use, lenalidomide for use, or mosunetuzumab and lenalidomide for use of any one of embodiments 11-24, wherein the CR or PR is determined by PET/computed tomography (CT).
26. The method, use, mosunetuzumab for use, lenalidomide for use, or mosunetuzumab and lenalidomide for use of embodiment 25, wherein the CR or PR is determined based on the Lugano Response Criteria for Malignant Lymphoma (Cheson et al., 2014).
27. The method, use, mosunetuzumab for use, lenalidomide for use, or mosunetuzumab and lenalidomide for use of any one of embodiments 1-10, wherein the efficacy response is an OS, wherein the OS or the reference OS is measured starting from the time from first treatment to death from any cause.
28. The method, use, mosunetuzumab for use, lenalidomide for use, or mosunetuzumab and lenalidomide for use of embodiment 27, wherein the OS or the reference OS is the median OS of the plurality of subjects receiving the corresponding treatment.
29. The method, use, mosunetuzumab for use, lenalidomide for use, or mosunetuzumab and lenalidomide for use of embodiment 27 or 28, wherein the improvement of the median OS is an increase in the OS compared to the reference OS of between 1 and 28 months.
30. The method, use, mosunetuzumab for use, lenalidomide for use, or mosunetuzumab and lenalidomide for use of embodiment 27, wherein the efficacy response in OS is non-inferior compared to the reference OS.
31. The method, use, mosunetuzumab for use, lenalidomide for use, or mosunetuzumab and lenalidomide for use of any one of embodiments 1-10, wherein the efficacy response is a PFS, and wherein the PFS or the reference PFS is measured starting from the time from first treatment to the time of a first occurrence of disease progression or death from any cause.
32. The method, use, mosunetuzumab for use, lenalidomide for use, or mosunetuzumab and lenalidomide for use of embodiment 31, wherein the PFS or the reference PFS is the median PFS of the plurality of subjects receiving the corresponding treatment.
33. The method, use, mosunetuzumab for use, lenalidomide for use, or mosunetuzumab and lenalidomide for use of embodiment 31 or 32, wherein the improvement of the median PFS is an increase in the PFS compared to the reference PFS of between 1 and 26 months.
34. The method, use, mosunetuzumab for use, lenalidomide for use, or mosunetuzumab and lenalidomide for use of embodiment 31, wherein the efficacy response in PFS is non-inferior compared to the reference PFS.
35. A method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).
36. Use of mosunetuzumab in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).
37. Use of lenalidomide in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).
38. Use of mosunetuzumab and lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).
39. Use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).
40. Use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).
41. Use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).
42. Mosunetuzumab for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).
43. Lenalidomide for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).
44. Mosunetuzumab and lenalidomide for use for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).
45. The method, use, mosunetuzumab for use, lenalidomide for use, or mosunetuzumab and lenalidomide for use of any one of embodiments 35-44, wherein the safety response is the rate of AE, and wherein the rate of AE or the reference rate of AE is the rate of AE in the plurality of subjects receiving the corresponding treatment.
46. The method, use, mosunetuzumab for use, lenalidomide for use, or mosunetuzumab and lenalidomide for use of embodiment 45, wherein the safety response in rate of AE is non-inferior compared to the reference rate of AE.
47. The method, use, mosunetuzumab for use, lenalidomide for use, or mosunetuzumab and lenalidomide for use of any one of embodiments 35-44, wherein the safety response is the rate of CRS, and wherein the rate of CRS or the reference rate of CRS is the rate of CRS in the plurality of subjects receiving the corresponding treatment.
48. The method, use, mosunetuzumab for use, lenalidomide for use, or mosunetuzumab and lenalidomide for use of embodiment 47, wherein the improved rate of CRS is a decrease in the rate of CRS compared to the reference rate of CRS of between 7% and 17%.
49. The method, use, mosunetuzumab for use, lenalidomide for use, or mosunetuzumab and lenalidomide for use of embodiment 48, wherein the improved rate of CRS is a decrease in the rate of CRS compared to the reference rate of CRS of about 12%.
50. The method, use, mosunetuzumab for use, lenalidomide for use, or mosunetuzumab and lenalidomide for use of embodiment 48 or 49, wherein the rate of CRS is the rate of Grade 1 or 2 CRS in the plurality of patients receiving the corresponding treatment.
51. The method, use, mosunetuzumab for use, lenalidomide for use, or mosunetuzumab and lenalidomide for use of embodiment 47, wherein the rate of Grade 3+ CRS is non-inferior compared to the reference rate of Grade 3+ CRS.
52. The method, use, mosunetuzumab for use, lenalidomide for use, or mosunetuzumab and lenalidomide for use of embodiment 50 or 51, wherein the Grade of CRS is determined based on the American Society of Transplantation and Cellular Therapy (ASTCT) Consensus Grading for Cytokine-Release Syndrome (Lee et al., 2019).
53. The method, use, mosunetuzumab for use, lenalidomide for use, or mosunetuzumab and lenalidomide for use of any one of embodiments 35-44, wherein the safety response is the rate of SAE, and wherein the rate of SAE or the reference rate of SAE is the rate of SAE in the plurality of subjects receiving the corresponding treatment.
54. The method, use, mosunetuzumab for use, lenalidomide for use, or mosunetuzumab and lenalidomide for use of embodiment 53, wherein the safety response in rate of SAE is non-inferior compared to the reference rate of SAE.
55. A method for treating follicular lymphoma (FL) in a subject in need thereof, comprising administering to the subject a combination treatment comprising subcutaneously administered mosunetuzumab and orally administered lenalidomide according to a dosing regimen comprising a first 21-day dosing cycle and three 28-day dosing cycles, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).
56. Use of mosunetuzumab in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).
57. Use of lenalidomide in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).
58. Use of mosunetuzumab and lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).
59. Use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).
60. Use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).
61. Use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).
62. Mosunetuzumab for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).
63. Lenalidomide for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).
64. Mosunetuzumab and lenalidomide for use for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).
65. The method, use, mosunetuzumab for use, lenalidomide for use, or mosunetuzumab and lenalidomide for use of any one of embodiments 55-64 wherein one or both of the scAUC₀₋₇₇ and the ivAUC₀₋₇₇ is determined using population PK (popPK) model-predicted individual Empirical Bayesian Estimates (EBEs).
66. The method, use, mosunetuzumab for use, lenalidomide for use, or mosunetuzumab and lenalidomide for use of any one of embodiments 1-65, wherein the combination treatment comprises subcutaneously administering mosunetuzumab and orally administering lenalidomide according to a dosing regimen comprising at least a first dosing cycle and a second dosing cycle, wherein the first dosing cycle is a 21-day dosing cycle and the second dosing cycle is a 28-day dosing cycle, and wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg.
67. The method, use, mosunetuzumab for use, lenalidomide for use, or mosunetuzumab and lenalidomide for use of embodiment 66, wherein the dosing regimen of the combination treatment further comprises one or more additional 28-day dosing cycles.
68. The method, use, mosunetuzumab for use, lenalidomide for use, or mosunetuzumab and lenalidomide for use of embodiment 67, wherein the dosing regimen of the combination treatment comprises ten additional 28-day dosing cycles.
69. The method, use, mosunetuzumab for use, lenalidomide for use, or mosunetuzumab and lenalidomide for use of embodiment 67 or 68, wherein each additional dosing cycle comprises:
   (a) a subcutaneous dose of mosunetuzumab administered on Day 1 of the additional dosing cycle, wherein the subcutaneous dose of mosunetuzumab is about 45 mg; and
   (b) twenty-one (21) oral doses of lenalidomide administered on Days 1-21 of the additional dosing cycle, wherein each oral dose of lenalidomide is about 20 mg.
70. The method, use, mosunetuzumab for use, lenalidomide for use, or mosunetuzumab and lenalidomide for use of embodiment 69, wherein the dosing regimen of the combination treatment further comprises one to nine subcutaneous maintenance doses of mosunetuzumab.
71. A method for treating follicular lymphoma (FL) in a subject in need thereof, comprising administering to the subject a combination treatment comprising subcutaneously administered mosunetuzumab and orally administered lenalidomide, wherein the combination treatment comprises subcutaneously administering mosunetuzumab and orally administering lenalidomide according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg; and
   (c) the ten additional dosing cycles each comprises:
      (i) a first subcutaneous dose (scC3D1-scC12D1) of mosunetuzumab administered on Day 1 of each additional dosing cycle, wherein the scC3D1-scC12D1 are each about 45 mg; and
      (ii) twenty-one (21) oral doses of lenalidomide administered on Days 1-21 of each additional dosing cycle, wherein each oral dose of lenalidomide is about 20 mg.
72. Use of mosunetuzumab in combination with lenalidomide for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg; and
   (c) the ten additional dosing cycles each comprises:
      (i) a first subcutaneous dose (scC3D1-scC12D1) of mosunetuzumab administered on Day 1 of each additional dosing cycle, wherein the scC3D1-scC12D1 are each about 45 mg; and
      (ii) twenty-one (21) oral doses of lenalidomide administered on Days 1-21 of each additional dosing cycle, wherein each oral dose of lenalidomide is about 20 mg.
73. Use of lenalidomide in combination with mosunetuzumab for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg; and
   (c) the ten additional dosing cycles each comprises:
      (i) a first subcutaneous dose (scC3D1-scC12D1) of mosunetuzumab administered on Day 1 of each additional dosing cycle, wherein the scC3D1-scC12D1 are each about 45 mg; and
      (ii) twenty-one (21) oral doses of lenalidomide administered on Days 1-21 of each additional dosing cycle, wherein each oral dose of lenalidomide is about 20 mg.
74. Use of mosunetuzumab and lenalidomide for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg; and
   (c) the ten additional dosing cycles each comprises:
      (i) a first subcutaneous dose (scC3D1-scC12D1) of mosunetuzumab administered on Day 1 of each additional dosing cycle, wherein the scC3D1-scC12D1 are each about 45 mg; and
      (ii) twenty-one (21) oral doses of lenalidomide administered on Days 1-21 of each additional dosing cycle, wherein each oral dose of lenalidomide is about 20 mg.
75. Use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg; and
   (c) the ten additional dosing cycles each comprises:
      (i) a first subcutaneous dose (scC3D1-scC12D1) of mosunetuzumab administered on Day 1 of each additional dosing cycle, wherein the scC3D1-scC12D1 are each about 45 mg; and
      (ii) twenty-one (21) oral doses of lenalidomide administered on Days 1-21 of each additional dosing cycle, wherein each oral dose of lenalidomide is about 20 mg.
76. Use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg; and
   (c) the ten additional dosing cycles each comprises:
      (i) a first subcutaneous dose (scC3D1-scC12D1) of mosunetuzumab administered on Day 1 of each additional dosing cycle, wherein the scC3D1-scC12D1 are each about 45 mg; and
      (ii) twenty-one (21) oral doses of lenalidomide administered on Days 1-21 of each additional dosing cycle, wherein each oral dose of lenalidomide is about 20 mg.
77. Use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg; and
   (c) the ten additional dosing cycles each comprises:
      (i) a first subcutaneous dose (scC3D1-scC12D1) of mosunetuzumab administered on Day 1 of each additional dosing cycle, wherein the scC3D1-scC12D1 are each about 45 mg; and
      (ii) twenty-one (21) oral doses of lenalidomide administered on Days 1-21 of each additional dosing cycle, wherein each oral dose of lenalidomide is about 20 mg.
78. Mosunetuzumab for use in combination with lenalidomide for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg;
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg; and
   (c) the ten additional dosing cycles each comprises:
      (i) a first subcutaneous dose (scC3D1-scC12D1) of mosunetuzumab administered on Day 1 of each additional dosing cycle, wherein the scC3D1-scC12D1 are each about 45 mg; and
      (ii) twenty-one (21) oral doses of lenalidomide administered on Days 1-21 of each additional dosing cycle, wherein each oral dose of lenalidomide is about 20 mg.
79. Lenalidomide for use in combination with mosunetuzumab for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg;
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg; and
   (c) the ten additional dosing cycles each comprises:
      (i) a first subcutaneous dose (scC3D1-scC12D1) of mosunetuzumab administered on Day 1 of each additional dosing cycle, wherein the scC3D1-scC12D1 are each about 45 mg; and
      (ii) twenty-one (21) oral doses of lenalidomide administered on Days 1-21 of each additional dosing cycle, wherein each oral dose of lenalidomide is about 20 mg.
80. Mosunetuzumab and lenalidomide for use for treating follicular lymphoma (FL) in a subject in need thereof, wherein mosunetuzumab is to be administered subcutaneously and lenalidomide is to be administered orally as a combination treatment according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
   (b) the second dosing cycle comprises:
      (i) a first subcutaneous dose (scC2D1) of mosunetuzumab administered on Day 1 of the second dosing cycle, wherein the scC2D1 is about 45 mg; and
      (ii) twenty-one (21) oral doses of lenalidomide administered on Days 1-21 of the second dosing cycle, wherein each oral dose of lenalidomide is about 20 mg; and
   (c) the ten additional dosing cycles each comprises:
      (i) a first subcutaneous dose (scC3D1-scC12D1) of mosunetuzumab administered on Day 1 of each additional dosing cycle, wherein the scC3D1-scC12D1 are each about 45 mg; and
      (ii) twenty-one (21) oral doses of lenalidomide administered on Days 1-21 of each additional dosing cycle, wherein each oral dose of lenalidomide is about 20 mg.
81. The method, use, mosunetuzumab for use, lenalidomide for use, or mosunetuzumab and lenalidomide for use of any one of embodiments 70-80, wherein the dosing regimen of the combination treatment further comprises nine subcutaneous maintenance doses of mosunetuzumab.
82. The method, use, mosunetuzumab for use, lenalidomide for use, or mosunetuzumab and lenalidomide for use of any one of embodiments 70-81, wherein the one to nine subcutaneous maintenance doses of mosunetuzumab are administered every eight weeks starting eight weeks after Day 1 of the tenth additional 28-day dosing cycle.
83. The method, use, mosunetuzumab for use, lenalidomide for use, or mosunetuzumab and lenalidomide for use of any one of embodiments 70-82, wherein each maintenance dose of mosunetuzumab is about 45 mg.
84. The method, use, mosunetuzumab for use, lenalidomide for use, or mosunetuzumab and lenalidomide for use of any one of embodiments 1-70 and 81-83, wherein the control treatment comprises intravenously administering mosunetuzumab and orally administering lenalidomide according to a dosing regimen comprising at least a first 21-day dosing cycle and a second 28-day dosing cycle, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second dosing cycle comprises:
      (i) a first intravenous dose (ivC2D1) of mosunetuzumab administered on Day 1 of the second dosing cycle, wherein the ivC2D1 is about 30 mg; and
      (ii) 21 oral doses of lenalidomide administered on Days 1-21 of the second dosing cycle, wherein each oral dose of lenalidomide is about 20 mg.
85. The method, use, mosunetuzumab for use, lenalidomide for use, or mosunetuzumab and lenalidomide for use of embodiment 84, wherein the dosing regimen of the control treatment further comprises one or more additional 28-day dosing cycles.
86. The method, use, mosunetuzumab for use, lenalidomide for use, or mosunetuzumab and lenalidomide for use of embodiment 85, wherein the dosing regimen of the control treatment comprises ten additional 28-day dosing cycles.
87. The method, use, mosunetuzumab for use, lenalidomide for use, or mosunetuzumab and lenalidomide for use of embodiment 85 or 86, wherein each additional dosing cycle comprises:
   (a) an intravenous dose of mosunetuzumab administered on Day 1 of the additional dosing cycle, wherein the intravenous dose of mosunetuzumab is about 30 mg; and
   (b) twenty-one (21) oral doses of lenalidomide administered on Days 1-21 of the additional dosing cycle, wherein each oral dose of lenalidomide is about 20 mg.
88. The method, use, mosunetuzumab for use, lenalidomide for use, or mosunetuzumab and lenalidomide for use of any one of embodiments 1-87, wherein the subject has an Eastern Cooperative Oncology Group (ECOG) Performance Status of 0, 1, or 2.
89. The method, use, mosunetuzumab for use, lenalidomide for use, or mosunetuzumab and lenalidomide for use of any one of embodiments 1-88, wherein the subject has not been previously treated for FL.
90. The method, use, mosunetuzumab for use, lenalidomide for use, or mosunetuzumab and lenalidomide for use of any one of embodiments 1-88, wherein the subject has relapsed after or is refractory to one or more prior lines of systemic therapy.
91. The method, use, mosunetuzumab for use, lenalidomide for use, or mosunetuzumab and lenalidomide for use of embodiment 90, wherein the one or more prior lines of systemic therapy comprises an immunotherapy or a chemoimmunotherapy.
92. The method, use, mosunetuzumab for use, lenalidomide for use, or mosunetuzumab and lenalidomide for use of embodiment 90, wherein the one or more prior lines of systemic therapy comprises an anti-CD20 antibody.
93. The method, use, mosunetuzumab for use, lenalidomide for use, or mosunetuzumab and lenalidomide for use of embodiment 92, wherein the anti-CD20 antibody is rituximab.
94. The method, use, mosunetuzumab for use, lenalidomide for use, or mosunetuzumab and lenalidomide for use of any one of embodiments 1-93, wherein the FL is Grade 1, 2, or 3a.
95. A method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).
96. Use of mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).
97. Use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).
98. Mosunetuzumab for use for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).
99. The method, use, or mosunetuzumab for use of any one of embodiments 95-98, wherein the efficacy response is a CRR, and wherein the CRR or reference CRR is the proportion of the subjects receiving the corresponding treatment whose best overall response is a complete response (CR).
100. The method, use, or mosunetuzumab for use of embodiment 99, wherein the improved response in CRR is an increase in CRR compared to the reference CRR of between 1% and 20%.
101. The method, use, or mosunetuzumab for use of embodiment 99, wherein the efficacy response in CRR is non-inferior compared to the reference rate CRR.
102. The method, use, or mosunetuzumab for use of any one of embodiments 95-98, wherein the efficacy response is an ORR, and wherein the ORR or reference ORR is the proportion of the subjects receiving the corresponding treatment whose best overall response is a CR or a partial response (PR).
103. The method, use, or mosunetuzumab for use of embodiment 102, wherein the improved response in ORR is an increase in ORR compared to the reference ORR of between 1% and 13%.
104. The method, use, or mosunetuzumab for use of embodiment 102, wherein the efficacy response in ORR is non-inferior compared to the reference ORR.
105. The method, use, or mosunetuzumab for use of any one of embodiments 95-98, wherein the efficacy response is a DOR, and wherein the DOR or the reference DOR is measured starting from the time from the first occurrence of a documented CR or PR to disease progression or relapse or death from any cause, whichever occurs first.
106. The method, use, or mosunetuzumab for use of embodiment 105, wherein the DOR or the reference DOR is the median DOR of the plurality of subjects receiving the corresponding treatment.
107. The method, use, or mosunetuzumab for use of embodiment 105 or 106, wherein the improvement of the median DOR is an increase in the DOR compared to the reference DOR of between 1 and 13 months.
108. The method, use, or mosunetuzumab for use of embodiment 105, wherein the efficacy response in DOR is non-inferior compared to reference DOR.
109. The method, use, or mosunetuzumab for use of embodiment 105, wherein the improvement of the DOR is an increase in the rate of DOR at 12 months compared to the rate of reference DOR at 12 months of between 1% and 31%.
110. The method, use, or mosunetuzumab for use of embodiment 109, wherein the improvement of the DOR is an increase in the rate of DOR at 12 months compared to the rate of reference DOR at 12 months of about 8%.
111. The method, use, or mosunetuzumab for use of any one of embodiments 95-98, wherein the efficacy response is a DOCR, and wherein the DOCR or the reference DOCR is measured starting from the time from the first occurrence of a documented CR to disease progression or relapse or death from any cause, whichever occurs first.
112. The method, use, or mosunetuzumab for use of embodiment 111, wherein the DOCR or the reference DOCR is the median DOCR of the plurality of subjects receiving the corresponding treatment.
113. The method, use, or mosunetuzumab for use of embodiment 111, wherein the improvement of the DOCR is an increase in the rate of DOCR at 12 months compared to the rate of reference DOCR at 12 months of between 1% and 27%.
114. The method, use, or mosunetuzumab for use of embodiment 111, wherein the efficacy response in DOCR is non-inferior compared to the reference DOCR.
115. The method, use, or mosunetuzumab for use of any one of embodiments 99-114, wherein the CR or PR determined by PET/computed tomography (CT).
116. The method, use, or mosunetuzumab for use of embodiment 115, wherein the CR or PR is determined based on the Revised Response Criteria for Malignant Lymphoma (Cheson et al., 2007).
117. The method, use, or mosunetuzumab for use of embodiment any one of embodiments 95-99, wherein the efficacy response is a PFS, and wherein the PFS or the reference PFS is measured starting from the time from first treatment to the time of a first occurrence of disease progression or death from any cause.
118. The method, use, or mosunetuzumab for use of embodiment 117, wherein the PFS or the reference PFS is the median PFS of the plurality of subjects receiving the corresponding treatment.
119. The method, use, or mosunetuzumab for use of embodiment 117 or 118, wherein the improvement of the median PFS is an increase in the PFS compared to the reference PFS of between 1 and 14 months.
120. The method, use, or mosunetuzumab for use of embodiment 118, wherein the efficacy response in PFS is non-inferior compared to the reference PFS.
121. The method, use, or mosunetuzumab for use of embodiment 117, wherein the improvement of the PFS is in an increase in the rate of PFS compared to the rate of reference PFS at 12 months of between 1% and 26%.
122. The method, use, or mosunetuzumab for use of embodiment 121, wherein the improvement of the PFS is in an increase in the rate of PFS compared to the rate of reference PFS at 12 months of about 5%.
123. The method, use, or mosunetuzumab for use of any one of embodiments 95-98, wherein the efficacy response is an OS, wherein the OS or the reference OS is measured starting from the time from first treatment to death from any cause.
124. The method, use, or mosunetuzumab for use of embodiment 123, wherein the improvement of the OS is an increase in the rate of OS compared to the rate of reference OS at 12 months of between 1% and 9%.
125. The method, use, or mosunetuzumab for use of embodiment 123, wherein the efficacy response in OS is non-inferior compared to the reference OS.
126. A method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).
127. Use of mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).
128. Use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).
129. Mosunetuzumab for use for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects r results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).
130. The method, use, or mosunetuzumab for use of any one of embodiments 126-129, wherein the safety response is the rate of AE, and wherein the rate of AE or the reference rate of AE is the rate of AE in the plurality of subjects receiving the corresponding treatment.
131. The method, use, or mosunetuzumab for use of embodiment 130, wherein the safety response in rate of AE is non-inferior compared to the reference rate of AE.
132. The method, use, or mosunetuzumab for use of any one of embodiments 126-129, wherein the improved rate of AE is a decrease in the rate of Grade 3 or 4 AE compared to the reference rate of Grade 3 or 4 AE of between 11% and 21%.
133. The method, use, or mosunetuzumab for use of embodiment 132, wherein the improved rate of AE is a decrease in the rate of Grade 3 or 4 AE compared to the reference rate of Grade 3 or 4 AE of about 16%. 134. The method, use, or mosunetuzumab for use of embodiment 130, wherein the rate of AE is the rate of Grade 5 AE in the plurality of subjects receiving the corresponding treatment, and wherein the rate of AE is non-inferior compared to the reference rate of AE.
135. The method, use, or mosunetuzumab for use of any one of embodiments 132-134, wherein the Grade of AE is determined based on the National Cancer Institute Common Terminology Criteria for Adverse Events (NCI CTCAE), v4.0.
136. The method, use, or mosunetuzumab for use of any one of embodiments 126-129, wherein the safety response is the rate of SAE, and wherein the rate of SAE or the reference rate of SAE is the rate of SAE in the plurality of subjects receiving the corresponding treatment.
137. The method, use, or mosunetuzumab for use of embodiment 136, wherein the improved safety response in rate of SAE is a decrease in the rate of SAE compared to the reference rate of SAE of between 9% and 19%.
138. The method, use, or mosunetuzumab for use of embodiment 137, wherein the improved safety response in rate of SAE is a decrease in the rate of SAE compared to the reference rate of SAE of about 14%.
139. The method, use, or mosunetuzumab for use of any one of embodiments 126-129, wherein the safety response is the rate of CRS, and wherein the rate of CRS or the reference rate of CRS is the rate of CRS in the plurality of subjects receiving the corresponding treatment.
140. The method, use, or mosunetuzumab for use of embodiment 139, wherein the improved rate of CRS is a decrease in the rate of CRS compared to the reference rate of CRS of between 9% and 19%.
141. The method, use, or mosunetuzumab for use of embodiment 140, wherein the improved rate of CRS is a decrease in the rate of CRS compared to the reference rate of CRS of about 14%.
142. The method, use, or mosunetuzumab for use of embodiment 139, wherein the improved rate of CRS is a decrease in the rate of Grade 2+ CRS compared to the reference rate of Grade 2+ CRS of between 3% and 13%.
143. The method, use, or mosunetuzumab for use of embodiment 142, wherein the improved rate of CRS is a decrease in the rate of Grade 2+ CRS compared to the reference rate of Grade 2+ CRS of about 8%.
144. The method, use, or mosunetuzumab for use of embodiment 142 or 143, wherein the Grade of CRS is determined based on the Modified Cytokine Release Syndrome Grading System (Lee et al., 2014).
145. A method for treating Non-Hodgkin Lymphoma (NHL) or chronic lymphocytic leukemia (CLL) in a subject in need thereof, comprising administering to the subject a treatment comprising subcutaneously administered mosunetuzumab according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and
wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).
146. Use of mosunetuzumab for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).
147. Use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and
wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).
148. Mosunetuzumab for use for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and
wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).
149. The method, use, or mosunetuzumab for use of any one of embodiments 145-148, wherein one or more of the scAUC₀₋₈₄ or the ivAUC₀₋₈₄ is determined using population PK (popPK) model-predicted individual Empirical Bayesian Estimates (EBEs).
150. The method, use, or mosunetuzumab for use of any one of embodiments 95-149, wherein the treatment comprises subcutaneously administering mosunetuzumab according to a dosing regimen comprising at least a first 21-day dosing cycle and a second 21-day dosing cycle, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second dosing cycle comprises a first subcutaneous dose (scC2D1) of mosunetuzumab administered on Day 1 of the second dosing cycle, wherein the scC2D1 is about 45 mg.
151. The method, use, or mosunetuzumab for use of embodiment 150, wherein the dosing regimen of the treatment further comprises one or more additional 21-day dosing cycles.
152. The method, use, or mosunetuzumab for use of embodiment 151, wherein the dosing regimen of the treatment comprises six, fifteen, or twenty-three additional 21-day dosing cycles.
153. The method, use, or mosunetuzumab for use of embodiment 151 or 152, wherein each additional dosing cycle comprises a subcutaneous dose of mosunetuzumab administered on Day 1 of the additional dosing cycle, wherein the subcutaneous dose of mosunetuzumab is about 45 mg.
154. The method, use, or mosunetuzumab for use of any one of embodiments 95-153, wherein the control treatment comprises intravenously administering mosunetuzumab according to a dosing regimen comprising at least a first 21-day dosing cycle and a second 21-day dosing cycle, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg; and
   (b) the second dosing cycle comprises a first intravenous dose (ivC2D1) of mosunetuzumab administered on Day 1 of the second dosing cycle, wherein the ivC2D1 is about 60 mg.
155. The method, use, or mosunetuzumab for use of embodiment 154, wherein the dosing regimen of the control treatment further comprises one or more additional 21-day dosing cycles.
156. The method, use, or mosunetuzumab for use of embodiment 155, wherein the dosing regimen of the control treatment comprises six, fifteen, or twenty-three additional 21-day dosing cycles.
157. The method, use, or mosunetuzumab for use of embodiment 155 or 156, wherein each additional dosing cycle comprises an intravenous dose of mosunetuzumab administered on Day 1 of the additional dosing cycle, wherein the intravenous dose of mosunetuzumab is about 30 mg.
158. The method, use, or mosunetuzumab for use of any one of embodiments 95-157, wherein the subject has an Eastern Cooperative Oncology Group (ECOG) Performance Status of 0 or 1.
159. The method, use, or mosunetuzumab for use of any one of embodiments 95-158, wherein the subject has relapsed after or is refractory to one or more prior lines of systemic therapy.
160. The method, use, or mosunetuzumab for use of embodiment 159, wherein the subject has relapsed after or is refractory to two or more prior lines of systemic therapy.
161. The method, use, or mosunetuzumab for use of embodiment 159 or 160, wherein the one or more prior lines of systemic therapy comprises an anti-CD20 antibody, an alkylating agent, a Bruton's tyrosine kinase (BTK) inhibitor, an anthracycline, or a combination thereof.
162. The method, use, or mosunetuzumab for use of any one of embodiments 95-161, wherein the NHL is a follicular lymphoma (FL), a marginal zone lymphoma (MZL), a diffuse large B cell lymphoma (DLBCL), a transformed FL (trFL), a high grade B cell lymphoma (HGBL), a primary mediastinal B cell lymphoma (PMLBCL), a transformed indolent NHL, a mantle cell lymphoma (MCL), a Richter's transformation, or a small lymphocytic lymphoma (SLL).
163. The method, use, or mosunetuzumab for use of embodiment 162, wherein the NHL is an FL, and wherein the FL is Grade 1-3b FL or a transformed FL.
164. The method, use, or mosunetuzumab for use of embodiment 163, wherein the FL is Grade 1-3a FL, and wherein the subject has relapsed after or is refractory to two or more prior lines of systemic therapy comprising an anti-CD20 antibody and an alkylating agent.
165. The method, use, or mosunetuzumab for use of embodiment 162, wherein the NHL is a DLBCL or a trFL.
166. The method, use, or mosunetuzumab for use of embodiment 165, wherein the NHL is a DLBCL or a trFL, and wherein the subject has relapsed after or is refractory to two or more prior lines of systemic therapy comprising an anthracycline and an anti-CD20 antibody.
167. The method, use, or mosunetuzumab for use of embodiment 162, wherein the NHL is a Richter's transformation.
168. The method, use, or mosunetuzumab for use of embodiment 167, wherein the NHL is a Richter's transformation, and wherein the subject has relapsed after or is refractory to one or more prior lines of therapy comprising an anthracycline and an anti-CD20 antibody.
169. The method, use, or mosunetuzumab for use of any one of embodiments 161-168, wherein the anti-CD20 antibody is rituximab.
170. The method, use, or mosunetuzumab for use of embodiment 162, wherein the NHL is an MCL.
171. The method, use, or mosunetuzumab for use of embodiment 170, wherein the NHL is an MCL, and wherein the subject has relapsed after or is refractory to one or more prior lines of systemic therapy comprising a BTK inhibitor.
172. The method, use, or mosunetuzumab for use of any one of embodiments 1-171, wherein the method comprises administering an additional therapeutic agent.
173. The method, use, or mosunetuzumab for use of embodiment 172, wherein the additional therapeutic agent comprises a corticosteroid, an antihistamine, an antipyretic, or an IL-6R antagonist.
174. The method, use, or mosunetuzumab for use of embodiment 173, wherein the additional therapeutic agent is a corticosteroid, and wherein the corticosteroid comprises prednisone, methylprednisolone, or dexamethasone.
175. The method, use, or mosunetuzumab for use of embodiment 173, wherein the additional therapeutic agent is an antihistamine, and wherein the antihistamine comprises diphenhydramine hydrochloride or equivalent.
176. The method, use, or mosunetuzumab for use of embodiment 173, wherein the additional therapeutic agent is an antipyretic, and wherein the antipyretic is acetaminophen.
177. The method, use, or mosunetuzumab for use of embodiment 173, wherein the additional therapeutic agent is an IL-6R antagonist, and wherein the IL-6R antagonist is tocilizumab.
178. A method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
179. Use of mosunetuzumab in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
180. Use of lenalidomide in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
181. Use of mosunetuzumab and lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
182. Use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
183. Use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
184. Use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS),
wherein the combination treatment comprises subcutaneously administering mosunetuzumab wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
185. Mosunetuzumab for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS),
wherein the combination treatment comprises subcutaneously administering mosunetuzumab wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
186. Lenalidomide for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
187. Mosunetuzumab and lenalidomide for use for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS),
wherein the combination treatment comprises subcutaneously administering mosunetuzumab and orally administering wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
188. A method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
189. Use of mosunetuzumab in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
190. Use of lenalidomide in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
191. Use of mosunetuzumab and lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
192. Use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
193. Use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
194. Use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
195. Mosunetuzumab for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
196. Lenalidomide for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
197. Mosunetuzumab and lenalidomide for use for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
198. A method for treating follicular lymphoma (FL) in a subject in need thereof, comprising administering to the subject a combination treatment comprising subcutaneously administered mosunetuzumab and orally administered lenalidomide according to a dosing regimen comprising a first 21-day dosing cycle and three 28-day dosing cycles, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1 D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
199. Use of mosunetuzumab in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
200. Use of lenalidomide in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
201. Use of mosunetuzumab and lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
202. Use of mosunetuzumab in the manufacture of a medicament for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
203. Use of lenalidomide in the manufacture of a medicament for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇),
wherein the combination treatment comprises subcutaneously administering mosunetuzumab wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
204. Use of mosunetuzumab and lenalidomide in the manufacture of a medicament for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇),
wherein the combination treatment comprises subcutaneously administering mosunetuzumab wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
205. Mosunetuzumab for use in combination with lenalidomide for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
206. Lenalidomide for use in combination with mosunetuzumab for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
207. Mosunetuzumab and lenalidomide for use for treating follicular lymphoma (FL) in a subject in need thereof, wherein mosunetuzumab is to be administered subcutaneously and lenalidomide is to be administered orally as a combination treatment, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇),
wherein the combination treatment comprises subcutaneously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
   (b) the second to twelfth dosing cycles each comprises:
      (i) a first subcutaneous dose (scC2D1-scC12D1) of mosunetuzumab administered on Day 1 of each of dosing cycles 2-12, wherein the scC2D1-scC12D1 is each about 45 mg; and
      (ii) twenty-one (21) oral doses of lenalidomide administered on Days 1-21 of dosing cycles 2-12, wherein each oral dose of lenalidomide is about 20 mg, and
wherein the control treatment comprises intravenously administering mosunetuzumab and orally administering lenalidomide according to a dosing regimen comprising twelve dosing cycles, wherein the first dosing cycle is a 21-day dosing cycle and the second to twelfth dosing cycles are 28-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
   (b) the second to twelfth dosing cycles each comprises:
      (i) a first intravenous dose (ivC2D1-ivC12D1) of mosunetuzumab administered on Day 1 of each of dosing cycles 2-12, wherein the ivC2D1-ivC12D1 is each about 30 mg; and
      (ii) 21 oral doses of lenalidomide administered on Days 1-21 of dosing cycles 2-12, wherein each oral dose of lenalidomide is about 20 mg.
208. The method, use, mosunetuzumab for use, lenalidomide for use, or mosunetuzumab and lenalidomide for use of any one of embodiments 178-207, wherein the dosing regimen of the combination treatment further comprises nine subcutaneous maintenance doses of mosunetuzumab.
209. The method, use, mosunetuzumab for use, lenalidomide for use, or mosunetuzumab and lenalidomide for use of embodiment 208, wherein the nine subcutaneous maintenance doses of mosunetuzumab are administered every eight weeks starting eight weeks after Day 1 of the tenth additional 28-day dosing cycle.
210. The method, use, mosunetuzumab for use, lenalidomide for use, or mosunetuzumab and lenalidomide for use of embodiment 208 or 209, wherein each maintenance dose of mosunetuzumab is about 45 mg.
211. A method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
(c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
212. Use of mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
(c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
213. Use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
(c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
214. Mosunetuzumab for use for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
(c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
215. A method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
   (c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
216. Use of mosunetuzumab for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
   (c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
217. Use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
   (c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
218. Mosunetuzumab for use for treating Non-Hodgkin Lymphoma (NHL) or chronic lymphocytic leukemia (CLL) in a subject in need thereof, wherein mosunetuzumab is to be administered subcutaneously as a treatment, wherein administering the treatment to a plurality of subjects r results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
   (c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
219. A method for treating Non-Hodgkin Lymphoma (NHL) or chronic lymphocytic leukemia (CLL) in a subject in need thereof, comprising administering to the subject a treatment comprising subcutaneously administered mosunetuzumab according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and
wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
   (c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
220. Use of mosunetuzumab for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and
wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
   (c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
221. Use of mosunetuzumab in the manufacture of a medicament for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and
wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄), wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
   (c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
222. Mosunetuzumab for use for treating Non-Hodgkin Lymphoma (NHL) or chronic lymphocytic leukemia (CLL) in a subject in need thereof, wherein mosunetuzumab is to be administered subcutaneously as a treatment according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄);
wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄),
wherein the treatment comprises subcutaneously administering mosunetuzumab according to a dosing regimen comprising eight or seventeen 21-day dosing cycles, wherein:
   (a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
   (b) the second to eighth dosing cycles or the second to seventeenth dosing cycles each comprises a first subcutaneous dose (scC2D1-scC8D1 or scC2D1-C17D1) of mosunetuzumab administered on Day 1 of each of dosing cycles 2-8 or 2-17, wherein the scC2D1-scC8D1 or scC2D1-C17D1 is each about 45 mg, and
wherein the control treatment comprises intravenously administering mosunetuzumab according to a dosing regimen comprising eight or seventeen 21-day dosing cycles, wherein:
   (a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg;
   (b) the second dosing cycle comprises a first intravenous dose (ivC2D1) of mosunetuzumab administered on Day 1 of the second dosing cycle, wherein the ivC2D1 is about 60 mg; and
   (c) the third to eighth dosing cycles or third to seventeenth dosing cycles each comprises a first intravenous dose (ivC3D1-ivC8D1 or ivC3D1-ivC17D1) of mosunetuzumab administered on Day 1 of each of dosing cycles 3-8 or 3-17, wherein the ivC3D1-ivC8D1 or ivC3D1-ivC17D1 is each about 30 mg.
223. The method, use, mosunetuzumab for use, lenalidomide for use, or mosunetuzumab and lenalidomide for use of embodiment 66, wherein the scC1D1 is 5 mg, the scC1D2 is 45 mg, and the scC1D3 is 45 mg; the scC2D1 is 45 mg; and each oral dose of lenalidomide is 20 mg.
224. The method, use, mosunetuzumab for use, lenalidomide for use, or mosunetuzumab and lenalidomide for use of embodiment 69, wherein the subcutaneous dose of mosunetuzumab is 45 mg; and each oral dose of lenalidomide is 20 mg.
225. The method, use, mosunetuzumab for use, lenalidomide for use, or mosunetuzumab and lenalidomide for use of embodiment 83, wherein each maintenance dose of mosunetuzumab is 45 mg.
226. The method, use, mosunetuzumab for use, lenalidomide for use, or mosunetuzumab and lenalidomide for use of embodiment 84, wherein the ivC1D1 is 1 mg, the ivC1D2 is 2 mg, and the ivC1D3 is 30 mg; the ivC2D1 is 30 mg; and each oral dose of lenalidomide is 20 mg.
227. The method, use, mosunetuzumab for use, lenalidomide for use, or mosunetuzumab and lenalidomide for use of embodiment 87, wherein the intravenous dose of mosunetuzumab is 30 mg; and each oral dose of lenalidomide is 20 mg.
228. The method, use, or mosunetuzumab for use of embodiment 150, wherein the scC1D1 is 5 mg, the scC1D2 is 15 mg or 45 mg, and the scC1D3 is 45 mg; and the scC2D1 is 45 mg.
229. The method, use, or mosunetuzumab for use of embodiment 228, wherein the scC1D2 is 45 mg.
230. The method, use, or mosunetuzumab for use of embodiment 153, wherein the subcutaneous dose of mosunetuzumab is 45 mg.
231. The method, use, or mosunetuzumab for use of embodiment 154, wherein the ivC1D1 is 1 mg, the ivC1D2 is 2 mg, and the ivC1D3 is 60 mg; and the ivC2D1 is 60 mg.
232. The method, use, or mosunetuzumab for use of embodiment 157, wherein the intravenous dose of mosunetuzumab is 30 mg.
233. The method of any one of embodiments 66-71, wherein administering the combination treatment to a plurality of subjects having FL results in an ORR in the plurality of subjects of between about 81% to about 99%.
234. The method of embodiment 233, wherein the ORR is about 90%.
235. The method of any one of embodiments 66-71, wherein administering the combination treatment to a plurality of subjects having FL results in a CRR in the plurality of subjects of between about 79% to about 97%.
236. The method of embodiment 235, wherein the CRR is about 88%.
237. The method of any one of embodiments 66-71, wherein administering the combination treatment to a plurality of subjects having FL results in a 12-month event-free rate for DOR in the plurality of subjects of between about 86% to about 100%. 238. The method of embodiment 237, wherein the 12-month event-free rate for DOR is about 94%.
239. The method of any one of embodiments 66-71, wherein administering the combination treatment to a plurality of subjects having FL results in a 12-month event-free rate for DOCR in the plurality of subjects of between about 86% to about 100%. 240. The method of embodiment 239, wherein the 12-month event-free rate for DOCR is about 94%.
241. The method of any one of embodiments 66-71, wherein administering the combination treatment to a plurality of subjects having FL results in a 12-month event-free rate for PFS in the plurality of subjects of between about 76% to about 98%. 242. The method of embodiment 241, wherein the 12-month event-free rate for PFS is about 87%.
243. The method of any one of embodiments 66-71, wherein administering the combination treatment to a plurality of subjects having FL results in a 12-month event-free rate for OS in the plurality of subjects of between about 92% to about 100%.
244. The method of embodiment 243, wherein the 12-month event-free rate for OS is about 97%. 245. The method of any one of embodiments 233-244, wherein the FL is previously untreated (1 L) FL.
246. The method of any one of embodiments 150-153, wherein administering the treatment to a plurality of subjects having R/R FL results in an ORR of between about 66% to about 85%.
247. The method of embodiment 246, wherein the ORR is about 77%.
248. The method of any one of embodiments 150-153, wherein administering the treatment to a plurality of subjects having R/R FL results in a CRR in the plurality of subjects of between about 51% to about 72%.
249. The method of embodiment 248, wherein the CRR is about 62%.
250. The method of any one of embodiments 150-153, wherein administering the treatment to a plurality of subjects having R/R FL results in an 18-month event-free rate for DOCR in the plurality of subjects of between about 57% to about 83%.
251. The method of embodiment 250, wherein the 18-month event-free rate for DOCR is about 70%,
252. The method of any one of embodiments 150-153, wherein administering the treatment to a plurality of subjects having R/R FL results in a median DOCR in the plurality of subjects of at least about 20 months.
253. The method of embodiment 252, wherein the median DOCR is about 35 months.
254. The method of any one of embodiments 150-153, wherein administering the treatment to a plurality of subjects having R/R FL results in an 18-month event-free rate for PFS in the plurality of subjects of between about 46% to about 68%.
255. The method of embodiment 254, wherein the 18-month event-free rate for PFS is about 57%.
256. The method of any one of embodiments 150-153, wherein administering the treatment to a plurality of subjects having R/R FL results in a median PFS in the plurality of subjects of at least 14 months.
257. The method of embodiment 256, wherein the median PFS is about 24 months.
258. The method of any one of embodiments 150-153, wherein administering the treatment to a plurality of subjects having 1 L FL results in an ORR of between about 76% to about 96%.
259. The method of embodiment 258, wherein the ORR is about 87%.
260. The method of any one of embodiments 150-153, wherein administering the treatment to a plurality of subjects having 1 L FL results in a CRR in the plurality of subjects of between about 55% to about 67%.
261. The method of embodiment 260, wherein the CRR is about 61%.
262. The method of any one of embodiments 150-153, wherein administering the treatment to a plurality of subjects having 1 L FL results in a 12-month event-free rate for PFS in the plurality of subjects of between about 73% to about 90%.
263. The method of embodiment 262, wherein the 12-month event-free rate for PFS is about 83%.
264. The method of any one of embodiments 150-153, wherein administering the treatment to a plurality of subjects having 1 L FL results in a median time to response (TTR) in the plurality of subjects exhibiting a response of between about 1 month to about 6 months.
265. The method of embodiment 264, wherein the median TTR is about 3 months.
266. The method of any one of embodiments 258-265, wherein the subjects of the plurality have a high tumor burden, per the Groupe d'Etude des Lymphomes Folliculaires (GELF) criteria.
267. The method of any one of embodiments 150-153, wherein administering the treatment to a plurality of subjects having 1 L MZL results in an ORR of between about 58% to about 88%.
268. The method of embodiment 267, wherein the ORR is about 75%.
269. The method of any one of embodiments 150-153, wherein administering the treatment to a plurality of subjects having 1 L MZL results in a CRR in the plurality of subjects of between about 44% to about 77%.
270. The method of embodiment 269, wherein the CRR is about 61%.
271. The method of any one of embodiments 150-153, wherein administering the treatment to a plurality of subjects having 1 L MZL results in a median TTR in the plurality of subjects exhibiting a response of between about 2 months to about 6 months.
272. The method of embodiment 271, wherein the median TTR is about 3 months.

### VIII. EXAMPLES

The following are examples of the methods of the invention. It is understood that various other embodiments may be practiced, given the general description provided above.

### Example 1. An Open-Label, Multicenter, Phase I/Ib Trial Evaluating the Safety, Efficacy, and Pharmacokinetics of Escalating Doses of Mosunetuzumab (BTCT4465A) as a Single Agent in Patients with Relapsed or Refractory B-Cell Non-Hodgkin's Lymphoma and Chronic Lymphocytic Leukemia

This Example describes GO29781, a Phase I/Ib, multicenter, open-label, dose-escalation study of mosunetuzumab administered as a single agent in patients with R/R hematologic malignancies expected to express CD20, including B-cell non-Hodgkin's lymphoma (NHL) and chronic lymphocytic leukemia (CLL). The study enrolls approximately 130-226 patients during the dose-escalation stage (100-166 patients with NHL and 30-60 patients with CLL) and approximately 290-520 patients during the expansion stage at approximately 45-50 investigative sites globally.

The study had four groups, A, B, D, and F. For the purposes of the disclosure, the results from Groups A and B, which were cohorts that were administered mosunetuzumab intravenously, was used as a basis of comparison with Groups D and F, who were administered mosunetuzumab subcutaneously. One measure of the mitigation of adverse events realized from subcutaneous administration was the rates of CRS in the different groups.

### Overview

### A. Objectives

This study evaluated the safety, tolerability, and pharmacokinetics of mosunetuzumab in patients with relapsed or refractory (R/R) non-Hodgkin's lymphoma (NHL) and chronic lymphocytic leukemia (CLL) as described below:
Administered intravenously (IV) as a single agent on a Cycle 1 non-fractionated dose schedule (Group A);
Administered IV as a single agent on a Cycle 1 step-up dose schedule (Group B);
Administered subcutaneously (SC) as a single agent on a Cycle 1 non-fractionated dose schedule (Group D);
Administered SC as a single agent on a Cycle 1 step-up dose schedule (Group F).

This study determined the maximum tolerated dose (MTD) and dose-limiting toxicities (DLTs) of mosunetuzumab in patients with R/R NHL and CLL as described below:
Administered IV on a Cycle 1 non-fractionated dose schedule (Group A);
Administered IV on a Cycle 1 step-up dose schedule (Group B);
Administered SC on a Cycle 1 non-fractionated dose schedule (Group D);
Administered SC as a single agent on a Cycle 1 step-up dose schedule (Group F).

This study identified, on the basis of safety, pharmacokinetic (PK), and pharmacodynamic data, the recommended Phase II dose(s) and schedule(s) of mosunetuzumab as a single agent in patients with R/R NHL and for CLL. In addition, this study evaluated the efficacy of mosunetuzumab using a Cycle 1 step-up dosing schedule as a single agent (Group B) in patients with R/R diffuse large B-cell lymphoma (DLBCL) and transformed follicular lymphoma (FL), and patients with R/R FL, as measured by Independent Review Facility-assessed complete response (CR) rate according to standard NHL response criteria.

This study assessed the incidence of anti-drug antibodies (ADAs) to mosunetuzumab and their relationship to relevant clinical outcomes.

Where evaluation of efficacy of mosunetuzumab as single agent was not a primary objective as described above, this study conducted a preliminary assessment of the anti-tumor activity of mosunetuzumab, as a single agent, in patients with R/R NHL and CLL.

### B. Study Design

### Description of Study

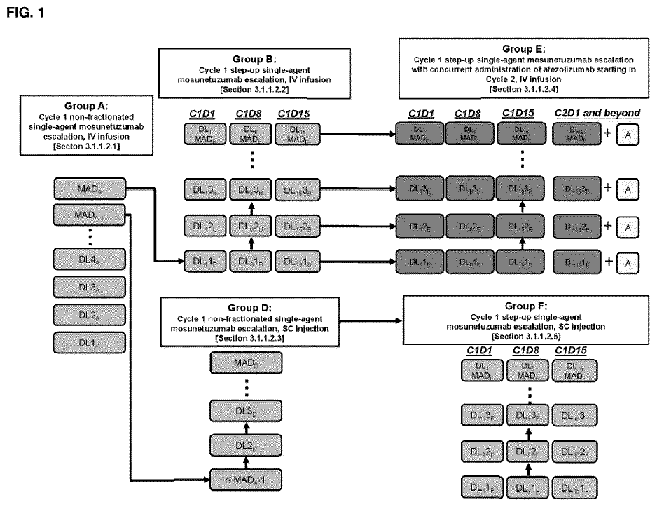

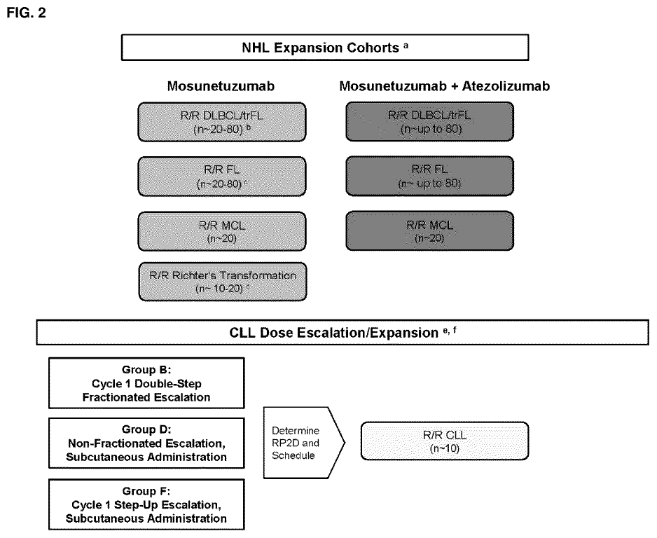

FIG. 1 provides an overview of the dose-escalation portion of the study, and FIG. 2 depicts the dose-expansion phase. Initially, mosunetuzumab was given as a single non-fractionated IV dose on Day 1 of each cycle (Group A). Cycle 1 dosing was subsequently modified such that Group A dose escalation stops and mosunetuzumab dose escalation was conducted as follows:
Mosunetuzumab dose escalation utilizing a Cycle 1 step-up IV dosing scheme (Group B)
Mosunetuzumab dose escalation utilizing a Cycle 1 non-fractionated SC dosing scheme (Group D)
Mosunetuzumab dose escalation utilizing a Cycle 1 step-up SC dosing scheme (Group F).

Enrollment into escalation Groups B, D, and F did not necessarily begin concurrently.

After the recommended Phase II doses (RP2Ds) and schedules had been identified for single-agent mosunetuzumab, further assessment of mosunetuzumab clinical activity as a single agent was conducted in indication-specific expansion cohorts (FIG. 2).

In addition to dose escalation and expansion in NHL, separate escalation and expansions in CLL was also conducted. The rules for dose escalation and dose expansion were identical to those described for NHL escalation and expansion.

### Dose-Escalation Stage

The dose-escalation stage of the study assessed the safety, tolerability, and pharmacokinetics of mosunetuzumab administered by IV infusion or SC injection. Up to five dose-escalation groups were enrolled (FIG. 1):
Group A: Cycle 1 non-fractionated single-agent mosunetuzumab escalation, IV infusion (enrollment into dose-escalation Group A was stopped to prioritize assessment of other dosing schedule and route of mosunetuzumab);
Group B: Cycle 1 step-up single-agent mosunetuzumab escalation, IV infusion;
Group D: Cycle 1 non-fractionated single-agent mosunetuzumab escalation, SC injection;
Group F: Cycle 1 step-up single-agent mosunetuzumab escalation, SC injection.

Dose-escalation Group C (Cycle 1 non-fractionated single-agent mosunetuzumab following a single dose of obinutuzumab; IV infusion) was removed.

Initially, dose-escalation cohorts in Group A consisted of 1 patient. Conversion to a standard 3+3 design occurred based on the criteria provided herein. Subsequently, dose-escalation cohorts consisted of at least 3 patients, unless DLTs was observed in the first 2 patients prior to enrollment of a third patient, according to a standard 3+3 design.

Dose-escalation cohorts in Groups B, D, and F were based on a standard 3+3 design from the outset.

For each dose-escalation cohort, treatment with the first dose of mosunetuzumab was staggered such that the second patient enrolled in the cohort received mosunetuzumab at least 72 hours after the first enrolled patient received the first dose of mosunetuzumab in order to assess for any severe and unexpected acute drug or infusion/injection-related toxicities; dosing in subsequent patients in each cohort was staggered by at least 24 hours from the end of the prior patients' administration. Staggered patient enrollment was not required for enrollment of additional patients to acquire additional safety and pharmacodynamic data at a dose level that was shown to not exceed the MTD.

Patients exhibiting acceptable safety and evidence of clinical benefit (as defined herein) continued to receive mosunetuzumab every 21 days up to a maximum of 8 or 17 cycles until confirmed objective disease progression or unacceptable toxicity, whichever occurred first. Re-treatment with mosunetuzumab proceeded based on clinical responses to initial treatment.

Prior clinical trial experience in hematologic malignancies suggested that different toxicity profiles were observed depending on the patient population treated. Specifically, the toxicity of mosunetuzumab in CLL patients was distinct from that of NHL patients due to the presence of a larger number of circulating tumor cells and/or differences in overall disease burden. Because of this potential difference in toxicity profile and/or MTD in CLL patients, separate dose escalations were provided for NHL and CLL patients. The initial dose escalation was conducted in NHL patients. Initiation of dose escalation in CLL patients was at the Sponsor's discretion and followed Group B dose-escalation rules or Group D dose-escalation rules as applicable. The cumulative Cycle 1 starting dose level in CLL was at least one dose level below the highest cumulative Cycle 1 dose that cleared the DLT assessment window in the corresponding NHL dose escalation.

Mosunetuzumab dose levels were independent of patient weight. The starting dose of 50 μg was based on the minimal anticipated biologic effect level (MABEL) in humans.

### Definition of Dose-Limiting Toxicity

Although CRS was graded according to the Modified Cytokine Release Syndrome Grading System (Table 2), for dose-escalation decisions, DLTs related to CRS were defined based on individual signs and symptoms and laboratory data according to the National Cancer Institute Common Terminology for Adverse Events (NCI CTCAE) v4.0. Dose-limiting toxicities (DLTs) were treated according to clinical practice and were monitored through their resolution. All adverse events were considered related to mosunetuzumab unless such events were clearly attributed by the investigator to another clearly identifiable cause (e.g., documented disease progression, concomitant medication, or pre-existing medical condition). Decreases in B cells, lymphopenia, and/or leukopenia due to decreases in B cells were not considered DLTs as they are expected pharmacodynamic outcomes of mosunetuzumab treatment.

TABLE 2
Modified cytokine release syndrome grading system

| Grade | Modified Cytokine Release Syndrome Grading System |
|---|---|
| Grade 1 | Symptoms are not life threatening and require symptomatic |
| | treatment only (e.g., fever, nausea, fatigue, headache, |
| | myalgia, malaise) |
| Grade 2 | Symptoms require and respond to moderate intervention |
| | Oxygen requirement <40%; or |
| | Hypotension responsive to fluids or low dose^{a} of one |
| | vasopressor; or Grade 2 organ toxicity |
| Grade 3 | Symptoms require and respond to aggressive intervention |
| | Oxygen requirement ≥40%; or |
| | Hypotension requiring high dose^{b} or multiple vasopressors; |
| | or Grade 3 organ toxicity or Grade 4 transaminitis |
| Grade 4 | Life-threatening symptoms |
| | Requirement for ventilation support or |
| | Grade 4 organ toxicity (excluding transaminitis) |
| Grade 5 | Death |

Lee 2014 criteria: Lee et al., Blood, 124: 188-195, 2014.
^{a}Low-dose vasopressor: single vasopressor at doses below that shown in Table 3.
^{b}High-dose vasopressor: as defined in Table 3.

TABLE 3
High-dose vasopressors
High-Dose Vasopressors (duration ≥3 hours)

| Pressor | Dose | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + norepinephrine |
| | equivalent of ≥10 g/min ^{a} |
| If on combination or vasopressors | Norepinephrine equivalent |
| (not vasopressin) | of ≥20 μg/min ^{a} |

min = minute; VASST = Vasopressin and Septic Shock Trial.
^{a} VASST vasopressor equivalent equation: norepinephrine equivalent dose = [norepinephrine (μg/min)] + [dopamine (μg/kg/min) ÷ 2] + [epinephrine (μg/min)] + [phenylephrine (μg/min) ÷ 10].

For dose-escalation purposes, the DLT assessment period was defined by the following time periods depending on the assigned dose-escalation group:
Groups A, B, D, and F: Cycle 1 Day 1 through Cycle 1 Day 21.
For treatment Groups B and F, the 21-day DLT assessment period was subdivided into three windows as described herein.
For treatment Groups B and F, in the case of dose delay for the Cycle 1 Day 8, and/or Cycle 1 Day 15 dosing, the DLT assessment period was extended accordingly to 7 days after the C1D15 administration date.

A DLT was defined as any of the following adverse events occurring during the DLT assessment period:
Any Grade 4 adverse events not considered by the investigator to be attributable to another clearly identifiable cause, with the following exceptions:
   Grade 4 neutropenia which was not accompanied by temperature elevation (as a single oral temperature of ≥38.3° C. (101° F.) or an oral temperature of ≥38.0° C. (100.4° F.) sustained for ≥1 hour) and improved to Grade≤2 (or to >80% of the baseline value, whichever is lower) within 1 week.
   Grade 4 lymphopenia, which was an expected outcome of therapy.
   Grade 4 leukopenia, which was an expected outcome of therapy.
   For CLL patients only: neutropenia was graded based on National Cancer Institute-sponsored Working Group (NCI-WG) definitions (Hallek et al., Blood, 111:5446-5456, 2008); absolute neutrophil count (ANC)<1000/mm³ due to bone marrow involvement prior to study treatment was not evaluable for DLT based on ANC
   For CLL patients only: thrombocytopenia was graded based on NCI-WG definitions (Hallek et al., Blood, 111:5446-5456, 2008); platelet counts <20,000/μL due to bone marrow involvement prior to study treatment was not evaluable for DLT based on platelet counts.
   For CLL patients only: anemia was graded based on NCI-WG definitions (Hallek et al., Blood, 111:5446-5456, 2008); Grade 4 anemia that improved to Grade 3 within 1 week and further improved to Grade 2 within another week without red blood cell (RBC) transfusion was not a DLT.
Any Grade 3 hematologic adverse event not considered by the investigator to be attributable to another clearly identifiable cause, with the following exceptions:
   Grade 3 lymphopenia, which was an expected outcome of therapy.
   Grade 3 leukopenia, which was an expected outcome of therapy.
   Grade 3 neutropenia that was not accompanied by temperature elevation (as a single oral temperature of ≥38.3° C. (101° F.) or an oral temperature of ≥38.0° C. (100.4° F.) sustained for ≥1 hour) and improved to Grade≤2 (or to >80% of the baseline value, whichever is lower) within 1 week.
      For CLL patients only: neutropenia was graded based on the NCI-WG Grading Scale for Chronic Lymphocytic Leukemia (Hallek et al., Blood, 111:5446-5456, 2008); ANC <1000/mm³ due to bone marrow involvement prior to study treatment was not evaluable for DLT based on ANC.
   Grade 3 thrombocytopenia that improved to Grade≤2 (or to ≥80% of the baseline value, whichever is lower) within 1 week without platelet transfusion and was not associated with bleeding that was considered clinically significant by the investigator.
      For CLL patients only: thrombocytopenia was graded based on NCI-WG definitions (Hallek et al., Blood, 111:5446-5456, 2008); a platelet count<20,000/μL due to bone marrow involvement prior to study treatment was not evaluable for DLT based on platelet counts.
   For CLL patients only: Grade 3 anemia (based on the NCI-WG Grading Scale for Chronic Lymphocytic Leukemia (Hallek et al., Blood, 111:5446-5456, 2008) that improved to Grade 2 within 1 week without RBC transfusion.
Any Grade 3 non-hematologic adverse event not considered by the investigator to be attributable to another clearly identifiable cause, with the following exceptions:
   Grade 3 nausea or vomiting in the absence of premedication or that was managed with resulting resolution to Grade≤2 with oral or IV anti-emetics within 24 hours. Grade 3 nausea or vomiting that required total parenteral nutrition or hospitalization were not excluded and were considered DLTs.
   Grade 3 fatigue lasting≤3 days.
   Grade 3 (NCI CTCAE v4) individual signs and symptoms of CRS that occurred in the context of Grade≤2 CRS (Table 2) and lasts <3 days were not considered DLTs.
   Grade 3 laboratory abnormality that was asymptomatic and deemed by the investigator not to be clinically significant.
Any hepatic function abnormality as defined by the following:
   Aspartate transaminase (AST) or alanine aminotransferase (ALT)>3×the upper limit of normal (ULN) AND total bilirubin >2×ULN. Any AST or ALT 3×the ULN and total bilirubin >2×ULN where no individual laboratory value exceeded Grade 3 and lasts <3 days was considered a DLT.
   Any Grade 3 AST or ALT elevation with the following exception: any Grade 3 AST or ALT elevation that lasted <3 days was considered a DLT.

### Dose-Escalation Rules and Determination of the Maximum Tolerated Dose

Specific rules for Groups A, B, D, and F dose escalations are detailed below. Initiation of individual group dose escalations was at the Sponsor's discretion. Relevant demographic, AE, laboratory, dose administration, and available PK and pharmacodynamic data (e.g., serum cytokines and markers of T-cell activation) were reviewed prior to each dose-escalation decision.

Determination of whether a patient was evaluable for DLT assessment was made in accordance with the following rules:
Patients who received study treatment and remained on study through the DLT assessment window were considered DLT-evaluable.
Patients who discontinued from treatment with single-agent mosunetuzumab prior to completing the DLT assessment window for reasons other than a DLT were considered non-evaluable for dose-escalation decisions and MTD determination and were replaced by an additional patient at that same dose level.
For patients enrolled into Groups B and F only: patients who had dose delays exceeding 7 days following the scheduled Cycle 1 Day 1, Cycle 1 Day 8, or Cycle 1 Day 15 dose for a non-DLT adverse event were DLT unevaluable and may have been replaced. For patients who had dose delays of 7 days or fewer, the DLT window extended until 7 days following the actual Cycle 1 Day 15 dose.
Patients who received supportive care during the DLT assessment window that confounded the evaluation of DLTs (not including supportive care described herein as part of the DLT definition) may have been replaced.

On the basis of a review of real-time safety data and available preliminary PK data, dose escalation was halted or modified as deemed appropriate.

To acquire additional safety and pharmacodynamic data to better fully inform the RP2D, additional patients were enrolled at a dose level that has been shown to not exceed the MTD based on the dose-escalation criteria described above, and at which there was evidence of anti-tumor activity and/or pharmacodynamic biomarker modulation. Up to approximately three additional patients per dose level may have been enrolled. For the purposes of dose-escalation decisions, these patients were included as part of the DLT-evaluable population.

### C. Inclusion Criteria

Patients met the following criteria for study entry:
Age ≥18 years.
Able to comply with the study protocol, in the investigator's judgment.
Eastern Cooperative Oncology Group Performance Status of 0 or 1
Life expectancy of at least 12 weeks.
History of one of the following histologically-documented hematologic malignancies that were expected to express the CD20 antigen who had relapsed after or failed to respond to at least one prior systemic treatment regimen and for whom there was no available therapy expected to improve survival (e.g., standard chemotherapy, autologous stem cell transplant (SCT), CAR-T):
   Dose-escalation:
      Grades 1-3b FL; marginal zone lymphoma (including splenic, nodal, and extra-nodal); transformed indolent NHL; Richter's transformation; DLBCL; primary mediastinal B-cell lymphoma; small lymphocytic lymphoma; or mantle cell lymphoma.
   Dose-expansion:
      DLBCL/transformed FL cohort: patients who had relapsed after or failed to respond to at least two prior systemic treatment regimens (including at least one prior regimen containing anthracycline, and at least one containing an anti-CD20-directed therapy). The number of transformed FL patients enrolled in the study could be limited. Transformed FL was an eligible diagnosis for enrollment in the DLBCL cohort but were relapsed or refractory to standard therapies for transformed FL.
      FL cohort: Grades 1-3a FL; patients who had relapsed after or failed to respond to at least two prior lines of systemic therapy and had received prior treatment with an anti-CD20-directed therapy and an alkylating agent. Patients in the FL expansion cohort may have been refractory to both anti-CD20-directed therapy and an alkylating agent.
      MCL cohort: patients who had relapsed after or failed to respond to at least one prior treatment regimen containing an approved Bruton's tyrosine kinase (BTK) inhibitor. If BTK inhibitor was received during participation in a clinical trial, patients had received treatment at a therapeutic dose level.
      Richter's transformation cohort: Patients had relapsed after or failed to respond to at least one prior systemic treatment regimen. Patients had received anthracycline and an anti-CD20-directed therapy in prior treatment regimen(s).
   CLL:
      A separate dose escalation could be initiated in CLL patients at the Sponsor's discretion after evidence of pharmacodynamic biomarker modulation and/or anti-tumor activity was observed in an NHL dose-escalation cohort in the absence of DLTs. CLL with Richter's transformation was an eligible diagnosis for enrollment in the CLL escalation cohorts.
NHL patients have at least one bi-dimensionally measurable lesion (>1.5 cm in its largest dimension for nodal lesions, or >1.0 cm in its largest dimension for extranodal lesions by computerized tomography (CT) scan or magnetic resonance imaging (MRI)).
For patients with DLBCL or transformed FL, the pathology report for the initial histopathology diagnosis was provided, if available. Patients with transformed FL also provided the pathology report at the time of disease transformation, if available. The results of all tests conducted on the tissue at initial diagnosis, including but not limited to tests assessing cell of origin, B-cell lymphoma 2 and MYC abnormalities, were provided if done.
Agreement to provide tumor samples as follows:
   For NHL patients with more than one bi-dimensionally measurable lesion (>1.5 cm in the largest dimension for nodal lesions, or >1.0 cm in its largest dimension for extranodal lesions by CT scan or MRI), agreement to undergo biopsy from a safely accessible site per investigator determination. Biopsies obtained at any time between the last dose of last prior anti-cancer therapy and the first dose of mosunetuzumab was acceptable.
   For patients with CLL: bone marrow biopsy and aspirate.
   Patients who were unable to undergo biopsy procedures could still be eligible for study enrollment. In such cases archival tumor tissue samples (paraffin blocks or at least 15 unstained slides) were made available.
Adverse events from prior anti-cancer therapy resolved to ≤Grade 1.
CLL patients only: had a circulating lymphocyte count of >5000/μL blood. Measurable disease by CT scan was not required.
Laboratory values as follows:
   Hepatic function
      AST and ALT≤3×the upper limit of normal (ULN) Total bilirubin≤1.5×ULN; patients with a documented history of Gilbert syndrome and in whom total bilirubin elevations were accompanied by elevated indirect bilirubin were eligible
   Hematologic function
      Platelet count >75,000/mm³ without transfusion within 14 days prior to first dose of mosunetuzumab
      ANC ≥1000/mm³
      Total hemoglobin ≥10 g/dL without transfusion within 21 days prior to first dose of mosunetuzumab.
      Patients who did not meet criteria for hematologic function because of extensive marrow involvement of NHL/CLL and/or disease-related cytopenias (e.g., immune thrombocytopenia) could be enrolled into the study.
   Serum creatinine≤ULN or estimated creatinine CL≥60 mL/min by Cockcroft-Gault method or other institutional standard methods (e.g., based on nuclear medicine renal scan).
Patients treated with alemtuzumab, fludarabine, cladribine, or pentostatin within 6 months before first mosunetuzumab administration could be enrolled only after confirming with the Medical Monitor.

### D. Exclusion Criteria

Patients who met any of the following criteria were excluded from the study:
Inability to comply with protocol-mandated hospitalization and activities restrictions
Pregnant or lactating, or intending to become pregnant during the study or within 3 months after the last dose of mosunetuzumab, and 3 months after the last dose of tocilizumab (if applicable)
   Women who were not postmenopausal (≤12 months of non-therapy-induced amenorrhea) or surgically sterile (removal of ovaries and/or uterus) must have had a negative serum pregnancy test result within 14 days prior to initiation of study drug.
      If a serum pregnancy test had not been performed within 14 days prior to receiving first study treatment, a negative urine pregnancy test result (performed within 7 days prior to study treatment) must have been available.
Prior use of any monoclonal antibody, radioimmunoconjugate or antibody-drug conjugate within 4 weeks before first mosunetuzumab administration.
Prior treatment with systemic immunotherapeutic agents for which the mechanism of action involved T cells, including but not limited to cytokine therapy and anti-CTLA-4, anti-PD-1 and anti-PD-L1 therapeutic antibodies, within 12 weeks or five half-lives of the drug, whichever was shorter, before first mosunetuzumab administration.
Treatment-emergent immune-related adverse events associated with prior immunotherapeutic agents (e.g., immune checkpoint inhibitor therapies) as follows:
   Grade ≥3 adverse events with the exception of Grade 3 endocrinopathy managed with replacement therapy
   Grade 1-2 adverse events that did not resolve to baseline after treatment discontinuation
   For certain prior treatments, such as CAR-T cell therapies, patients with prior immune-related Grade ≥3 adverse events (e.g., CRS) could be allowed to enroll after discussion with and confirmation by the Medical Monitor.
Treatment with any chemotherapeutic agent, or treatment with any other anti-cancer agent (investigational or otherwise) within 4 weeks or five half-lives of the drug, whichever was shorter, prior to first mosunetuzumab administration.
Treatment with radiotherapy within 2 weeks prior to the first mosunetuzumab administration.
If patients have received radiotherapy within 4 weeks prior to the first mosunetuzumab administration, patients must have had at least one measurable lesion outside of the radiation field. Patients who had only one measurable lesion that was previously irradiated but subsequently progressed were eligible
Autologous SCT within 100 days prior to first mosunetuzumab administration
Prior treatment with CAR-T therapy within 30 days before first mosunetuzumab administration
Current eligibility for autologous SCT in patients with R/R DLBCL or R/R transformed FL
Prior allogeneic SCT
Prior solid organ transplantation
History of autoimmune disease, including but not limited to myocarditis, pneumonitis, myasthenia gravis, myositis, autoimmune hepatitis, systemic lupus erythematosus, rheumatoid arthritis, inflammatory bowel disease, vascular thrombosis associated with antiphospholipid syndrome, Wegener's granulomatosis, Sjögren's syndrome, Guillain-Barre syndrome, multiple sclerosis, vasculitis, or glomerulonephritis
   Patients with a remote history of, or well-controlled autoimmune disease, were eligible to enroll after discussion with and confirmation by the Medical Monitor. Patients with controlled Type 1 diabetes mellitus who were on an insulin regimen were eligible for the study.
   Patients with a history of autoimmune-related hypothyroidism on a stable dose of thyroid replacement hormone were eligible for this study.
   Patients with a history of disease-related immune thrombocytopeniaurpura or autoimmune hemolytic anemia were eligible for this study.
   Patients with eczema, psoriasis, lichen simplex chronicus, or vitiligo with dermatologic manifestations only (e.g., patients with psoriatic arthritis are excluded) were eligible for the study provided all of following conditions were met:
Rash must cover <10% of body surface area
Disease was well controlled at baseline and required only low-potency topical corticosteroids No occurrence of acute exacerbations of the underlying condition requiring psoralen plus ultraviolet A radiation, methotrexate, retinoids, biologic agents, oral calcineurin inhibitors, or high potency or oral corticosteroids within the previous 12 months.
Patients with history of macrophage activation syndrome (MAS)/hemophagocytic lymphohistiocytosis (HLH).
Patients with history of confirmed progressive multifocal leukoencephalopathy.
History of severe allergic or anaphylactic reactions to monoclonal antibody therapy (or recombinant antibody-related fusion proteins).
History of other malignancy that could affect compliance with the protocol or interpretation of results
   Patients with a history of curatively treated basal or squamous cell carcinoma of the skin or in situ carcinoma of the cervix are allowed.
   Patients with a malignancy that has been treated with curative intent were also allowed if the malignancy has been in remission without treatment for >2 years prior to first mosunetuzumab administration.
Current or past history of CNS lymphoma.
Current or past history of CNS disease, such as stroke, epilepsy, CNS vasculitis, or neurodegenerative disease
   Patients with a history of stroke who had not experienced a stroke or transient ischemic attack in the past 2 years and have no residual neurologic deficits as judged by the investigator were allowed.
   Patients with a history of epilepsy who have had no seizures in the past 2 years while not receiving any anti-epileptic medications were allowed in the expansion cohorts only.
Significant cardiovascular disease such as New York Heart Association Class III or IV cardiac disease, myocardial infarction within the last 6 months, unstable arrhythmias, or unstable angina)
Significant active pulmonary disease (e.g., bronchospasm and/or obstructive pulmonary disease).
Known active bacterial, viral, fungal, mycobacterial, parasitic, or other infection (excluding fungal infections of nail beds) at study enrollment, or any major episode of infection requiring treatment with IV antibiotics or hospitalization (relating to the completion of the course of antibiotics) within 4 weeks prior to first mosunetuzumab administration.
Known or suspected chronic active Epstein Barr Virus infection.
Recent major surgery within 4 weeks prior to first mosunetuzumab administration
   Protocol-mandated procedures (e.g., tumor biopsies and bone marrow biopsies) were permitted.
Positive serologic or polymerase chain reaction (PCR) test results for acute or chronic hepatitis B virus (HBV) infection
   Patients whose HBV infection status could not be determined by serologic test results (cdc.gov) must be negative for HBV by PCR to be eligible for study participation.
   Acute or chronic hepatitis C virus (HCV) infection
   Patients who were positive for HCV antibody must be negative for HCV by PCR to have been eligible for study participation.
Positive serologic test results for HIV infection.
Administration of a live, attenuated vaccine within 4 weeks before first dose of study treatment or anticipation that such a live attenuated vaccine was required during the study
   Patients must not have received live, attenuated vaccines (e.g., FluMist®) while receiving study treatment or after the last dose until B-cell recovery to the normal ranges. Killed vaccines or toxoids should be given at least 4 weeks prior to the first dose of study treatment to allow development of sufficient immunity.
   Inactivated influenza vaccination could be given during influenza season only.
   Investigators reviewed the vaccination status of potential study patients being considered for this study and follow the U.S. Centers for Disease Control and Prevention guidelines for adult vaccination with any other non-live vaccines intended to prevent infectious diseases prior to study.

### Drug Products

### Mosunetuzumab

Dosing independent of body weight was used for mosunetuzumab. The dose of mosunetuzumab for each patient depended on the dose level assignment as detailed in the protocol.

Mosunetuzumab was administered to patients either by IV infusion or SC injection using standard medical syringes and syringe pumps or IV bags where applicable. Compatibility testing had shown that mosunetuzumab is stable in extension sets and polypropylene syringes. When administered IV, the Drug

Product was delivered by syringe pump via an IV infusion set or IV bag with a final mosunetuzumab volume determined by the dose. When administered SC, the Drug Product was delivered by medical syringe with a final mosunetuzumab volume not to exceed 2.0 mL.

Mosunetuzumab was administered in a setting with immediate access to trained critical care personnel and facilities equipped to respond to and manage medical emergencies.

Mosunetuzumab was administered to well-hydrated patients. Corticosteroid premedication consisting of dexamethasone 20 mg IV or methylprednisolone 80 mg IV was administered 1 hour prior to the administration of each mosunetuzumab dose. This administration of corticosteroid premedication was optional for Cycle 3 and beyond for patients in Groups B, D, and F, or for Cycle 4 based on investigator's assessment. However, if the patient experienced CRS, premedication with steroids was administered for the subsequent doses until no additional CRS events are observed. For patients receiving mosunetuzumab via SC injection, corticosteroid premedication consisting of 20 mg of dexamethasone or 80 mg of methylprednisolone could be administered orally 2 hours prior to the administration of each mosunetuzumab dose, in lieu of IV administration. In addition, premedication with oral acetaminophen or paracetamol (e.g., 500-1000 mg) and/or 50-100 mg diphenhydramine could be administered per standard institutional practice prior to administration of mosunetuzumab. Decisions to modify the requirement for corticosteroid premedication was made based on the recommendation of the IMC.

The recommended management of CRS is detailed in Table 4.

TABLE 4
Management of cytokine release syndrome for patients receiving mosunetuzumab

| | Action with Current | | Anti-IL-6/ | |
|---|---|---|---|---|
| | Mosunetuzumab | | Corticosteroid | Action for Next |
| CRS Grade^{a} | Infusion | Supportive Care | Therapy | Mosunetuzumab Dose |
| Grade 1 | Slow infusion to ≤50% | Symptomatic | For prolonged CRS (>2 | Administer |
| Symptoms | or interrupt | management of | days) in patients with | premedications for next |
| not life- | infusion until | constitutional symptoms. | significant symptoms and/or | dose. |
| threatening | symptoms resolve; | Consider empiric broad- | comorbidities (per | Consider 50% (or lower) |
| and require | re-start at same rate. | spectrum antibiotics. | investigator discretion, e.g., | rate of infusion for next |
| symptomatic | If symptoms recur | Consider G-CSF if | impaired cardiovascular | step-up dose in Cycle 1 or |
| treatment | with rechallenge, | neutropenic. | function, reduced | 50% rate of infusion if next |
| only | interrupt study | Maintenance IV fluids | pulmonary reserve), | dose is same dose level |
| | treatment, do not | for hydration. | consider tocilizumab and | (beyond Cycle 1). |
| | resume, and | Consider hospitalization | corticosteroids as per | Consider hospitalization |
| | manage per Grade | until symptoms | Grade 2. | for next dose |
| | 2. | completely resolve. | | |
| Grade 2 | Hold further study | Symptomatic | Consider tocilizumab. ^{b} | May receive the next |
| Symptoms | treatment until | management of | For persistent refractory | dose of mosunetuzumab if |
| require and | symptoms resolved; | constitutional symptoms | hypotension after 1-2 doses | symptoms resolve to |
| respond to | consider re-starting | and organ toxicities. | of anti-IL-6 therapy, | Grade ≤1 for 3 |
| moderate | infusion at 50% rate. | Consider ICU | consider dexamethasone | consecutive days with |
| intervention O₂ | If symptoms recur | admission for | 10 mg IV every 6 hours | approval of Medical |
| requirement <40% | with rechallenge at | hemodynamic monitoring. | (or equivalent). | Monitor. |
| OR | decreased infusion | For hypotension: IV | Manage per Grade 3 if no | Consider enhanced |
| hypotension | rate, interrupt study | fluid bolus as needed; for | improvement within 24 | premedications for next |
| responsive to | treatment, do not | persistent refractory | hours after starting | dose. |
| fluids or low | resume, and | hypotension (e.g., after | tocilizumab. | Consider 50% (or lower) |
| dose of one | manage per Grade | two fluid boluses and | | rate of infusion for next |
| vasopressor | 3. | anti-IL-6 therapy), start | | step-up dose in Cycle 1 or |
| OR | | vasopressors and | | 50% rate of infusion if next |
| Grade 2 | | manage per Grade 3. | | dose is same dose level |
| organ toxicity | | Rule out other | | (beyond Cycle 1). |
| | | inflammatory conditions | | Consider hospitalization |
| | | which can mimic severe | | for next dose. |
| | | CRS (e.g., infections/ | | |
| | | sepsis). | | |
| | | Consider empiric broad- | | |
| | | spectrum antibiotics. | | |
| | | If no improvement | | |
| | | within 24 hours, initiate | | |
| | | work up and assess for | | |
| | | signs and symptoms of | | |
| | | HLH. | | |
| Grade 3 | Stop infusion, do | Symptomatic | Administer tocilizumab. ^{b} | May receive the next |
| Symptoms | not resume. | management of organ | Dexamethasone 10 mg IV | dose of mosunetuzumab if |
| require and | | toxicities, admit to ICU for | every 6 hours (or | CRS event was |
| respond to | | hemodynamic monitoring. | equivalent). If refractory, | responsive to treatment |
| aggressive | | For hypotension: IV | manage as per Grade 4. ^{b} | (i.e., clinical improvement |
| intervention O₂ | | fluid bolus and | Manage per Grade 4 if no | within 8-12 hours following |
| requirement ≥40% | | vasopressors as needed. | improvement within 18-24 | tocilizumab/corticosteroids |
| OR | | Rule out other | hours after second dose of | administration) and |
| hypotension | | inflammatory conditions | tocilizumab. | symptoms resolve to |
| requiring high | | which can mimic severe | | Grade ≤1 for 3 |
| dose or | | CRS (e.g., | | consecutive days with |
| multiple | | infections/sepsis). | | approval of Medical |
| vasopressors | | Consider empiric broad- | | Monitor: |
| OR | | spectrum antibiotics. | | Enhanced premedications |
| Grade 3 | | If no improvement | | for next dose |
| organ toxicity | | within 24 hours, initiate | | Decrease to 50% (or |
| or Grade 4 | | work up and assess for | | lower) rate of infusion for |
| transaminitis | | signs and symptoms of | | next step-up dose in Cycle |
| | | HLH. | | 1, or 50% rate of infusion if |
| | | | | next dose is same dose |
| | | | | level (beyond Cycle 1) |
| | | | | Hospitalization for next |
| | | | | dose |
| | | | | The next dose should be |
| | | | | reduced to the next lower |
| | | | | dose level that has been |
| | | | | previously cleared during |
| | | | | dose escalation. ^{f} |
| | | | | Subsequent doses may |
| | | | | not be re-escalated with |
| | | | | signs/symptoms of Grade |
| | | | | 3 or higher CRS at the |
| | | | | reduced dose. |
| | | | | If the reduced dose is |
| | | | | tolerated with no |
| | | | | signs/symptoms of Grade |
| | | | | 3 or higher CRS, the |
| | | | | patient may return to the |
| | | | | next higher dose that has |
| | | | | been previously cleared |
| | | | | during dose escalation. |
| | | | | If Grade 3 CRS recurs |
| | | | | with subsequent doses, |
| | | | | permanently discontinue |
| | | | | mosunetuzumab. ^{g} |
| Grade 4 | Stop infusion, do | ICU admission and | Administer tocilizumab. ^{b} | Permanently discontinue |
| Life- | not resume. | hemodynamic monitoring. | For patients refractory to | mosunetuzumab. ^{g} |
| threatening | | Mechanical ventilation | tocilizumab, consider | |
| symptoms | | as needed. | siltuximab, anakinra, and | |
| Requirement | | IV fluids and | emapalumab, based on | |
| for ventilator | | vasopressors as needed. | discretion of the | |
| support | | Symptomatic | investigator; management | |
| OR | | management of organ | should be discussed with | |
| Grade 4 | | toxicities. | the Medical Monitor. ^{c} | |
| organ toxicity | | Rule out other | Dexamethasone 10 mg IV | |
| (excluding | | inflammatory conditions | every 6 hours (or | |
| transaminitis) | | which can mimic severe | equivalent). | |
| | | CRS (e.g., | If refractory, consider | |
| | | Infections/sepsis) | methylprednisolone 1000 | |
| | | Consider empiric broad- | mg/day IV. ^{d, e} | |
| | | spectrum antibiotics. | | |
| | | If no improvement | | |
| | | within 24 hours, initiate | | |
| | | work up and assess for | | |
| | | signs and symptoms of | | |
| | | HLH. | | |

BiPAP = bilevel positive airway pressure; CPAP = continuous positive airway pressure; CRS = cytokine release syndrome; G-CSF = granulocyte colony stimulating factor; HLH = hemophagocytic lymphohistiocytosis.
^{a} CRS grading per Lee et al., Blood, 124: 188-195, 2014.
^{b} Tocilizumab was administered at a dose of 8 mg/kg IV (8 mg/kg for participants at a weight of ≥30 kg only; 12 mg/kg for participants at a weight of <30 kg; doses exceeding 800 mg per infusion was not recommended); repeat every 8 hours as necessary (up to a maximum of 4 doses).
^{c} Riegler et al., Ther Clin Risk Manag, 15: 323-335, 2019.
^{d} Antifungal prophylaxis was strongly considered in patients receiving steroids for treatment of CRS.
^{e} For example, methylprednisolone IV 1000 mg/day for 3 days, followed by rapid taper at 250 mg every 12 hours for 2 days, 125 mg every 13 hours for 2 days, and 60 mg every 12 hours for 2 days.
^{f} If Grade 3 CRS occurs in the step-up dosing cohorts following mosunetuzumab administration at Cycle 1 Day 1 or Cycle 1 Day 8, the next mosunetuzumab dose was discussed with the Medical Monitor and a dose reduction was consideredExceptions may be considered to repeat the same step-up dose based on individual risk-benefit assessment.
^{g} Resumption of mosunetuzumab was considered in patients who were deriving benefit and have fully recovered from the adverse eventPatients could be re-challenged with mosunetuzumab only after approval had been documented by both the investigator (or an appropriate delegate) and the Medical MonitorFurther treatment was not considered unless all the criteria below were met: Individual risk-benefit assessment by principal investigator/treating physician favored continued treatment; The patient had recovered from previous toxicities and had sufficient organ function/reserve to receive subsequent doses; The patient had been adequately consented for risks associated with continued treatment and decided to receive subsequent doses; The above risk-benefit assessment and evaluation of patient's were discussed with the Sponsor; Subsequent doses were well planned with precautionary measures, including dose reduction, slow infusion rate at 50% or lower, mandatory hospitalizations, and enhanced premedication.

For IV mosunetuzumab administration, initially, mosunetuzumab was infused over 4 hours±15 minutes. The infusion was slowed or interrupted for patients experiencing infusion-associated symptoms.

Following each mosunetuzumab dose, patients were observed at least 90 minutes for fever, chills, rigors, hypotension, nausea, or other signs and symptoms of CRS. In the absence of infusion related adverse events, the infusion time of mosunetuzumab in Cycle 2 and beyond could be reduced to 2 hours±15 minutes. Patients who underwent intra-patient dose escalation received the first higher infusion of mosunetuzumab over a minimum of 4 hours.

For SC administration, mosunetuzumab was administered by qualified staff over 30 seconds to 2 minutes. The recommended management of injection-site reactions is detailed in Table 5. All patients had IV access in place prior to mosunetuzumab SC administration for at least the first two cycles. Placement of IV access may be optional in Cycle 3 and beyond for patients in Groups D and F and was considered for patients who continue to experience, or remain at risk, for CRS.

TABLE 5
Management guidelines for injection-site reactions

| Grade | Management |
|---|---|
| Grade 1 | Consider treatment with topical steroids. |
| | Continue mosunetuzumab in subsequent cycles. |
| Grade 2 | Initiate treatment with topical steroids. |
| | If progressive after 24 hours, consider |
| | prednisone or equivalent 10-30 mg/day. |
| | Continue mosunetuzumab in subsequent cycles |
| | after improvement to Grade ≤1. |
| Grade 3 | Withhold mosunetuzumab. |
| | Initiate prednisone 1 mg/kg/day or equivalent. |
| | Consult dermatology. |
| | Taper steroids after improvement to Grade ≤1. |
| | Continue mosunetuzumab in subsequent cycles |
| | after improvement to Grade ≤1. |
| Grade 4 | Management as for Grade 3. |
| | Permanently discontinue SC mosunetuzumab. |
| | Consider continuing study treatment with |
| | IV mosunetuzumab with approval by Medical Monitor. |

### Tocilizumab

Tocilizumab was formulated, prepared, and handled according to standard practice.

### E. Concomitant Therapy

Concomitant therapy included any medication (e.g., prescription drugs, over-the-counter drugs, herbal or homeopathic remedies, nutritional supplements) used by a patient from 7 days prior to screening to the study completion/discontinuation visit.

Patients who used oral contraceptives, hormone-replacement therapy, or other maintenance therapy continued their use.

Concomitant use of hematopoietic growth factors such as erythropoietin, granulocyte/macrophage colony-stimulating factor (sargramostim), or thrombopoietin (oprelvekin, eltrombopag) was not initiated or increased in dose from the start of the screening period until the completion of the DLT assessment period in the absence of a DLT. After the DLT assessment period was complete or after a DLT was documented, initiation or dose and schedule modifications of hematopoietic growth factors were allowed in accordance with instructions provided in the package inserts, institutional practice and/or published guidelines.

Prophylactic and therapeutic use of G-CSF (filgrastim, pegfilgrastim) was allowed in accordance with instructions provided in the package inserts, institutional practice, and/or published guidelines (Smith et al. 2015). Growth factor support was started when absolute neutrophil count (ANC) was <500/mm³, unless medically contraindicated; if growth factor was contraindicated; this was discussed with the Medical Monitor.

Anti-infective prophylaxis for viral, fungal, bacterial or pneumocystis infections was permitted and was instituted per institutional practice.

Patients who experienced mosunetuzumab infusion-related symptoms were treated symptomatically as described herein. Treatment of severe CRS or HLH according to published recommendations and/or institutional practice was permitted.

Given the expected pharmacology of mosunetuzumab, the transient release of cytokines could suppress CYP450 enzymes and cause drug-drug interactions. Based on nonclinical models, cytokine levels were likely to be highest during the first 24 hours of the first cycle. During subsequent cycles, with decreasing number of CD20+ cells, it was anticipated that the cytokine levels would be substantially reduced. Patients who were of highest risk of a drug-drug interaction were those receiving concomitant medications that were CYP450 substrates and had a narrow therapeutic index (Table 6). Such concomitant medications were monitored for toxicity, and dose adjusted accordingly.

TABLE 6
Examples of sensitive in vivo CYP substrates and
CYP substrates with narrow therapeutic range

| | | Substrates With Narrow |
|---|---|---|
| CYP Enzymes | Sensitive Substrates ^{a} | Therapeutic Range ^{b} |
| CYP1A2 | Alosetron, caffeine, duloxetine, | Theophylline, tizanidine |
| | melatonin, ramelteon, tacrine, | |
| | tizanidine | |
| CYP2B6 ^{c} | Bupropion, efavirenz | |
| CYP2C8 | Repaglinide ^{d} | Paclitaxel |
| CYP2C9 | Celecoxib | Warfarin, phenytoin |
| CYP2C19 | Lansoprazole, omeprazole, | S-mephenytoin |
| | S-mephenytoin | |
| CYP3A ^{e} | Alfentanil, aprepitant, budesonide, | Alfentanil, astemizole, ^{f} cisapride, ^{f} |
| | buspirone, conivaptan, darifenacin, | cyclosporine, dihydroergotamine, |
| | darunavir, dasatinib, dronedarone, | ergotamine, fentanyl, pimozide, |
| | eletriptan, eplerenone, everolimus, | quinidine, sirolimus, tacrolimus, |
| | felodipine, indinavir, fluticasone, | terfenadine ^{f} |
| | lopinavir, lovastatin, lurasidone, | |
| | maraviroc, midazolam, nisoldipine, | |
| | quetiapine, saquinavir, sildenafil, | |
| | simvastatin, sirolimus, tolvaptan, | |
| | tipranavir, triazolam, vardenafil | |
| CYP2D6 | Atomoxetine, desipramine, | Thioridazine |
| | dextromethorphan, | |
| | metoprolol, nebivolol, perphenazine, | |
| | tolterodine, venlafaxine | |

AUC = area under the concentration-time curve; P-g = P-glycoprotein.
^{a} Sensitive CYP substrates refered to drugs whose plasma AUC values had been shown to increase 5-fold or higher when co-administered with a known CYP inhibitor.
^{b} CYP substrates with narrow therapeutic range refered to drugs whose exposure-response relationship indicated that small increases in their exposure levels by the concomitant use of CYP inhibitors could lead to serious safety concerns (e.g., Torsades de Pointes).
^{c} The AUC of these substrates did not increase by 5-fold or more with a CYP2B6 inhibitor, but they represented the most sensitive substrates studied with available inhibitors evaluated to date.
^{d} Repaglinide was also a substrate for OATP1B1, and it was only suitable as a CYP2C8 substrate if the inhibition of OATP1B1 by the investigational drug had been ruled out.
^{e} Because a number of CYP3A substrates (e.g., darunavir, maraviroc) were also substrates of P-gp, the observed increase in exposure could be due to inhibition of both CYP3A and P-gp.
^{f} Withdrawn from the United States market because of safety reason.

CYP450 enzymes in the liver were down-regulated by infection and inflammatory stimuli, including cytokines such as IL-6. Inhibition of IL-6 signaling in patients with rheumatoid arthritis who were treated with tocilizumab may restore CYP450 activities to higher levels than those patients not treated with tocilizumab, leading to increased metabolism of drugs that are CYP450 substrates. In vitro studies showed that tocilizumab had the potential to affect expression of multiple CYP enzymes, including CYP1A2, CY2B6, CYP2C9, CYP2C19, CYP2D6, and CYP3A4. The effects of tocilizumab on CYP2C8 or transporters were unknown. In vivo studies with omeprazole (metabolized by CYP2C19 and CYP3A4) and simvastatin (metabolized by CYP3A4) showed up to a 28% and 57% decrease in exposure 1 week following a single dose of tocilizumab, respectively.

The effect of tocilizumab on CYP enzymes could be clinically relevant for CYP450 substrates with narrow therapeutic index (Table 6), where the dose was individually adjusted:
Upon initiation or discontinuation of tocilizumab in patients being treated with these types of medicinal products, therapeutic monitoring of effect (e.g., warfarin) or drug concentration (e.g., cyclosporine or theophylline) was performed and the individual dose of the medicinal product adjusted as needed.
Prescribers exercised caution when tocilizumab was co-administered with CYP3A4 substrate drugs where a decrease in effectiveness is undesirable (e.g., oral contraceptives, lovastatin, atorvastatin).
The effect of tocilizumab on CYP450 enzyme activity could persist for several weeks after stopping therapy.

### F. Rationale for Using a Modified Grading Scale for CRS

In this study, grading and treatment of the adverse event of CRS arising from mosunetuzumab treatment was based on published criteria of Lee et al., Blood, 124:188-195, 2014 and is described in Table 2. For dose-escalation decisions, DLTs related to CRS were defined based on individual signs and symptoms and laboratory data according to NCI CTCAE v4.0.

The NCI CTCAE v4.0 CRS grading scale was based on characterizations of CRS following treatment with monoclonal antibodies (Lee et al., Blood, 124:188-195, 2014). T-cell directed therapies, including bispecific antibodies such as mosunetuzumab and adoptive cell therapies such as engineered T-cells expressing CARs, resulted in pharmacodynamic profiles of cytokine release from T-cell activation distinct from those associated with conventional monoclonal antibodies. Consequently, the clinical features of CRS as defined by NCI CTCAE v4.0 were applicable to those following T-cell directed therapy.

Several alternate grading scales had been proposed and published which were specifically geared toward evaluation of CRS for T-directed therapies (Davila et al., Sci Transl Med, 6: 224ra25, 2014; Lee et al., Blood, 124:188-195, 2014; Porter et al., Sci Transl Med, 7: 303ra139, 2015). The grading system of Lee et al. was based on CRS arising from treatment with CD19 directed CAR-T cell and blinatumomab. It was a modification of NCI CTCAE v4.0, which provided further diagnostic detail including accounting for transient elevations in liver transaminases that may have occured in the setting of CRS. In addition to diagnostic criteria, recommendations on management of CRS based on its severity, including early intervention with corticosteroids and/or anti-cytokine therapy, are provided below. Incorporation of the CRS grading scale therefore allowed for alignment between reporting and management guidelines that have been published and widely adopted.

### G. Outcome Measures

### Safety Outcome Measures

The safety and tolerability of mosunetuzumab were assessed using the following primary safety outcome measures:
Incidence and nature of DLTs when mosunetuzumab was given as a single agent IV or SC.

Safety and tolerability were additionally assessed using the following secondary safety outcome measures:
Incidence, nature, and severity of adverse events (AEs).
Incidence of anti-drug antibodies (ADAs) against mosunetuzumab, and their relationship to clinical outcomes.
Changes in vital signs and clinical laboratory values.

### Pharmacokinetic Outcome Measures

The following PK parameters were derived from the serum concentration-time profiles of mosunetuzumab following administration, when appropriate as data allowed:
Total exposure (area under the concentration-time curve [AUC])
Maximum serum concentration (Cₘₐₓ)
Minimum serum concentration (Cₘᵢₙ)
Clearance (CL)
Volume of distribution at steady state (Vₛₛ).

Serum trough and maximum concentrations for tocilizumab, where applicable, were summarized, as appropriate and as data allow. Compartmental, non-compartmental, and/or population methods could be considered. Other parameters, such as accumulation ratio, t_{1/2}, and dose proportionality, could also be calculated.

### Activity Outcome Measures

The activity outcome measures for this study were as follows:
Investigator-assessed objective response, defined as a partial response (PR) or complete response (CR), as assessed by the investigator using standard criteria for NHL (Cheson et al., J Clin Oncol, 25:579-586 2007) and CLL (Hallek et al., Blood, 111:5446-5456, 2008).
Investigator-assessed duration of objective response, defined as the first occurrence of a documented, objective response until the time of disease progression or relapse as assessed by the investigator, or death from any cause, whichever occurred first.
Investigator-assessed PFS, defined as the time from the first study treatment to the first occurrence of disease progression as assessed by the investigator, or death from any cause, whichever occurs first.

Additional activity outcome measures in the Group B R/R DLBCL and transformed FL expansion cohorts, and R/R FL expansion cohorts, were as follows:
Objective response by an Independent review facility (IRF), defined as a PR or CR, as assessed by an IRF using standard criteria for NHL (Cheson et al., J Clin Oncol, 25:579-586 2007).
IRF-assessed duration of objective response, defined as the first occurrence of a documented, objective response until the time of disease progression or relapse as assessed by an IRF, or death from any cause, whichever occurred first.
IRF-assessed duration of CR, defined as the first occurrence of a documented CR until the time of disease progression or relapse as assessed by an IRF, or death from any cause, whichever occurred first.
Investigator-assessed duration of CR, defined as the first occurrence of a documented CR until the time of disease progression or relapse as assessed by the investigator, or death from any cause, whichever occurred first.
IRF-assessed PFS, defined as the time from the first study treatment to the first occurrence of disease progression as assessed by an IRF or death from any cause, whichever occurred first.
OS, defined as the time from the first study treatment to the date of death from any cause.

### Patient-Reported Outcome Measures

The health-related quality of life (HRQoL) and health status measures that were used in NHL expansion cohorts to evaluate patient-reported outcomes (PROs) were as follows:
Summary statistics and change from baseline in HRQoL based on the European Organization for Research and Treatment of Cancer Quality of Life Questionnaire Core 30 (EORTC QLQ-C30).
Summary statistics and change from baseline in disease-related symptoms based on the Functional Assessment of Cancer Therapy-Lymphoma (FACT-Lym) subscale.
Descriptive results of the EQ-5D-5 L data during patients' participation in the study.

### Study Design

### Group B: Cycle 1 Step-Up, Single-Agent Mosunetuzumab Escalation (IV Infusion)

Patients enrolled in dose-escalation Group B received mosunetuzumab by IV infusion on Days 1, 8, and 15 of Cycle 1. In Cycle 2 and beyond, mosunetuzumab was given on Day 1 of each 21-day cycle, with Day 1 of Cycle 2 being 7 days after the Cycle 1 Day 15 dose. Mosunetuzumab was given up to ±1 day from the scheduled date for Cycle 2 (i.e., with a minimum of 6 days after Cycle 1 Day 15 dosing), and ±2 days from the scheduled date for Cycle 3 and beyond (i.e., with a minimum of 19 days between doses) for logistic/scheduling reasons.

### Dose Escalation in Group B Used a Standard 3+3 Design.

Based on cumulative clinical data, the Group B recommended Phase II dose (RP2D) was determined to be Jan. 2, 1960/60/30 mg with the IV Cycle 1 step-up dosing (Cycle 1 Day 1:1 mg; Cycle 1 Day 8:2 mg; Cycle 1 Day 15 and Cycle 2 Day 1:60 mg; Cycle 3 Day 1 and beyond: 30 mg); four histology-specific expansion cohorts were opened to enroll patients with R/R DLBCL or transformed FL, R/R FL, R/R MCL and R/R Richter's transformation at the Group B RP2D. There were also interim expansion cohorts at the lower dose levels in Group B, or other dosing groups to provide additional data to further evaluate the safety and antitumor activity.

### Group D: Cycle 1 Non-Fractionated, Single-Agent Mosunetuzumab Escalation (SC Infusion)

Group D dose escalation in the GO29781 study was conducted independently of Group B dose escalations. Patients enrolled into dose-escalation Group D received mosunetuzumab subcutaneously (SC) on Day 1 of each 21-day cycle. Mosunetuzumab was given up to +2 days from the scheduled date (i.e., with a minimum of 19 days between doses) for logistic/scheduling reasons.

Group D escalation used a standard 3+3 dose-escalation design using the dose-escalation rules as defined for Group A 3+3 dose escalation. The initial dose of mosunetuzumab was 1.6 mg SC, a dose level that was previously assessed in Group A escalation and demonstrated to be safe and tolerable in treated patients. The DLT assessment period was from Cycle 1 Day 1 through Cycle 1 Day 21 of mosunetuzumab treatment, and dose escalation continued until the MTD or MAD was identified. On the basis of review of real-time safety data and available preliminary PK data, dose increments were recommended by the IMC based on review of totality of the data in dose escalation and expansion, as long as the recommended dose level did not exceed the maximum assessed cumulative Cycle 1 dose for mosunetuzumab in Group B.

Should an individual patient at any time develop unacceptable localized injection site reaction toxicity following SC administration of mosunetuzumab, conversion to IV mosunetuzumab administration was considered following discussion with and approval by the Medical Monitor. In these cases, IV dosing followed the Group B step-up schedule based on the highest cleared dose and schedule in Group B dose escalation.

### Group F: Cycle 1 Step-Up, Single-Agent Mosunetuzumab Escalation (SC Injection)

Group F of the GO29781 study proceeded as an independent dose-escalation group to test the combination of step-up dosing and SC injection to further mitigate cytokine-driven toxicities.

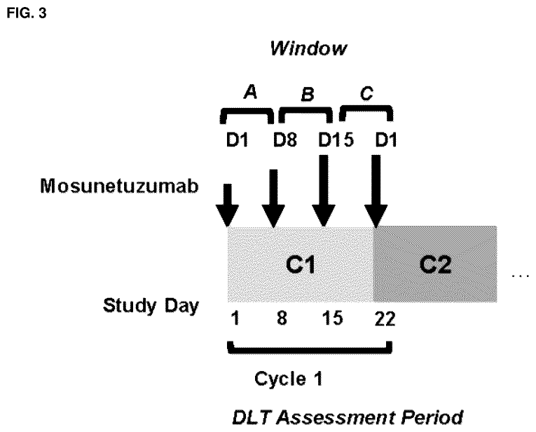

Patients enrolled in dose-escalation Group F received mosunetuzumab by SC injection on Days 1, 8, and 15 of Cycle 1 (FIG. 3). In Cycle 2 and beyond, mosunetuzumab was given on Day 1 of each 21-day cycle, with Cycle 2, Day 1 being 7 days after the Cycle 1, Day 15 dose. Mosunetuzumab was given up to ±1 day from the scheduled date for Cycle 2 (i.e., with a minimum of 6 days after Cycle 1, Day 15 dosing), and ±2 days from the scheduled date for Cycle 3 and beyond (i.e., with a minimum of 19 days between doses) for logistic/scheduling reasons.

### Dose Escalation in Group F Used a Standard 3+3 Design.

DLT assessment for patients enrolled in Cohort F1 (5 mg/15 mg/45 mg; Cycle 1 Day 1:5 mg, Cycle 1 Day 8:15 mg; Cycle 1 Day 15:45 mg; Cycle 2±Day 1:45 mg) and F2 (5 mg/45 mg/45 mg; Cycle 1 Day 1:5 mg, Cycle 1 Day 8:45 mg; Cycle 1 Day 15:45 mg; Cycle 2±Day 1:45 mg) in Group F was cleared by the IMC without any DLT event, and the Group F MTD has not been exceeded.

### Treatment Following Disease Progression

A. Treatment of Non-Hodgkin's Lymphoma after Disease Progression

Experience with cancer immunotherapy for solid tumors had demonstrated that responding tumors may initially increase in size due to the influx of immune cells, a phenomenon known as “pseudoprogression” (Wolchok et al., Clin Can Res, 15 (23): 7412-7420, 2009). Pseudoprogression has not been described in the context of lymphoma immunotherapy, but it was possible that mosunetuzumab therapy may have initially increased tumor size and metabolic activity by inducing the influx of T cells into the tumor. Given this, a repeat tumor biopsy, if clinical disease progression was observed, was strongly encouraged. Additionally, if the study investigator believed that an NHL patient was deriving clinical benefit despite radiographic evidence of progressive disease as defined by the Revised Response Criteria for Malignant Lymphoma (Cheson et al., J Clin Oncol, 25:579-586, 2007), that patient could continue study treatment provided the following criteria were met:
There was an absence of symptoms and signs (including worsening of laboratory values) indicating unequivocal progression of disease.
There was no decline in Eastern Cooperative Oncology Group (ECOG) Performance Status.
There was an absence of tumor progression at critical anatomical sites including the central airway, the great vessels, and other organs or tissues where compromised function secondary to tumor progression would be expected to result acutely in severe and/or irreversible disability or death.

Patients continuing study treatment despite apparent radiographic progression were strongly encouraged to undergo a repeat tumor biopsy to assess whether increases in tumor volume were due to immune cell infiltration or neoplastic proliferation, provided that such a biopsy can be performed safely on a non-target lesion. If true progression was suspected based on the investigator's judgment, clinical factors, or biopsy findings that were consistent with neoplastic proliferation, or if radiographic disease progression was confirmed at a subsequent tumor assessment, the patient was ineligible to receive further study treatment under the currently assigned treatment group.

### B. Mosunetuzumab Treatment Duration and Re-Treatment Following Disease Progression

Patients who initially responded or had stable disease to mosunetuzumab as a single agent could benefit from additional cycles beyond the initial eight cycles of study treatment, depending on anti-tumor responses to initial treatment.

To test these hypotheses, patients were eligible for mosunetuzumab re-treatment or continued study treatment beyond the initial eight cycles as described herein. The study re-treatment dose and schedule was one that had been previously demonstrated in dose escalation to be safe, provided the following criteria were met:
Pertinent eligibility criteria were met at the time that mosunetuzumab treatment was re-initiated, with the following exceptions:
   Prior therapy with mosunetuzumab was allowed
   Serology tests to demonstrate human immunodeficiency virus (HIV), hepatitis C virus (HCV), and hepatitis B virus (HBV) status did not need to be repeated unless clinically indicated. EBV and cytomegalovirus (CMV) quantitative polymerase chain reaction (PCR) were repeated.
   Manageable and reversible immune related adverse events with initial study treatment were allowed and did not constitute an exclusionary history of autoimmune disease.
Patients had not experienced Grade 4 non-hematologic adverse events that were not considered by the investigator to be attributable to another clearly identifiable cause during initial study treatment, with the possible exception of TLS.
Patients who experienced Grade 2 or Grade 3 AEs that were not considered by the investigator to be attributable to another clearly identifiable cause during initial treatment have resolved these toxicities to ≤Grade 1.
Patients may have required hospitalization following the first re-treatment administration.
No intervening systemic anti-cancer therapy was administered between the completion of initial study treatment and re-initiation of study treatment.

Patients proceeding to re-treatment following disease progression were strongly encouraged to undergo a repeat tumor biopsy from a safely accessible site to assess: 1) CD20 expression status and 2) changes/status of the tumor and immune microenvironment. Patients who provided written informed consent but had no lesion amenable for biopsy at disease progression could still be considered for study drug re-treatment following a discussion between the study investigator and the Medical Monitor.

The dose and schedule of study treatment to be administered for patients receiving re-treatment was determined by the Medical Monitor and was on a previously tested dose and schedule that had cleared the DLT observation period.

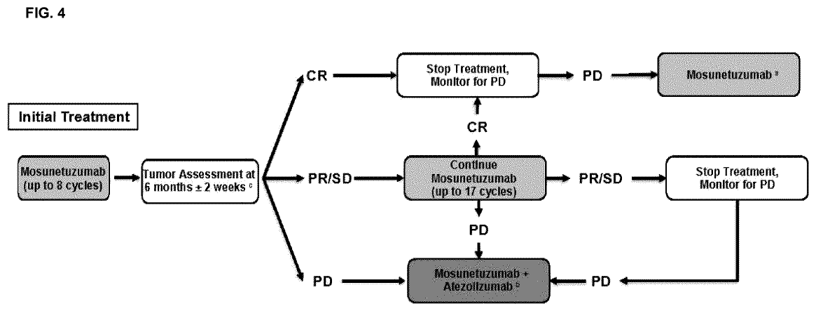

The duration of initial study treatment and options for re-treatment or continued study treatment beyond the initial eight cycles of study treatment are described in FIG. 4. The dose and schedule/route of administration of mosunetuzumab based on the nature and timing of study treatment is described in Table 7.

For patients initially receiving single-agent mosunetuzumab:
Mosunetuzumab is given for eight cycles unless PD or unacceptable toxicity was observed prior to completion of the eight cycles.
The tumor assessment at 6 months (±2 weeks) could be scheduled after Cycle 8 Day 1 but before Cycle 9 Day 1 in order to determine the duration of study treatment.
Patients who achieved a complete response (CR) after receiving eight cycles of treatment did not receive any additional cycles of mosunetuzumab and were monitored; if PD following completion of initial single-agent mosunetuzumab treatment was observed, single-agent mosunetuzumab re-treatment could be initiated. Treatment could continue with mosunetuzumab for at least eight additional cycles.
Patients who achieved a PR or maintain stable disease (SD) after receiving eight cycles of treatment continue single-agent mosunetuzumab for up to a total of 17 cycles unless PD or unacceptable toxicity was observed.
   If CR was achieved after 17 cycles of treatment, monitoring as described for patients who achieved a CR with mosunetuzumab was followed.
   If PR or SD was achieved after 17 cycles of treatment, patients continue to be monitored.

TABLE 7
Mosunetuzumab treatment/re-treatment: dose and schedule/route of administration

| Initial treatment | | Re-treatment upon progressive disease (PD) with initial treatment | | | |
|---|---|---|---|---|---|
| | Adminis- | Treatment-free | | Adminis- | |
| Agent(s) | tration | interval | Agent(s) | tration | Dose/schedule |
| mosunetuzumab | IV | ≥6 weeks | mosunetuzumab | IV | Highest cleared Group B |
| | | | | | dose/schedule including |
| | | | | | Cycle 1 step-up dosing |
| mosunetuzumab | IV | <6 weeks | mosunetuzumab | IV | Highest cleared Group B |
| | | | | | Cycle 2 dose every 21 days; |
| | | | | | no mosunetuzumab step-up |
| | | | | | dosing |
| mosunetuzumab | SC | any | mosunetuzumab | SC | Highest cleared Group D |
| | (Group D) | | | | dose/schedule |
| mosunetuzumab | SC | ≥6 weeks | mosunetuzumab | SC | Highest cleared Group F |
| | (Group F) | | | | dose/schedule including |
| | | | | | Cycle 1 step-up dosing |
| mosunetuzumab | SC | <6 weeks | mosunetuzumab | SC | Highest cleared Group F |
| | (Group F) | | | | Cycle 2 dose every 21 days; |
| | | | | | no mosunetuzumab step-up |
| | | | | | dosing |

Study treatment could be discontinued at any time for unacceptable toxicity. At the discretion of the Medical Monitor and in consultation with the investigators, patients could be eligible for additional study re-treatment provided the aforementioned criteria for re-treatment continue to be met.

The schedule of assessments for patients who received re-treatment followed the schedule of assessments currently implemented in dose escalation or expansion. For example, patients being re-treated following a Cycle 1 double-step fractionated schedule followed the Group B schedule of assessments.

The rules for study treatment duration and re-treatment applied both to dose-escalation and dose-expansion cohorts.

### Dose-Expansion Stage

The dose-expansion stage of this study was designed to obtain additional safety, tolerability, PK, and preliminary clinical activity data with study treatment at doses up to the MTD/maximal assessed dose (MAD).

All available safety data from the expansion cohorts was evaluated on an ongoing basis to assess the tolerability of the dose levels studied. At no time did a mosunetuzumab dose level studied in the expansion stage exceed the highest dose level that qualified as an MTD in the dose-escalation stage. Additionally, for each expansion cohort, interim analyses were conducted in order to guide potential early stopping of enrollment in the event of excess toxicity.

Patients exhibiting acceptable safety and evidence of clinical benefit as described in the protocol continued to receive mosunetuzumab every 21 days up to a maximum of 8 or 17 cycles (See FIG. 3) until objective disease progression was documented or unacceptable toxicity, whichever occurred first.

Evaluations for safety and efficacy were conducted according to the schedules of assessments. Additional assessments after the final dose of study treatment for patients who discontinue study treatment for reasons other than disease progression, including patients who complete initial mosunetuzumab treatment, were performed as outlined in the post-treatment schedule of assessments. Patients who completed study treatment continued to have tumor assessments until disease progression and were eligible for mosunetuzumab re-treatment as described herein.

### A. Single-Agent Mosunetuzumab Dose-Expansion in NHL

Patients were enrolled to receive mosunetuzumab in the following separate indication-specific expansion cohorts:
R/R DLBCL and transformed FL: expansion cohorts testing doses at or below the MTD from Groups A, B, D, and F escalation could be enrolled. Each cohort enrolled up to approximately 20 patients, except the expansion cohort based on Group B RP2D, which enrolled approximately 80 patients, assuming sufficient safety and activity as defined herein.
R/R FL: expansion cohorts testing doses at or below the MTD from Groups A, B, D, and F escalation could be enrolled. Each cohort could enroll up to approximately 20 patients, except the expansion cohort based on Group B RP2D which could enroll approximately 80 patients, assuming sufficient safety and activity as defined herein.
R/R MCL: expansion cohorts testing doses at or below the MTD from Groups A, B, D, and F escalation could be enrolled. Each cohort could enroll up to approximately 20 patients assuming sufficient safety and activity as defined herein.
R/R Richter's transformation: An expansion cohort testing doses at or below the MTD from Group B escalation could be enrolled. The cohort could enroll approximately 10-20 patients assuming sufficient safety and activity as defined herein.

Mosunetuzumab doses and schedules assessed in dose expansion were determined by the IMC in consultation with the investigators (if necessary and possible from a timing perspective) following a review of cumulative safety data in dose escalation. More than one mosunetuzumab dose level and schedule could be assessed. Expansion cohorts could be initiated, prior to the identification of the RP2D, at doses previously determined to be safe and demonstrating evidence of clinical activity during dose escalation.

### B. Dose Escalation and Expansion in CLL

Dose-escalation rules for CLL patients are the same as those used for NHL patients. The starting dose for CLL patients was no higher than one dose level below that has cleared the DLT assessment window in the corresponding NHL dose escalation.

Expansion cohorts of up to approximately 10 patients assessing mosunetuzumab in CLL at doses determined to be safe and demonstrating of clinical activity during CLL Groups B, D, and F dose escalation was tested. The CLL expansion cohorts occurred independently and potentially with different doses and schedules from those for NHL patients.

### Assessment Of Safety

The following information describing management of safety concerns was based on anticipated pharmacology and mechanism of action, results from nonclinical studies, preliminary safety findings from the single-agent dose-escalation study, and published data on similar molecules.

Measures were taken to ensure the safety of patients participating in this trial, including the use of stringent inclusion and exclusion criteria and close monitoring, as described below. Enrollment of patients for DLT evaluation purposes was staggered such that the first 2 patients in each dose-escalation cohort had respective Cycle 1, Day 1 treatments administered >72 hours apart. Subsequent patients in each cohort were staggered such that their Cycle 1, Day 1 treatments were administered ≥24 hours apart.

All patients were monitored closely for toxicity. Patients were assessed clinically for toxicity prior to each dose using the NCI CTCAE v4.0 grading scale unless otherwise stated. CRS severity was graded according to the Modified Cytokine Release Syndrome Grading System (Table 2). All adverse events and serious adverse events were recorded during the trial and for up to 90 days after the last dose of study treatment or until the initiation of another systemic anti-cancer therapy, whichever occurs first. To mitigate potential unknown risks, at least in part, dosing beyond Cycle 1 was limited to patients who did not demonstrate unacceptable toxicity or compelling evidence of disease progression.

Specific anticipated or potential toxicities associated with administration of mosunetuzumab, as well as the measures taken intended to avoid or minimize such toxicities in this trial, are described herein.

### A. Mosunetuzumab Administrations and Hospitalization

Administration of mosunetuzumab was performed in a clinical setting with immediate access to a critical care unit and staff who are trained to monitor for and respond to medical emergencies. Neurology consultation services was readily available to address any neurologic adverse events that arose as a result of mosunetuzumab treatment, and nephrology consultation with acute dialysis capabilities was readily available to address any renal toxicity that might accompany tumor lysis syndrome (TLS).

All patients enrolled in Groups A and D dose escalation required inpatient monitoring, including hospitalization during or following mosunetuzumab administration, for the first mosunetuzumab administration through at least 72 hours after the completion of mosunetuzumab administration on Cycle 1 Day 1.

All patients enrolled in Group B dose escalation cohorts that received mosunetuzumab on a double-step-fractionated schedule required inpatient monitoring, including hospitalization during or following mosunetuzumab administration, for mosunetuzumab infusion through at least 72 hours after the completion of mosunetuzumab infusion for any individual dose exceeding any previously tested dose level.

This dose generally corresponded to the dose of mosunetuzumab administered on Cycle 1 Day 15. Hospitalization for administration of previously assessed doses was not required unless clinically indicated at the study investigator's discretion and in consultation with the Medical Monitor. Examples where such hospitalization were warranted include but were not limited to prior observed Grade ≥2 adverse events potentially attributable to mosunetuzumab (e.g., CRS, HLH, elevated liver enzymes (e.g., aspartate aminotransferase (AST), alanine aminotransferase (ALT), or total bilirubin elevations that occurred concurrently with signs or symptoms consistent with CRS or HLH and did not resolve within 72 hours and were not considered by the investigator to be attributable to another clearly identifiable cause), neurologic toxicity, TLS, worsening neutropenia and/or thrombocytopenia) at the same or similar dose, and TLS monitoring and prophylaxis.

Based on available clinical safety data, for patients who received mosunetuzumab at a dose level that had been tested to be safe and tolerable, hospitalization was not mandatory after any dosing day. This applied to patients enrolled in Group B dose-escalation backfill slots, Group B expansion cohorts, and to those patients who received study the need for hospitalization, and patients were hospitalized after mosunetuzumab administration whenever clinically indicated.

All patients enrolled in Group F dose escalation cohorts received mosunetuzumab on a Cycle 1 step-up dosing schedule receive inpatient monitoring, including hospitalization during or following mosunetuzumab administration, through at least 72 hours after the completion of mosunetuzumab injection on Cycle 1 Day 15 (see above). Additionally, if the Cycle 1 Day 1 and/or Cycle 1 Day 8 dose exceeded any previously highest tested dose level in Group D or Group F, 72-hour hospitalization was required following completion of Cycle 1 Day 1 and/or Cycle 1 Day 8 injections.

Hospitalization requirements during subsequent cycles were determined on the basis of the clinical course during the first cycle; patients with Grade 3 CRS, injection-site reactions, or TLS during Cycle 1 were also hospitalized through at least 72 hours after the end of the administration of the subsequent dose, with considerations for dose reduction as described herein.

For all treatment groups, decisions to modify or discontinue the requirement for hospitalization in expansion cohorts were made based on the recommendation of the IMC and in consultation with study investigators (if necessary and possible from a timing perspective). For patients enrolled in Group F dose-expansion cohorts, hospitalization was not mandatory after any dosing day if no Grade 3 CRS was observed during Group F dose escalation and approved by the IMC. The investigator actively assessed the need for hospitalization, and patients were hospitalized after mosunetuzumab administration whenever clinically indicated.

### B. Dose and Schedule Modifications

Mosunetuzumab dosing occurred only if a patient's clinical assessment and laboratory test values were acceptable. If scheduled dosing coincided with a holiday that precludes dosing, dosing commenced on the nearest following date, with subsequent dosing continuing on a 21-day schedule as applicable.

Study treatment was delayed as appropriate for management of toxicity. Specific guidelines for single-agent mosunetuzumab therapy dose modifications are described below. Management guidelines including study treatment dose and schedule modifications for specific adverse events are described below.

### Mosunetuzumab Dose and Schedule Modifications.

For patients who experienced CRS with the first dose of mosunetuzumab or were at increased risk of recurrent CRS with subsequent doses, the time of infusion could be extended to up to 8 hours.
Patients who experienced an adverse event that either met the definition of a DLT, a Grade 3 adverse event or a serious adverse event were allowed to delay mosunetuzumab dosing for up to 2 weeks in order to recover from the toxicity.
   During Cycle 1 double-step fractionation in Groups B and F, mosunetuzumab was administered despite hematologic laboratory abnormalities if no clinically significant symptoms were present; for anemia and thrombocytopenia, no transfusions were required.
   For those adverse events that were not considered by the investigator to be attributable to another clearly identifiable cause, (e.g., documented disease progression, concomitant medication, or pre-existing medical condition), patients continued to receive additional doses of mosunetuzumab, provided that the toxicity had resolved to Grade≤1 within the time period stated above.
      For decreased lab values, the abnormality resolved to the lower limit of Grade≤1, or return to >80% of the baseline value, whichever was lower.
      For neutropenia, the ANC resolved to Grade≤2 or return to >80% of the baseline value, whichever was lower.
      For increased lab values the abnormality resolved to the upper limit of Grade≤1, or return to ≥120% of the baseline value, whichever was higher.

The dose for subsequent administration of mosunetuzumab in such patients was generally reduced, e.g., to the next highest cleared dose level assessed during dose escalation. If the reduction was to a dose level where there was no evidence of mosunetuzumab pharmacodynamic activity, e.g., no evidence of changes in serum cytokine levels, the patient could be discontinued from study treatment.
Decisions on continued treatment at a reduced dose following a DLT or other study treatment related Grade 3 toxicity was made following a careful assessment and discussion of risk versus benefit with the patient by the investigator and approval from the Medical Monitor with the following exceptions:

If an elevation of AST or ALT >3×ULN and/or total bilirubin >2×ULN, with no individual laboratory value exceeding Grade 3, occurred in the context of Grade≤2 CRS (Table 2) which lasts <3 days, mosunetuzumab dosing continued without dose reduction with approval of the Medical Monitor.

For Grade 3 CRS (Table 2), the next mosunetuzumab dose was reduced, and subsequent doses was increased if the lower dose was tolerated. If Grade 3 CRS occurs in the step-up dosing cohorts following mosunetuzumab administration at Cycle 1 Day 1 or Cycle 1 Day 8, the next mosunetuzumab dose was discussed with the Medical Monitor, and a dose reduction was considered (Table 4).

For Grade 3 (NCI CTCAE v4) individual signs and symptoms of CRS that occurred in the context of Grade≤2 CRS (Table 2) which lasts <3 days, mosunetuzumab dosing continued without dose reduction with approval of the Medical Monitor.

For patients enrolled into expansion cohorts, decisions regarding dose and schedule modifications were made following individual benefit-risk assessment by the investigator and in consultation with the Medical Monitor.

Any patient in whom similar toxicity recurred at a reduced dose was discontinued from further mosunetuzumab treatment.

Patients could not be re-treated at the dose of mosunetuzumab at which the adverse event occurred unless the adverse event was attributed to another clearly identifiable cause.
Patients who did not fulfill the criteria for dosing after the additional 2 weeks elapsed were discontinued from study treatment and followed for safety outcomes. Exceptions to this on the basis of ongoing clinical benefit was allowed following investigator assessment of risk versus benefit with approval from the Medical Monitor. Delay of therapy because of toxicities not attributed to mosunetuzumab did not require discontinuation following investigator assessment of risk versus benefit with approval from the Medical Monitor.
   For patients in Groups B and F, if dose delay resulted in a treatment-free interval of 6 weeks or longer, double-step fractionation of mosunetuzumab was required on Days 1, 8, and 15 for the first cycle after the dose delay.
For patients receiving mosunetuzumab on a Cycle 1 step-up schedule (Groups B and F escalation or expansion), if a serious adverse event or adverse event of special interest occurred following Cycle 1, Day 1, Cycle 1, Day 8, and/or Cycle 1, Day 15 dosing, a treatment delay of mosunetuzumab up to 14 days and/or modification of the subsequent mosunetuzumab dose occurred at the discretion of the Medical Monitor following consultation with the treating investigator physician.

In the event that a patient has a toxicity in Cycle 1 necessitating mosunetuzumab interruption for >7 days, the Medical Monitor was notified, and the patient was required to repeat mosunetuzumab at the highest dose previously tolerated prior to resuming the planned treatment schedule.

Patients who discontinued study treatment for reasons other than PD were continued to be followed.

C. Risks Associated with Mosunetuzumab

On the basis of clinical data to date with mosunetuzumab, the following known and suspected risks are described below.

Known Risks Associated with Mosunetuzumab

### Cytokine Release Syndrome

The mechanism of action of mosunetuzumab is immune cell-activation against CD20-positive cells; therefore, a spectrum of events involving infusion-related reactions (IRRs), target-mediated cytokine release, and/or hypersensitivity with or without emergent ADAs, may occur. Other CD20-directed therapies and immunomodulatory therapies have been associated with IRRs, cytokine release syndrome (CRS), and/or hypersensitivity (RITUXAN® United States Package Insert (USPI); GAZYVA® USPI; BLINCYTO® USPI). CRS following mosunetuzumab administration has been reported in Study GO29781.

While CRS is a known risk associated with mosunetuzumab, comprehensive characterization is ongoing with accumulating clinical data. To date, CRS observed with mosunetuzumab have been mostly mild to moderate in severity, and include symptoms such as fever, headache, and myalgia, and respond to symptomatic treatment with analgesics, anti-pyretics, and antihistamines as indicated.

Severe or life-threatening presentations of CRS, such as hypotension, tachycardia, dyspnea, or chest discomfort, should be treated aggressively with supportive and resuscitative measures as indicated, including the use of tocilizumab and/or high dose corticosteroids, IV fluids, and other supportive measures per institutional practice. Severe CRS may be associated with other clinical sequelae such as disseminated intravascular coagulation, capillary leak syndrome, or may manifest as hemophagocytic lymphohistiocytosis (HLH). Standard of care for severe or life threatening CRS resulting from immune-based monoclonal antibody therapy has not been established; case reports and recommendations for CD19 CAR-T have been published (Teachey et al., Blood, 121 (26): 5154-5157, 2013; Lee et al., Blood, 124 (2): 188-195, 2014; Maude et al., New Engl J Med, 371 (16): 1507-1517, 2014; Neelapu et al., Nat Rev Clin Oncol, 15:47-62, 2018; also see FDA approval for two products describing risk management for CRS (YESCARTA® USPI; KYMRIAH® USPI)).

Disease-related factors that may be associated with an increased risk of severe CRS following chimeric antigen receptor (CAR)-T-cell therapy, and therefore, potentially other T-cell engaging therapies, include (but are not limited to) lymphoma bone marrow involvement, extranodal disease, Richter's transformation, B cell lymphocytosis, and the presence of circulating peripheral malignant cells.

### Neutropenia

Neutropenia has a known class effect associated with other CD20-directed therapies as well as blinatumomab (BLINCYTO® USPI), and is a known risk for mosunetuzumab. Reversible neutropenia has been observed following mosunetuzumab treatment in Study GO29781. Some patients developing neutropenia have received growth factor support and/or temporary treatment holds.

Patients who experience Grade 3-4 neutropenia should be closely monitored with more frequent assessments as applicable. For treatment-emergent neutropenia events that are Grade 3 or higher, dose delay and/or dose modification as described herein should be considered.

Potential Risks Associated with Mosunetuzumab

### Hemophagocytic Lymphohistiocytosis

CRS with features of adult-onset secondary or reactive macrophage activation syndrome/hemophagocytic lymphohistiocytosis (MAS/HLH) has been reported with blinatumomab as well as CAR adoptive T-cell therapy (BLINCYTO® USPI; Teachey et al., Blood, 121 (26): 5154-5157, 2013; Lee et al., Blood, 124 (2): 188-195, 2014). (Note: for the purposes of the GO29781 protocol, MAS and HLH are considered to be synonymous terms.) A fatal case of secondary HLH, in a patient with evidence of chronic active EBV infection (positive for EBV as assessed by EBV-encoded small RNA in situ hybridization), has been reported in Study GO29781.

While severe CRS and secondary HLH have overlapping presentation and symptoms, secondary HLH may be precipitated by other conditions including infections, autoimmune disease and malignancies (Ramos-Casals et al., Lancet, 383:1503-1516, 2014). The prevalence of these conditions in the study patient population makes the distinction between severe CRS and HLH and identification of inciting factors challenging. For example, in one series, B-cell malignancies were the most common malignancy associated with reactive HLH (Rivière et al., Am J Med, 127:1118-1125, 2014). Furthermore, active infection with EBV is one of the most common infectious causes of HLH (Hashemi-Sadraei et al., Case Rep Hematol 2015, 491567, 2015; Schram and Berliner, Blood, 125:2908-2914, 2015), while reactivation of latent EBV may occur in patients with CLL (Rath et al., Haematologica, 93:1424-1426, 2008), which in turn may lead to HLH (Lim et al., Leuk Lymphoma, 55:2938-2941, 2014). It remains unknown whether mosunetuzumab treatment may further increase the risk of developing HLH in patients who have additional risk factors.

In the setting of T-cell engaging therapies including mosunetuzumab, CRS is much more likely compared with secondary HLH. Considering the overlapping presentation of symptoms, management of these patients should be primarily focused on treatment of CRS (see Table 4).

In atypical cases such as late onset CRS (past completion of step-up dosing with mosunetuzumab) or CRS that is refractory to treatment, work up for HLH was initiated

The supportive management of HLH was generally similar to that of CRS. Specific diagnostic, monitoring and management guidelines for HLH were described below.

A patient should be classified as having HLH if five of the following eight criteria were met:
Fever ≥38.5° C.
Splenomegaly
Peripheral blood cytopenia consisting of at least two of the following:
   Hemoglobin <90 g/L (9 g/dL) (<100 g/L (10 g/dL) for infants <4 weeks old)
   Platelet count<100×10⁹/L (100,000/μL)
   ANC <1.0×10⁹/L (1000/μL)
Fasting triglycerides >2.992 mmol/L (265 mg/dL) and/or fibrinogen <1.5 g/L (150 mg/dL).
Hemophagocytosis in bone marrow, spleen, lymph node, or liver
Low or absent natural killer cell activity
Ferritin >500 mg/L (500 ng/mL)
Soluble interleukin 2 (IL-2) receptor (soluble CD25) elevated ≥2 standard deviations above age-adjusted laboratory-specific norms

Patients were hospitalized with the following diagnostic and monitoring measures initiated:
Frequent (e.g., every 4 hours) vital signs and physical examination including evaluation for splenomegaly;
Serial (at least daily) monitoring of serum chemistries, complete blood counts, liver function tests (LFTs), ferritin, PT/PTT, fibrinogen, D-dimer and triglycerides;
Consideration of bone marrow and/or lymph node biopsy to assess for hemophagocytosis and active infection, including assessment of EBV protein localization in T/B/NK cells;
Complete infectious disease work-up including:
   Blood cultures (bacterial and fungal)
   Urine cultures and urinalysis
   Radiographic assessments (e.g., chest X-ray or CT scan)
   Assessment for active viral infections, including but not limited to EBV and CMV.
If available, assessment for soluble CD25 and assessment of NK cell function.
DNA for exploratory genetic testing of mutations potentially associated with HLH, e.g., PRF1, MUNC13-4, STXBP2 was considered (Zhang et al., Blood, 118:5794-5798, 2011).

Patients with suspected HLH were treated according to the guidelines in Table 8. In the case of confirmed HLH, study treatment was permanently discontinued.

TABLE 8
Management guidelines for suspected
hemophagocytic lymphohistiocytosis

| Event | Management |
|---|---|
| Suspected HLH | Withhold study treatment. |
| | Consider patient referral to hematologist. |
| | Initiate supportive care, including intensive |
| | care monitoring if indicated per institutional |
| | guidelines. |
| | Consider treatment for HLH with appropriate |
| | therapy. |
| Confirmed HLH | Permanently discontinue study treatment. |
| | Refer patient to a hematologist. |
| | Institute appropriate supportive care, including |
| | intensive care monitoring, if indicated per the |
| | institutional guidelines. |
| | Treat with appropriate HLH therapy according to |
| | institutional standards or published references |
| | (Schram and Berliner, Blood, 125: 2908-2914, 2015; |
| | Vallurupalli and Berliner, Blood, 134(21): |
| | 1783-1786, 2019). |

### Injection Site Reactions

Localized injection-site reactions following SC administration of the anti-CD20 monoclonal antibody rituximab have been observed (Assouline et al., Lancet Haematol, c128-338, 2016). Most of these were mild to moderate in severity (MABTHERA® European Medicines Agency, Summary of Product Characteristics (EMA SPC)). As CD4+ and CD8+ T-cells (Mueller et al., Frontiers in Immunology, 332, 2014) as well as B cells (Egbuniwe et al., Trends Immunol, 36:102-111, 2015) reside in the skin, localized reactions following mosunetuzumab SC administration may occur. Consequently, the risk of injection-site reactions with mosunetuzumab is unknown. Patients who experience localized injection-site reactions following SC administration of mosunetuzumab were managed according to the guidelines detailed in Table 5.

### Neurologic Toxicity

Encephalopathy has been observed in in the setting of CRS and/or elevation in liver function tests (LFTs) following mosunetuzumab treatment.

Neurologic toxicity has been reported in cynomolgus monkeys administered mosunetuzumab and was frequently reported in patients treated with blinatumomab and CD19 CAR T-cell therapy (BLINCYTO® USPI; Kochenderfer et al., J Clin Oncol, published online before print Aug. 25, 2014; Maude et al., New Engl J Med, 371 (16): 1507-1517, 2014). Reported symptoms in patients treated with blinatumomab or CAR T-cell therapy have included headache, confusion, aphasia, encephalopathy, tremor, seizure, and other neurologic events. The etiology of toxicity in these settings is uncertain and may not be responsive to cytokine directed therapy such as tocilizumab, but has generally improved with treatment discontinuations and corticosteroids (BLINCYTO® USPI; Viardot et al., American Society of Hematology Annual Meeting 2010, Abstract 2880, 2010; Kochenderfer et al., J Clin Oncol, published online before print Aug. 25, 2014). In patients with B-cell ALL treated with blinatumomab, neurologic toxicities were observed in approximately 50% of patients; Grade ≥3 neurologic toxicity was observed in approximately 15% of patients. The majority of neurologic adverse events resolved following interruption of blinatumomab, with some patients requiring treatment discontinuation (BLINCYTO® USPI). Based on available clinical data, neurologic adverse events observed with mosunetuzumab have been mild in severity with early onset. The most frequent neurologic events include headache, dizziness, and insomnia.

### Tumor Lysis Syndrome

Tumor lysis syndrome (TLS) is a known pharmacodynamic effect of anti-tumor therapy in hematologic malignancies including NHL. TLS has been reported with blinatumomab, CAR T-cell therapy, and other CD20 directed therapy (BLINCYTO® USPI; GAZYVA® USPI; RITUXAN® USPI; Porter et al., N Engl J Med, 365 (8): 725-733, 2011). The inherent risk of TLS is dependent on the malignancy being treated and individual patient characteristics (Coiffier et al., J Clin Oncol, 26:2767-2778, 2008). There is the theoretical risk of TLS if treatment with mosunetuzumab resulted in the rapid destruction of a large number of tumor cells.

The risk of TLS with mosunetuzumab in NHL patients was predicted to be highest for those with bulky disease (defined in the context of TLS as any lesion >10 cm on the screening CT scan) and elevated pretreatment lactate dehydrogenase (LDH) levels, particularly in the presence of dehydration or compromised renal function. The risk of TLS with mosunetuzumab in CLL patients was predicted to be highest in patients with absolute lymphocyte counts >25×10⁹/L or those with any nodal lesion≥10 cm, especially in the presence of dehydration or compromised renal function. While DLBCL, transformed lymphomas, and MCLs could be at higher risk of TLS as compared with follicular, marginal, and small cell lymphomas (Cairo et al., Br J Haematol, 149:578-586, 2010), any risk stratification based on tumor type were to be considered along with the effectiveness of therapy (Howard et al., New Engl J Med, 364 (19): 1844-1854, 2011).

As mosunetuzumab has the potential for potent B-cell killing, all patients received prophylaxis for TLS based on the prophylaxis guidelines below.

Upon hospital admission for Cycle 1 study treatment administration or hospitalization following dose escalation, the patient's serum chemistry and hematology laboratory samples were obtained and reviewed and prophylactic measures initiated according to the guidelines described below.

All patients received prophylaxis for TLS prior to each mosunetuzumab administration at C1D1 for Groups A and D, at Cycle 1 Day 1, Cycle 1 Day 8, and Cycle 1 Day 15 for Groups B and F. Prophylaxis guidelines included the following:
Hydration, consisting of a fluid intake of approximately 2-3 L/day starting 24-48 hours prior to the first dose of mosunetuzumab.
   If a patient was hospitalized for the administration of study treatment, IV hydration at a rate of 150-200 mL/hour began at the conclusion of mosunetuzumab administration and continued for at least 24 hours thereafter.
   If a patient received study treatment in the outpatient setting, fluid intake was maintained at 2-3 L/day for at least 24 hours after mosunetuzumab administration.
   Modification of fluid rate should be considered for individuals with specific medical needs.
Administration of an agent to reduce uric acid:
   Allopurinol (e.g., 300 mg/day orally beginning 72 hours prior to dose and continuing for 3-7 days afterwards) for those patients judged to be of low or intermediate risk of developing TLS per the investigator's discretion.
   For patients with elevated uric acid levels prior to mosunetuzumab treatment, or considered to be at high risk for TLS: rasburicase (e.g., 0.2 mg/kg IV over 30 minutes prior to first dose mosunetuzumab and daily for up to 5 days thereafter) was administered, unless contraindicated (ELITEK® USPI).
   Treatment with allopurinol/rasburicase should continue as specified above, or if laboratory evidence of TLS is observed until normalization of serum uric acid or other lab parameters.
   If treatment with allopurinol or rasburicase is contraindicated or is otherwise inappropriate in the view of the investigator, the Medical Monitor was contacted for further guidance.

### Infections

Due to its anticipated mode of action resulting in profound B-cell depletion, mosunetuzumab may be associated with an increased risk of infections. Infections have been reported in patients receiving other CD20 directed therapies as well as blinatumomab (BLINCYTO® USPI; GAZYVA® USPI; RITUXAN® USPI). Therefore, mosunetuzumab was be administered in the presence of active severe infections.

Investigators exercised caution when considering the use of mosunetuzumab in patients with history of recurring or chronic infections or with underlying conditions that may predispose patients to infections. Signs and symptoms of infection resulted in prompt evaluation and appropriate samples for bacteriological investigation prior to starting antibiotic or other treatment.

Particular attention was given to patients who have had significant prior immunosuppressive treatment such as high dose chemotherapy. Progressive multifocal leukoencephalopathy (PML) has been associated with treatment with CD20 directed therapies including rituximab and obinutuzumab. The diagnosis of PML was considered in any patient presenting with new-onset neurologic manifestations and consultation with a neurologist and diagnostic procedures including brain MRI and lumbar puncture should be performed as clinically indicated. Note, however, that new onset neurologic adverse events following initial doses of mosunetuzumab was more likely due to acute effects of mosunetuzumab, as PML associated with rituximab generally occurred following long-term exposure (Carson et al., Blood, 113 (20): 4834-4840, 2009).

### Thrombocytopenia

Thrombocytopenia is associated with other CD20 directed therapies as well as blinatumomab (BLINCYTO® USPI). Reversible thrombocytopenia has been observed following mosunetuzumab treatment in Study GO29781.

In nonclinical testing of mosunetuzumab in cynomolgus monkeys, hematology findings included transiently decreased WBC, lymphocyte, monocyte, eosinophil, basophil, and platelet counts within the first day of mosunetuzumab exposure, followed by recovery or rebound recovery between Days 4-8.

Patients were closely monitored for thrombocytopenia; regular laboratory tests were performed until the event resolves. Transfusion of blood products (e.g., platelet transfusion) according to institutional practice was at the discretion of the treating physician. Use of all concomitant therapies, which could possibly worsen thrombocytopenia-related events such as platelet inhibitors and anticoagulants, was be taken into consideration.

For treatment-emergent thrombocytopenia events that are Grade 3 or higher, dose delay and/or dose modification was considered.

### Elevated Liver Enzymes and Hepatic Events

Elevated liver enzymes have been reported with blinatumomab (BLINCYTO® USPI), usually but not exclusively in the setting of CRS. Grade ≥3 liver enzyme elevations occurred in approximately 6% of patients outside the setting of CRS. Nearly all liver enzyme elevations resolved either with blinatumomab treatment interruption or while treatment continued. Some patients with resolved liver enzyme elevations were successfully rechallenged, suggesting a first-dose effect rather than direct toxicity (BLINCYTO® Drug Approval Package). Transient Grade 3 AST elevation in the setting of Grade 2 CRS as well as Grade 3 hepatic encephalopathy/Grade 4 elevation in LFTs have been observed following mosunetuzumab treatment.

In nonclinical testing with mosunetuzumab in cynomolgus monkeys, dose-dependent increases in serum total bilirubin along with CRP, fibrinogen, PT, and aPTT were observed, consistent with mosunetuzumab-induced cytokine release and an acute phase protein response, with minimal activation of the coagulation system. Possible drug-related microscopic findings in the liver included single-cell hepatocyte degeneration/necrosis and immune cell infiltration in the portal area. All findings showed evidence of reversibility.

### Immunogenicity (Anti-Drug Antibodies)

As with any recombinant antibody, mosunetuzumab may elicit an immune response, and patients may develop antibodies against the molecule. Patients were closely monitored for any potential immune response to mosunetuzumab, which could have an impact on the benefit-risk profile of the agent.

### Tumor Inflammation/Flare

Adverse events associated with tumor inflammation/flare had been reported in Study GO29781. Consistent with the mechanism of action of mosunetuzumab, tumor flare was likely due to the influx of T cells into tumor sites following mosunetuzumab administration and could be associated with pseudoprogression. Tumor flare-associated adverse events observed to date have had a short time to onset following initial mosunetuzumab administration. Tumor flare may additionally occur in patients who are retreated with mosunetuzumab following disease progression. On the basis of emerging safety data, tumor flare has manifested as tumor pain, increase in size of known nodal or extranodal lesions by clinical or radiographic assessment, as well as new or worsening pleural effusions. In addition, depending on tumor size and anatomic location, tumor flare may potentially result in mass effects on vital structures including airways, major blood vessels, gastrointestinal tract (risk of perforation and hemorrhage), and/or major organs. If such manifestations are temporally associated with early mosunetuzumab dosing, the treating physician/study investigator should consider those events to be tumor flare and report as “tumor flare” or “tumor inflammation”. For patients with tumors at critical anatomic locations, the treating physician/study investigator contacted the Medical Monitor to discuss risk assessment and mitigation strategies prior to mosunetuzumab treatment and patients were closely monitored for tumor flare.

### D. Assessment of Severity and Causality of Adverse Events

The adverse event severity grading scale for the NCI CTCAE (v4.0) was used for assessing adverse event severity unless otherwise specified. Table 9 was used for assessing severity for adverse events that were not specifically listed in the NCI CTCAE.

TABLE 9
Adverse event severity grading scale for events
not specifically listed in NCI CTCAE

| Grade | Severity |
|---|---|
| 1 | Mild; asymptomatic or mild symptoms; clinical or |
| | diagnostic observations only; or intervention not |
| | indicated |
| 2 | Moderate; minimal, local, or non-invasive intervention |
| | indicated; or limiting age-appropriate instrumental |
| | activities of daily living^{a} |
| 3 | Severe or medically significant, but not immediately |
| | life-threatening; hospitalization or prolongation of |
| | hospitalization indicated; disabling; or limiting |
| | self-care activities of daily living^{b} |
| 4 | Life-threatening consequences or urgent intervention |
| | indicated |
| 5 | Death related to adverse event ^{d} |

NCI CTCAE = National Cancer Institute Common Terminology Criteria for Adverse Events.
Note:
Based on the most recent version of NCI CTCAE (v5.0).
^{a}Instrumental activities of daily living refer to preparing meals, shopping for groceries or clothes, using the telephone, managing money, etc.
^{b}Examples of self-care activities of daily living include bathing, dressing and undressing, feeding oneself, using the toilet, and taking medications, as performed by patients who are not bedridden.

### A. Safety Analyses

The safety analyses included all patients who received any amount of study treatment. Safety was assessed through summaries of adverse events, changes in laboratory test results, changes in ECGs, changes in ADAs, and changes in vital signs.

All collected adverse event data were listed by assigned dose level and patient number. All adverse events occurring on or after treatment on Cycle 1 Day 1 were summarized by mapped term, appropriate thesaurus levels, and NCI CTCAE v4.0 toxicity grade. In addition, all serious adverse events, including deaths, were listed separately and summarized. DLTs and adverse events leading to treatment discontinuation were also separately listed.

### B. Pharmacokinetic Analyses

Individual and mean serum concentration of mosunetuzumab versus time data were tabulated and plotted by dose level. The pharmacokinetics of mosunetuzumab were summarized by estimating total AUC, Cₘₐₓ, Cₘᵢₙ, CL, and Vₛₛ (as appropriate for data collected). Estimates for these parameters were tabulated and summarized. Inter-patient variability and drug accumulation were evaluated.

Serum trough and maximum concentration for tocilizumab, where applicable, were summarized, as appropriate and as data allow. Compartmental, non-compartmental, and/or population methods were considered. Additional PK analyses were conducted as appropriate.

### C. Activity Analyses

Response assessment data, PFS, and duration of response, all assessed by the investigator, were summarized for all patients by dose level, schedule, and arm. The objective response rate (ORR) was estimated. Investigator-assessed objective response was defined as a CR or PR as determined by investigator assessment using standard criteria. Patients with missing or no response assessments were classified as non-responders.

Among patients with an investigator-assessed objective response, duration of response was defined as the time from the initial CR or PR to the time of disease progression as determined by the investigator, or death. If a patient did not experience disease progression or death from any cause before the end of the study, duration of response was censored at the day of the last tumor assessment.

Investigator-assessed PFS was defined as the time from the first day of study treatment (Cycle 1, Day 1) to disease progression as determined by the investigator, or death, whichever occurs first. If a patient had not experienced PD or death, PFS was censored at the day of the last tumor assessment.

### Secondary Efficacy Endpoints

The secondary efficacy endpoints included:
Investigator-assessed CR rate, defined as the proportion of patients whose best overall response was a CR based upon investigator assessment using standard criteria for NHL (Cheson et al., J Clin Oncol, 25: 579-586, 2007). The exact 95% confidence intervals using the Clopper-Pearson method for CR rate were provided.
ORR, defined as the proportion of patients whose best overall response was a PR or CR using standard criteria for NHL (Cheson et al., J Clin Oncol, 25:579-586, 2007). ORR was assessed by the IRF and by the investigator. The exact 95% confidence intervals using the Clopper-Pearson method for ORR were provided.
Duration of complete response, defined as the time from the initial occurrence of a documented CR until documented disease progression or death due to any cause, whichever occurs first. Duration of complete response was assessed by the IRF and by the investigator, using standard criteria for NHL. The Kaplan-Meier estimate was provided. The Brookmeyer-Crowley method was used to construct the 95% confidence interval for the median duration of complete response.
Duration of response, defined as the time from the initial occurrence of a documented PR or CR until documented disease progression or death due to any cause, whichever occurred first. Duration of response was assessed by the IRF and by the investigator, using standard criteria for NHL. The Kaplan-Meier estimate was provided. The Brookmeyer-Crowley method was used to construct the 95% confidence interval for the median duration of response.
PFS, defined as the time from the first study treatment to the first occurrence of disease progression or death from any cause, whichever occurred first. PFS was assessed by the IRF and by the investigator, using standard criteria for NHL. The Kaplan-Meier estimate was provided. The Brookmeyer-Crowley method was used to construct the 95% confidence interval for the median PFS. Kaplan-Meier method was used to estimate 6-month PFS and 1-year PFS, along with the standard error and the corresponding 95% Cls using Greenwood's formula.
OS, defined as the time from the first study treatment to the date of death from any cause. The Kaplan-Meier estimate was provided. The Brookmeyer-Crowley method was used to construct the 95% confidence interval for the median OS. Kaplan-Meier method was used to estimate 6-month OS and 1-year OS, along with the standard error and the corresponding 95% Cls using Greenwood's formula.

### Example 2. A Phase Ib/II, Open-Label, Non-Randomized, Multicenter Study Evaluating the Safety, Tolerability, and Pharmacokinetics of Intravenous Versus Subcutaneous Mosunetuzumab in Combination with Lenalidomide in Patients with Follicular Lymphoma

### Objectives

The primary objective was to assess the PK non-inferiority of the mosunetuzumab SC regimen compared to the reference mosunetuzumab IV regimen, each in combination with lenalidomide. The secondary objective of this study was to evaluate treatment efficacy by measuring response and long-term efficacy outcomes. Response was determined by the investigator in the non-randomized stage and by the investigator and Independent Review Committee (IRC) in the randomized stage through use of positron emission tomography (PET)-computed tomography (CT) scans, using the Lugano 2014 criteria.

PK non-inferiority was assessed by comparing the observed PK between Arm A (IV Mosun+Len) and Arm B (SC Mosun+Len) based on the following PK parameters:

### Co-Primary PK Endpoints:

Cumulative AUC over Cycles 1-3 (AUC_{C1-3}) (i.e., calculated as AUC from Day 1 to Day 78)
Serum trough concentration at steady state, approximated by Cycle 4 (C_{trough. C4}) (i.e., the predose value on Day 106)

### Secondary PK Endpoints:

Cumulative AUC over Cycles 1-2 (AUC_{C1-2}) (i.e., calculated as AUC from Day 1 to Day 50)
Serum trough concentration in Cycle 2 (C_{trough. C2}) (i.e., the predose value on Day 50)
AUC at steady state (AUCₛₛ) (i.e., estimated by the AUC during Cycle 4)

The efficacy of treatment with Mosun+Len, was assessed based on the following endpoints:
CRR, defined as the proportion of patients whose best overall response is a CR during the study.
ORR, defined as the proportion of patients whose best overall response is a PR or a CR during the study.
DOR, defined as the time from the first occurrence of a documented objective response (CR or PR) to disease progression or relapse or death from any cause, whichever occurs first.
Duration of complete response (DOCR) was defined as the time from the first occurrence of a documented CR to disease progression or relapse or death from any cause, whichever occurs first.

Patients with no response assessment were counted as non-responders. Patients who had not progressed or died at time of analysis were censored at the date of last tumor assessment.

The additional secondary objectives were to compare safety and tolerability between Arm A (IV Mosun+Len) and Arm B (SC Mosun+Len) based on the following endpoints:
Incidence, nature, and severity of physical findings and adverse events, with severity determined according to the National Cancer Institute Common Terminology Criteria for Adverse Events version 5 (NCI CTCAE v5.0); for CRS, severity determined according to the ASTCT CRS grading criteria
Change from baseline in targeted vital signs.
Change from baseline in targeted clinical laboratory test results.
Tolerability, as assessed by the incidence of dose interruptions, dose reductions and dose intensity, and treatment discontinuation not due to efficacy events

### Study Design

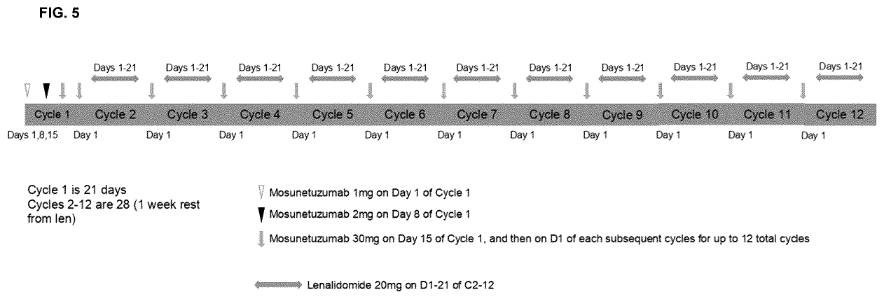

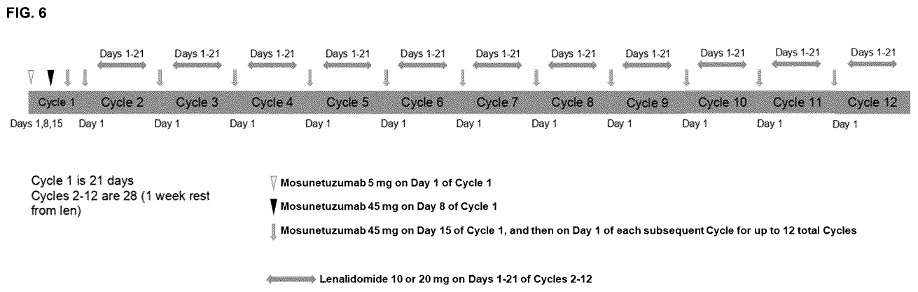

Study CO41942 is a Phase Ib/II, open-label, multicenter study, and includes evaluation of the safety, tolerability, and pharmacokinetics of intravenous (IV) or subcutaneous (SC) mosunetuzumab administration in combination with lenalidomide in patients with follicular lymphoma (FL). The study design of the present study is summarized in FIG. 5 (IV mosun+Len) and FIG. 6 (SC+Len).

Study treatment was administered for 12 cycles; the duration of dosing cycle (Cycle) 1 was 21 days, and the duration of Cycle 2-12 is 28 days. Mosunetuzumab was administered together with lenalidomide in Cycle 2 after patients complete Cycle 1 step-up dosing.

### Inclusion Criteria

Age ≥18 years
Eastern Cooperative Oncology Group (ECOG) Performance Status of 0, 1, or 2 (see Table 10 below).
R/R FL after treatment with at least one prior systemic lymphoma therapy, which included prior immunotherapy or chemoimmunotherapy; For patients who had only received one prior line of systemic therapy, patients must either have had FLIPI score 2-5, have been refractory to prior anti-CD20 MAb, or have had progression of disease within 24 months after initiation of prior therapy (POD24); these restrictions did not apply for patients who had received two or more prior lines of systemic therapy.
Previously untreated patients with follicular lymphoma (FL) required systemic therapy assessed by investigator based on Groupe d'Etude des Lymphomes Folliculaires (GELF) criteria (Brice et al. J Clin Oncol. 15 (3): 1110-1117, 1997).
Histologically documented FL of Grade 1, 2, or 3a (Swerdlow S H, et al. Blood 2016; 127:2375-90), and that expressed CD20
Fluorodeoxyglucose-avid lymphoma (i.e., positron emission tomography (PET)-positive lymphoma)
At least one bi-dimensionally measurable nodal lesion (>1.5 cm in its largest dimension by PET-computed tomography (CT) scan), or at least one bi-dimensionally measurable extranodal lesion (>1.0 cm in its largest dimension by PET-CT scan)
Adequate hematologic function (without growth factors or blood product transfusion within 14 days of first dose of study drug administration) defined as follows:
   Hemoglobin ≥9 g/dl
   Absolute neutrophil count (ANC) ≥1.0×10⁹/L
   Platelet count ≥75×10⁹/L.
Measured or estimated creatinine clearance >50 mL/min by institutional standard method
Aspartate aminotransferase (AST) or alanine transaminase (ALT)<2.5×upper limit of normal (ULN)
Serum total bilirubin <1.5×ULN (or <3×ULN for patients with Gilbert syndrome)
For women of childbearing potential: agreement to remain abstinent (refrain from heterosexual intercourse) or use two adequate methods of contraception, including at least one method with a failure rate of <1% per year, for at least 28 days prior to Day 1 of Cycle 1, during the treatment period (including periods of treatment interruption), and for at least 28 days after the last dose of lenalidomide, 3 months after the final dose of tocilizumab, and 3 months after the final dose of mosunetuzumab. Women must refrain from donating eggs during this same period.
For men: agreement to remain abstinent (refrain from heterosexual intercourse) or use contraceptive measures and agreement to refrain from donating sperm.

TABLE 10
ECOG Performance Status Scale

| Grade | Description |
|---|---|
| 0 | Fully active; able to carry on all pre-disease |
| | performance without restriction. |
| 1 | Restricted in physically strenuous activity but |
| | ambulatory and able to carry out work of a light |
| | or sedentary nature (e.g., light housework or |
| | office work). |
| 2 | Ambulatory and capable of all self-care but unable |
| | to carry out any work activities. Up and about |
| | >50% of waking hours. |
| 3 | Capable of only limited self-care; confined to a |
| | bed or chair >50% of waking hours. |
| 4 | Completely disabled. Cannot carry on any self- |
| | care. Totally confined to bed or chair. |
| 5 | Dead. |

### Exclusion Criteria

Any history of Grade 3b FL
Any history of transformation and/or diffuse large B cell lymphoma (DLBCL)
Active or history of central nervous system (CNS) lymphoma or leptomeningeal infiltration
Documented refractoriness to lenalidomide, defined as no response (PR or CR) within 6 months of therapy
Prior standard or investigational anti-cancer therapy as specified below:
   Lenalidomide exposure within 12 months prior to Day 1 of Cycle 1
   Fludarabine or alemtuzumab within 12 months prior to Day 1 of Cycle 1
   Radioimmunoconjugate within 12 weeks prior to Day 1 of Cycle 1
   Prior anti-lymphoma treatment with monoclonal antibody or antibody-drug conjugate within 4 weeks prior to Day 1 of Cycle 1
   Treatment with any chemotherapeutic agent, or treatment with any other anti-cancer agent (investigational or otherwise) within 4 weeks or 5 half-lives of the drug, whichever was shorter, prior to first dose of study treatment.
Clinically significant toxicity (other than alopecia) from prior treatment that has not resolved to Grade≤2 (per NCI CTCAE v5.0) prior to Day 1 of Cycle 1
Known history of idiopathic pulmonary fibrosis, organizing pneumonia (e.g., bronchiolitis obliterans), drug-induced pneumonitis or evidence of active pneumonitis on screening chest CT scan
Treatment with systemic immunosuppressive medications, including, but not limited to, prednisone, azathioprine, methotrexate, thalidomide, and anti-tumor necrosis factor agents within 2 weeks prior to Day 1 of Cycle 1
   The use of inhaled corticosteroids was permitted.
   The use of mineralocorticoids for management of orthostatic hypotension was permitted.
   The use of physiologic doses of corticosteroids for management of adrenal insufficiency was permitted.
   If corticosteroid treatment is urgently required for lymphoma symptom control prior to the start of study treatment, 100 mg of prednisone or equivalent can be given for a maximum of 5 days.
History of solid organ transplantation.
History of severe allergic or anaphylactic reaction to humanized, chimeric or murine monoclonal antibodies
Known hypersensitivity to biopharmaceuticals produced in Chinese hamster ovary cells or any component of the mosunetuzumab, lenalidomide, or thalidomide formulation, including mannitol.
History of erythema multiforme, Grade ≥3 rash, or blistering following prior treatment with immunomodulatory derivatives
Known active bacterial, viral, fungal, or other infection, or any major episode of infection requiring treatment with IV antibiotics within 4 weeks of Day 1 of Cycle 1
Known or suspected chronic active Epstein-Barr virus (EBV) infection
Known or suspected hemophagocytic syndrome.
Active hepatitis B infection: patients who are hepatitis B surface antigen (HBsAg) negative and hepatitis B core antibody (HBcAb) positive, must be negative for hepatitis B virus (HBV) polymerase chain reaction (PCR) to be eligible for study participation.
Active hepatitis C infection: patients who are positive for hepatitis C virus (HCV) antibody must be negative for HCV by PCR to be eligible for study participation.
Known history of human immunodeficiency virus (HIV) positive status: for patients with unknown HIV status, HIV testing is performed at screening if required by local regulations.
History of progressive multifocal leukoencephalopathy (PML)
Administration of a live, attenuated vaccine within 4 weeks before first dose of study treatment or anticipation that such a live attenuated vaccine is required during the study
   Patients must not receive live, attenuated vaccines (e.g., FluMist®) while receiving study treatment or after the last dose until B-cell recovery to the normal ranges. Killed vaccines or toxoids should be given at least 4 weeks prior to the first dose of study treatment to allow development of sufficient immunity.
   Approved non-live COVID-19 vaccine was permitted
   Inactivated influenza vaccination was given during influenza season only.
Other malignancy that could affect compliance with the protocol or interpretation of results, with the exception of the following:
   Any of the following malignancies previously curatively treated: carcinoma in situ of the cervix, good-prognosis ductal carcinoma in situ of the breast, basal, or squamous cell skin cancer
   Stage I melanoma, low-grade, early-stage localized prostate cancer, or any other previously treated malignancy that has been in remission without treatment for ≥2 years prior to enrollment
Active autoimmune disease requiring treatment
   Patients with a history of autoimmune-related hypothyroidism on a stable dose of thyroid-replacement hormone could be eligible.
   Patients with controlled Type 1 diabetes mellitus who were on an insulin regimen were eligible for the study.
   Patients with a history of disease-related immune thrombocytopeniaurpura, or autoimmune hemolytic anemia, or other stable autoimmune diseases could be eligible.
   Patients with a history of autoimmune disease, including, but not limited to, myasthenia gravis, myositis, autoimmune hepatitis, systemic lupus erythematosus, rheumatoid arthritis, inflammatory bowel disease, vascular thrombosis associated with antiphospholipid syndrome, Wegener granulomatosis, Sjögren syndrome, Guillain-Barré syndrome, multiple sclerosis, vasculitis, or glomerulonephritis, with treatment-free interval from immunosuppressive therapy for 12 months, could be eligible.
Prior allogeneic hematopoietic stem cell transplant
Grade ≥2 neuropathy
Evidence of any significant, uncontrolled concomitant disease that could affect compliance with the protocol or interpretation of results, including, but not limited to significant cardiovascular disease (e.g., New York Heart Association Class III or IV cardiac disease, myocardial infarction within the previous 6 months, unstable arrhythmia, or unstable angina) or significant pulmonary disease (such as obstructive pulmonary disease or history of bronchospasm).
Major surgical procedure other than for diagnosis within 28 days prior to Day 1 of Cycle 1 or anticipation of a major surgical procedure during the course of the study.
Clinically significant history of liver disease, including viral or other hepatitis, current alcohol abuse, or cirrhosis.
Pregnant or lactating or intending to become pregnant during the study

### Study Treatments

Study treatment was administered for 12 cycles; the duration of Cycle 1 was 21 days, and the duration of Cycle 2-12 was 28 days.

### Mosunetuzumab Administration

For IV mosunetuzumab+lenalidomide, mosunetuzumab was administered intravenously following a double-step fractionated dose escalation regimen: in Cycle 1 (21-day cycle), patients received 1 mg on Day 1 (C1D1 dose); 2 mg on Day 8 (C1D2 dose); and 30 mg on Day 15 (C1D3 dose). In Cycles 2-12 (28-day cycles), patients received 30 mg on Day 1 (C2D1-C12D1 doses). In Cycle 1, mosunetuzumab was administered via intravenous infusion over 4 hours (+15 min). If no CRS occurs in Cycle 1, infusion time could be shortened to 2 hours (+15 min).

For SC mosunetuzumab+lenalidomide, mosunetuzumab was administered subcutaneously (SC) following a step-up dosing regimen: in Cycle 1 (21-day dosing cycle), patients received 5 mg on Day 1 (C1D1 dose); 45 mg on Day 8 (C1D2 dose); and 45 mg on Day 15 (C1D3 dose). In Cycles 2-12 (28-day dosing cycles), patients received 45 mg on Day 1 (C2D1-C12D1 doses). Hospitalization was not required for patients who received subcutaneous mosunetuzumab unless clinically indicated. Patients treated with SC mosunetuzumab+lenalidomide could choose to receive maintenance therapy, which was administered as 45 mg SC mosunetuzumab every 8 weeks for an additional 9 dosing cycles (8 weeks each). Maintenance therapy began (e.g., dosing cycle 13) 8 weeks after Cycle 12, Day 1.

In the event that a patient had a non-hematologic toxicity prior to Cycle 1 Day 15 with IV dose that necessitated mosunetuzumab interruption for >7 days, the patient was required to repeat mosunetuzumab at the highest dose previously tolerated prior to resuming planned treatment schedule. In the event that a patient had a toxicity necessitating SC mosunetuzumab interruption for >7 days prior to the Cycle 1 Day 8 dose, the patient was required to repeat the 5 mg dose prior to resuming the planned treatment schedule (7 days after the administration of the 5 mg dose). If dose delay results in a treatment-free interval of 6 weeks or longer, step up dosing of SC mosunetuzumab was required on Days 1 (5 mg), and 8 (45 mg) for the first cycle after the dose delay. Corticosteroid prophylaxis was administered on days when repeat doses are administered to mitigate cytokine release syndrome (CRS) risks.

Corticosteroid premedication (prophylaxis) consisting of dexamethasone 20 mg or methylprednisolone 80 mg was administered orally or intravenously (IV) prior to the administration of each mosunetuzumab dose. In addition, premedication with oral or IV analgesic/antipyretic (e.g., 500-1,000 mg acetaminophen or paracetamol) and/or with oral or IV antihistamine (e.g., 50-100 mg diphenhydramine) was administered per standard institutional practice prior to administration of mosunetuzumab. Premedication with analgesic/antipyretic and antihistamine occurred at least 30 minutes prior to administration of mosunetuzumab. Premedication was administered to all patients in Cycle 1, and was optional from Cycle 2 and beyond. However, if a patient experienced CRS in earlier doses, premedication with corticosteroids was administered for subsequent doses.

Additionally, for patients at risk of tumor lysis syndrome (TLS; e.g., because of bulky disease or renal impairment (creatinine clearance <60 mL/min)), allopurinol or a suitable alternative such as rasburicase was used as premedication.

### Lenalidomide Administration

Lenalidomide was administered orally (PO) 20 mg once daily on Days 1-21 of Cycles 2-12 (28-day cycles). Lenalidomide was not administered on the last 7 days of each of the 28-day Cycles 2-12. If creatinine clearance was <60 mL/min, lenalidomide was instead started at a dose of 10 mg/day. Lenalidomide was provided as 5-, 10-, 15-, and 20-mg capsules.

On the days when lenalidomide was administered with mosunetuzumab, lenalidomide was administered first, followed by mosunetuzumab infusion or subcutaneous mosunetuzumab injection. Lenalidomide was administered at approximately the same time each day. If a dose of lenalidomide was missed and it had been≤12 hours since the time of the scheduled dose, the patient was allowed to take the missed dose. If it had been >12 hours, the dose was skipped and the next dose was taken at the regularly scheduled time. Two doses were not to be taken at the same time. If a dose was vomited, the dose was not re-taken.

Granulocyte colony-stimulating factor (G-CSF) could be administered per American Society of Clinical Oncology, European Organisation for Research and Treatment of Cancer (EORTC), and European Society for Medical Oncology guidelines (Smith et al. J. Clin. Oncol. 33 (28): 3199-3212; 2015).

Prophylactic treatment with antibiotics was administered as per standard practice.

Lenalidomide increased the risk of thromboembolism (TE). Anticoagulation prophylaxis could be given after a careful assessment of a patient's underlying risk factors. All patients were recommended to receive daily low-dose aspirin (81-100 mg) during lenalidomide treatment and until 28 days after the last dose of lenalidomide. Patients who were unable to tolerate aspirin, who had a history of TE, and who were at high risk of TE could receive warfarin or low-molecular-weight heparin or novel oral anticoagulant in accordance with institutional practice.

### Tocilizumab Administration

Tocilizumab was administered when necessary to patients who experienced a CRS event. Tocilizumab was administered at a dose of 8 mg/kg IV (for participants at or above 30-kg weight only) or 12 mg/kg (for participants less than 30 kg weight) for maximum of 4 doses; doses exceeding 800 mg per infusion were not recommended.

### Dose Modification

Mosunetuzumab dosing was not modified in this study. Patients receiving mosunetuzumab who experience a Grade 4, related, non-hematologic adverse event discontinued study treatment.

Lenalidomide dosing was modified for patients individually based on toxicity rules described below and further in detail in Table 11. The lenalidomide dose was reduced in 5-mg increments (e.g., 20 mg reduced to 15 mg; 15 mg reduced to 10 mg, 10 mg reduced to 5 mg). There was no more than one dose reduction per dosing cycle. If the lenalidomide dose was reduced, re-escalation was not permitted. If lenalidomide was started at a dose of 10 mg/day for creatinine clearance ≥50 but <60 mL/min, and creatinine clearance remained above 50 mL/min after 2 cycles, the lenalidomide dose could be increased to 15 mg if the patient had tolerated therapy. Doses that were missed, due to toxicity or any other reasons, were not rescheduled or readministered.

TABLE 11
Toxicity-Based Dose Modification for Lenalidomide

| NCI CTCAE | |
|---|---|
| Toxicity Grade | Action Required |
| Grade 3 or 4 anemia ^{a, b} | For patients on a lenalidomide dose ≥10 mg |
| | who have had one or no prior lenalidomide dose |
| | reductions |
| | Withhold lenalidomide and check full blood count |
| | at least every 7 days. |
| | Administer RBCs or platelets as required. |
| | If the event is ongoing on Day 1 of the next |
| | cycle but improves to Grade ≤2 or baseline |
| | ≤14 days after the scheduled date of the cycle, |
| | resume lenalidomide at current dose or reduced |
| | dose if the patient received <21 days of |
| | lenalidomide due to toxicity. |
| | If the event is ongoing on Day 1 of the next |
| | cycle but improves to Grade ≤2 or baseline |
| | 15-21 days after the scheduled date for the next |
| | cycle, resume lenalidomide at a reduced dose for |
| | current and subsequent cycles. ^{a, b} |
| | For patients on a lenalidomide dose of 5 mg and |
| | patients who have had 3 prior lenalidomide dose |
| | reductions: |
| | Permanently discontinue lenalidomide. |
| Renal Toxicity | For patients with creatinine clearance at study |
| | entry of ≥50 but <60 mL/min, lenalidomide dosing |
| | starts at 10 mg. If patient tolerates the therapy, |
| | and creatinine clearance remains >50 ml/min after |
| | 2 cycles, the lenalidomide dose may be increased |
| | to 15 mg. |
| | For patients with creatinine clearance that decrease |
| | during treatment to above 30 but <60 mL/min, |
| | lenalidomide dose is reduced to 10 mg, dose is not |
| | re-escalated |
| | If creatinine clearance is <30 mL/min and dialysis |
| | is not required, lenalidomide should be given at a |
| | dose of 5 mg/day. |
| | If creatinine clearance is <30 mL/min and dialysis |
| | is required, lenalidomide should be given at a dose |
| | of 5 mg/day. On dialysis days, lenalidomide is |
| | administered after dialysis. |

Rash

| | |
|---|---|
| Grade 2 or Grade 3 without | Hold treatment with lenalidomide (interrupt dose). |
| desquamating | Possibly administer steroid prophylaxis. When resolved |
| | (Grade ≤1), restart lenalidomide at the same dose |
| | level |
| Desquamating (blistering) | Permanently discontinue lenalidomide. |
| Grade ≥ 3 or Non- | |
| desquamating Grade 4 | |

Allergic Reaction or Hypersensitivity

| | |
|---|---|
| Grade 2 | If allergic reaction has occurred on or after Day 15 |
| | hold (interrupt dose) for the rest of the cycle and |
| | follow at least every 7 days |
| | If allergic reaction has occurred before Day 15 and |
| | resolved to Grade 1 restart at same dose level for the |
| | rest of the cycle |
| | In both cases, restart subsequent cycle at next lower |
| | dose and please contact the Medical Monitor to discuss |
| | any further steroid prophylaxis |
| Grade 3-4 | Permanently discontinue lenalidomide. |

Constipation

| | |
|---|---|
| Grade 1-2 | Maintain lenalidomide at full dose |
| Grade ≥3 | If constipation has occurred on or after Day 15, hold |
| | lenalidomide (interrupt dose) for the rest of the cycle |
| | and follow at least every 7 days |
| | If constipation has occurred before Day 15 and resolved |
| | to Grade ≤2 restart lenalidomide at same dose level |
| | for the rest of the cycle |
| | In both cases, restart subsequent cycle at next lower |
| | dose |

Venous thrombosis/embolism

| | |
|---|---|
| Grade ≥3 | Hold lenalidomide (interrupt dose) and start |
| | anticoagulation; restart at Investigator's discretion |
| | (maintain dose level) |

Peripheral neuropathy

| | |
|---|---|
| Grade 2 or 3 | If peripheral neuropathy is considered likely related |
| | to lenalidomide and there is improvement to Grade ≤1 |
| | or baseline, resume lenalidomide at a reduced dose for |
| | current and subsequent cycles. |
| Grade 4 | Permanently discontinue lenalidomide |
| Other lenalidomide-related | If AE has occurred on or after Day 15, hold lenalidomide |
| non-hematologic AEs Grade | (interrupt dose) for the rest of the cycle and follow at |
| ≥3 | least every 7 days. |
| | If AE has occurred before Day 15 and resolved to Grade ≤2, |
| | resume lenalidomide at same dose level for the rest of the |
| | cycle. |
| | In both cases, restart subsequent cycle of lenalidomide at |
| | next lower dose. |

AE = adverse event; NSAID = nonsteroidal anti-inflammatory drug; TLS = tumor lysis syndrome; ULN = upper limit of normal.
^{a} Dose modifications apply only to events that are considered to be related to lenalidomide.
^{b} Graded according to National Cancer Institute Common Terminology Criteria for Adverse Events version 5 (NCI CTCAE Version 5.0; cancer.gov).

### Prohibited Therapies

Use of the therapies including, but not limited to, those listed below was prohibited during the study:
Investigational or unlicensed/unapproved agents
Administration of live vaccines.
Cytotoxic chemotherapy other than study treatments intended for treatment of lymphoma
Radiotherapy for treatment of lymphoma (except pre-planned radiotherapy).
Immunotherapy other than study treatments for treatment of lymphoma
Immunosuppressive therapy (except medications indicated per protocol, including corticosteroids and tocilizumab).
Hormone therapy (other than contraceptives, hormone-replacement therapy, or megestrol acetate).
Adjuvant endocrine therapy for non-metastatic, hormone receptor-positive breast cancer is permitted.
Biologic or targeted agents for treatment of lymphoma
Herbal therapies intended as treatment of lymphoma.
Any therapies intended for the treatment of lymphoma, whether approved by local regulatory authorities or investigational

Patients who required the use of any of these agents were discontinued from study treatment. Patients who were discontinued from study treatment were followed for safety outcomes for 90 days following the patient's last dose of study treatment or until the patient received another anti-cancer therapy, whichever occurred first.

### Safety

Safety was assessed through summaries of adverse events and summaries of changes in laboratory test results. All adverse events, serious adverse events, adverse events leading to death, adverse events of special interest, and adverse events leading to study treatment discontinuation, occurring on or after first study treatment were summarized by mapped terms, appropriate thesaurus levels, and NCI CTCAE v5.0 toxicity grade (cancer.gov). All serious adverse events were listed separately and summarized.

### Adverse Events (AEs)

According to the International Conference on Harmonization (ICH) guideline for Good Clinical Practice (ich.org), an adverse event is any untoward medical occurrence in a clinical investigation subject administered a pharmaceutical product, regardless of causal attribution. An adverse event can therefore be any of the following:
Any unfavorable and unintended sign (including an abnormal laboratory finding), symptom, or disease temporally associated with the use of a medicinal product, whether or not considered related to the medicinal product.
Any new disease or exacerbation of an existing disease (a worsening in the character, frequency, or severity of a known condition).
Recurrence of an intermittent medical condition (e.g., headache) not present at baseline.
Any deterioration in a laboratory value or other clinical test (e.g., electrocardiogram (ECG), X-ray) that is associated with symptoms or leads to a change in study treatment or concomitant treatment or discontinuation from study drug.
Adverse events that are related to a protocol-mandated intervention, including those that occur prior to assignment of study treatment (e.g., screening invasive procedures such as biopsies)

### Serious Adverse Events (SAEs)

A serious adverse event is any adverse event that meets any of the following criteria:
Is fatal (i.e., the adverse event actually causes or leads to death).
Is life-threatening (i.e., the adverse event places the patient at immediate risk of death)

This does not include any adverse event that, had it occurred in a more severe form or was allowed to continue, might have caused death.
Requires or prolongs inpatient hospitalization.
Results in persistent or significant disability/incapacity (i.e., the adverse event results in substantial disruption of the patient's ability to conduct normal life functions)
Is a congenital anomaly/birth defect in a neonate/infant born to a mother exposed to study drug
May jeopardize the patient or may require medical/surgical intervention to prevent one of the outcomes listed above

The terms “severe” and “serious” are not synonymous. Severity refers to the intensity of an adverse event (e.g., rated as mild, moderate, or severe, or according to NCI CTCAE; cancer.gov); the event itself may be of relatively minor medical significance (e.g., severe headache without any further findings).

Severity and seriousness was independently assessed for each adverse event.

### Adverse Events of Special Interest (AESIs)

Adverse events of special interest for this study were as follows:
Cases of potential drug-induced liver injury that include an elevated alanine transaminase (ALT) or aspartate aminotransferase (AST) in combination with either an elevated bilirubin or clinical jaundice, as defined by Hy's Law (an elevated ALT or AST (>3×upper limit of normal (ULN)) in combination with either an elevated total bilirubin (>2×ULN) or clinical jaundice in the absence of cholestasis or other causes of hyperbilirubinemia is considered to be an indicator of severe liver injury).
Suspected transmission of an infectious agent by the study drug, as defined below:

Any organism, virus, or infectious particle (e.g., prion protein transmitting transmissible spongiform encephalopathy), pathogenic or non-pathogenic, was considered an infectious agent. A transmission of an infectious agent may be suspected from clinical symptoms or laboratory findings that indicated an infection in a patient exposed to a medicinal product. This term applied only when contamination of the study drug was suspected.

Adverse events of special interest (AESI) for mosunetuzumab include the following:
Grade ≥2 cytokine release syndrome (CRS)
Grade ≥2 neurologic adverse event
Grade ≥2 injection site reaction (for subcutaneous (SC) mosunetuzumab).
Any suspected Hemophagocytic lymphohistiocytosis (HLH).
Tumor lysis syndrome (TLS; ≥Grade 3 by definition)
Febrile neutropenia (minimum Grade 3 by definition).
Grade ≥2 AST, ALT, or total bilirubin elevation.
Any Grade disseminated intravascular coagulation (minimum Grade 2 by definition).
Grade ≥2 tumor inflammation or flare (e.g., manifestation of signs/symptoms associated with an increase in size of known nodal or extranodal lesions by clinical or radiographic assessment, new onset, or worsening of preexisting pleural effusions).
Any Grade pneumonitis/interstitial lung disease (excluding pneumonia of infectious etiology)

Adverse events of special interest (AESI) for lenalidomide include the following:
Embryo-fetal toxicity
Febrile neutropenia (minimum Grade 3 by definition).
Venous and arterial thromboembolic events
Grade ≥3 severe skin reactions
Grade ≥3 renal impairment
Grade ≥3 thyroid disorder
Grade ≥3 peripheral neuropathy
Second primary malignancies

### Efficacy Analyses

The efficacy of treatment of previously untreated FL with mosunetuzumab and lenalidomide was assessed on the basis of the following endpoints using standard criteria for NHL, assessed by the investigator using the Lugano classification (Cheson B D, et al. J Clin Oncol 2014; 32:1-9). Responses were assessed based on positron emission tomography/computer tomography (PET-CT) scans.

CRR, defined as the proportion of patients whose best overall response was a CR during the study. The exact 95% confidence intervals using the Clopper-Pearson method was provided.

ORR, defined as the proportion of patients whose best overall response was a CR or PR during the study. The exact 95% Cls using the Clopper-Pearson method was provided.

DOR, defined as the time from the first occurrence of a documented objective response to disease progression or relapse or death from any cause, whichever occurred first. The Kaplan-Meier estimate was provided. The Brookmeyer-Crowley method was used to construct the 95% CI for the median DOR. Duration of response included all patients with a CR or PR according to the investigator.

DOCR, defined as the time from the first occurrence of a documented complete response to disease progression or relapse or death from any cause, whichever occurred first. The Kaplan-Meier estimate was provided. The Brookmeyer-Crowley method was used to construct the 95% CI for the median DOR. DOCR included all patients with a CR according to the investigator.

### Pharmacokinetics (PK) Analyses

Individual and mean serum mosunetuzumab concentration versus time data was tabulated and plotted. The plasma PK of mosunetuzumab was summarized by estimating the Cₘₐₓ, Cₘᵢₙ, and AUC, if appropriate. These parameters were tabulated and summarized (mean, standard deviation, coefficient of variation, median, minimum, and maximum).

Additional PK analyses were conducted as appropriate. In addition, these data were analyzed using population PK modeling.

Serum concentrations of lenalidomide were measured. The concentration of lenalidomide was summarized using descriptive statistics as described above.

### Example 3. Results for An Open-Label, Multicenter, Phase I/II Trial Evaluating The Safety, Efficacy, And Pharmacokinetics Of Escalating Doses Of Mosunetuzumab (BTCT4465A) As A Single Agent And Combined With Atezolizumab In Patients With Relapsed Or Refractory B-Cell Non-Hodgkin's Lymphoma And Chronic Lymphocytic Leukemia

The primary objective was to demonstrate PK non-inferiority of subcutaneously administered (SC) mosunetuzumab (mosun) (5/45/45 mg dosing) compared to intravenously administered (IV) mosun (1/2/60/60/30 mg dosing) at RP2D:
n=94 patients with 3 L+R/R FL in pivotal cohort treated with SC mosun monotherapy at F2 dose level (5/45/45 mg Q3W dosing, comprising 21-day dosing cycles, wherein 5 mg, 45 mg, and 45 mg mosunetuzumab was administered on Days 1, 8, and 15, respectively of dosing cycle 1 (C1D1, C1D2, and C1D3 dose, respectively), and 45 mg mosunetuzumab was administered on Day 1 of subsequent dosing cycles (e.g., C2D1+doses)).
Compared to the pivotal cohort for IV mosun (historical control, B11 FL 3 L+FL expansion cohort, Jan. 2, 1960//60/30 mg Q3W dosing, comprising 21-day dosing cycles, n=90; wherein 1 mg, 2 mg, and 60 mg mosunetuzumab was administered on Days 1, 8, and 15, respectively of dosing cycle 1 (C1D1, C1D2, and C1D3 dose, respectively), 60 mg mosunetuzumab was administered on Day 1 of dosing cycle 2 (C2D1), and 30 mg mosunetuzumab was administered on Day 1 of subsequent dosing cycles (e.g., C3D1+)).

The co-primary PK endpoints were met:
Model predicted cumulative AUC₀₋₈₄ Days: geometric mean ratio (GMR) 1.06 (90% CI: 0.92-1.21)
Observed C_{trough} (Cycle 3): GMR 1.39 (90% CI: 1.20-1.61).
Efficacy was largely consistent with mosunetuzumab IV (see Table 12)

TABLE 12
Efficacy of SC Mosunetuzumab vs IV Mosunetuzumab

| | Mosun IV | Mosun SC |
|---|---|---|
| ORR by IRF | 80.0% | 74.5% |
| [95% CI] | [70.25, 87.69] | [64.43, 82.91] |
| CRR by IRF | 60.0% | 58.5% |
| [95% CI] | [49.13, 70.19] | [47.88, 68.59] |
| DOR event-free at | 61.83% | 69.94% |
| 12 mo by IRF | [49.95, 73.71] | [58.52, 81.36] |
| [95% CI] | | |
| PFS at 12 mo by | 57.65% | 62.28% |
| IRF [95% CI] | [46.87, 68.43] | [52.05, 72.52] |

Safety Summary: No unexpected safety findings were observed. Higher frequency of fatal AEs and AEs leading to discontinuation in SC cohorts compared with IV cohorts, which was mainly driven by COVID-19/COVID-19 pneumonia. This was likely attributed to SC cohort enrollment during COVID pandemic whereas IV cohorts were enrolled mostly prior to the pandemic. CRS was generally lower in frequency and severity in SC cohorts compared with IV cohorts. Serious AEs, Grade 3-4 AEs, AESI and selected AEs were less frequent in SC cohorts compared with IV cohorts. Injection site reaction is tolerable and manageable (all low grade, non-serious events and no event leading to treatment discontinuation).

The disease characteristics of the patients are reported below in Table 13.

TABLE 13
Disease characteristics

| | | B11 exp | B11 exp | Group F2 | F2 3L+ |
|---|---|---|---|---|---|
| | | (PR2D) | 3L+ FL | RP2D | FL exp |
| | | (n = 218) | (n = 90) | (n = 139) | (n = 94) |
| ECOG | 0 | 100 | 53 | 83 | 63 |
| | | (45.9%) | (58.9%) | (59.7%) | (67.0%) |
| | 1 | 118 | 37 | 56 | 31 |
| | | (54.1%) | (41.1%) | (40.3%) | (33.0%) |
| Diagnosis at | FL | 90 | 90 | 95 | 94 |
| Study Entry | | (41.3%) | (100%) | (68.3%) | (100%) |
| | DLBCL | 65 | 0 | 31 | 0 |
| | | (29.8%) | | (22.3%) | |
| | trFL | 23 | 0 | 12 | 0 |
| | | (10.6%) | | (8.6%) | |
| | MCL | 25 | 0 | 1 | 0 |
| | | (11.5%) | | (0.7%) | |
| | Richter's | 14 | 0 | 0 | 0 |
| | | (6.4%) | | | |
| | Other | 1 | 0 | 0 | 0 |
| | | (0.5%) | | | |
| Ann Arbor Stage | III | 49 | 25 | 43 | 32 |
| at Study Entry | | (23.0%) | (27.8%) | (30.9%) | (34.0%) |
| (I/II not shown) | IV | 129 | 44 | 72 | 50 |
| | | (60.6%) | (48.9%) | (51.8%) | (53.2%) |
| FLIPI1 at Study | 3 | | 21 | | 30 |
| Entry | | | (23.3%) | | (31.9%) |
| (1 & 2 not | 4 | | 18 | | 14 |
| shown) | | | (20.0%) | | (14.9%) |
| | 5 | | 1 | | 9 |
| | | | (1.1%) | | (9.6%) |

Prior treatment received by the patients is summarized in Table 14.

TABLE 14
Prior Treatments

| | | B11 exp | B11 exp | Group F2 | F2 3L+ |
|---|---|---|---|---|---|
| | | (RP2D) | 3L+ FL | RP2D | FL exp |
| | | N = 218 | N = 90 | n = 139 | n = 94 |
| Prior Lines | Median (range) | 3 | 3 | 3 | 3 |
| of Therapy | | (1-13) | (2-10) | (2-10) | (2-9) |
| | 2 | 33.9% | 37.8% | 44.6% | 46.8% |
| | 3 | 29.8% | 31.1% | 17.3% | 19.1% |
| | >3 | 33.9% | 31.1% | 38.1% | 34.0% |
| Prior Cancer | Prior auto-SCT | | 31.1%* | 15.8% | 20.2% |
| Therapy Regimen | Prior CAR-T | 13.8% | 3.3% | 18% | 4.3% |
| | Prior IMID | 10.6% | 14.4% | 25.2% | 26.6% |
| | Prior PI3K | 10.1% | 18.9% | 10.8% | 11.7% |
| Relapse/Refractory | Refractory to Last | 75.7% | 68.9% | 71.2% | 62.8% |
| Status | Prior Therapy | | | | |
| | Refractory to Any | 80.3% | 78.9% | 74.8% | 67.0% |
| | Prior Anti-CD20 | | | | |
| | Double Refractory | | 53.3% | | 45.7% |
| | to Prior Anti-CD20 | | | | |
| | and Alkylator | | | | |

| | | |
|---|---|---|
| <24 months from start of first | 52.2% | 43.6% |
| systemic therapy to PD | | |

Total exposure to study treatment is reported below in Table 15.

TABLE 15
Total Exposure to Study Treatment

| | B11 | B11 Exp | F2 | F2 Exp |
|---|---|---|---|---|
| | (IV RP2D) | 3L+ FL | (SC RP2D) | 3L+ FL |
| | (N = 218) | (N = 90) | (N = 139) | (N = 94) |
| No. cycles received | | | | |
| (median, range) | 8 | 8 | 8 | 8 |
| | (1-17) | (1-17) | (1-17) | (1-17) |
| N (%) received | | | | |
| <8 cycles | 105 | 21 | 50 | 21 |
| | (48.2%) | (23.3%) | (36.0%) | (22.3%) |
| =8 cycles | 80 | 53 | 66 | 59 |
| | (36.7%) | (58.9%) | (47.5%) | (62.8%) |
| >8 cycles & | 16 | 5 | 15 | 8 |
| <17 cycles | (7.3%) | (5.6%) | (10.8%) | (8.5%) |
| =17 cycles | 17 | 11 | 8 | 6 |
| | (7.8%) | (12.2%) | (5.8%) | (6.4%) |
| Relative dose | 99.4% | 98.7% | 96.8% | 95.6% |
| intensity (median) | | | | |
| % received >90% | 178 | 73 | 116 | 79 |
| dose intensity | (81.7%) | (81.1%) | (83.5%) | (84.0%) |
| Treatment duration, | 148 | 150 | 150 | 152 |
| days | (1-421) | (1-401) | (1-540) | (15-540) |
| (median, range) | | | | |
| Time on study, | 14.3 | 18.3 | 19.3 | 20.7 |
| months | (0.1-27.9) | (2-27.5) | (1-37) | (1-34) |
| (median, range) | | | | |

The primary endpoint for PK, i.e., the non-inferiority of subcutaneously administered mosunetuzumab (5/45/45 mg dosing) compared to intravenously administered mosunetuzumab (1/2/60/60/30 mg dosing), was met. Model predicted cumulative AUC₀₋₈₄ days exhibited a geometric mean ratio (GMR) between SC mosun to IV mosun of 1.06 (0.92-1.21). Observed C_{trough} (Cycle 3) exhibited a GMR between SC mosun to IV mosun of 1.39 (1.20-1.61).

Results of secondary PK endpoints are reported below in Table 16.

TABLE 16
Secondary PK Endpoints

| | IV B11 | SC F2 |
|---|---|---|
| | Exp FL | Exp FL |
| n | 90 | 68 |
| | Model Predicted C_{trough Cycle 2} (μg/mL) | |
| Geometric | 2.07 | 2.50 |
| Mean (% CV) | (124.2) | (66.2) |
| Median | 2.65 | 2.71 |
| (min, max) | (0.0158, 5.08) | (0.140, 7.72) |
| | Model Predicted C_{trough Cycle 3} (μg/mL) | |
| Geometric | 1.53 | 2.37 |
| Mean (% CV) | (105.1) | (49.6) |
| Median | 1.84 | 2.47 |
| (min, max) | (0.0134, 3.52) | (0.280, 6.55) |
| | Model Predicted Steady-State AUC (day*μg/mL) | |
| Geometric | 56.0 | 73.3 |
| Mean (% CV) | (51.6) | (38.0) |
| Median | 61.3 | 76.8 |
| (min, max) | (5.60, 106) | (20.3, 178) |
| | Observed C_{trough Cycle 2} (μg/mL) | |
| n | 54 | 43 |
| Geometric | 2.41 | 2.53 |
| Mean (% CV) | (42.0) | (65.5) |
| Median | 2.39 | 2.80 |
| (min, max) | (0.372, 5.60) | (0.160, 6.78) |

Results for response rates and durability are shown below in Table 17.

TABLE 17
Response Rates and Durability

| | Mosun IV | Mosun SC |
|---|---|---|
| | n = 90 | n = 94 |
| ORR | 80.0% | 74.5% |
| [95% CI] | [70.25, 87.69] | [64.43, 82.91] |
| CRR | 60.0% | 58.5% |
| [95% CI] | [49.13, 70.19] | [47.88, 68.59] |
| DOR event-free | 61.83% | 69.94% |
| at 12 mo. | [49.95, 73.71] | [58.52, 81.36] |
| [95% CI] | | |
| DOCR event-free | 71.42% | 72.35% |
| at 12 mo. | [57.94, 84.90] | [59.87, 84.84] |
| [95% CI] | | |
| PFS at 12 mo. | 57.65% | 62.28% |
| [95% CI] | [46.87, 68.43] | [52.05, 72.52] |

ORR: overall response rate; CRR: complete response rate; DOR: duration of response; DOCR: duration of complete response; PFS: progression free survival.

Best overall response difference between SC mosunetuzumab and IV mosunetuzumab administration exhibited an odds ratio (OR) of 0.73 [0.36, 1.46] (mean [95% CI]), with an adjusted OR (MV) of 0.55 [0.25, 1.19], and an adjusted ratio (IPTW) of 0.59 [0.29, 1.20]. Complete response difference between SC mosunetuzumab and IV mosunetuzumab administration exhibited an odds ratio (OR) of 0.94 [0.52, 1.69] (mean [95% CI]), with an adjusted OR (MV) of 0.81 [0.42, 1.55], and an adjusted ratio (IPTW) of 0.79 [0.44, 1.44].

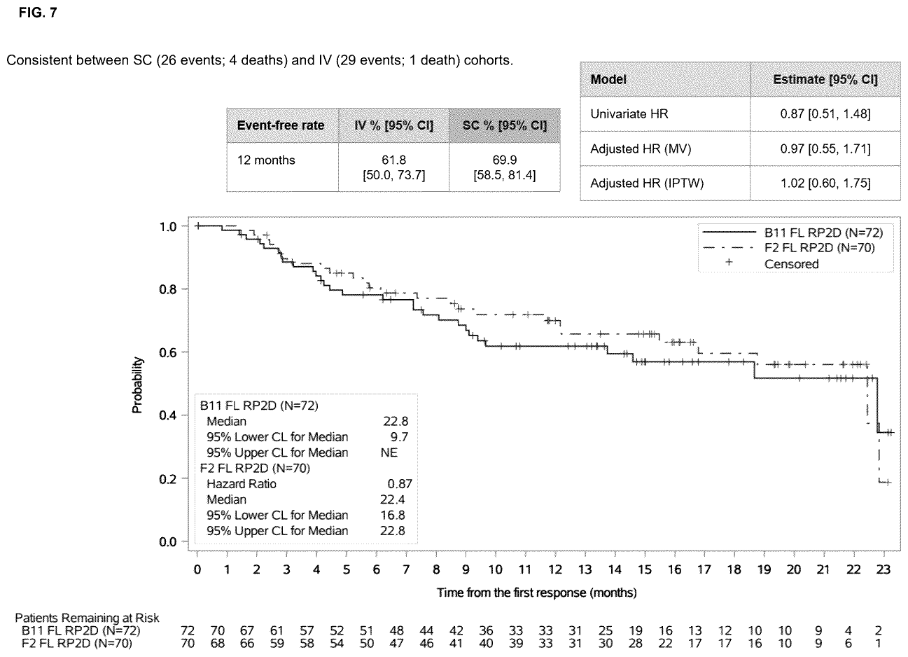

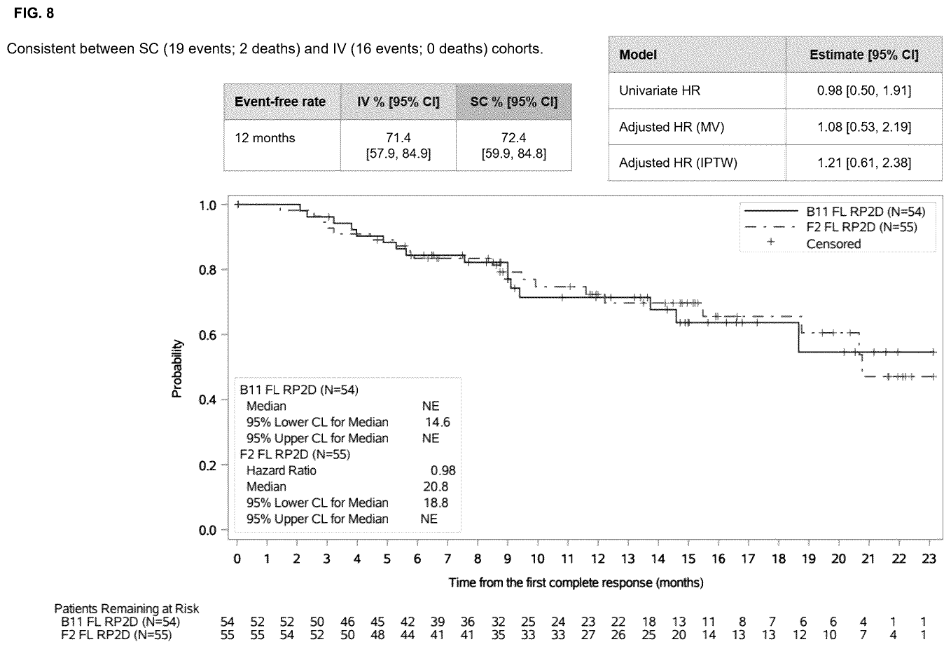

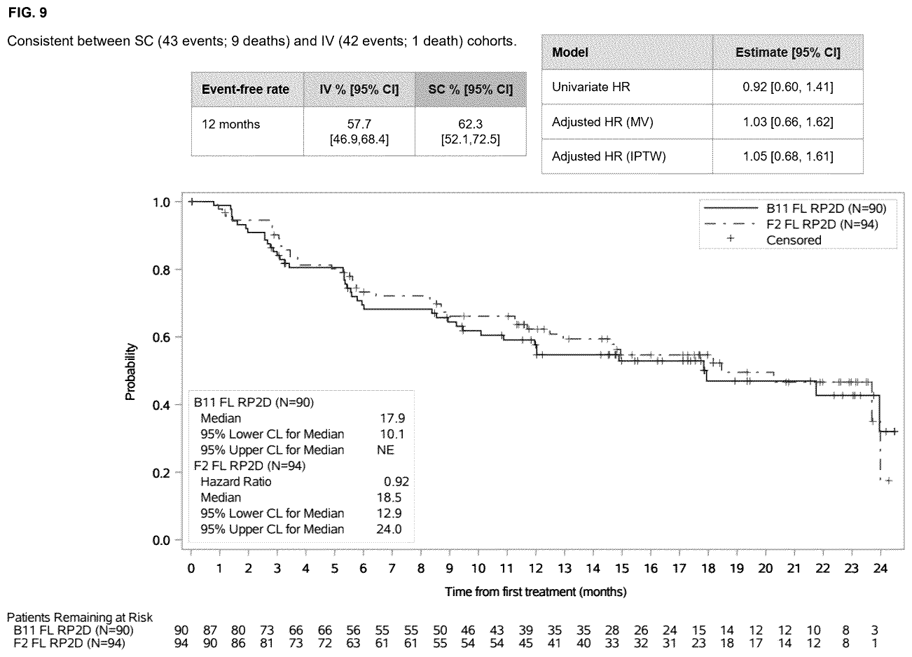

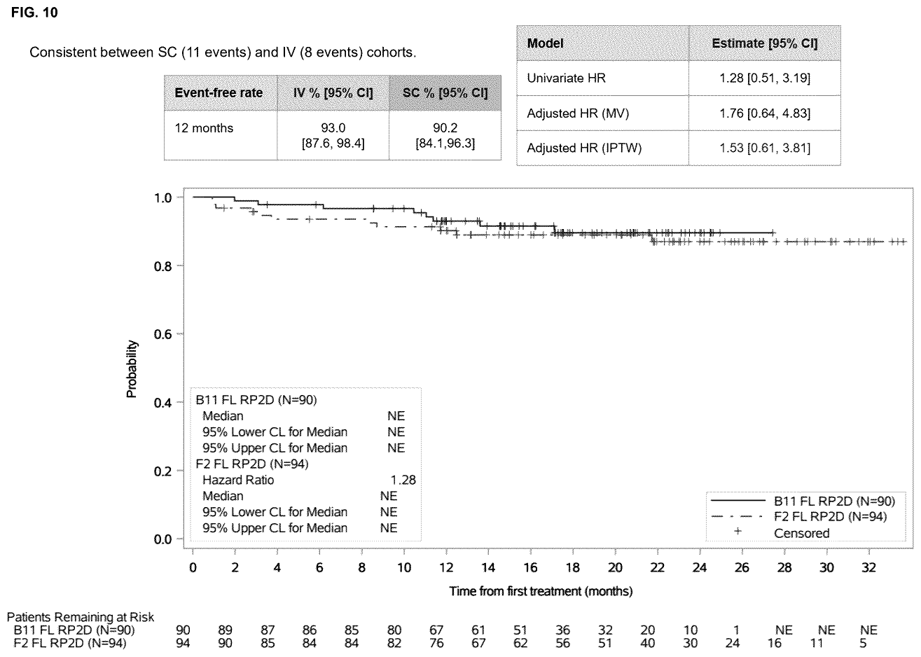

Duration of response results are summarized in FIG. 7. Duration of complete response results are summarized in FIG. 8. Progression free survival results are summarized in FIG. 9. Overall survival results are summarized in FIG. 10.

Tables 18-21 show comparison between mosunetuzumab treatment (SC and IV) with treatment using other therapies in FL patients as a 3^{rd} line or more (3 L+) treatment.

TABLE 18
Mosunetuzumab Compared to Epcoritamab (another anti-
CD3/anti-CD20 bispecific antibody) in 3L+ FL Patients

| | | Mosun SC | | Epcor SC* | |
|---|---|---|---|---|---|
| | Mosun | Minimal/ | Mosun SC | Pivotal | Optimized |
| | IV | Target | Actual profile | Cohort | SUD |
| Efficacy | n = 90 | Profile | n = 94 | (n = 128) | (n = 50) |
| CR | 60% | 55-60% | 58.8% | 63% | |
| ORR | 80% | 75-80% | 74.5% | 82% | |
| mDOR | 22.8 mo | 20-22.8 mo | 22.4 mo | NR | |
| % CRs | 71.4% | 65-71% | 72.35% | 85% | |
| maintained @ | | | | | |
| 12 mo | | | | | |
| PFS: % | 57.7% | 52-57% | 62.28% | ~60% | |
| event-free @ | | | | | |
| 12 mo | | | | | |
| Safety | n = 218 | | n = 139 | | |
| CRS | 39.4% | 40% | 25.9% | | |
| Grade 1 | 22.5% | ≤23% | 17.3% | 66% | 48% |
| Grade 2 | 14.2% | 10-14% | 7.2% | 40% | 40% |
| Grade 3/4 | 2.8% | 1-3% | 1.4% | 25% | 8% |
| Serious CRS | 20.6% | 18-21% | 11.5% | 2% | 0% |
| Neutropenia | | | | | |
| Grade 3/4 | 24.3% | 24-30% | 20.1% | 26% | |
| Febrile Neutropenia | 2.3% | <3% | 1.4% | 3% | |
| ICANS/NAE | | | | | |
| Grade 3/4 | 0% | <3% | 0% | 0% | 0% |
| Injection-site | | | | | |
| reactions | | | | | |
| Grade 2 | N/A | 10-15% | 7.9% | (57% Gr 1-2) | |
| Grade 3/4 | | <2% | 0% | 0% | |
| Discontinuation for | 1.8% | <3% | 3.6% | 4% | |
| related AEs | | | | (1% Covid | |
| | | | | related) | |

*Linton et. al, ASH2023, Poster Abstract #1655 3.

TABLE 19
Summary of Other Chemotherapy-free Options in 3L+ FL

| | Mosun IV | Mosun SC | Zanu + G | | |
|---|---|---|---|---|---|
| | IV Mono × | SC Mono × | Zanu PO to PD | | |
| | 8-17 cycles | 8-17 cycles | G × 6 cycles + | Tazemetostat | |
| | fixed-duration | fixed-duration | 2-yr maintenance | PO to PD | |
| N | 90 | 94 | 145 | 45 | 54 |
| | | | | (EZH2^{mut}) | (EZH2^{wt}) |
| Median age, | 60 | 65 | 63 | 62 | 61 |
| years (range) | (29-90) | (35-84) | (31-84) | | |
| Median prior | 3 | 3 | 3 | 2 | 3 |
| lines of therapy | (2-10) | (2-9) | (2-11) | | |
| (range) | | | | | |
| Refractory to | 68.9% | 62.8% | 32% | 49% | 41% |
| last therapy | | | | | |
| Double- | 53.3% | 45.7% | — | 20% | 28% |
| refractory | | | | | |
| POD24 | 52.2% | 43.6% | 34% | 42% | 59% |
| ORR (%) | 80% | 74.5% | 69 | 69 | 35 |
| CR (%) | 60% | 58.5% | 39 | 13 | 4 |
| Follow-up (mo) | 18.3 | 20.7 | 20.2 | 22.0 | 35.9 |
| mDOR | 22.8 | 22.4 | NR | 10.9 | 13 |
| mPFS | 17.9 | 18.5 | 28 | 13.8 | 11.1 |
| mOS | NR | NR | NR | NR | NR |
| 18 mo | 89.6% | 88.95% | 85.4% | — | — |
| Neutropenia | | | | | |
| Gr3+ | 26.2% | 18% | 24% | 4% | 3% |
| Febrile | 0 | 2.1% | 2% | — | — |
| Neutropenia | | | | | |
| Infections Any/ | 51%/17%/0% | 54.3%/19.2%/3.2% | 55% /—/— | 58%/2%/— | |
| G3+/Gr5 | | | | | |
| Fatal AE | 1.1% | 5.3% | 8% | 0% | 0% |

| | | | | |
|---|---|---|---|---|
| Discontinuation | 4% | 7.4% | 16% | 8% |
| due to AEs | | | | |
| | | | Zinzani | Morschhauser |
| | | | JCO 2023 | Lancet Oncol |
| | | | | 2020 USPI |

TABLE 20
Comparison to CAR-T Therapy for 3L+ FL

| | | | Liso-cel | | |
|---|---|---|---|---|---|
| | | | (TRANSCEND | Tisa-cel | Axi-cel |
| | Mosun IV | Mosun SC | FL) | (ELARA) | (ZUMA-5) |
| N | 90 | 94 | 107 | 97 | 127 |
| | | | (treated set) | (infused) | |
| | | | 101 | 94 | |
| | | | (efficacy set) | (efficacy set) | |
| Median age, | 60 | 65 | 62 | 57 | 60 |
| years (range) | (29-90) | (35-84) | (23-80) | (29-73) | (34-79) |
| Median lines of | 3 | 3 | 3 | 4 | 3 |
| therapy (range) | (2-10) | (2-9) | (2-10) | (2-13) | (1-10) |
| Refractory to | 68.9% | 62.8% | 67% | 78% | 69% |
| last therapy | | | | | |
| Double- | 53.3% | 45.7% | 64% | 68% | 44% |
| refractory | | | | | |
| POD24 | 52.2% | 43.6% | 54% | 63% | 56% |
| ORR | 80% | 74.5% | 97% | 86%^{†} | 94% |
| CR | 60% | 58.5% | 94% | 68%^{†} | 79% |
| | | | (73% per | | |
| | | | USPI) | | |
| Follow-up (mo) | 18.3 | 20.7 | 18.9 | 40.6 | 53.7^{††} |
| mDOR (mo) | 22.8 | 22.4 | NR | NR | 55.5^{††} |
| mPFS (mo) | 17.9 | 18.5 | NR | 37 | 57.3^{††} |
| PFS at 12 mo | 57.7% | 62.3% | 80.7% | 67% | 81.2%^{§} |
| mOS | NR | NR | NR | NR | NR |
| OS at 12 mo | 93.0% | 90.2% | 92.1% | 95% | 96.9%^{§} |
| CRS Any | 44.4% | 29.8% | 59% | 50% | 78%^{§} |
| Gr1 | 25.6% | 20.2% | 42% | — | 72%^{§} |
| Gr2 | 16.7% | 7.4% | 15% | — | |
| Gr3+ | 2.2% | 2.1% | 1% | 1% | 6%^{§} |
| | (1 Grade 3, | | | | |
| | 1 Grade 4) | | | | |
| Neutropenia | | | | | |
| Gr3+ | 26.2% | 18% | 60% | 43% | 60%^{§} |
| Febrile | 0 | 2.1% | 5% | 10.3%^{‡} | 2%^{§} |
| Neutropenia | | | | | |
| NAE***, Any/ | 68.9%/4.4% | 53.2%/1.1% | 31%/10% | (ICANS: 4%/1%^{‡}) | 56%/15%^{§} |
| G3+ | (ICANS: 0%) | (ICANS: 0%) | (ICANS 0%) | | |
| Infections Any/ | 51%/17%/0% | 54.3%/19.2%/3.2% | —/7%/— | 18.6%/5.2%/0%^{‡} | 52%/15%/1%^{§} |
| G3+/Gr5 | | | | | |
| Fatal AE (not | 1.1%* | 5.3% | — | 9% | 1%^{§} |
| incl. PD) | | | | | |
| | | | Morschhauser | Schuster ASH | Neelapu Blood |
| | | | ICML 2023 | 2023 | 2024 |
| | | | Morschhauser | ^{‡}Fowler Nature | ^{†}Neelapu ASH |
| | | | ASH 2023 | 2022 | 2023 |
| | | | | | ^{§}Jacobson |
| | | | | | Lancet 2022 |

*Malignant neoplasm progression (n = 1, onset study D 94) and unexplained death (n = 1, onset study D 60);
^{†}median follow-up 29 mo;
^{§}n = 124, mFU 17.5 mo
**mFU 18.3mo;
***Neurological AEs, unless otherwise specified.

TABLE 21
Comparison to Therapies with FDA Regular Approval for
Patients with R/R FL, Sub-Analysis in 3 L + population

| | | | Rituximab + | Rituximab + | Bendamustine + | |
|---|---|---|---|---|---|---|
| | Mosun | Mosun | Len | Len | Obinutuzumab | Bendamustine |
| | IV | SC | (MAGNIFY) | (AUGMENT) | (GADOLIN) | (GADOLIN) |
| N | 90 | 94 | 115 | 67 | 79 | 94 |
| Prior | 3 | 3 | 3 | 3 | 2 | |
| therapies, | (2-10) | (2-9) | (2-10) | (2-12) | (2-10) | |
| median | | | | | | |
| (range) | | | | | | |
| R- | 78.9% | 67.0% | 47% | 0 | 100% | |
| refractory | | | | | | |
| ORR | 80% | 74.5% | 57% | 81% | 75% | 79% |
| (95% CI) | (70.25, | (64.43, | (48, 66) | (69, 89) | (63, 84) | (69, 86) |
| | 87.69) | 82.91) | | | | |
| CR | 60.0% | 58.5% | 13% | 34% | 20% | 20% |
| | (49.13, | (47.88, | | | | |
| | 70.19) | 68.59) | | | | |
| Median | 22.8 | 22.4 | 5.1 | NR | NR | 13 |
| DOR, | (9.7, | (16.8, | (2.3, 8.5) | (19.6, —) | (19.5, —) | (8.9, 16.8) |
| months | NR) | 22.8) | | | | |
| (95% CI) | | | | | | |

| | | | | | |
|---|---|---|---|---|---|
| Response | Cheson | Cheson | IWG 1999 | Cheson 2007 | Cheson 2007 |
| criteria | 2007 | 2007 | CT/MRI | CT/MRI | CT/MRI |

No unexpected safety findings were observed for mosunetuzumab SC. More frequency of fatal AEs and AEs leading to discontinuation was observed in SC cohorts compared with IV cohorts, which was mainly driven by COVID-19/COVID-19 pneumonia. This was likely attributed to SC cohorts enrollment during COVID pandemic whereas IV cohorts were enrolled mostly prior to the pandemic. CRS was generally less frequent and lower in severity in SC cohorts compared with IV cohorts. Serious AEs, Grade 3-4 AEs, AESI and selected AEs were less frequent in SC cohorts compared with IV cohorts. Injection site reaction from SC mosunetuzumab administration was tolerable and manageable (all low grade, non-serious events and no event leading to treatment discontinuation). Overall safety summary is shown below in Table 22.

TABLE 22
Overall Safety Summary

| Patients with | IV exp | IV exp | F2 | F2 exp |
|---|---|---|---|---|
| at least | (RP2D) | 3L+ FL | RP2D | 3L+ FL |
| one event of: | (N = 218) | (N = 90) | (N = 139) | (N = 94) |
| Patients with at | 214 | 90 | 137 | 93 |
| least one AE | (98.2%) | (100%) | (98.6%) | (98.9%) |
| Related to mosun | 188 | 83 | 124 | 86 |
| | (86.2%) | (92.2%) | (89.2%) | (91.5%) |
| Serious AEs | 114 | 42 | 53 | 37 |
| | (52.3%) | (46.7%) | (38.1%) | (39.4%) |
| Related to mosun | 75 | 30 | 26 | 21 |
| | (34.4%) | (33.3%) | (18.7%) | (22.3%) |
| Grade 3-4 AEs | 145 | 63 | 70 | 46 |
| | (66.5%) | (70.0%) | (50.4%) | (48.9%) |
| Related to mosun | 103 | 46 | 39 | 27 |
| | (47.2%) | (51.1%) | (28.1%) | (28.7%) |
| Grade 5 AEs (not | 4 | 1 | 9 | 5 |
| incl. PD) | (1.8%) | (1.1%) | (6.5%) | (5.3%) |
| Related to mosun | 1 | 0 | 3 | 2 |
| | (0.5%) | | (2.2%) | (2.1%) |
| AE leading to | 9 | 4 | 12 | 7 |
| discon from mosun | (4.1%) | (4.4%) | (8.6%) | (7.4%) |
| Related to mosun | 4 | 2 | 5 | 3 |
| | (1.8%) | (2.2%) | (3.6%) | (3.2%) |

Deaths that occurred in both SC mosunetuzumab and IV mosunetuzumab groups are reported in Table 23.

TABLE 23
Overview of Deaths in Study

| | IV exp | IV exp | F2 | F2 exp |
|---|---|---|---|---|
| | (RP2D) | 3L+ FL | RP2D | 3L+ FL |
| | (N = 218) | (N = 90) | (N = 139) | (N = 94) |
| Total number | 82 | 8 | 41 | 11 |
| of deaths | (37.6%) | (8.9%) | (29.5%) | (11.7%) |
| Cause of deaths | | | | |
| Progressive | 40 | 5 | 20 | 2 |
| disease | (18.3%) | (5.6%) | (14.4%) | (2.1%) |
| Fatal AE PD | 28 | 1 | 7 | 3 |
| | (12.8%) | (1.1%) | (5.0%) | (3.2%) |
| Fatal AE | 4 | 1 | 9 | 5 |
| (excl PD) | (1.8%) | (1.1%) | (6.5%) | (5.3%) |
| excl. COVID | 4 | 1 | 2 | 2 |
| related AEs | (1.8%) | (1.1%) | (1.4%) | (2.1%) |
| Other | 10 | 1 | 5 | 1 |
| | (4.6%) | (1.1%) | (3.6%) | (1.1%) |

PD: progressive disease

Table 24 summarizes AE events that occurred in SC mosun patients with incidence rate of at least 10%.

TABLE 24
AEs with Incidence Rate of at Least 10% in Either F2 Cohort

| | B11 exp | B11 exp | F2 | F2 exp |
|---|---|---|---|---|
| | (RP2D) | 3L+ FL | RP2D | 3L+ FL |
| | n = 218 | n = 90 | N = 139 | n = 94 |
| Patients with at | | | | |
| least one AE: | | | | |
| Injection | — | — | 86 | 57 |
| site reaction | | | (61.9%) | (60.6%) |
| CRS (by ASTCT) | 86 | 40 | 36 | 28 |
| | (39.4%) | (44.4%) | (25.9%) | (29.8%) |
| Fatigue | 70 | 33 | 42 | 33 |
| | (32.1%) | (36.7%) | (30.2%) | (35.1%) |
| Neutropenia | 60 | 26 | 33 | 20 |
| | (27.5%) | (28.9%) | (23.7%) | (21.3%) |
| Headache | 44 | 28 | 24 | 16 |
| | (20.2%) | (31.1%) | (17.3%) | (17.0%) |
| Diarrhea | 38 | 15 | 24 | 19 |
| | (17.4%) | (16.7%) | (17.3%) | (20.2%) |
| COVID-19 | 3 | 3 | 24 | 18 |
| | (1.4%) | (3.3%) | (17.3%) | (19.1%) |
| Insomnia | 23 | 11 | 22 | 14 |
| | (10.6%) | (12.2%) | (15.8%) | (14.9%) |
| Constipation | 36 | 16 | 21 | 13 |
| | (16.5%) | (17.8%) | (15.1%) | (13.8%) |
| Anemia | 33 | 12 | 20 | 12 |
| | (15.1%) | (13.3%) | (14.4%) | (12.8%) |
| Rash | 42 | 14 | 19 | 10 |
| | (19.3%) | (15.6%) | (13.7%) | (10.6%) |
| Pyrexia | 53 | 26 | 18 | 10 |
| | (24.3%) | (28.9%) | (12.9%) | (10.6%) |
| Cough | 33 | 16 | 18 | 11 |
| | (15.1%) | (17.8%) | (12.9%) | (11.7%) |
| Nausea | 38 | 15 | 18 | 13 |
| | (17.4%) | (16.7%) | (12.9%) | (13.8%) |
| Back pain | 17 | 9 | 17 | 11 |
| | (7.8%) | (10.0%) | (12.2%) | (11.7%) |
| Rash maculo- | 9 | 3 | 14 | 10 |
| papular | (4.1%) | (3.3%) | (10.1%) | (10.6%) |
| Chills | 23 | 12 | 13 | 10 |
| | (10.6%) | (13.3%) | (9.4%) | (10.6%) |
| Dry skin | 27 | 14 | 12 | 10 |
| | (12.4%) | (15.6%) | (8.6%) | (10.6%) |
| Arthralgia | 15 | 10 | 12 | 12 |
| | (6.9%) | (11.1%) | (8.6%) | (12.8%) |
| Oedema | 30 | 10 | 10 | 10 |
| peripheral | (13.8%) | (11.1%) | (7.2%) | (10.6%) |

Table 25 summarizes Grade 3-4 AE events that occurred in at least 3 SC mosun patients.

TABLE 25
Grade 3-4 AEs that Occurred in at Least 3 F2 Cohort Patients

| | B11 exp | B11 exp | F2 | F2 exp |
|---|---|---|---|---|
| | (RP2D) | 3L+ FL | RP2D | 3L+ FL |
| | n = 218 | n = 90 | n = 139 | n = 94 |
| Patients with at | | | | |
| least one AE: | | | | |
| Rash maculo-papular | — | — | 3 | 3 |
| | | | (2.2%) | (3.2%) |
| COVID-19 | 2 | 2 | 3 | 2 |
| | (0.9%) | (2.2%) | (2.2%) | (2.1%) |
| COVID-19 pneumonia | — | — | 4 | 2 |
| | | | (2.9%) | (2.1%) |
| Pneumonia | 5 | 3 | 4 | 2 |
| | (2.3%) | (3.3%) | (2.9%) | (2.1%) |
| Neutropenia | 44 | 28 | 28 | 17 |
| | (20.2%) | (31.1%) | (20.1%) | (18.0%) |
| Anemia | 18 | 7 | 10 | 6 |
| | (8.3%) | (7.8%) | (7.2%) | (6.4%) |
| Thrombocytopenia | 15 | 4 | 5 | 4 |
| | (6.9%) | (4.4%) | (3.6%) | (4.3%) |
| Hypophosphataemia | 32 | 15 | 7 | 5 |
| | (14.7%) | (16.7%) | (5.0%) | (5.3%) |
| Hyperglycaemia | 12 | 7 | 4 | 3 |
| | (5.5%) | (7.8%) | (2.9%) | (3.2%) |

Occurrence of cytokine release syndrome (CRS) of different Grades (as determined by ASTCT (American Society for Transplantation and Cellular Therapy)) is summarized in Table 26. Median time to onset from the most recent mosunetuzumab dose in the F2 cohort was 2 days (range: 1-8 days). Median duration of CRS in F2 cohort was 2 days (range: 1-15 days). 100% of CRS events in F2 cohort was resolved.

TABLE 26
CRS Events by ASTCT Grade

| | IV exp | IV exp | F2 | F2 exp |
|---|---|---|---|---|
| | (RP2D) | 3L+ FL | RP2D | 3L+ FL |
| | (N = 218) | (N = 90) | (N = 139) | (N = 94) |
| Any Gr (by | 86 | 40 | 36 | 28 |
| ASTCT) | (39.4%) | (44.4%) | (25.9%) | (29.8%) |
| Gr 1 | 49 | 23 | 24 | 19 |
| | (22.5%) | (25.6%) | (17.3%) | (20.2%) |
| Gr 2 | 31 | 15 | 10 | 7 |
| | (14.2%) | (16.7%) | (7.2%) | (7.4%) |
| Gr 3 | 5 | 1 | 2 | 2 |
| | (2.3%) | (1.1%) | (1.4%) | (2.1%) |
| Gr 4 | 1 | 1 | 0 | 0 |
| | (0.5%) | (1.1%) | | |
| Serious CRS | 45 | 21 | 16 | 14 |
| | (20.6%) | (23.3%) | (11.5%) | (14.9%) |

RP2D: recommended Phase 2 dose.

CRS events were also evaluated based on the timing of their occurrence in the dosing regimen, as shown below in Table 27.

TABLE 27
CRS Events by Dose/Cycle

| | F2 (N = 139) | | | | |
|---|---|---|---|---|---|
| | C1D1-7 | C1D8-14 | C1D15+ | C2 | C3+ |
| | N = 139 | N = 137 | N = 137 | N = 132 | N = 116 |
| Any Grade | 22 | 16 | 3 | 1 | 0 |
| | (15.8%) | (11.7%) | (2.2%) | (0.8%) | |
| 1 | 18 | 10 | 1 | 1 | 0 |
| | (12.9%) | (7.3%) | (0.7%) | (0.8%) | |
| 2 | 4 | 6 | 0 | 0 | 0 |
| | (2.9%) | (4.4%) | | | |
| 3 | 0 | 0 | 2 | 0 | 0 |
| | | | (1.5%) | | |
| 4 | 0 | 0 | 0 | 0 | 0 |
| | F2 exp 3L+ FL (N = 94) | | | | |
| | C1D1-7 | C1D8-14 | C1D15+ | C2 | C3+ |
| | N = 94 | N = 94 | N=94 | N = 91 | N = 88 |
| Any Grade | 18 | 12 | 2 | 0 | 0 |
| | (19.1%) | (12.8%) | (2.1%) | | |
| 1 | 15 | 8 | 0 | 0 | 0 |
| | (16.0%) | (8.5%) | | | |
| 2 | 3 | 4 | 0 | 0 | 0 |
| | (3.2%) | (4.3%) | | | |
| 3 | 0 | 0 | 2 | 0 | 0 |
| | | | (2.1%) | | |
| 4 | 0 | 0 | 0 | 0 | 0 |

96 (69.1%) patients treated with SC mosunetuzumab exhibited an injection site reaction (IRR), with 85 (61.2%) patients being Grade 1, 11 (7.9%) patients being Grade 2, and 0 Grade 3+IRRs. Median time to onset of IRR from the most recent SC mosunetuzumab dose was 2 days (range: 1-8 days). Median duration of IRR was 8 days (range: 1-181 days). 252 out of 253 total IRR events resolved.

### Example 4. A Phase Ib/II, Open-Label, Multicenter Study With A Non-Randomized Stage Evaluating The Safety, Pharmacokinetics, And Efficacy Of Mosunetuzumab (Mosun) Plus Lenalidomide (+Len), And A Randomized Stage Evaluating The Safety, Tolerability, And Pharmacokinetics Of SC Versus IV Mosunetuzumab+Len In Patients With Follicular Lymphoma

For IV mosunetuzumab (mosun)+lenalidomide (len), mosunetuzumab was administered intravenously following a double-step fractionated dose escalation regimen: in Cycle 1 (21-day cycle), patients received 1 mg on Day 1 (C1D1 dose); 2 mg on Day 8 (C1D2 dose); and 30 mg on Day 15 (C1D3 dose). In Cycles 2-12 (28-day cycles), patients received 30 mg on Day 1 (C2D1-C12D1 doses). In Cycle 1, mosunetuzumab was administered via intravenous infusion over 4 hours (+15 min). If no CRS occurs in Cycle 1, infusion time could be shortened to 2 hours (+15 min).

For SC mosunetuzumab+lenalidomide, mosunetuzumab was administered subcutaneously (SC) following a step-up dosing regimen: in Cycle 1 (21-day dosing cycle), patients received 5 mg on Day 1 (C1D1 dose); 45 mg on Day 8 (C1D2 dose); and 45 mg on Day 15 (C1D3 dose). In Cycles 2-12 (28-day dosing cycles), patients received 45 mg on Day 1 (C2D1-C12D1 doses). Hospitalization was not required for patients who received subcutaneous mosunetuzumab unless clinically indicated. Patients treated with SC mosunetuzumab+lenalidomide could choose to receive maintenance therapy, which was administered as 45 mg SC mosunetuzumab every 8 weeks for an additional 9 dosing cycles (8 weeks each). Maintenance therapy began (e.g., dosing cycle 13) 8 weeks after Cycle 12, Day 1.

Lenalidomide was administered orally (PO) 20 mg once daily on Days 1-21 of Cycles 2-12 (28-day cycles). Lenalidomide was not administered on the last 7 days of each of the 28-day Cycles 2-12.

The primary objective was to show pharmacokinetic (PK) non-inferiority (PKNI) of SC (subcutaneous) mosunetuzumab (5/45/45 mg dose) compared to IV (intravenous) mosunetuzumab (1/2/30 mg dose), both in combination with lenalidomide (20 mg dose) based on the co-primary endpoints. Primary objective was met. In particular, the geometric mean ratio (GMR) of the model predicted cumulative AUC_{C1-3} of SC mosunetuzumab vs IV mosunetuzumab was 1.76 (90% CI: 1.55-2.00) and the GME of the Observed Crouch (Cycle 4) was 1.91 (90% CI: 1.54-2.36). Secondary PK results are reported in Table 28.

TABLE 28
Secondary PK Results

| | Arm A: Mosun | Arm B: Mosun |
|---|---|---|
| | IV + Len | SC + Len |
| n | 39 | 78 |
| | Model Predicted Cumulative (AUC_{C1-2})(day*μg/mL) | |
| Geometric | 62.8 | 123 |
| Mean (% CV) | (53.5%) | (45.0%) |
| Median | 72.6 | 133 |
| (min, max) | (12.0, 133) | (24.3, 232) |
| | Model Predicted AUC at steady state during Cycle 4 | |
| | (AUCₛₛ) (day*μg/mL) | |
| Geometric | 50.2 | 68.7 |
| Mean (% CV) | (22.3%) | (28.7%) |
| Median | 49.8 | 68.9 |
| (min, max) | (26.7, 76.2) | (29.1, 119) |
| | Observed Serum trough concentration in Cycle 2 | |
| | (C_{trough, C2}) (μg/mL) | |
| n | 28 | 56 |
| Geometric | 0.53 | 1.39 |
| Mean (% CV) | (96.2%) | (53.2%) |
| Median | 0.61 | 1.47 |
| (min, max) | (0.03, 1.29) | (0.20, 3.58) |

PK of lenalidomide administered in combination with SC or IV mosunetuzumab was also compared. Results are reported in Table 29.

TABLE 29
Lenalidomide PK Results
Cycle 2 Day 1 (first dose of lenalidomide), 2 hours post lenalidomide dose

| | Arm A Mosun | Arm B Mosun |
|---|---|---|
| | IV + Len | SC + Len |
| n/N | 31/32 | 62/63 |
| Mean | 236 | 234 |
| (SD) (ng/ml) | (99.5) | (131) |
| Geometric Mean | 158 | 98.3 |
| (% CV) (ng/mL) | (376.2) | (2436.3) |
| Median | 232 | 243 |
| (Min-Max) (ng/ml) | (0.100-429) | (0.100-549) |

Both mosunetuzumab and lenalidomide PK were comparable between SC Mosun+Len (Arm B) and IV Mosun+Len (Arm A) groups.

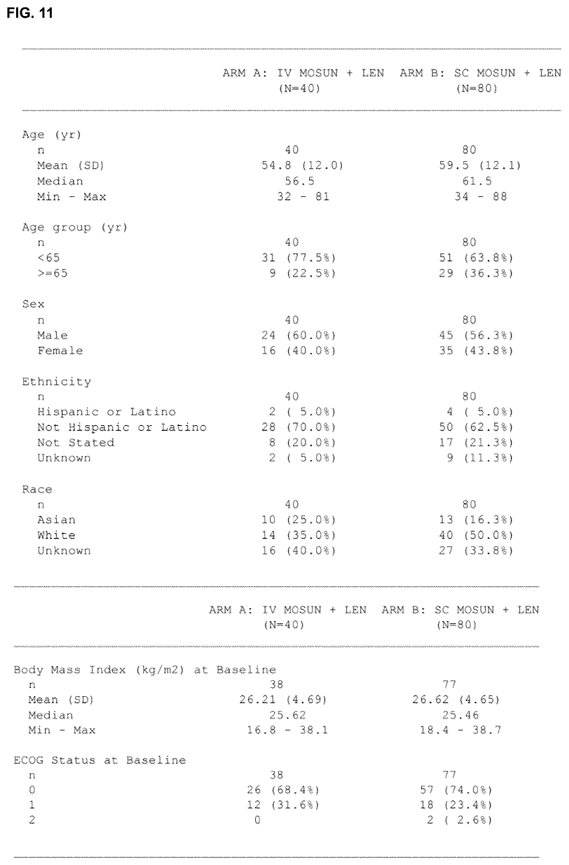

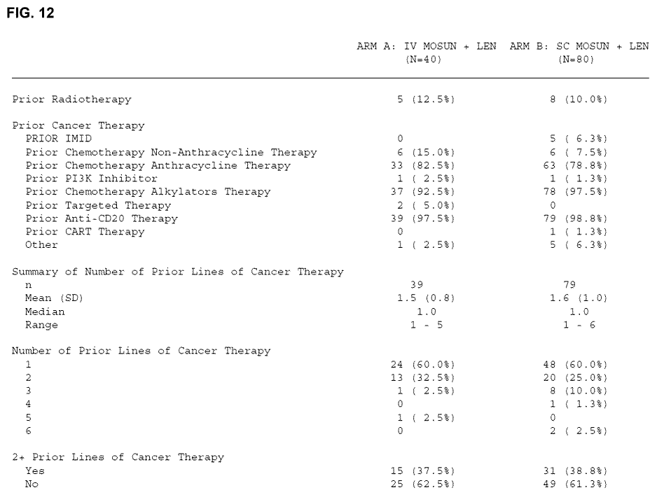

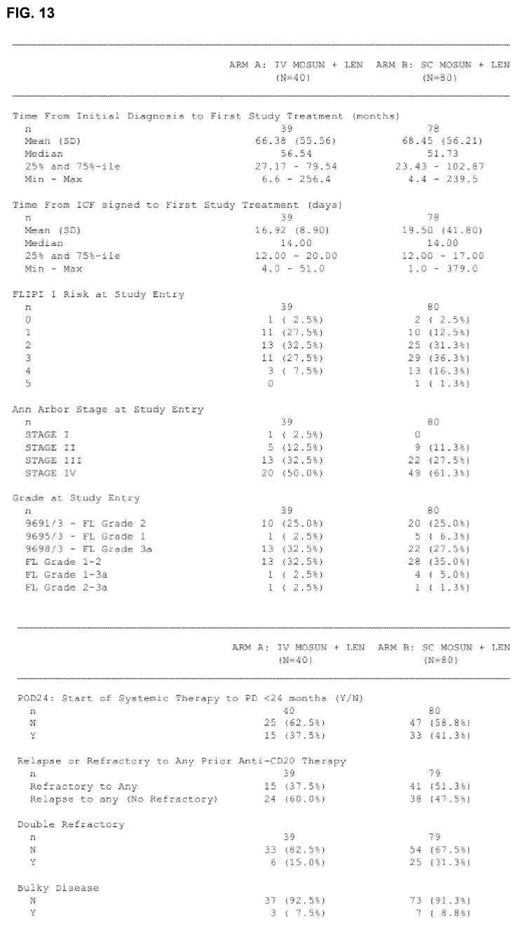

Demographics of patients are shown in FIG. 11. Previous cancer therapy history of the patients is shown in FIG. 12. Baseline disease characteristics of the patients are shown in FIG. 13.

Of the 40 patients treated with IV mosun+len, 32 (80.0%; 95% CI: [66.35%, 93.65%]) exhibited a complete response. Of the 80 patients treated with SC mosun+len, 56 (70.0%; 95% CI: [59.33%, 80.67%]) exhibited a complete response. Between the two groups, the difference in complete response rate was −10.00% (95% CI:-27.37%, 7.37%), corresponding to a p-value of 0.2498 and an odds ratio of 0.59 (95% CI: 0.24, 1.46).

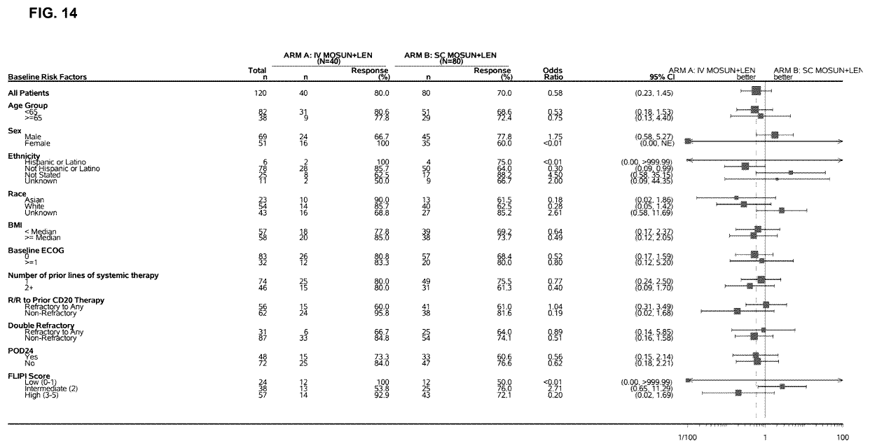

When stratified for only patients who had received 2 L+prior lines of therapy, of the 15 patients treated with IV mosun+len, 12 (80.0%; 95% CI: [56.42%, 100.00%]) exhibited a complete response. Similarly, of the 31 patients treated with SC mosun+len, 19 (61.3%; 95% CI: [42.53%, 80.05%]) exhibited a complete response. Between the two groups, the difference in complete response rate was −18.71% (95% CI:-49.30%, 11.88%), corresponding to a p-value of 0.3167 and an odds ratio of 0.40 (95% CI: 0.09, 1.70). Additional analysis was conducted for various subgroups, and the results are summarized in FIG. 14.

Of the 40 patients treated with IV mosun+len, 37 (92.5%; 95% CI: [79.61%, 98.43%]) exhibited an overall response. Of the 80 patients treated with SC mosun+len, 67 (83.8%; 95% CI: [73.82%, 91.05%]) exhibited an overall response. Between the two groups, the difference in overall response rate was −8.75% (95% CI:-27.37%, 7.37%), corresponding to a p-value of 0.1888 and an odds ratio of 0.42 (95% CI: 0.11, 1.57).

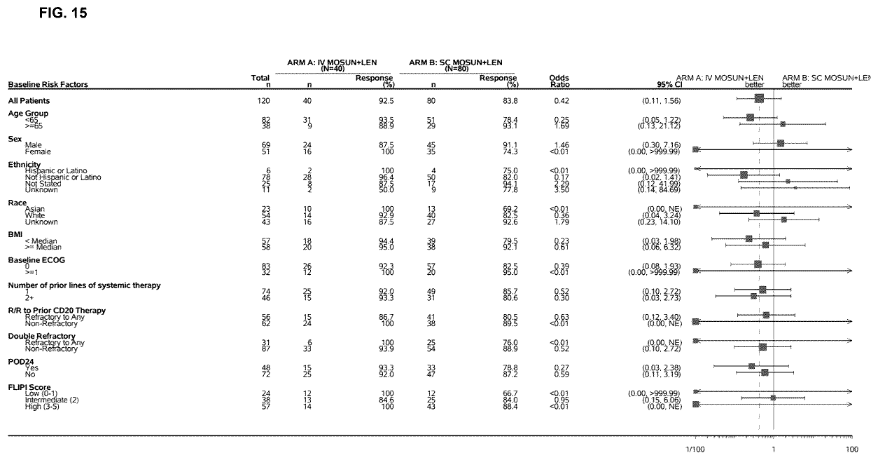

When stratified for only patients who had received 2 L+prior lines of therapy, of the 15 patients treated with IV mosun+len, 14 (93.3%; 95% CI: [68.05%, 99.83%]) exhibited an overall response. Similarly, of the 31 patients treated with SC mosun+len, 25 (80.6%; 95% CI: [62.53%, 92.55%]) exhibited an overall response. Between the two groups, the difference in overall response rate was −12.69% (95% CI: (-35.27%, 9.90%), corresponding to a p-value of 0.3992 and an odds ratio of 0.30 (95% CI: 0.03, 2.73). Additional analysis was conducted for various subgroups, and the results are summarized in FIG. 15.

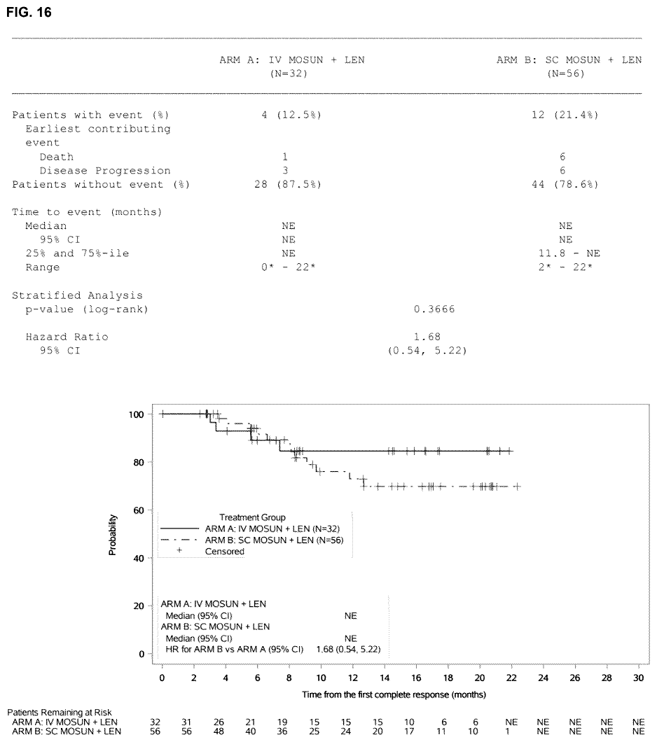

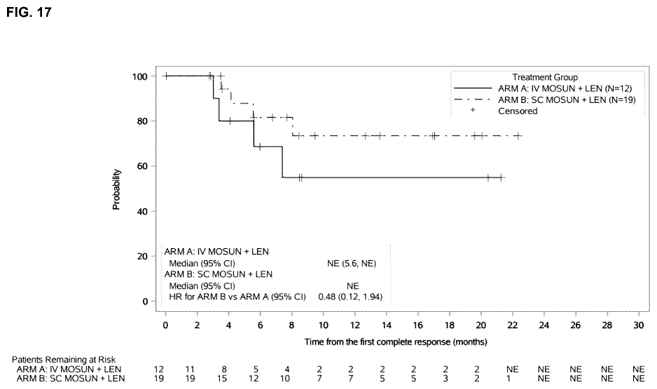

Duration of complete response (DOCR) results and DOCR results stratified for patients treated with 2 L+prior lines of therapy are shown in FIG. 16 and FIG. 17, respectively.

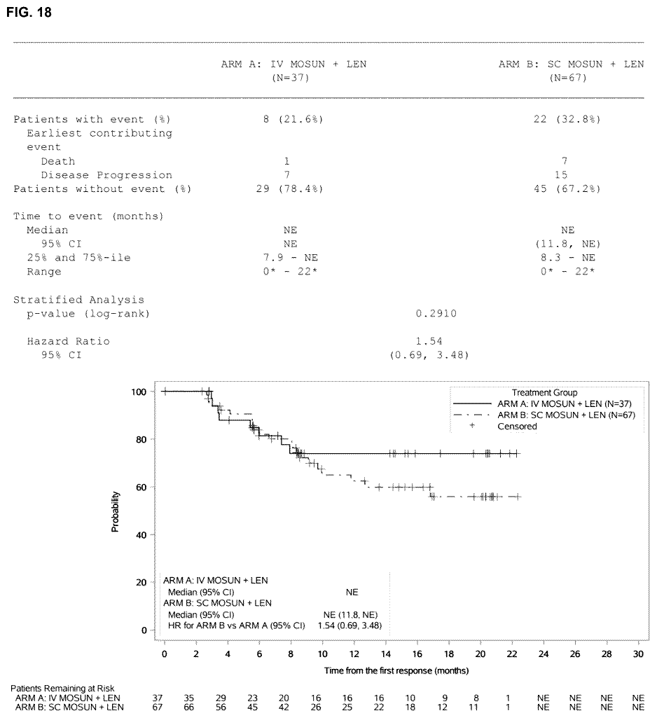

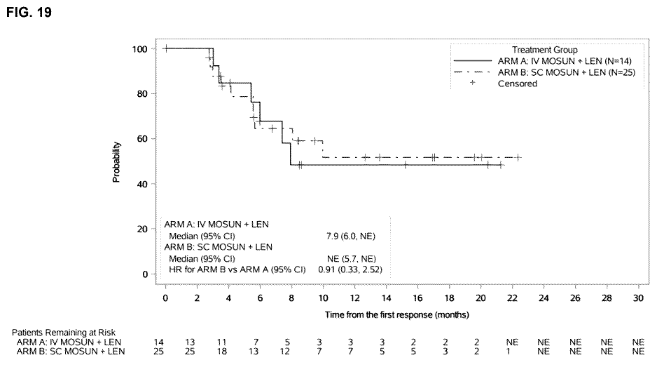

Duration of response (DOR) results and DOR results stratified for patients treated with 2 L+prior lines of therapy are shown in FIG. 18 and FIG. 19, respectively.

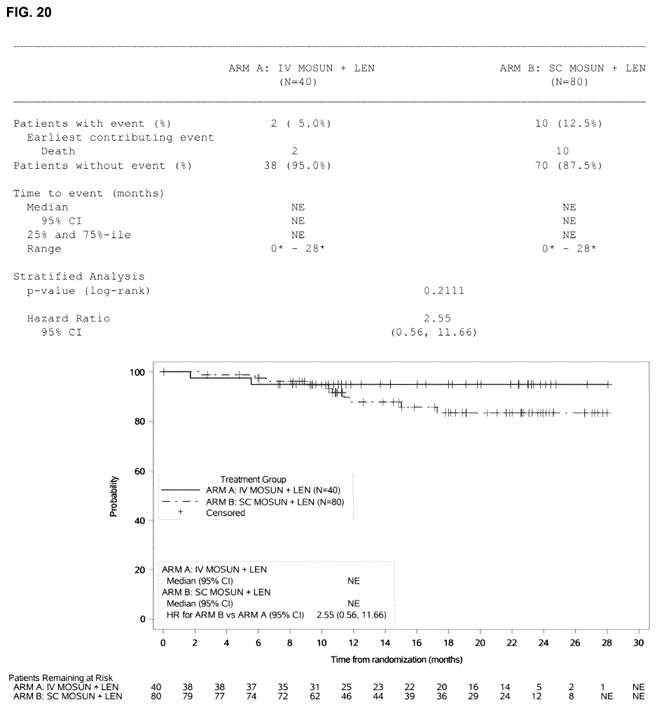

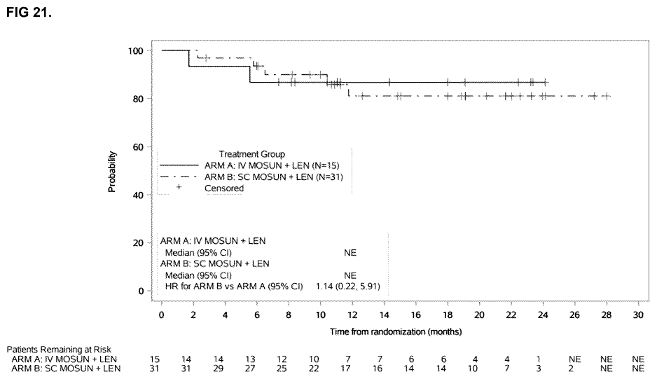

Overall survival (OS) results and OS results stratified for patients treated with 2 L+prior lines of therapy are shown in FIG. 20 and FIG. 21, respectively.

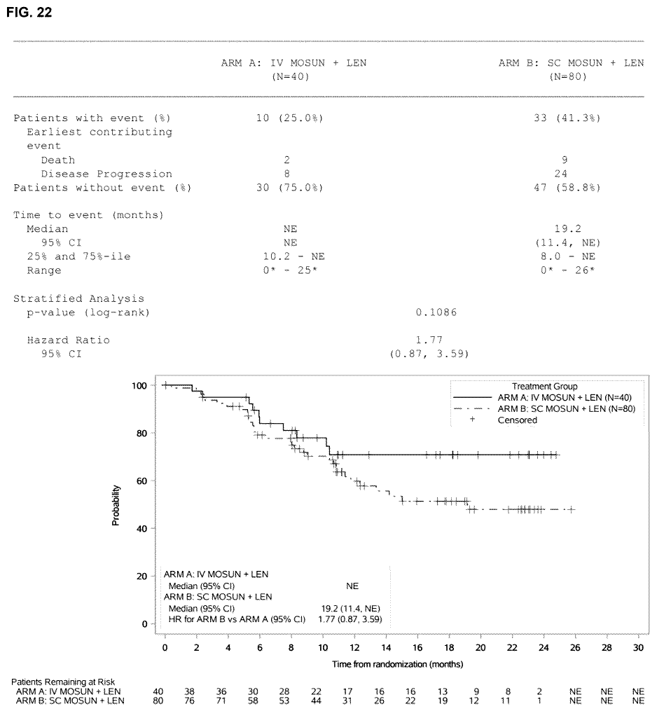

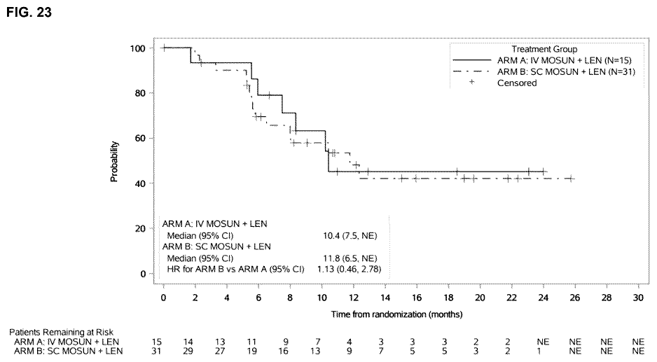

Progression free survival (PFS) results and PFS results stratified for patients treated with 2 L+prior lines of therapy are shown in FIG. 22 and FIG. 23, respectively.

SAE rate, AESI rate, grade 3-4 rate, and overall incidence of AEs were comparable between Arms A (IV mosun+len) and B (SC mosun+len). More deaths were observed due to adverse events in Arm B (SC mosun+len) vs Arm A (IV mosun+len). These deaths were primarily COVID events: 2 Grade 5 COVID AEs in Arm A, 6 in Arm B. Increased rates of neutropenia, febrile neutropenia, and infections were observed in Arm B (SC mosun+len). Reduced CRS rates were observed in Arm B SC mosun+len), CRS duration was comparable between the two Arms, Injection site reactions (i.e., in Arm B) were all Grade 1-2. No new safety concern occurred for SC mosun+len compared to the known safety profile of mosunetuzumab and lenalidomide.

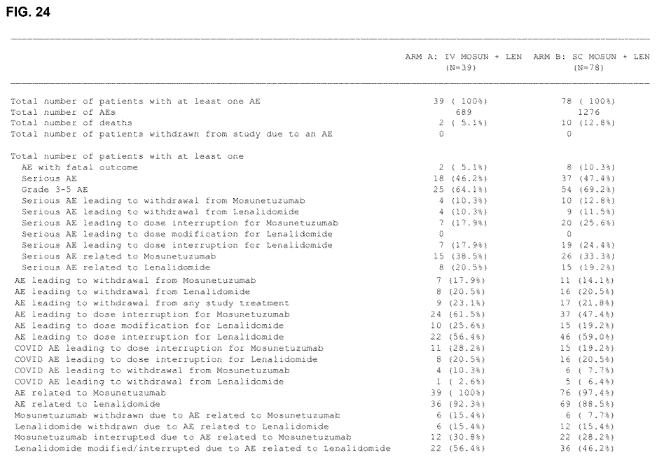

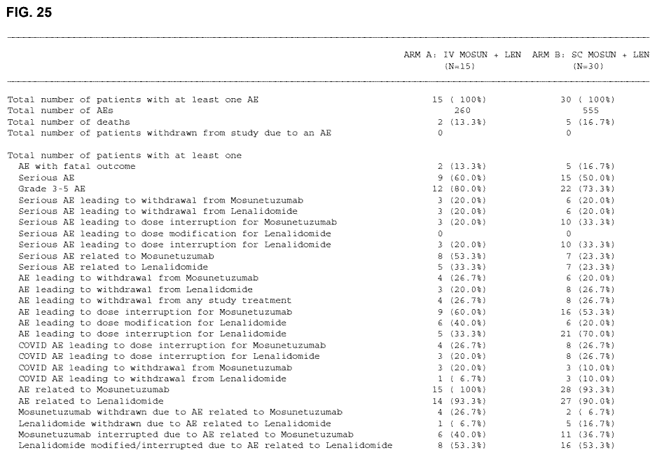

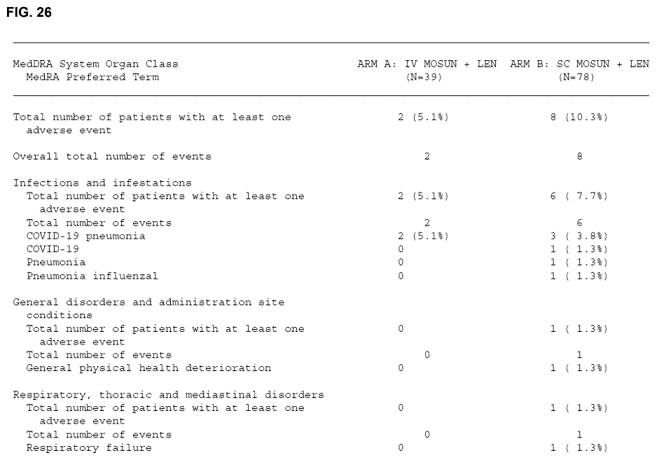

An overall safety summary is shown in FIG. 24. The safety summary for patients who had received two or more prior lines (2 L+) of therapy is shown in FIG. 25. A breakdown of Grade 5 adverse events (death) is shown in FIG. 26.

Of the 78 patients who received SC mosun, 50 (64.1%) exhibited an injection site reaction (ISR). Of these 50 patients, 40 (51.3%) exhibited a Grade 1 ISR and 10 (12.8%) exhibited a Grade 2 ISR. Median time to ISR onset was 3.5 days (range: 1-137 days), and mean time to ISR onset was 13.02+23.31 days. Median duration of ISR was 8 days (range: 1-332 days), and mean duration of ISR was 20.71+39.23 days, based on 126 total ISRs that occurred.

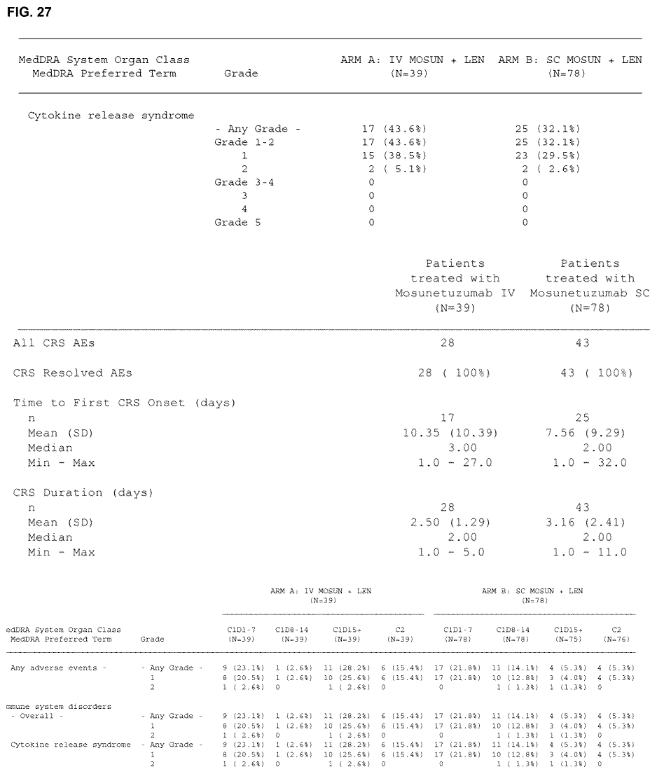

FIG. 27 summarizes CRS events that occurred in Arm A and Arm B, and time to first CRS event and duration of CRS event, as well as a summary of CRS events by dose/cycle. In both IV and SC mosun patients, no Grade 3+ CRS event occurred.

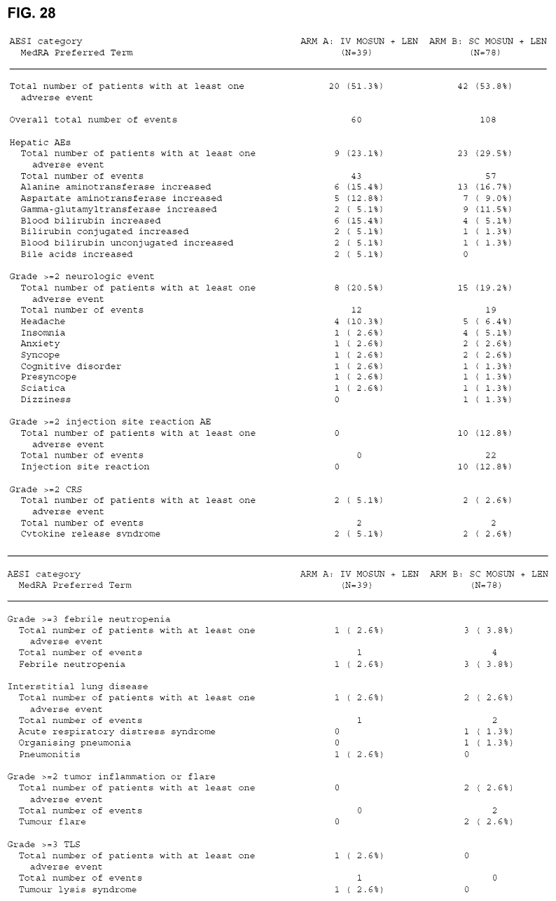

Rates of adverse events of special interest are summarized in FIG. 28.

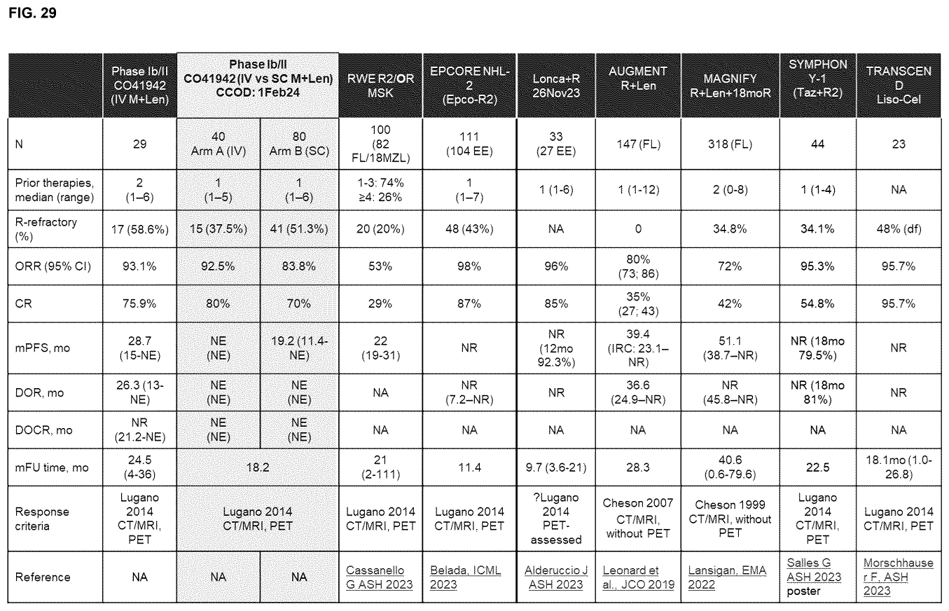

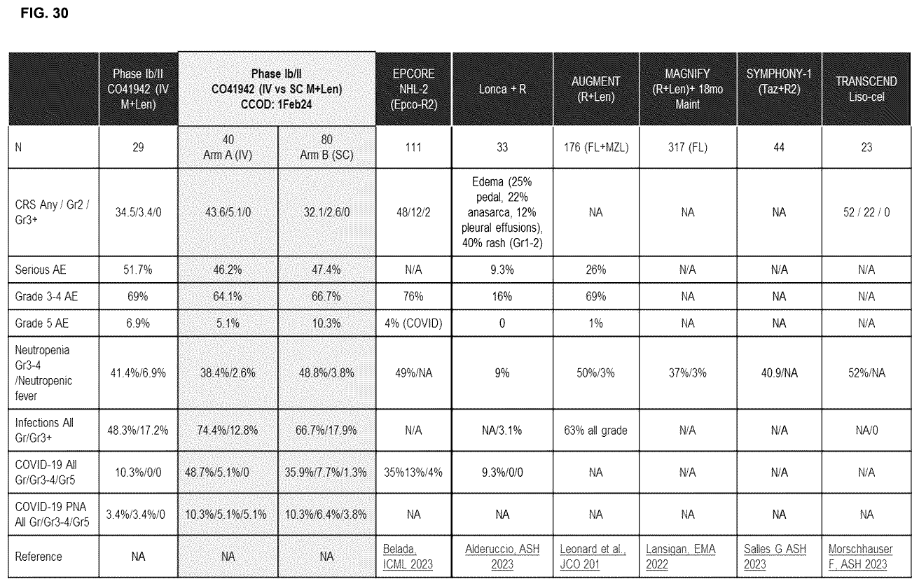

A comparison of the efficacy of IV mosun+len and SC mosun+len with other treatments for patients who had received 2 L+prior lines of therapy is shown in FIG. 29. A comparison of the safety of IV mosun+len and SC mosun+len with other treatments for patients who had received 2 L+prior lines of therapy is shown in FIG. 30.

### Example 5. Fixed-Duration Subcutaneous Mosunetuzumab Demonstrates Clinically Relevant Efficacy in Patients With Relapsed/Refractory Follicular Lymphoma with High-Risk Features: Pivotal Phase II Study Update

Updated analyses are provided from the pivotal Phase II study of subcutaneously administered mosunetuzumab (mosun SC), including in high-risk subgroups of patients (pts) with follicular lymphoma (FL) that had relapsed after or were refractory to three or more prior lines of therapy (pts with 3 L+R/R FL), and adverse events (AEs) of infection.

Methods: Eligible pts had 3 L+R/R FL, Grade (Gr) 1-3a; all pts provided informed consent. Mosun SC was administered as an investigational agent in 21-day cycles with step-up dosing in Cycle (C) 1 (C1 Day 1, 5 mg (C1 dose 1; C1D1); Cycle 1 Day 2 (C1 dose 2; C1D2), 45 mg; Cycle 1 Day 15 (C1 dose 3; C1D3), 45 mg; Cycle 2 Day 1 (C2 dose 1; C2D1) and onwards, 45 mg). Corticosteroid prophylaxis (e.g., dexamethasone 4 mg, 8 mg, 10 mg, 12 mg, or 20 mg orally or IV; 650 mg acetaminophen or paracetamol administered orally; and/or 50 mg diphenhydramine administered orally or IV) was required during C1; hospitalization was not mandatory. Pts with a complete response (CR) by C8 completed treatment (tx); pts with a partial response or stable disease continued tx for up to 17 cycles. Efficacy endpoints (CR rate, duration of CR [DOCR], and progression-free survival [PFS]) were assessed by an Independent Review-Committee (Cheson 2007 criteria). Safety endpoints were also assessed.

Results: In total, 94 pts were enrolled. As of Jul. 24, 2024, median time on study was 26.1 months (mo; range: 1-40). Median age was 65 years (range: 35-84). At baseline, 87% of pts had Ann Arbor stage III/IV disease, 47% were double refractory to prior anti-CD20 and alkylator (e.g., alkylating agent) tx, 44% had progressed within 24 mo from start of first-line tx (POD24), 29% had elevated LDH and 23% had bulky disease (>7 cm).

In the overall population, the CR rate was 62% (Table 30, which includes 95% confidence levels and corresponding confidential intervals for each reported endpoint), and was 61%, 59% and 56% in pts with Ann Arbor stage III/IV, bulky disease, and POD24, respectively. CR rates of 50% and 43% were observed in pts double refractory to prior tx and pts with elevated serum lactate dehydrogenase (LDH), respectively. Overall, the median DOCR was 34.6 mo; median was not reached in pts with elevated LDH, was 22.6 mo in pts with Ann Arbor stage III/IV, 22.6 mo in pts with POD24, 20.8 mo in pts double refractory to prior tx, and 18.8 mo in pts with bulky disease. Overall, the 18-mo PFS rate was 57%, and was 57% in pts with Ann Arbor stage III/IV, and 54% in pts with POD24. For pts with bulky disease, elevated LDH and pts double-refractory to prior tx, 18-mo PFS rates were 51%, 51% and 50%, respectively.

TABLE 30
Efficacy Outcomes in the Overall R/R FL Population and in High-Risk Subgroups

| | | | Bulky | | | |
|---|---|---|---|---|---|---|
| | Overall | Ann Arbor | Disease | | Elevated | Double |
| | Population | Stage III/IV | (>7 cm) | POD24 | LDH | Refractory |
| | N = 94 | N = 82 | N = 22 | N = 41 | N = 28 | N = 44 |
| ORR, n (%) | 72 (76.6) | 63 (76.8) | 16 (72.7) | 30 (73.2) | 17 (60.7) | 66 (77.0) |
| [95% CI] | [66.7-84.7] | [66.2-85.4] | [49.8-89.3] | [57.0-86.0] | [40.6-78.5] | [55.0-83.0] |
| CR, n (%) | 58 (61.7) | 50 (61.0) | 13 (59.1) | 23 (56.1) | 12 (42.9) | 22 (50.0) |
| [95% CI] | [51.1-71.5] | [49.6-71.6] | [36.4-79.3] | [40.0-72.0] | [24.5-62.8] | [35.0-65.0] |
| 18-month | 69.7 | 71.9 | 67.1 | 66.4 | 65.6 | 69.1 |
| DOCR, % | [57.1-82.3] | [58.9-84.9] | [40.7-93.6] | [43.4-89.4] | [51.3-79.8] | [48.3-90.0] |
| [95% CI] | | | | | | |
| Median | 34.6 | 22.6 | 18.8 | 22.6 | NR | 20.8 |
| DOCR, % | [20.7-NE] | [20.7-NE] | [8.48-NE] | [15.5-NE] | [NE-NE] | [15.5-NE] |
| [95% CI] | | | | | | |
| 18-month | 56.8 | 56.7 | 51.0 | 53.8 | 50.8 | 50.1 |
| PFS, % | [46.4-67.2] | [45.5-67.9] | [29.2-72.8] | [38.0-69.6] | [30.8-70.8] | [34.5-65.8] |
| [95% CI] | | | | | | |
| Median | 23.7 | 23.7 | 20.3 | 24.0 | NR | 18.5 |
| PFS, % | [14.6-NE] | [11.3-35.9] | [5.7-NE] | [8.3-35.9] | [3.25-NE] | [5.8-24.0] |
| [95% CI] | | | | | | |

As described in Bartlett et al. (Blood. 142 (Supplement_1): 4397, 2023), most common all-grade adverse events (AEs) were injection-site reactions (ISRs; 61%), fatigue (35%), and cytokine release syndrome (CRS; 30%). ISRs were all Gr 1/2 and non-serious; none led to treatment discontinuation. CRS events (Gr 1/2 28%; Gr 3 2%) were manageable; serious CRS events occurred in 15% of pts. All CRS events occurred in C1, mostly on C1D1 and C1D8; all resolved. Median time to CRS onset was 2.5 days; median duration was 2 days. Of 28 pts with CRS, 8 received tocilizumab and 6 received steroids. Suspected immune effector cell-associated neurotoxicity syndrome occurred in 3 pts (lethargy, n=2; memory impairment, n=1), with 2 events concurrent with CRS. All events were Gr 1, occurred during C1, and resolved without steroid use. Gr 3/4 and serious AEs were reported in 49% and 39% of pts, respectively. Mosun-related AEs led to treatment discontinuation in 3 pts. Gr 5 AEs (excluding PD) were reported in 5 pts (COVID-19 pneumonia, n=2; COVID-19, n=1; hemophagocytic lymphohistiocytosis, n=1 [with active Epstein-Barr virus, cytomegalovirus, and lymphoma transformation]; general physical health deterioration, n=1).

Infections occurred in 54% of pts (Gr 1/2, 35%; Gr 3/4, 16%; Gr 5, 3% [n=2, COVID-19 pneumonia; n=1, COVID-19]). The most common infections presented secondary to viral infections (36%) and included COVID-19 (19%) and COVID-19 pneumonia (7%), followed by infections with an unspecified pathogen (28%) including upper respiratory tract infections (5%). Serious infections occurred in 17% of pts, most commonly COVID-19 pneumonia (4%). B-cell depletion and recovery after mosun SC were similar to those for mosun IV.

Conclusion: Fixed-duration mosun SC achieved clinically relevant efficacy with durable remissions in subgroups of pts with 3 L+R/R FL and high-risk features (POD24 and double-refractory disease). Most infections were low grade; COVID-19 was the most common serious infection.

### Example 6. Morning Sun: Open-Label Phase II Trial of the Efficacy and Safety of Subcutaneous Mosunetuzumab (Mosun SC) in Previously Untreated Symptomatic Patients (Pts) with Marginal Zone Lymphoma (1 L MZL)

Efficacy and safety data are presented for mosun SC monotherapy in pts with 1 L MZL.

Methods: Pts had symptomatic, histologically confirmed MZL (splenic, nodal, and extranodal) and Eastern Cooperative Oncology Group performance status (ECOG PS) of 0-2. Pts were previously untreated, with an indication to start systemic therapy. Mosun SC injection was administered with step-up dosing in Cycle (C) 1 (Day 1, 5 mg; Day 8, 45 mg; Day 15, 45 mg), then 45 mg on Day 1 of each 21-day dosing cycle. Pts were treated for up to 17 cycles unless disease progression or unacceptable toxicity occurred. Hospitalization was not mandatory for monitoring CRS. Primary endpoint was objective response rate (ORR), defined as investigator-assessed complete or partial metabolic response (CMR; PMR; e.g., a CR or PR) by Lugano 2014 Criteria. All pts provided informed consent.

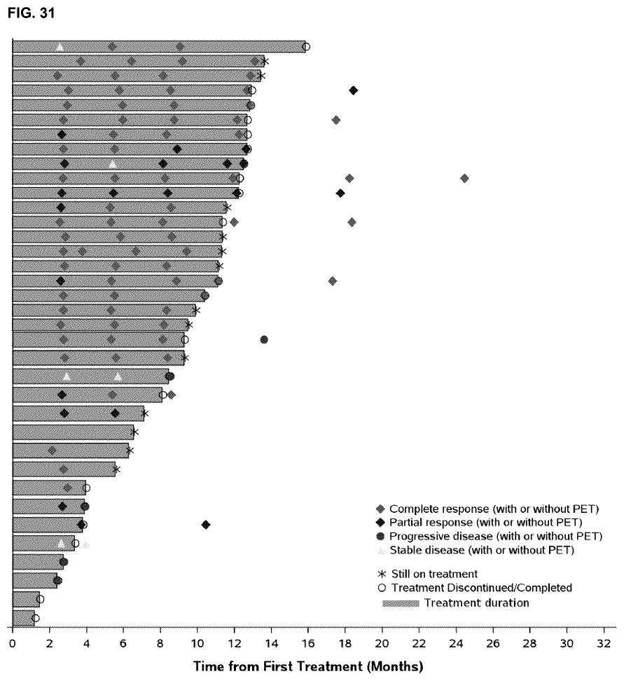

Results: As of May 29, 2024, 36 pts were enrolled in US community (n=23, 64%) and academic (n=13, 36%) centers. Median age was 68.9 years (range: 36-81). Most pts had an ECOG PS of 0 (56%) or 1 (36%); 8% of pts had an ECOG PS of 2. Twenty pts (56%) had extranodal involvement and 6 (17%) had B symptoms. Median duration of follow-up was 11.3 months (95% confidence interval [CI]: 9.7-13.1). Median number of cycles was 14.5 (range: 1-17). The ORR was 75% (95% CI: 58-88; n=27) and CMR was 61% (95% CI: 44-77; n=22). Median time to response was 2.8 months (range: 2.1-5.4). At data cut-off, 13 pts (36%) remained on treatment, 10 (28%) had completed treatment and 13 (36%) had discontinued treatment early (adverse event [AE; n=4, 11%], physician decision [n=3, 8%], progressive disease [n=3, 8%], withdrawal by pt [n=2, 6%] or lost to follow-up [n=1, 3%]; FIG. 31).

Serious AEs occurred in 13 pts (36%); the most common being CRS (8%). Grade (Gr) ≥3 AEs occurred in 22 pts (61%). The most common any-grade AE was injection-site reaction (70%); all events were Gr 1 (58%) or Gr 2 (11%). Other common AEs included fatigue (56%) and headache (33%). CRS by ASTCT was reported in 13 pts (36%); all were low grade (Gr 1, 22%; Gr 2, 14%) and occurred in C1. The median time from last mosun SC dose to first CRS onset was 2 days (range: 1-5); median CRS duration was 2.5 days (range: 1-7). CRS treatment included tocilizumab (n=5), steroids (n=5), tocilizumab+steroids (n=2), and supportive care (fluids, n=5; low-flow oxygen, n=1). All CRS events resolved. Gr 3/4 AEs in ≥5% of pts were neutropenia (39%), anemia (31%), thrombocytopenia (28%), leukopenia (25%) and lymphopenia (8%). Infections occurred in 28 pts (78%; Gr 3-4, 17%), the most common being COVID-19 (22%; Gr 3, 3%). No immune effector cell-associated neurotoxicity syndrome events or fatal AEs were reported.

Conclusions: In this study investigating Mosun SC with a dedicated cohort for pts with 1 L MZL, clinically meaningful efficacy was reported with an ORR of 75% and CMR of 61%. A manageable safety profile was observed, demonstrating that Mosun SC can be safely administered in community practices and outpatient settings.

### Example 7. Fixed Duration Subcutaneous (SC) Mosunetuzumab (Mosun) in Patients with Previously Untreated High-Tumor Burden Follicular Lymphoma (FL): Interim Results from the Phase II Morning Sun Study

Efficacy and safety results of Mosun SC in patients with previously untreated high-tumor burden FL in the Phase II Morning Sun study (NCT05207670) are reported.

Methods: Patients with previously untreated high-tumor burden FL, per Groupe d'Etude des Lymphomes Folliculaires (GELF) criteria, were enrolled into this cohort. Mosun SC was administered with step-up dosing in Cycle (C) 1 (Day 1, 5 mg; Day 8, 45 mg; Day 15, 45 mg) then 45 mg on Day 1 for up to 17 cycles (1 year; 21-day cycles). Patients with a partial (PMR; e.g., a partial response) or complete metabolic response (CMR; e.g., a complete response) after C17 could receive additional Mosun maintenance therapy (45 mg every 8 weeks for up to 1 year). Corticosteroid prophylaxis to reduce the risk of cytokine release syndrome (CRS) was mandatory in C1-2 and optional thereafter. The primary endpoint was progression-free survival (PFS) rate at 24 months. Key secondary endpoints included objective response rate (ORR), time to response (TTR), and safety.

Results: As of May 29, 2024, 102 patients were enrolled; 55 patients had completed initial treatment (17 cycles), 31 had discontinued (most commonly due to progressive disease [n=19] and adverse events [AEs; n=4]), and 16 were ongoing initial treatment. Forty-two patients received maintenance treatment, and this was ongoing in 38 patients. Median age was 65 years (range: 24-86); 52.0% of patients were female. Most patients had Ann Arbor stage III/IV (91.2%) and a FLIPI score ≥2 (78.4%). Median duration of follow-up was 13.9 months.

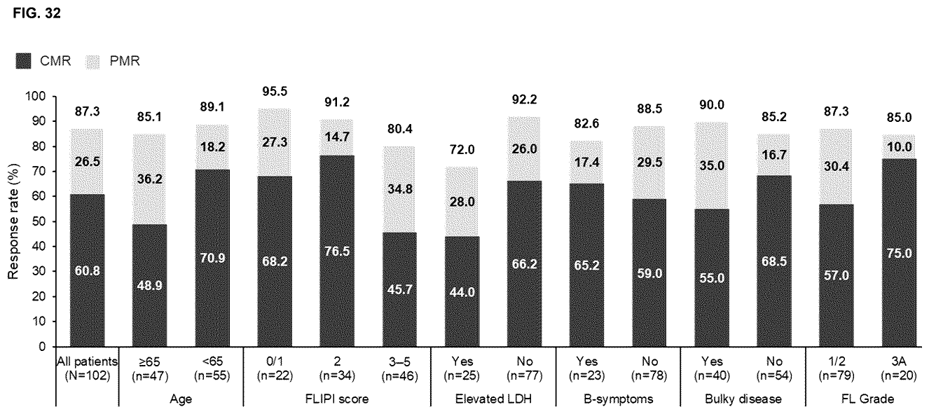

The 12-month PFS rate was 82.8% (95% confidence interval [CI]: 73.0-89.3). ORR was 87.3%; 60.8% of patients had a CMR. Subgroup analysis for these patients (divided by age (≥65 years or <65 years); FLIPI score (0 or 1, 2, or 3-5); elevated LDH (yes or no); B symptoms (yes or no); bulky disease (yes or no), or FL Grade (1-2 or 3a)) is shown in FIG. 32. Among the 89 patients with a response, the median TTR was 2.7 months (range: 1.2-5.8).

The most common AEs (>30%) were injection-site reaction (64.7%), fatigue (42.2%), CRS (34.3%), headache (31.4%), and nausea (30.4%). Grade ≥3 AEs and serious AEs (SAEs) were reported in 44.1% and 29.4% of patients, respectively. CRS events were all Grade 1/2 and SAE CRS occurred in 10.8% of patients (Table 31); all events resolved.

TABLE 31
Summary of CRS Safety Events

| | | Among Patients Who |
|---|---|---|
| | | Experienced at Least One |
| | Patients | CRS Event of Any Grade |
| CRS events, n (%) | N = 102 | N = 35 |
| Any grade | 35 | 35 |
| | (34.3) | (100%) |
| Grade | | |
| 1 | 30 | 30 |
| | (29.4) | (85.7) |
| 2 | 5 | 5 |
| | (4.9) | (14.3) |
| SAE | 11 | 11 |
| | (10.8) | (31.4) |
| Management | | |
| Steroids | 8 | 8 |
| | (7.8) | (22.9) |
| Tocilizumab | 6 | 6 |
| | (5.9) | (17.1) |
| Steroids + | 3 | 3 |
| tocilizumab | (2.9) | (8.6) |
| Fluids | 3 | 3 |
| | (2.9) | (8.6) |
| ICU admission | 2 | 2 |
| | (2.0) | (5.7) |

Conclusions: Mosun SC demonstrated promising efficacy in patients with previously untreated high-tumor burden FL. The manageable safety profile, including rate of CRS, supports the administration of fixed duration Mosun SC in an outpatient setting.

### Example 8. Safety and Efficacy of Mosunetuzumab Subcutaneous Maintenance Following Mosunetuzumab Plus Lenalidomide (M+Len) Induction Therapy in Previously Untreated (1 L) Follicular Lymphoma (FL)

Updated data are presented for M+Len induction and initial efficacy and safety of subsequent mosunetuzumab (M) subcutaneous (SC) maintenance.

Methods: Pts with 1 L FL requiring treatment per GELF criteria and an ECOG PS of 0-2 were included. Pts received 12 cycles (Cycle [C] 1, 21 days; C2-12, 28 days) of M+Len (M SC: C1 Day [D] 1, 5 mg; C1D8, C1D15, and C2-12 D1, 45 mg; lenalidomide (Len) [oral]: D1-21 of C2-12, 20 mg). Responders could receive optional M SC (45 mg) monotherapy maintenance every 8 weeks for 9 cycles (C13-21). Anti-infective prophylaxis followed institutional standards. The primary objective was to evaluate the efficacy of M+Len induction followed by M SC maintenance.

Results: As of Sep. 23, 2024, 40 pts received induction therapy and 16 pts proceeded to M maintenance. Median age was 62 years (range: 28-83), 55% were male and 45% had a FLIPI score of 3/4. Median number of M doses during induction and maintenance was 19 (range: 16-21).

The overall response rate (intent-to-treat population, N=40) was 90% and complete response (as best response) rate was 88%; 4 pts had stable or progressive disease (PD). After a median follow-up of 18 months (range: 6-23), median duration of response (DOR), duration of complete response (DOCR), progression-free survival (PFS) and overall survival (OS) were not reached. The 12-month event-free rates for DOR, DOCR, PFS and OS were 94% (95% CI: 86-100), 94% (95% CI: 86-100), 87% (95% CI: 76-98), and 97% (95% CI: 92-100), respectively. Sustained responses were observed in pts who received maintenance (no pts had PD post-induction), and in pts who were eligible but did not receive maintenance and were observed post-induction (one pt had PD post-induction).

During induction (n=40), all pts reported ≥1 adverse event (AE; serious AEs [SAE], 43%; Grade [Gr] 3/4, 70%). The most common AEs were injection site reactions (ISRs; 68%; all Gr 1/2), neutropenia (63%; Gr 3/4, 50%), and cytokine release syndrome (CRS; 53%; all Gr 1/2). Gr 3/4 infections occurred in 13% of pts.

During maintenance (n=16), 69% of pts reported AEs (SAEs, 13%; Gr 3/4, 25%). ISRs occurred in 13% of pts (all Gr 1/2) and neutropenia in 25% (Gr 3/4, 19%); no CRS or Gr 3/4 infections occurred. One pt discontinued maintenance due to an AE.

No pts experienced Gr 5 AEs, febrile neutropenia, or ICANS during induction or maintenance.

Conclusions: M+Len induction with M maintenance showed durable responses and manageable safety, with low rates of AEs during maintenance in pts with high tumor burden 1 L FL. These results highlight the promise of this chemotherapy-free regimen, although further follow-up is needed to confirm these findings.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab and orally administered lenalidomide, and wherein the efficacy response is a complete response rate (CRR), an objective response rate (ORR), a duration of response (DOR), a duration of complete response (DOCR), an overall survival (OS), or a progression free survival (PFS).

2. The method of claim 1, wherein:
(a) the efficacy response is a CRR, wherein the CRR or reference CRR is the proportion of the subjects receiving the corresponding treatment whose best overall response is a complete response (CR), and wherein:
(i) the improved response in CRR is an increase in CRR compared to the reference CRR of between 1% and 25%; or
(ii) the efficacy response in CRR is non-inferior compared to the reference CRR;
(b) the efficacy response is an ORR, wherein the ORR or reference ORR is the proportion of the subjects receiving the corresponding treatment whose best overall response is a CR or a partial response (PR), and wherein:
(i) the improved response in ORR is an increase in ORR compared to the reference ORR of between 1% and 24%; or
(ii) the efficacy response in ORR is non-inferior compared to the reference ORR;
(c) the efficacy response is a DOR, wherein the DOR or the reference DOR is measured starting from the time from the first occurrence of a documented CR or PR to disease progression or relapse or death from any cause, whichever occurs first, wherein the DOR or the reference DOR is the median DOR of the plurality of subjects receiving the corresponding treatment, and wherein:
(i) the improvement of the median DOR is an increase in the DOR compared to the reference DOR of between 1 and 22 months; or
(ii) the efficacy response in DOR is non-inferior compared to the reference DOR;
(d) the efficacy response is a DOCR, wherein the DOCR or the reference DOCR is measured starting from the time from the first occurrence of a documented CR to disease progression or relapse or death from any cause, whichever occurs first, wherein the DOCR or the reference DOCR is the median DOCR of the plurality of subjects receiving the corresponding treatment, and wherein:
(i) the improvement of the median DOCR is an increase in the DOCR compared to the reference DOCR of between 1 and 22 months; or
(ii) the efficacy response in DOCR is non-inferior compared to the reference DOCR;
(e) the efficacy response is an OS, wherein the OS or the reference OS is measured starting from the time from first treatment to death from any cause, wherein the OS or the reference OS is the median OS of the plurality of subjects receiving the corresponding treatment, and wherein:
(i) the improvement of the median OS is an increase in the OS compared to the reference OS of between 1 and 28 months; or
(ii) the efficacy response in OS is non-inferior compared to the reference OS; or
(f) the efficacy response is a PFS, wherein the PFS or the reference PFS is measured starting from the time from first treatment to the time of a first occurrence of disease progression or death from any cause, wherein the PFS or the reference PFS is the median PFS of the plurality of subjects receiving the corresponding treatment, and wherein:
(i) the improvement of the median PFS is an increase in the PFS compared to the reference PFS of between 1 and 26 months; or
(ii) the efficacy response in PFS is non-inferior compared to the reference PFS.

3.-15. (canceled)

16. The method of claim 2, wherein the CR or PR is determined by PET/computed tomography (CT); and/or the CR or PR is determined based on the Lugano Response Criteria for Malignant Lymphoma (Cheson et al., 2014).

17-25. (canceled)

26. A method for treating follicular lymphoma (FL) in a subject in need thereof, comprising administering to the subject a combination treatment comprising subcutaneously administered mosunetuzumab and orally administered lenalidomide, wherein administering the combination treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab and orally administered lenalidomide, and wherein the safety response is the rate of adverse event (AE), the rate of cytokine release syndrome (CRS), or the rate of serious AE (SAE).

27. The method of claim 26, wherein;
(a) the safety response is the rate of AE, wherein the rate of AE or the reference rate of AE is the rate of AE in the plurality of subjects receiving the corresponding treatment, and wherein the safety response in rate of AE is non-inferior compared to the reference rate of AE;
(b) the safety response is the rate of CRS, wherein the rate of CRS or the reference rate of CRS is the rate of CRS in the plurality of subjects receiving the corresponding treatment, and wherein:
(i) the rate of CRS is the rate of Grade 1 or 2 CRS in the plurality of patients receiving the corresponding treatment, and wherein the improved rate of CRS is a decrease in the rate of CRS compared to the reference rate of CRS of between 7% and 17%; or of about 12%;
(ii) the rate of Grade 3+ CRS is non-inferior compared to the reference rate of Grade 3+ CRS; and/or
(iii) the Grade of CRS is determined based on the American Society of Transplantation and Cellular Therapy (ASTCT) Consensus Grading for Cytokine-Release Syndrome (Lee et al., 2019); or
(c) the safety response is the rate of SAE, wherein the rate of SAE or the reference rate of SAE is the rate of SAE in the plurality of subjects receiving the corresponding treatment, and wherein the safety response in rate of SAE is non-inferior compared to the reference rate of SAE.

28.-36. (canceled)

37. A method for treating follicular lymphoma (FL) in a subject in need thereof, comprising administering to the subject a combination treatment comprising subcutaneously administered mosunetuzumab and orally administered lenalidomide according to a dosing regimen comprising a first 21-day dosing cycle and three 28-day dosing cycles, wherein administering the combination treatment to a plurality of subjects results in a non-inferior pharmacokinetic (PK) endpoint of the plurality of subjects as compared to a reference PK endpoint, wherein the PK endpoint is an observed dosing cycle 2 serum trough concentration (scConc_{troughCYC2-OBS}) or a cumulative area under the concentration-time curve over 0-77 days (scAUC₀₋₇₇); and
wherein the reference PK endpoint is the PK endpoint of a plurality of subjects who have received a control treatment comprising intravenously administered mosunetuzumab and orally administered lenalidomide according to a dosing regimen comprising a first 21-day dosing cycle and three 28-day dosing cycles, wherein the reference PK endpoint is an observed dosing cycle 2 serum trough concentration (ivConc_{troughCYC2-OBS}) or a cumulative area under the concentration-time curve over 0-77 days (ivAUC₀₋₇₇).

38. The method of claim 37, wherein one or both of the scAUC₀₋₇₇ and the ivAUC₀₋₇₇ is determined using population PK (popPK) model-predicted individual Empirical Bayesian Estimates (EBEs).

39. The method of claim 1, wherein;
(I) the combination treatment comprises subcutaneously administering mosunetuzumab and orally administering lenalidomide according to a dosing regimen comprising at least a first dosing cycle and a second dosing cycle, wherein the first dosing cycle is a 21-day dosing cycle and the second dosing cycle is a 28-day dosing cycle, and wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg; and
(b) the second dosing cycle comprises:
(i) a first subcutaneous dose (scC2D1) of mosunetuzumab administered on Day 1 of the second dosing cycle, wherein the scC2D1 is about 45 mg; and
(ii) twenty-one (21) oral doses of lenalidomide administered on Days 1-21 of the second dosing cycle, wherein each oral dose of lenalidomide is about 20 mg; and/or
(II) the control treatment comprises intravenously administering mosunetuzumab and orally administering lenalidomide according to a dosing regimen comprising at least a first 21-day dosing cycle and a second 28-day dosing cycle, wherein:
(a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 30 mg; and
(b) the second dosing cycle comprises:
(i) a first intravenous dose (ivC2D1) of mosunetuzumab administered on Day 1 of the second dosing cycle, wherein the ivC2D1 is about 30 mg; and
(ii) 21 oral doses of lenalidomide administered on Days 1-21 of the second dosing cycle, wherein each oral dose of lenalidomide is about 20 mg.

40. The method of claim 39, wherein;
(a) the dosing regimen of the combination treatment further comprises one or more additional 28-day dosing cycles or ten additional 28-day dosing cycles; and/or
(b) the dosing regimen of the control treatment further comprises one or more additional 28-day dosing cycles or ten additional 28-day dosing cycles.

41. (canceled)

42. The method of claim 40, wherein;
(I) each additional dosing cycle of the dosing regimen of the combination treatment comprises:
(a) a subcutaneous dose of mosunetuzumab administered on Day 1 of the additional dosing cycle, wherein the subcutaneous dose of mosunetuzumab is about 45 mg; and
(b) twenty-one (21) oral doses of lenalidomide administered on Days 1-21 of the additional dosing cycle, wherein each oral dose of lenalidomide is about 20 mg; and/or
(II) each additional dosing cycle of the dosing regimen of the control treatment comprises:
(a) an intravenous dose of mosunetuzumab administered on Day 1 of the additional dosing cycle, wherein the intravenous dose of mosunetuzumab is about 30 mg; and
(b) twenty-one (21) oral doses of lenalidomide administered on Days 1-21 of the additional dosing cycle, wherein each oral dose of lenalidomide is about 20 mg.

43. The method of claim 42, wherein the dosing regimen of the combination treatment further comprises one to nine subcutaneous maintenance doses of mosunetuzumab.

44. A method for treating follicular lymphoma (FL) in a subject in need thereof, comprising administering to the subject a combination treatment comprising subcutaneously administered mosunetuzumab and orally administered lenalidomide, wherein the combination treatment comprises subcutaneously administering mosunetuzumab and orally administering lenalidomide according to a dosing regimen comprising a first dosing cycle and a second dosing cycle, wherein the first dosing cycle is a 21-day dosing cycle and the second dosing cycle is a 28-day dosing cycle; ten additional 28-day dosing cycles; and one to nine subcutaneous maintenance doses of mosunetuzumab, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 45 mg, and the scC1D3 is about 45 mg;
(b) the second dosing cycle comprises:
(i) a first subcutaneous dose (scC2D1) of mosunetuzumab administered on Day 1 of the second dosing cycle, wherein the scC2D1 is about 45 mg; and
(ii) twenty-one (21) oral doses of lenalidomide administered on Days 1-21 of the second dosing cycle, wherein each oral dose of lenalidomide is about 20 mg; and
(c) the ten additional dosing cycles each comprises:
(i) a first subcutaneous dose (scC3D1-scC12D1) of mosunetuzumab administered on Day 1 of each additional dosing cycle, wherein the scC3D1-scC12D1 are each about 45 mg; and
(ii) twenty-one (21) oral doses of lenalidomide administered on Days 1-21 of each additional dosing cycle, wherein each oral dose of lenalidomide is about 20 mg.

45. The method of claim 43, wherein:
(a) the dosing regimen of the combination treatment further comprises nine subcutaneous maintenance doses of mosunetuzumab;
(b) the one to nine subcutaneous maintenance doses of mosunetuzumab are administered every eight weeks starting eight weeks after Day 1 of the tenth additional 28-day dosing cycle; and/or
(c) each maintenance dose of mosunetuzumab is about 45 mg.

46.-51. (canceled)

52. The method of claim 1, wherein the subject:
(a) has an Eastern Cooperative Oncology Group (ECOG) Performance Status of 0, 1, or 2;
(b) has not been previously treated for FL or has relapsed after or is refractory to one or more prior lines of systemic therapy; and/or
(c) has Grade 1, 2, or 3a FL.

53.-54. (canceled)

55. The method of claim 52, wherein the one or more prior lines of systemic therapy comprises an immunotherapy, a chemoimmunotherapy, or an anti-CD20 antibody.

56. (canceled)

57. The method of claim 55, wherein the anti-CD20 antibody is rituximab.

58. (canceled)

59. A method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved efficacy response of the plurality of subjects as compared to a reference efficacy response, wherein the reference efficacy response is the efficacy response of a plurality of subjects who have received a control treatment comprising intravenously administered mosunetuzumab, and wherein the efficacy response is a complete response rate (CRR), an objective response rate (ORR), a duration of response (DOR), a duration of complete response (DOCR), progression free survival (PFS), or overall survival (OS).

60. The method of claim 59, wherein:
(a) the efficacy response is a CRR, and wherein the CRR or reference CRR is the proportion of the subjects receiving the corresponding treatment whose best overall response is a complete response (CR), and wherein the improved response in CRR is an increase in CRR compared to the reference CRR of between 1% and 20%; or the efficacy response in CRR is non-inferior compared to the reference CRR;
(b) the efficacy response is an ORR, wherein the efficacy response is an ORR, and wherein the ORR or reference ORR is the proportion of the subjects receiving the corresponding treatment whose best overall response is a CR or a partial response (PR), and wherein the improved response in ORR is an increase in ORR compared to the reference ORR of between 1% and 13%; or the efficacy response in ORR is non-inferior compared to the reference ORR:
(c) the efficacy response is a DOR, wherein the DOR or the reference DOR is measured starting from the time from the first occurrence of a documented CR or PR to disease progression or relapse or death from any cause, whichever occurs first, and wherein:
(i) the DOR or the reference DOR is the median DOR of the plurality of subjects receiving the corresponding treatment, and wherein the improvement of the median DOR is an increase in the DOR compared to the reference DOR of between 1 and 13 months; or the efficacy response in DOR is non-inferior compared to reference DOR; or
(ii) the improvement of the DOR is an increase in the rate of DOR at 12 months compared to the rate of reference DOR at 12 months of between 1% and 31%; or of about 8%;
(d) the efficacy response is a DOCR, and wherein the DOCR or the reference DOCR is measured starting from the time from the first occurrence of a documented CR to disease progression or relapse or death from any cause, whichever occurs first, and wherein:
(i) the DOCR or the reference DOCR is the median DOCR of the plurality of subjects receiving the corresponding treatment; or
(ii) the improvement of the DOCR is an increase in the rate of DOCR at 12 months compared to the rate of reference DOCR at 12 months of between 1% and 27%; or the efficacy response in DOCR is non-inferior compared to the reference DOCR;
(e) the efficacy response is a PFS, and wherein the PFS or the reference PFS is measured starting from the time from first treatment to the time of a first occurrence of disease progression or death from any cause, and wherein:
(i) the PFS or the reference PFS is the median PFS of the plurality of subjects receiving the corresponding treatment, and wherein the improvement of the median PFS is an increase in the PFS compared to the reference PFS of between 1 and 14 months; or the efficacy response in PFS is non-inferior compared to the reference PFS: or
(ii) the improvement of the PFS is in an increase in the rate of PFS compared to the rate of reference PFS at 12 months of between 1% and 26%; or of about 5%; or
(f) the efficacy response is an OS, wherein the OS or the reference OS is measured starting from the time from first treatment to death from any cause, and wherein the improvement of the OS is an increase in the rate of OS compared to the rate of reference OS at 12 months of between 1% and 9%; or the efficacy response in OS is non-inferior compared to the reference OS.

61.-75. (canceled)

76. The method of claim 60, wherein the CR or PR determined by PET/computed tomography (CT), and/or based on the Revised Response Criteria for Malignant Lymphoma (Cheson et al., 2007).

77.-86. (canceled)

87. A method for treating Non-Hodgkin Lymphoma (NHL) or chronic lymphocytic leukemia (CLL) in a subject in need thereof, comprising administering to the subject a treatment comprising subcutaneously administered mosunetuzumab, wherein administering the treatment to a plurality of subjects results in a non-inferior or improved safety response of the plurality of subjects as compared to a reference safety response, wherein the reference safety response is the safety response of a plurality of subjects who have received a control treatment comprising intravenously administered mosunetuzumab, and wherein the safety response is the rate of adverse event (AE), the rate of cytokine release syndrome (CRS), or the rate of serious AE (SAE).

88. The method of claim 87, wherein:
(a) the safety response is the rate of AE, wherein the rate of AE or the reference rate of AE is the rate of AE in the plurality of subjects receiving the corresponding treatment, and wherein:
(i) the improved rate of AE is a decrease in the rate of Grade 3 or 4 AE compared to the reference rate of Grade 3 or 4 AE of between 11% and 21%; or the safety response in rate of AE is non-inferior compared to the reference rate of AE;
(ii) the improved rate of AE is a decrease in the rate of Grade 3 or 4 AE compared to the reference rate of Grade 3 or 4 AE of between 11% and 21%; or of about 16%; or
(iii) the rate of AE is the rate of Grade 5 AE in the plurality of subjects receiving the corresponding treatment, and wherein the rate of AE is non-inferior compared to the reference rate of AE:
(b) the safety response is the rate of SAE, wherein the rate of SAE or the reference rate of SAE is the rate of SAE in the plurality of subjects receiving the corresponding treatment, and wherein the improved safety response in rate of SAE is a decrease in the rate of SAE compared to the reference rate of SAE of between 9% and 19%; or of about 14%; or
(c) the safety response is the rate of CRS, wherein the rate of CRS or the reference rate of CRS is the rate of CRS in the plurality of subjects receiving the corresponding treatment, and wherein:
(i) the improved rate of CRS is a decrease in the rate of CRS compared to the reference rate of CRS of between: 9% and 19%; or
(ii) the improved rate of CRS is a decrease in the rate of Grade 2+ CRS compared to the reference rate of Grade 2+ CRS of between 3% and 13%; or of about 8%.

89.-92. (canceled)

93. The method of claim 88, wherein;
(a) the Grade of AE is determined based on the National Cancer Institute Common Terminology Criteria for Adverse Events (NCI CTCAE), v4.0; or
(b) the Grade of CRS is determined based on the Modified Cytokine Release Syndrome Grading System (Lee et al., 2014).

94.-102. (canceled)

103. A method for treating Non-Hodgkin Lymphoma (NHL) or chronic lymphocytic leukemia (CLL) in a subject in need thereof, comprising administering to the subject a treatment comprising subcutaneously administered mosunetuzumab according to a dosing regimen comprising four 21-day dosing cycles, wherein administering the combination treatment to a plurality of subjects results in a non-inferior or improved pharmacokinetic (PK) endpoint of the plurality of subjects as compared to a reference PK endpoint, wherein the PK endpoint is an observed dosing cycle 3 serum trough concentration (scConc_{troughCYC3-OBS}) or a cumulative area under the concentration-time curve over 0-84 days (scAUC₀₋₈₄); and
wherein the reference PK endpoint is the PK endpoint of a plurality of subjects who have received a control treatment comprising intravenously administered mosunetuzumab according to a dosing regimen comprising four 21-day dosing cycles, wherein the reference PK endpoint is an observed dosing cycle 3 serum trough concentration (ivConc_{troughCYC3-OBS}) or a cumulative area under the concentration-time curve over 0-84 days (ivAUC₀₋₈₄).

104. The method of claim 103, wherein one or more of the scAUC₀₋₈₄ or the ivAUC₀₋₈₄ is determined using population PK (popPK) model-predicted individual Empirical Bayesian Estimates (EBEs).

105. The method of claim 59, wherein:
(I) the treatment comprises subcutaneously administering mosunetuzumab according to a dosing regimen comprising at least a first 21-day dosing cycle and a second 21-day dosing cycle, wherein:
(a) the first dosing cycle comprises a first subcutaneous dose (scC1D1), a second subcutaneous dose (scC1D2), and a third subcutaneous dose (scC1D3) of mosunetuzumab administered on Days 1, 8, and 15, respectively, of the first dosing cycle, wherein the scC1D1 is about 5 mg, the scC1D2 is about 15 mg or about 45 mg, and the scC1D3 is about 45 mg; and
(b) the second dosing cycle comprises a first subcutaneous dose (scC2D1) of mosunetuzumab administered on Day 1 of the second dosing cycle, wherein the scC2D1 is about 45 mg; and/or
(II) the control treatment comprises intravenously administering mosunetuzumab according to a dosing regimen comprising at least a first 21-day dosing cycle and a second 21-day dosing cycle, wherein:
(a) the first dosing cycle comprises a first intravenous dose (ivC1D1), a second intravenous dose (ivC1D2), and a third intravenous dose (ivC1D3) of mosunetuzumab administered on Days 1, 8, and 15, respectively, of the first dosing cycle, wherein the ivC1D1 is about 1 mg, the ivC1D2 is about 2 mg, and the ivC1D3 is about 60 mg; and
(b) the second dosing cycle comprises a first intravenous dose (ivC2D1) of mosunetuzumab administered on Day 1 of the second dosing cycle, wherein the ivC2D1 is about 60 mg.

106. The method of claim 105, wherein;
(a) the dosing regimen of the treatment further comprises one or more additional 21-day dosing cycles; or six, fifteen, or twenty-three additional 21-day dosing cycles; and/or
(b) the dosing regimen of the control treatment further comprises one or more additional 21-day dosing cycles; or six, fifteen, or twenty-three additional 21-day dosing cycles.

107. (canceled)

108. The method of claim 106, wherein;
(a) each additional dosing cycle of the treatment comprises a subcutaneous dose of mosunetuzumab administered on Day 1 of the additional dosing cycle, wherein the subcutaneous dose of mosunetuzumab is about 45 mg; and/or
(b) each additional dosing cycle of the control treatment comprises an intravenous dose of mosunetuzumab administered on Day 1 of the additional dosing cycle, wherein the intravenous dose of mosunetuzumab is about 30 mg.

109.-112. (canceled)

113. The method of claim 59, wherein the subject:
(a) has an Eastern Cooperative Oncology Group (ECOG) Performance Status of 0 or 1;
(b) has relapsed after or is refractory to one, two, or more prior lines of systemic therapy; and/or
(c) has a follicular lymphoma (FL), a marginal zone lymphoma (MZL), a diffuse large B cell lymphoma (DLBCL), a transformed FL (trFL), a high grade B cell lymphoma (HGBL), a primary mediastinal B cell lymphoma (PMLBCL), a transformed indolent NHL, a mantle cell lymphoma (MCL), a Richter's transformation, or a small lymphocytic lymphoma (SLL).

114.-115. (canceled)

116. The method of claim 113, wherein:
(a) the one or more prior lines of systemic therapy comprises an anti-CD20 antibody, an alkylating agent, a Bruton's tyrosine kinase (BTK) inhibitor, an anthracycline, or a combination thereof; and/or
(b) the subject:
(i) has a Grade 1-3b FL or a transformed FL;
(ii) has a DLBCL or a trFL;
(iii) has a Richter's transformation; or
(iv) has an MCL.

117.-118. (canceled)

119. The method of claim 116, wherein the subject:
(a) has a Grade 1-3a FL, and wherein the subject has relapsed after or is refractory to two or more prior lines of systemic therapy comprising an anti-CD20 antibody and an alkylating agent;
(b) has a DLBCL or a trFL, and wherein the subject has relapsed after or is refractory to two or more prior lines of systemic therapy comprising an anthracycline and an anti-CD20 antibody;
(c) has a Richter's transformation, and wherein the subject has relapsed after or is refractory to one or more prior lines of therapy comprising an anthracycline and an anti-CD20 antibody; or
(d) has an MCL, and wherein the subject has relapsed after or is refractory to one or more prior lines of systemic therapy comprising a BTK inhibitor.

120.-123. (canceled)

124. The method of claim 116, wherein the anti-CD20 antibody is rituximab.

125.-126. (canceled)

127. The method of claim 1, wherein the combination treatment and/or control treatment comprises administering an additional therapeutic agent.

128. The method of claim 127, wherein the additional therapeutic agent comprises a corticosteroid, an antihistamine, an antipyretic, or an IL-6R antagonist.

129. The method of claim 128, wherein:
(a) the additional therapeutic agent is a corticosteroid, and wherein the corticosteroid comprises prednisone, methylprednisolone, or dexamethasone;
(b) the additional therapeutic agent is an antihistamine, and wherein the antihistamine comprises diphenhydramine hydrochloride or equivalent:
(c) the additional therapeutic agent is an antipyretic, and wherein the antipyretic is acetaminophen; or
(d) the additional therapeutic agent is an IL-6R antagonist, and wherein the IL-6R antagonist is tocilizumab.

130.-188. (canceled)

189. The method of claim 44, wherein administering the combination treatment to a plurality of subjects having FL results in:
(a) an ORR in the plurality of subjects of between about 81% to about 99%; or of about 90%;
(b) a CRR in the plurality of subjects of between about 79% to about 97%; or of about 88%;
(c) a 12-month event-free rate for DOR in the plurality of subjects of between about 86% to about 100%; or of about 94%;
(d) a 12-month event-free rate for DOCR in the plurality of subjects of between about 86% to about 100%; or of about 94%
(e) a 12-month event-free rate for PFS in the plurality of subjects of between about 76% to about 98%; or of about 87%; and/or
(f) a 12-month event-free rate for OS in the plurality of subjects of between about 92% to about 100%; or of about 97%.

190.-200. (canceled)

201. The method of claim 189, wherein the FL is previously untreated (1 L) FL.

202. The method of claim 105, wherein administering the treatment to a plurality of subjects having R/R FL results in:
(a) an ORR of between about 66% to about 85%; or of about 77%;
(b) a CRR in the plurality of subjects of between about 51% to about 72%; or of about 62%;
(c) an 18-month event-free rate for DOCR in the plurality of subjects of between about 57% to about 83%; or of about 70%;
(d) a median DOCR in the plurality of subjects of at least about 20 months; or of about 35 months;
(e) an 18-month event-free rate for PFS in the plurality of subjects of between about 46% to about 68%; or of about 57%; or
(f) a median PFS in the plurality of subjects of at least 14 months; or of about 24 months.

203.-213. (canceled)

214. The method of claim 105, wherein administering the treatment to a plurality of subjects having 1 L FL results in:
(a) an ORR of between about 76% to about 96%; or of about 87%;
(b) a CRR in the plurality of subjects of between about 55% to about 67%; or of about 61%;
(c) a 12-month event-free rate for PFS in the plurality of subjects of between about 73% to about 90%: or of about 83%; or
(d) a median time to response (TTR) in the plurality of subjects exhibiting a response of between about 1 month to about 6 months; or of about 3 months.

215.-221. (canceled)

222. The method of claim 214, wherein the subjects of the plurality have a high tumor burden, per the Groupe d'Etude des Lymphomes Folliculaires (GELF) criteria.

223. The method of claim 105, wherein administering the treatment to a plurality of subjects having 1 L MZL results in:
(a) an ORR of between about 58% to about 88%; or of about 75%;
(b) a CRR in the plurality of subjects of between about 44% to about 77%; or of about 61%; or
(c) a median TTR in the plurality of subjects exhibiting a response of between about 2 months to about 6 months; or of about 3 months.

224.-228. (canceled)


SEQUENCE LISTING (36 sequences)

SEQ ID NO: 1 - protein, 10 aa, synthetic construct
GYTFTSYNMH

SEQ ID NO: 2 - protein, 17 aa, synthetic construct
AIYPGNGDTSYNQKFKG

SEQ ID NO: 3 - protein, 13 aa, synthetic construct
VVYYSNSYWYFDV

SEQ ID NO: 4 - protein, 10 aa, synthetic construct
RASSSVSYMH

SEQ ID NO: 5 - protein, 7 aa, synthetic construct
APSNLAS

SEQ ID NO: 6 - protein, 9 aa, synthetic construct
QQWSFNPPT

SEQ ID NO: 7 - protein, 122 aa, synthetic construct [sequence omitted]

SEQ ID NO: 8 - protein, 106 aa, synthetic construct [sequence omitted]

SEQ ID NO: 9 - protein, 5 aa, synthetic construct
NYYIH

SEQ ID NO: 10 - protein, 17 aa, synthetic construct
WIYPGDGNTKYNEKFKG

SEQ ID NO: 11 - protein, 10 aa, synthetic construct
DSYSNYYFDY

SEQ ID NO: 12 - protein, 17 aa, synthetic construct
KSSQSLLNSRTRKNYLA

SEQ ID NO: 13 - protein, 7 aa, synthetic construct
WASTRES

SEQ ID NO: 14 - protein, 8 aa, synthetic construct
TQSFILRT

SEQ ID NO: 15 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 17 - protein, 25 aa, synthetic construct [sequence omitted]

SEQ ID NO: 18 - protein, 14 aa, synthetic construct
WVRQAPGKGLEWVG

SEQ ID NO: 19 - protein, 32 aa, synthetic construct
RFTISVDKSKNTLYLQMNSLRAEDTAVYYCAR

SEQ ID NO: 20 - protein, 11 aa, synthetic construct
WGQGTLVTVSS

SEQ ID NO: 21 - protein, 23 aa, synthetic construct
DIQMTQSPSSLSASVGDRVTITC

SEQ ID NO: 22 - protein, 15 aa, synthetic construct
WYQQKPGKAPKPLIY

SEQ ID NO: 23 - protein, 32 aa, synthetic construct
GVPSRFSGSGSGTDFTLTISSLQPEDFATYYC

SEQ ID NO: 24 - protein, 10 aa, synthetic construct
FGQGTKVEIK

SEQ ID NO: 25 - protein, 30 aa, synthetic construct
EVQLVQSGAEVKKPGASVKVSCKASGYTFT

SEQ ID NO: 26 - protein, 14 aa, synthetic construct
WVRQAPGQGLEWIG

SEQ ID NO: 27 - protein, 32 aa, synthetic construct
RATLTADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQQKPGQPPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC